(19) United States
(12) Patent Application Publication
(10) Pub. No.: US 2025/0152629 A1
(43) Pub. Date: May 15, 2025

(54) COMPOSITIONS INCLUDING KILLER INNATE-LIKE T CELLS AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ming Li, New York, NY (US); Chun Chou, New York, NY (US); Xian Zhang, New York, NY (US); Saida Dadi, New York, NY (US); Briana G. Nixon, New York, NY (US); Jing Zhang, New York, NY (US)

(21) Appl. No.: 18/838,592

(22) PCT Filed: Feb. 14, 2023
(86) PCT No.: PCT/US2023/062546

§ 371 Date: Aug. 14, 2024

Related U.S. Application Data
(60) Provisional application No. 63/310,213, filed on Feb. 15, 2022.

(51) Int. Cl. A61K 35/17; A61K 38/20; A61K 40/15; A61K 40/31; A61K 40/42; A61K 45/06; A61P 35/00; C07K 16/28; C12N 5/0783; C12N 15/86; C12Q 1/6886

(52) U.S. Cl. CPC A61K 35/17; A61K 38/2086; A61K 40/15; A61K 40/31; A61K 40/4211; A61K 45/06; A61P 35/00; C07K 16/2803; C12N 5/0646; C12N 15/86; C12Q 1/6886; A61K 2239/13; A61K 2239/21; A61K 2239/22; C12N 2740/10043; C12Q 2600/106

(57) ABSTRACT

Described herein are compositions including killer innate-like T cells (ILTCks), methods for preparing IL TCks for adoptive cell therapy, an methods of using IL TCks to treat cancer. Further, wherein an engineered killer innate-like T cell (ILTCk) comprises a non-endogenous expression vector including a mammalian IL-15 nucleic acid sequence or a mammalian STAT5B nucleic acid sequence is disclosed.

### CROSS-REFERENCE TO RELATED APPLICATIONS

This application is the U.S. National Stage Application of International Application No. PCT/US2023/062546, filed Feb. 14, 2023, which claims the benefit of and priority to U.S. Provisional Patent Application No. 63/310,213, filed Feb. 15, 2022, the contents of each of which is incorporated herein by reference in its entirety.

### STATEMENT OF GOVERNMENT SUPPORT

This invention was made with government support under CA243904 awarded by the National Institutes of Health. The government has certain rights in the invention.

### SEQUENCE LISTING

The instant application contains a Sequence Listing which has been submitted electronically in .xml format and is hereby incorporated by reference in its entirety. Said, .xml copy, created on Aug. 13, 2024, is named 115872-3063 PCT.xml and is 389,120 bytes in size.

### TECHNICAL FIELD

The present disclosure provides compositions including killer innate-like T cells (ILTCks), methods for preparing ILTCks for adoptive cell therapy, and methods of using ILTCks to treat cancer.

### BACKGROUND

The following description of the background of the present technology is provided simply as an aid in understanding the present technology and is not admitted to describe or constitute prior art to the present technology.

The concept of cancer immunosurveillance ascribes a role of cellular immunity in eliminating transformed cells with cytotoxic CD8⁺ T cells being a primary mediator^{1,2,3}. Cancer cells can express neoantigens to prime conventional CD8⁺ T cells⁴ which nonetheless transition to a dysfunctional state of exhaustion characterized by high expression of the immune checkpoint co-inhibitory receptor PD-1 (Ref⁵). Despite the clinical success of anti-PD-1 to revive cancer immunity, many patients do not respond to checkpoint blockade therapies^{6,7,8}.

### SUMMARY OF THE PRESENT TECHNOLOGY

In one aspect, the present disclosure provides an engineered killer innate-like T cells (ILTCks) comprising a non-endogenous expression vector including a mammalian IL-15 nucleic acid sequence or a mammalian STAT5B nucleic acid sequence, wherein the IL-15 nucleic acid sequence or the STAT5B nucleic acid sequence is operably linked to an expression control sequence. The expression control sequence may comprise an inducible promoter, a constitutive promoter, a native IL-15 or STAT5B promoter, or a heterologous promoter. In certain embodiments, the IL-15 nucleic acid sequence encodes the amino acid sequence of SEQ ID NO: 19 or SEQ ID NO: 20. In other embodiments, the STAT5B nucleic acid sequence encodes the amino acid sequence of SEQ ID NO: 9 or SEQ ID NO: 23. Additionally or alternatively, in some embodiments, the non-endogenous expression vector is a plasmid, a cosmid, a bacmid, a bacterial artificial chromosome (BAC), a yeast artificial chromosome (YAC), a viral vector, or a retroviral vector. The engineered ILTCks may be derived from an autologous donor or an allogenic donor.

Additionally or alternatively, in some embodiments, the engineered ILTCk further comprises a chimeric antigen receptor (CAR) that binds to a tumor antigen and/or a nucleic acid encoding the CAR. In some embodiments, the CAR comprises (i) an extracellular antigen binding domain; (ii) a transmembrane domain; and (iii) an intracellular domain comprising one or more co-stimulatory domains, wherein the extracellular antigen binding domain binds to the tumor antigen. In any of the preceding embodiments, the heterologous promoter is induced by binding of the CAR to the tumor antigen, optionally wherein binding of the CAR to the tumor antigen results in antigen-dependent JAK-STAT5 pathway activation.

In another aspect, the present disclosure provides an engineered killer innate-like T cells (ILTCks) comprising a chimeric antigen receptor (CAR) that binds to a tumor antigen, wherein the CAR comprises (i) an extracellular antigen binding domain that binds to the tumor antigen; (ii) a transmembrane domain; and (iii) an intracellular domain comprising a truncated cytoplasmic domain of IL-2RPΔ and one or more co-stimulatory domains. In some embodiments, the truncated cytoplasmic domain of IL-2RPΔ comprises the amino acid sequence of SEQ ID NO: 7.

In any and all embodiments of the engineered ILTCks disclosed herein, the extracellular antigen binding fragment of the CAR comprises a single-chain variable fragment (scFv), preferably a human scFv. Examples of tumor antigens include, but are not limited to, 5T4, alpha 501-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pml/RARa, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAPI, B7H3, Polysialic Acid, OX40, OX40-ligand, and peptide MHC complexes (with peptides derived from TP53, KRAS, MYC, EBNA1-6, PRAME, tyronsinase, MAGEAI-A6, pmel17, LMP2, or WT1).

Additionally or alternatively, in some embodiments of the engineered ILTCks disclosed herein, the CAR transmembrane domain comprises a CD8 transmembrane domain, a CD28 transmembrane domain, a NKG2D transmembrane domain, a CD3ζ transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, or a BTLA transmembrane domain.

Additionally or alternatively, in certain embodiments of the engineered ILTCks disclosed herein, the one or more CAR co-stimulatory domains may be selected from among a CD28 co-stimulatory domain, a 4-1BB co-stimulatory domain, an OX40 co-stimulatory domain, an ICOS co-stimulatory domain, a DAP-10 co-stimulatory domain, a PD-1 co-stimulatory domain, a CTLA-4 co-stimulatory domain, a LAG-3 co-stimulatory domain, a 2B4 co-stimulatory domain, a BTLA co-stimulatory domain, a NKG2C co-stimulatory domain, a NKG2D co-stimulatory domain, or any combination thereof. In some embodiments, the one or more co-stimulatory domains comprise a DAP-10 co-stimulatory domain and a 2B4 co-stimulatory domain.

In one aspect, the present disclosure provides a composition comprising an effective amount of any and all embodiments of the engineered ILTCks described herein and a pharmaceutically acceptable carrier.

In another aspect, the present disclosure provides a method of preparing immune cells for adoptive cell therapy (ACT) comprising: (a) isolating killer innate-like T cells (ILTCks) from a donor subject, (b) transducing the ILTCks with a nucleic acid encoding IL-15 or STAT5B or an expression vector comprising said nucleic acid, and (c) administering the transduced ILTCks to a recipient subject. In certain embodiments, the nucleic acid encodes the amino acid sequence of SEQ ID NO: 19, SEQ ID NO: 20, SEQ ID NO: 9 or SEQ ID NO: 23. Additionally or alternatively, in some embodiments, the method further comprises transducing the ILTCks with a nucleic acid encoding a chimeric antigen receptor (CAR) that binds to a tumor antigen. Also disclosed herein is a method of preparing immune cells for adoptive cell therapy (ACT) comprising: (a) isolating killer innate-like T cells (ILTCks) from a donor subject, (b) transducing the ILTCks with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen; (ii) a transmembrane domain; and (iii) an intracellular domain comprising a truncated cytoplasmic domain of IL-2RPΔ and one or more co-stimulatory domains, and (c) administering the transduced ILTCks to a recipient subject. In some embodiments of the ACT methods described herein, the donor subject and the recipient subject are the same or different. Additionally or alternatively, in some embodiments of the ACT methods described herein, isolating ILTCks from the donor subject comprises isolating a population of immune cells from the donor subject, and collecting FCER1G⁺ cells from the isolated population of immune cells.

In yet another aspect, the present disclosure provides a method for treating cancer or inhibiting tumor growth in a subject in need thereof comprising administering to the subject an effective amount of any and all embodiments of the engineered ILTCks described herein or the pharmaceutical compositions described herein. The cancer or tumor may be selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Additionally or alternatively, in some embodiments of the methods disclosed herein, the engineered ILTCk is administered pleurally, intravenously, subcutaneously, intranodally, intratumorally, intrathecally, intrapleurally or intraperitoneally. In certain embodiments, the methods of the present technology further comprise sequentially, separately, or simultaneously administering to the subject an additional cancer therapy. Examples of additional cancer therapy include, but are not limited to, chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

Also disclosed herein are kits comprising an expression vector that includes a nucleic acid sequence encoding a mammalian IL-15 or STAT5B amino acid sequence, such as SEQ ID NO: 19, SEQ ID NO: 20, SEQ ID NO: 9 or SEQ ID NO: 23, and instructions for transducing ILTCks with the expression vector. Additionally or alternatively, the kits of the present technology may comprising a vector encoding any and all embodiments of CAR constructs described herein or another cell-surface ligand that binds to a tumor antigen.

In yet another aspect, the present disclosure provides a method for selecting a subject suffering from cancer for adoptive cell therapy with killer innate-like T cells (ILTCks) comprising (a) identifying a cancer subject harboring tumors with low mutation burden; and (b) administering to the cancer subject an effective amount of killer innate-like T cells (ILTCks). In some embodiments, the tumors are refractory to immune checkpoint blockade therapy or adoptive cell therapy with CD8⁺ T cell therapy. Additionally or alternatively, in some embodiments, the tumors with low mutation burden are identified via next-generation sequencing using a tumor biopsy sample or cell-free DNA (cfDNA) sample obtained from the cancer subject.

In one aspect, the present disclosure provides a method for treating cancer or inhibiting tumor growth in a subject in need thereof comprising administering to the subject an effective amount of killer innate-like T cells (ILTCks). The ILTCks may be native ILTCks, genetically engineered ILTCks, or a combination thereof. In some embodiments, the ILTCks are isolated from a donor subject and/or expanded ex vivo or in vitro.

In any and all embodiments of the methods disclosed herein, the methods further comprise separately, simultaneously, or sequentially administering an effective amount of IL-15 to the subject. The IL-15 may be administered to the subject prior to, during, or subsequent to administration of the ILTCks.

In any and all embodiments of the methods disclosed herein, the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Additionally or alternatively, in some embodiments of the methods disclosed herein, the ILTCks are administered pleurally, intravenously, subcutaneously, intranodally, intratumorally, intrathecally, intrapleurally or intraperitoneally. In certain embodiments, the methods of the present technology further comprise sequentially, separately, or simultaneously administering to the subject an additional cancer therapy. Examples of additional cancer therapy include, but are not limited to chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

Also disclosed herein are methods for preparing killer innate-like T cells (ILTCks) for adoptive cell therapy comprising isolating a population of immune cells from a donor subject, and collecting FCER1G⁺ cells from the isolated population of immune cells. The FCER1G⁺ cells may comprise FCER1G⁺CD122⁺ cells, FCER1G⁺ NK1.1⁺GzmB^{+/-} cells, FCER1G⁺NK1.1⁻GzmB⁻ cells, and/or FCER1G⁺PD-1⁺ cells.

### BRIEF DESCRIPTION OF THE DRAWINGS

FIGS. 1A-1E: Characterization of tumor-infiltrating CD8⁺ T cells. FIG. 1A: Uniform manifold approximation and projection (UMAP) of CD45⁺TCRβ⁺CD8α⁺ lymphocytes in breast tumor tissues from PyMT mice. Clusters (C) are denoted by color. FIG. 1B: Violin plots comparing expression of signature genes of indicated clusters. FIG. 1C: Three-dimensional diffusion embedding generated using clusters C1-C5 cells. FIG. 1D: Visualization of diffusion component (DC) analysis comparing the gene expression programs of cells from C2-C5 to that of C1 cells. FIG. 1E: Diffusion distance from C2-C5 cells to C1 cells. Statistical differences between the distribution of diffusion distances for each pair of clusters were calculated using a two-sided Wilcoxon test with P<2.2e-16 between C2 and C4, C2 and C3, and C2 and C5.

FIGS. 2A-2I: αβILTCk differentiation diverges from conventional CD8⁺ T cells during thymic development in a TCR-specificity dependent manner. FIG. 2A: CDR3 analysis of TCR pairs isolated from the indicated cell populations shown as pie charts with the number of unique CDR3 analyzed for indicated cell types denoted in the middle of each pie chart. Data are pooled from three mice. FIG. 2B: A schematic diagram of the TCR-reporter assay. FIG. 2C: Frequency of GFP⁺ among CD8⁺ reporter cells expressing indicated TCRs after overnight co-culturing with primary PyMT cancer cells. Data are pooled from three independent experiments. FIG. 2D: A schematic diagram showing the TCR ‘swapping’ experiment. Endogenously rearranged TCRs of naïve CD8⁺ T cells were substituted with indicated NK1.1⁺ αβILTCk-derived TCRs. Engineered cells were adoptive transferred into tumor-bearing mice and analyzed seven days later. FIGS. 2E-2F: Expression of PD-1 and NK1.1 by donor conventional CD8⁺ T cells engineered to express indicated NK1.1⁺ αβILTCk-derived TCR one week post transfer. Data are pooled from two independent experiments with one to two recipient mice in each experiment. FIG. 2G: Generation of TCR ‘retrogenic’ bone marrow chimeras by reconstitution of lethally irradiated PyMT mice with a mixture of Rag1^{-/-} Lineage⁻c-Kit⁺Sca1⁻ (LSK) cells which were transduced with retroviruses expressing the indicated NK1.1⁺ αβILTCk-derived TCRs and RagI^{+/+} total bone marrow cells. FIGS. 2H-2I: Expression of PD-1 and NK1.1 by donor-derived tumor-infiltrating CD8⁺ T cells expressing indicated monoclonal or polyclonal TCRs in the TCR ‘retrogenic’ bone marrow chimeras. Data are pooled with three ‘retrogenic’ chimeras generated for each TCR. All statistical data are shown as mean±S.D. ***P<0.001.

FIGS. 3A-3F: Thymocytes bearing αβILTCk-TCRs undergo ‘agonist’ selection, and continually repopulate tumor. FIG. 3A: Expression of CD4 and CD8α by donor-derived TCRβ⁺ thymocytes bearing the indicated monoclonal or polyclonal TCRs in the TCR ‘retrogenic’ bone marrow chimeras. FIG. 3B: Frequency of CD4⁺CD8⁺, CD4^{-/dull}CD8^{-/dull}, CD4⁺, and CD8⁺ T cells among donor-derived TCRβ⁺ thymocytes expressing the indicated TCRs. FIG. 3C: Surface levels of PD-1 and CD122 on donor-derived TCRβ⁺ thymocytes expressing a monoclonal TCR or on donor-derived TCRβ⁺CD4⁺CD8α CD1d⁻ thymocytes with a polyclonal TCR repertoire. FIG. 3D: Frequency of PD-1⁺CD122⁺ cells among donor-derived TCRβ⁺ thymocytes with indicated TCRs in the TCR ‘retrogenic’ bone marrow chimeras. Pooled data are shown with three ‘retrogenic’ chimeras generated for each TCR. FIG. 3E: Frequency of donor αβILTCk progenitor-derived cells, identified as CD45.1⁺TCRβ⁺ present in the tumor and small intestinal (S.I.) epithelium from CD45.2⁺ Rag1^{-/-} or Rag1^{+/-} PyMT recipient mice four to five weeks post adoptive transfer. The bottom panel shows the expression pattern of PD-1 and NK1.1 on CD45.1⁺TCRβ⁺ donor cells. FIG. 3F: Statistical analysis of the contribution of donor αβILTCk progenitors to the tumor αβILTCk and SI IEL compartments in indicated recipient mice. Data are pooled from two independent experiments with one to two recipients in each experiment. All statistical data are shown as mean±S.D. ***P<0.001 and n.s.: not significant.

FIGS. 4A-4E: FCER1G expression marks cells of the αβILTCk-lineage. FIG. 4A: Flow cytometric analysis of FCER1G, CD122, CD4, CD8α, CD80, PD-1, NK1.1, and granzyme B expression in TCRβ⁺CD1d⁻NK1.1⁻ thymocytes and TCRβ⁺CD4⁻CD1d⁻ tumor-infiltrating T cells from PyMT mice. FIG. 4B: Frequency of thymic FCER1G⁺CD122⁺ αβILTCk progenitor (αβILTCkP), intratumoral αβILTCk, thymic CD8 single positive (CD8SP), and intratumoral CD8αβ⁺ T cells expressing indicated combination of markers. Data are pooled from two independent experiments with one to two mice in each experiment. FIG. 4C: Expression of FCER1G and PD-1 by CD45⁺TCRβ⁺CD4⁻ cells in tumor tissues or adjacent normal colon from patients with colorectal carcinoma. FIG. 4D: Frequency and number of FCER1G- or PD-1-expressing TCRβ⁺CD4 cells in tumor tissues or adjacent normal colon. FIG. 4E: Granzyme B expression in FCER1G⁺TCRβ⁺ and PD-1⁺TCRβ⁺CD4⁻ cells in tumor tissues from patients with colorectal carcinoma. Data are representative of and pooled from eight patient samples. All statistical data are shown as mean±S.D. *P<0.05; **P<0.01; ***P<0.001 and ****P<0.0001.

FIGS. 5A-5K: αβILTCks sense cancer cell-expressed IL-15, and inducible hyperactivation of IL-15 signaling in αβILTCks suppresses tumor growth. FIG. 5A: Flow cytometric analysis of FCER1G and CD122 expression in TCRβ⁺CD1d⁻NK1.1⁺CD4⁻ CD8α⁻ thymocytes from Il15^{-/-} and Il15^{+/+} mice. FIG. 5B: Frequency of FCER1G⁺CD122⁺ cells among TCRβ⁺CD1d⁻NK1.1⁻CD4⁻CD8α⁻ thymocytes from Il15^{-/-} and Il15^{+/+} mice. Data are pooled from two independent experiments. FIG. 5C: GFP expression by CD45-EpCAM⁺ cells in transformed and normal mammary tissues from IL-15-GFP reporter control and PyMT mice. FIG. 5D: Correlation between frequency of FCER1G⁺ cells among CD45⁺TCRβ⁺CD4⁻ cells and IL-15 expression level in tumor tissues from patients with colorectal carcinoma. Each dot denotes an independent patient sample. FIG. 5E: Flow cytometric analysis of FCER1G and CD122 expression in TCRβ⁺CD1d⁻NK1.1⁻CD4⁻CD8α⁻ thymocytes and CD45⁺TCRβ⁺CD4⁻ tumor-infiltrating cells from S100a8-CreR15^{fl/fl}PyMT or control PyMT mice. Bottom panel shows expression of NK1.1 and granzyme B in FCER1G⁺CD122⁺ cells. FIG. 5F: Frequencies of FCER1G⁺CD122⁺ among TCRβ⁺CD1d⁻NK1.1⁻CD4⁻CD8α⁻ thymocytes and among CD45⁺TCRβ⁺CD4⁻ tumor-infiltrating cells from mice of indicated genotypes. Data are representative of and pooled from two independent experiments with two to three mice of each genotype in each experiment. FIG. 5G: Total tumor burden in mice of indicated genotypes. Data are pooled from two independent experiments. Control PyMT (n=4) and S100a8-CreIl15^{fl/fl}PyMT (n=8). FIG. 5H: A schematic diagram showing αβILTCk-based adoptive cellular transfer experiment. A constitutively active form of Stat5b (Stat5b-CA) was induced in thymic CD45.1⁺CD45.2⁺ αβILTCk progenitors (αβILTCkP) or CD8 single positive T cells (CD8SP) by tamoxifen administration one week after adoptive transfer into congenically distinct CD45.2⁺ PyMT recipient mice. FIG. 5I: Flow cytometric analysis showing the frequency of adoptively transferred CD45.1⁺CD45.2⁺ cells among TCRβ⁺CD8α⁺ tumor-infiltrating cells and NK1.1 as well as granzyme B expression in donor cells as gated in the panel on the left. FIG. 5J: Frequency of adoptively transferred donor CD45.1⁺CD45.2⁺ T cells among TCRβ⁺CD8α⁺ tumor-infiltrating cells and frequency of NK1.1⁺Granzyme B⁺ among donor cells. Data are representative of and pooled from two independent experiments with two to four hosts mice receiving indicated progenitors in each experiment. FIG. 5K: Total tumor burden in mice adoptively transferred with no cells, Ubc-CreERRosa26^{LSL-Stat5b-CA/+} thymic CD8SPs or αβILTCkPs. Data are pooled from two independent experiments. No transfer (n=5), Ubc-CreERRosa26^{LSL-Stat5b-CA/+} CD8SP (n=8), and Ubc-CreERRosa26^{LSL-Stat5b-CA/+} αβLTTCkP (n=5). All statistical data are shown as mean±S.D. *P<0.05; **P<0.01; ***P<0.001 and n.s.: not significant.

FIG. 6: scRNA-seq analysis of tumor-infiltrating CD8⁺ T cells. Heat map of the expression levels of selected genes in different clusters.

FIGS. 7A-7K: Distinct developmental trajectories underlie the evolutionarily conserved αβILTCk responses. FIGS. 7A-7B: Monocle pseudotime analysis showing hypothetical developmental trajectories of indicated tumor-infiltrating CD8⁺ T cell subsets using recently activated cells as a starting population. FIG. 7C: Uniform manifold approximation and projection (UMAP) of CD45⁺TCRβ⁺CD8α⁺ lymphocytes isolated from breast tumor tissues of PyMT mice. FIG. 7D: Violin plots showing the enrichment of αβILTCk gene signature in the indicated cell clusters from PyMT mice. FIG. 7E: Enrichment of αβILTCk signature genes in cluster C3 from PyMT mice. FIG. 7F: UMAP of CD8⁺ T cells isolated from prostate tumor tissues of TRAMP mice. FIG. 7G: Enrichment of αβILTCk gene signature in the indicated cell clusters from TRAMP mice. FIG. 7H: Enrichment of αβILTCk signature genes in cluster C3 from TRAMP mice. FIG. 7I: UMAP of CD8⁺TCRαβ⁺ T cells present in a previously published human colorectal carcinoma dataset¹². FIG. 7J: Violin plots showing enrichment of αβILTCk gene signature in various cell clusters from the human colorectal carcinoma tissues. FIG. 7K: Enrichment of αβILTCk signature genes in cluster C3 from the human colorectal carcinoma dataset.

FIGS. 8A-8D: Recognition of cancer cell antigens by NK1.1⁺ αβILTCk-TCRs. FIG. 8A: A representative flow plot showing the gating strategy used to isolate NK1.1⁺ αβILTCks and PD-1⁺ T cells (TCs) from PyMT mice. NK1.1⁺ αβILTCks are identified as CD45⁺TCRβ⁺CD8α⁺PD-1⁻NK1.1⁺ whereas PD-1⁺ TCs are defined as CD45⁺TCRβ⁺CD8α⁺PD-1⁺NK1.1⁻. FIG. 8B: A histogram showing the distribution of CDR3 lengths of TCRα and TCRβ pairs isolated from αβILTCks and PD-1⁺ TCs. Data are pooled from two independent experiments. FIG. 8C: Flow cytometric analysis showing the frequency of GFP⁺ cells among CD8⁺ and CD8⁻ reporter cell line expressing indicated TCRs 24 hours after co-culturing with primary PyMT cancer cells or splenocytes pulsed with the SIINFEKL (SEQ ID NO: 68) peptide (OVA₂₅₇₋₂₆₄). Data are representative of at least three independent experiments. FIG. 8D: Frequency of GFP⁺ cells among CD8⁻ reporter cell line expressing indicated NK1.1⁺ αβILTCk-derived TCRs 24 hours after co-culturing with primary PyMT cancer cells. Data are pooled from three independent experiments.

FIGS. 9A-9H: The majority of αβILTCks recognize tumor-associated antigens presented in the context of classical MHC-I. FIG. 9A: A representative flow plot showing the expression of classical MHC-I (H2-K^{b} and H2-D^{b}) on PyMT cancer cells, defined as CD45⁻CD31⁻EpCAM⁺ from mice of indicated genotypes. Data are representative of two independent experiments. FIG. 9B: Frequency of GFP⁺ cells among CD8⁺ reporter cell line expressing indicated TCRs 24 hours after co-culturing with primary PyMT cancer cells doubly deficient for H2-K^{b} and H2-D^{b}. Data are pooled from three independent experiments. FIG. 9C: Flow cytometric analysis showing the frequency of GFP⁺ cells among CD8⁺ and CD8⁻ reporter cell line expressing the indicated TCRs 24 hours after co-culturing with primary PyMT cancer cells lacking β2m. Data are representative of two independent experiments. FIG. 9D: Frequency of TCRβ⁺CD8α⁺ cells among CD45⁺ tumor-infiltrating cells in mice lacking classical MHC-I (H2-K1^{-/-}H2-D^{-/-}), both classical and non-classical MHC-I (B2m^{-/-}) or control wild-type mice. The profiles of PD-1 and NK1.1 expression by TCRβ⁺CD8α⁺ are shown in the bottom panel. FIG. 9E: Statistical analysis of frequency of PD-1⁺ T cells and αβILTCks among tumor-infiltrating CD45⁺ cells in mice of indicated genotypes. Data are pooled from two independent experiments with one to four mice of each genotypes in each experiment. FIG. 9F: Flow cytometric analysis showing H-2D^{b} expression on control, TAP1- or β2m-deficient PyMT cell lines generated by Cas9-mediated genome editing with single guide RNA (sgRNA). FIG. 9G: Expression of GFP in reporter cell lines expressing the indicated TCRs 24 hours after co-culture with control or TAP1-deficient PyMT cells lines in the presence of absence of SIINFEKL (SEQ ID NO: 68) peptide. FIG. 9H: Frequency of GFP⁺ cells among TCR-expressing reporter cells after co-culture with PyMT cell lines of indicated genotypes with or without SIINFEKL (SEQ ID NO: 68) peptide. Data are pooled from three independent experiments. All statistical data are shown as mean±S.D. **P<0.01; ***P<0.001 and n.s.: not significant.

FIGS. 10A-10F: Generation of CD8⁺ T cells with altered specificity via TCR ‘swapping.’ FIGS. 10A-10B: A schematic diagram showing the experimental design. Cas9-mediated deletion of endogenously rearranged TCR in CD8⁺ T cells is achieved by the introduction of three distinct TCR-loci-targeting single guide RNAs (sgRNAs) expressed from a single retrovirus (TTTTTTGGGGGTCCTAACTT (SEQ ID NO: 381), TAAATCCGGCTACTTTCAGC (SEQ ID NO: 382), and AGACCCTCAGGCGGCTGCTC (SEQ ID NO: 383)). A TCR of interest is introduced into the TCR-deleted CD8⁺ T cells by another retrovirus. FIG. 10C: A timeline outlining the steps to generate CD8⁺ T cells with altered antigen receptor specificity. FIG. 10D: Representative flow plots showing the successful deletion of both the TCRα and TCRβ chains after transduction with sgRNA-expressing retrovirus. FIG. 10E: Flow plots showing the re-expression of surface TCR after retrovirus-mediated introduction of a TCR of interest. FIG. 10F: Representative flow plots showing the colonization of tumor tissues in CD45.2⁺ recipient mice by CD45.1⁺CD45.2⁺ donor CD8⁺ T cells, which have undergone the ‘swap’ procedure to express the indicated TCRs seven days post adoptive transfer. Data are representative of two independent experiments.

FIGS. 11A-11D: αβILTCk development is cDC1-independent. FIG. 11A: Flow cytometric analysis of TCRβ, CD8α, PD-1, and NK1.1 expression in tumor-infiltrating CD45⁺ cells from Batf3^{-/-}PyMT and control Batf3^{+/+}PyMT mice. FIG. 11B: Frequency of tumor-infiltra-ting PD-1⁺ T cells and αβILTCks in mice of indicated genotypes. FIG. 11C: Flow cytometric analysis of TCRβ, CD8α, PD-1, and NK1.1 expression in tumor-infiltrating CD45⁺ cells from Itgax-CreIrf8^{fl/fl}PyMT and control Itgax-CreIrf8^{+/+} PyMT mice. FIG. 11D: Frequency of tumor-infiltrating PD-1⁺ T cells and αβILTCks in mice of indicated genotypes. Data from four independent experiments with two to three mice of each genotype per experiment are shown. All statistical data are shown as mean±S.D. ****P<0.0001 and n.s.: not significant.

FIGS. 12A-12N: Recognition of thymus-derived self-antigens by αβILTCk-TCRs specifies αβILTCk lineage commitment from DP thymocytes. FIG. 12A: Frequency of TCRβ⁺ cells among donor-derived (GFP⁺) tumor-infiltrating cells in ‘retrogenic’ TCR bone marrow chimeras. FIG. 12B: Expression of PD-1 and NK1.1 by donor-derived tumor-infiltrating T cells expressing indicated αβILTCk-derived TCRs in the TCR ‘retrogenic’ bone marrow chimeras. FIG. 12C: Frequency of PD-1⁺ or NK1.1⁺ cells among donor-derived tumor-infiltrating cells expressing the indicated TCRs. Data are pooled with three ‘retrogenic’ bone marrow chimeras generated for each TCR. FIG. 12D: Expression of CD4 and CD8α by donor-derived TCRβ⁺ thymocytes bearing the indicated TCRs in the TCR ‘regtrogenic’ bone marrow chimeras. FIG. 12E: Frequency of CD4⁺CD8⁺, CD4^{-/dull}CD8^{-/dull}, CD4⁺, and CD8⁺ T cells among donor-derived TCRβ⁺ thymocytes expressing the indicated TCRs. Data are pooled with three ‘retrogenic’ bone marrow chimeras generated for each TCR. FIG. 12F: Expression of tdTomato by splenic CD19⁺ cells and intratumoral PD-1⁺ T cells (TCs) as well as αβILTCks in Rorc-CreRosa26^{LSL-tdTomato}PyMT mice. Data are representative of two independent experiments. FIG. 12G: Frequency of tdTomato⁺ cells among each indicated lymphocyte compartment. Data are pooled from two independent experiment with one to two mice in each experiment. FIG. 12H: Expression of YFP by splenic CD19⁺ cells, intratumoral PD-1⁺ TCs as well as αβILTCks, and thymic iNKT cells in PyMT mice reconstituted with YFP⁻ bone marrow from Zbtb16-CreRosa26^{LSL-YFP} mice. FIG. 12I: Frequency of YFP⁺ cells among indicated lymphocyte compartment. Data are pooled from two independent experiments with two bone marrow chimeras analyzed in each experiment. FIG. 12J: Surface expression of PD-1 and CD122 on donor-derived TCRβ⁺ thymocytes bearing the indicated monoclonal TCR in the TCR ‘retrogenic’ bone marrow chimeras. Data are representative of three independent experiments. FIG. 12K: Frequency of PD-1⁺CD122⁺ T cells among donor-derived TCRβ⁺ thymocytes with indicated TCR. Data are pooled with three to four ‘retrogenic’ bone marrow chimeras generated for each TCR. FIG. 12L: Frequency of GFP⁺ cells among CD8⁺ reporter cell line expressing indicated TCRs 24 hours after co-culturing with a cortical thymic epithelial cell line. Data are pooled from two independent experiments. FIG. 12M: Frequency of αβILTCk progenitors (αβILTCkPs) among donor-derived thymocytes expressing the NK139 or NK186 TCRs in B2m^{+/+} or B2m^{-/-} recipient mice reconstituted with Rag1^{-/-}B2m^{+/+} bone marrow cells. Data are pooled from two independent experiments with one to two ‘retrogenic’ TCR bone marrow chimeras generated for each TCR. FIG. 12N: Frequency of αβILTCkPs among donor-derived thymocytes in B2m^{+/+} or B2m^{-/-} recipient mice reconstituted with B2m^{+/+} or B2m^{-/-} bone marrow cells. Data are pooled from two independent experiments with one to two bone marrow chimeras of each genotype analyzed per experiment. All statistical data are shown as mean±S.D. **P<0.01; ***P<0.001 and n.s.: not significant.

FIGS. 13A-13L: Continuous replenishment of the intratumoral αβILTCk compartment by circulating progenitors. FIG. 13A: Flow cytometric analysis of CD8α and CD80 co-receptor expression on donor-derived T cells bearing the indicated monoclonal TCR in the tumor and small intestinal (S.I.) epithelium from TCR ‘retrogenic’ bone marrow chimeras. Data are representative of three independent experiments. FIG. 13B: Frequency of CD8αα⁻ and CD8αβ⁺ cells among donor TCRβ⁺ cells in the small intestine epithelium. FIG. 13C: Frequency of CD8αα⁺ and CD8αβ⁺ cells among donor TCRβ⁺ cells in PyMT tumors. Data are pooled from three independent experiments. FIG. 13D: A gating strategy for isolating the putative αβILTCk-committed thymic progenitors (αβILTCkPs). αβILTCkPs are defined as CD4^{-/dull}CD8^{-/dull}TCRβ⁺CD1d⁻CD25⁻PD-1⁺CD122⁺NK1.1⁻. Data are representative of three independent experiments. FIG. 13E: A schematic diagram showing the experimental setup to assess the differentiation potential of putative αβILTCkPs. FIG. 13F: Tracking the contribution of adult bone marrow hematopoiesis to various lymphocyte lineages. Fgd5-CreERRosa26^{LSL-tdTomato}PyMT mice were administered 5 mg of Tamoxifen via oral gavage, and lymphocytes in multiple organs were analyzed 21 weeks later for tdTomato expression. FIG. 13G: Flow plots showing tdTomato expression in bone marrow lineage⁻c-Kit⁺Sca1⁺ (LSK) hematopoietic stem cells from Fgd5-CreERRosa26^{LSL-tdTomato}PyMT mice 21 weeks post Tamoxifen administration. FIG. 13H: Flow cytometric analysis showing the expression of tdTomato in indicated thymic progenitors as gated in the left panel. FIG. 13I: Frequency of Fgd5-CreER-fate-mapped cells among indicated thymic progenitor populations. FIG. 13J: Flow cytometric analysis showing the expression of tdTomato in PD-1⁺ T cells (TCs) and αβILTCk as gated in the left panel. FIG. 13K: Expression of tdTomato and CDβ0 in TCRβ⁺CD8α⁺ cells isolated from the S.I. epithelium. FIG. 13L: Frequency of tdTomato-expressing cells among the indicated lymphocyte populations isolated from the tumor and S.I. epithelium. Data are representative of and pooled from three independent experiments with one to two animals in each experiment. All statistical data are shown as mean±S.D. *P<0.05; ***P<0.001 and n.s.: not significant.

FIGS. 14A-14O: FCER1G and CD122 co-expression identifies αβILTCks in mouse and human. FIG. 14A: A scatter plot comparing the gene expression program differentiating αβILTCk-committed thymic progenitors (αβILTCkPs) from CD8 single positive T cells (SPs) (x-axis) to that distinguishing intratumoral αβILTCks from PD-1⁺ T cells (TCs) (y-axis). Each dot denotes a gene with the axes representing fold change of the gene expression level. FIG. 14B: Pathway analysis of genes upregulated in thymic αβILTCkPs but subsequently downregulated in intratumoral αβILTCks. FIG. 14C: A heatmap showing genes downregulated in thymic αβILTCkPs relative to their CD8 SP counterparts. FIG. 14D: A heatmap showing genes upregulated in thymic αβILTCkPs relative to their CD8 SP counterparts. FIG. 14E: Pathway analysis of genes upregulated in intratumoral αβILTCks compared to their thymic precursors. FIG. 14F: A heat map showing differentially expressed genes among intratumoral PD-1⁺ TCs, NK1.1⁺ and NK1.1⁻ αβILTCks isolated from ‘retrogenic’ bone marrow chimeras generated with NK139 TCR-expressing Rag1^{-/-} bone marrow cells. FIG. 14G: Uniform manifold approximation and projection (UMAP) of CD45⁺TCRβ⁺ lymphocytes in breast tumor tissues from PyMT mice showing Fcer1g-expressing cells. FIG. 14H: UMAP of CD45⁺TCRβ⁺ lymphocytes in prostate tumor tissues from TRAMP mice showing Fcer1g-expressing cells. FIG. 14I: UMAP of CD45⁺TCRβ⁺ lymphocytes present in a previously published human colorectal carcinoma dataset¹², showing FCER1G-expressing cells. FIG. 14J: Flow cytometric analysis of FCER1G and CD122 expression in CD4-CD8α TCRβ⁺CD1d and CD8α⁺TCRβ⁺CD1d thymocytes as well as TCRβ⁺CD4⁻CD1d⁻CD8α⁺NK1.1⁺ and TCRβ⁺CD4⁻CD1d⁻CD8α⁺PD-1*⁺ tumor-infiltrating cells from PyMT mice. FIG. 14K: Expression levels of FCER1G and CD122 in indicated cell populations. Data are representative of and pooled from two independent experiments with two mice in each experiment. FIG. 14L: Flow cytometric analysis of CD4, CD8α, and CD8β expression in FCER1G⁺CD122⁺ and FCER1G⁺CD122⁺ tumor-infiltrating TCRβ⁺CD1d⁻ cells from PyMT mice. FIG. 14M: Frequency of FCER1G⁺CD122⁺ and FCER1G⁻CD122⁻ tumor-infiltrating TCRβ⁺CD1d⁻ cells expressing indicator combination of markers. Data are representative of and pooled from three mice. FIG. 14N: Flow cytometric analysis of CD4 and CD8α expression by FCER1G⁺ and FCER1G⁻ CD45⁺HLA-DR⁻TCRβ⁺ cells in tumor tissues from patients with colorectal carcinoma. FIG. 14O: Frequency of FCER1G⁺CD45⁺HLA-DR⁻ TCRβ⁺ and FCER1G⁻CD45⁺HLA-DR⁻TCRβ⁺ tumor-infiltrating cells expressing indicated combination of markers. Data are representative of and pooled from three patient samples. All statistical data are shown as mean±S.D. *P<0.05; **P<0.01; ***P<0.001 and n.s.: not significant.

FIGS. 15A-15J: Adoptive transfer of Stat5b-CA-armed αβILTCks deters tumor growth. FIG. 15A: Immunofluorescence images showing expression of IL-15 and CDH1 in tumor tissues from patients with colorectal carcinoma (left panel). Flow cytometric analysis of FCER1G and PD-1 expression in TCRβ⁺CD4⁻ cells from the same tumor tissue (right panel). Data are representative of two independent experiments. FIG. 15B: Correlation between frequency of PD-1⁺ cells among CD45⁺TCRβ⁺CD4⁻cells and IL-15 expression level in tumor tissues from patients with colorectal carcinoma. Each dot denotes an independent patient sample. FIG. 15C: Statistical analysis showing relative mRNA expression of Il15 in sorted CD45⁻EpCAM⁺ cancer cells from S100a8-CreIl5^{fl/fl}PyMT and control PyMT mice. Data are pooled from two independent experiments. FIG. 15D: Flow cytometric analysis of PD-1, NK1.1, and granzyme B expression in thymic αβILTCk progenitors (αβILTCkPs) one, three, or five days after culturing in the presence of 100 ng/ml IL-15/IL-15Ra complex. Data are representative of three independent experiments. FIG. 15E: A schematic diagram showing αβILTCk-based adoptive cellular transfer experiment. A constitutively active form of Stat5b (Stat5b-CA) was induced in thymic αβILTCkPs by tamoxifen administration one week after adoptive transfer into lymphocyte-deficient PyMT recipient mice with a total tumor burden of 300-400 mm³. FIG. 15F: Expression of Ly6G and TCRβ by tumor-infiltrating CD45⁺ cells. FIG. 15G: Statistical analysis of the frequency of donor-derived TCRβ⁺ cells among tumor-infiltrating CD45⁺ cells. FIG. 15H: Flow cytometric analysis of NK1.1 and granzyme B expression by donor-derived TCRβ⁺ cells in the tumor. FIG. 15I: Frequency of NK1.1⁺Granzyme B⁺ cells among transferred TCRβ⁺ cells. Data are pooled from two independent experiments with two to three mice in each experiment. FIG. 15J: Total tumor burden in mice adoptively transferred with no cells or thymic αβILTCkPs of indicated genotypes. Data are pooled from three independent experiments. No transfer (n=4), Ubc-CreERRosa26^{+/+} (n=8), and Ubc-CreERRosa26^{Stat5b-cA/+} (n=7). All statistical data are shown as mean±S.D. *P<0.05; **P<0.01; ***P<0.001; ****P<0.0001 and n.s.: not significant.

FIGS. 16A-16C: Human type 1 innate lymphocytes armed with IL-2Rβ chain STAT5 chimeric antigen receptor (CAR) signaling show enhanced cytotoxicity to target cells. FIG. 16A: Schematic diagram of anti-CD19 CAR constructs with or without an IL-2Rβ chain STAT5 signaling domain. FIG. 16B: CD107a expression on NK-92MI cells transduced with indicated CARs in the presence or absence of target Raji cells. FIG. 16C: Specific cytotoxicity of NK-92MI cells transduced with indicated CARs to target Raji cells at 10:1 effector to target ratio. Data are shown as mean±S.D. were shown. *P<0.05. These results demonstrate that antigen-dependent JAK-STAT pathway activation strategies can be successfully extrapolated to ILTCks to achieve anti-tumor effects.

FIGS. 17A-17B: Validation of chimeric antigen receptor (CAR) constructs to temporarily drive expression of a constitutively active form of STAT5. FIG. 17A: Schematic diagram of a synthetic Notch-based CAR system for inducible expression of a constitutively active form of STAT5b (STAT5bCA). Following EpCAM recognition by an anti-EpCAM synthetic Notch CAR results in cleavage and translocation of a TetRVP64 transcription factor complex to the nucleus to drive expression of STAT5bCA in killer innate-like T cells (ILTCks). Red color denotes expression of CAR, and surface expression of a truncated form of EGF receptor (hEGFRt) denotes integration of the STAT5bCA construct. FIG. 17B: A 58α-β-cell line was transduced with retrovirus expressing the Myc-EpCAM-scFV-Notch-TetRVP64 and TetRE-Stat5bCA-mNeonGreen-PGK-hEGFRt constructs and co-cultured with 293T cells for 2 days. Engagement of EpCAM-scFV with EpCAM triggers cleavage of Notch intracellular domain, releasing TetVP64 to localize into the nucleus, transactivating transcription of Stat5bCA. mNeonGreen as a reporter of Stat5bCA expression was shown. These results demonstrate that induction of JAK-STAT activation can be successfully extrapolated to ILTCks to achieve anti-tumor effects.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

For the past two decades, the cancer immunology field has focused on defining tumor-associated neoantigens and enhancing adaptive T cell immunity for cancer cell elimination, culminating in the clinical success of checkpoint blockade therapies. However, the majority of cancers are refractory to such conventional CD8⁺ T cell-centered therapeutic modalities. Disclosed herein are methods of adoptive cell therapy including αβILTCks, which are useful to treat cancers that are refractory to conventional CD8⁺ T cell-centered therapeutic modalities.

Here, in an unbiased survey of CD8⁺ T cells present in murine and human malignancies, we rediscovered a population of FCER1G⁺ innate-like T cells that possess high cytotoxic potential and are resistant to exhaustion¹³, hereon termed ‘killer innate-like T cells’, or ILTCks. While tumor-infiltrating CD8⁺PD-1⁺ T cells were oligoclonal, ILTCks were polyclonal with broad reactivity to unmutated tumor-associated antigens. ILTCks were not differentiated from conventional CD8⁺ T cells, but arose from ILTCk-committed progenitors following early encounter with cognate antigens presented mostly by classical major histocompatibility molecule I (MHC-I) molecules during thymocyte development. Committed ILTCk thymic progenitors were continuously generated throughout life, and replenished the ILTCk compartment during tumor progression. Notably, expansion and effector differentiation of intratumoral ILTCks depended on high expression of IL-15 in cancer cells, and inducible activation of IL-15 signaling in adoptively transferred ILTCk progenitors suppressed tumor growth. Thus, antigen receptor self-reactivity and unconventional ontogeny distinguish ILTCks from CD8⁺PD-1⁺ T cells as a new class of tumor-elicited immune response. Strategies targeting ILTCks may enhance and complement current conventional CD8⁺ T cell-based cancer immunotherapies, in particular against tumors with low mutation burden or refractory to checkpoint blockade modalities.

### Definitions

As it would be understood, the section or subsection headings as used herein is for organizational purposes only and are not to be construed as limiting and/or separating the subject matter described.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1.0 or 0.1, as appropriate or alternatively by a variation of +/−20% or +/−15%, or alternatively 10% or alternatively 5% or alternatively 2%. As will be understood by one skilled in the art, for any and all purposes, all ranges disclosed herein also encompass any and all possible subranges and combinations of subranges thereof. Furthermore, as will be understood by one skilled in the art, a range includes each individual member.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

As used herein “adoptive cell therapeutic composition” refers to any composition comprising cells suitable for adoptive cell transfer. In exemplary embodiments, the adoptive cell therapeutic composition comprises killer innate-like T cells (ILTCks), genetically engineered ILTCks (e.g., comprising non-endogenous expression vectors encoding IL-15 or STAT5B), CAR (i.e. chimeric antigen receptor) modified ILTCks (e.g., CAR ILTCks). In one embodiment, the adoptive cell therapeutic composition comprises ILTCks.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen. It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341: 544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85 5879-5883 (1988). These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having GGGGSGGGGSGGGGS (SEQ ID NO: 37). In certain embodiments, the nucleic acid sequence encoding the amino acid sequence of SEQ ID NO: 37 is ggcggcggcggatctggaggtggtggctcaggtggcggaggctcc (SEQ ID NO: 38).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife eta., J Clin Invst 116(8):2252-61 (2006); Brocks et al., Immunotechnology 3(3): 173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell (such as on the cell surface) or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue. In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

In some embodiments, the cancer is selected from: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. In some embodiments, the cancer is a colon cancer, colorectal cancer or rectal cancer. In some embodiments, the cancer is a lung cancer. In some embodiments, the cancer is a pancreatic cancer. In some embodiments, the cancer is an adenocarcinoma, an adenocarcinoma, an adenoma, a leukemia, a lymphoma, a carcinoma, a melanoma, an angiosarcoma, or a seminoma.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, a “cleavable peptide”, which is also referred to as a “cleavable linker,” means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5. As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 39), wherein X refers to any amino acid generally thought to be self-cleaving.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “composition” is intended to mean a combination of active agent and another compound or composition, inert (for example, a nanoparticle, detectable agent or label) or active, such as an adjuvant, diluent, binder, stabilizer, buffers, salts, lipophilic solvents, preservative, adjuvant or the like and include carriers, such as pharmaceutically acceptable carriers. In some embodiments, the carrier (such as the pharmaceutically acceptable carrier) comprises, or consists essentially of, or yet further consists of a nanoparticle, such as an polymeric nanoparticle carrier or an lipid nanoparticle that can be used alone or in combination with another carrier, such as an adjuvant or solvent. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol. A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include 2B4, CD27, CD28, 4-1BB (CD137), OX40 (CD134), CD30, CD40, ICOS (CD278), LFA-1, CD2, CD7, LIGHT, NKG2C, NKG2D, B7-H2 and a ligand that specifically binds CD83. Accordingly, while the present disclosure provides exemplary co-stimulatory domains derived from CD28 and 4-1BB, other co-stimulatory domains are contemplated for use with the CARs described herein. The intracellular signaling and co-stimulatory signaling domains can be linked in any order in tandem to the carboxyl terminus of the transmembrane domain.

As used herein, the phrase “derived” means isolated, purified, mutated, or engineered, or any combination thereof. For example, an ILTCk derived from a donor refers to the ILTCk isolated from a biological sample of the donor and optionally engineered.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, a “host cell” is a cell that is used to receive, maintain, reproduce and amplify an expression vector. A host cell also can be used to express the polypeptide encoded by the expression vector. The nucleic acid contained in the expression vector is replicated when the host cell divides, thereby amplifying the nucleic acids. In some embodiments, the host cell as disclosed herein is a eukaryotic cell or a prokaryotic cell. In some embodiments, the host cell is a human cell. In some embodiments, the host cell is a cell line, such as a human embryonic kidney 293 cell (HEK 293 cell or 293 cell), or a 293T cell. These cells are commercially available, for example, from the American Type Culture Collection (ATCC).

As used herein, “killer innate-like T cells” or “ILTCks” refer to alpha-beta lineage T cells that are selected by agonistic antigens, express the signaling molecule FCER1G, the IL-2/IL-15 receptor beta chain IL2RB (CD122), but not CD4, exhibit epithelial tissue-residency properties, and can exert lytic granule-mediated cytotoxicity against cancer cells. ILTCks are distinct from conventional cytotoxic T lymphocytes.

As used herein, the term “linker” refers to any amino acid sequence comprising from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (G4S)n (SEQ ID NO: 40), wherein n is 1, or 2, or 3, or 4, or 5, or 6, or 7, or 8, or 9, or 10, or 11, or 12, or 13, or 14, or 15.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the “percent homology” between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

“Pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as an polymeric nanoparticle carrier or an lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They can be selected with respect to the intended form of administration, that is, oral tablets, capsules, elixirs, syrups and the like, and consistent with conventional pharmaceutical practices.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, “regulatory sequence” or “regulatory region” or “expression control sequence” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Polymerase II and III are examples of promoters. A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors. In some embodiments, the promoter is a constitutive promoter. As used herein, the term “constitutive promoter” refers to a promoter that allows for continual transcription of the coding sequence or gene under its control in all or most tissues of a subject at all or most developing stages. Non-limiting examples of the constitutive promoters include a CMV promoter, a simian virus 40 (SV40) promoter, a polyubiquitin C (UBC) promoter, an EF1-alpha promoter, a PGK promoter and a CAG promoter. In some embodiments, the promoter is a conditional promoter, which allows for continual transcription of the coding sequence or gene under certain conditions. In further embodiments, the conditional promoter is an immune cell specific promoter, which allows for continual transcription of the coding sequence or gene in an immune cell. Non-limiting examples of the immune cell specific promoters include a promoter of a B29 gene promoter, a CD14 gene promoter, a CD43 gene promoter, a CD45 gene promoter, a CD68 gene promoter, a IFN-β gene promoter, a WASP gene promoter, a T-cell receptor j-chain gene promoter, a V9 γ (TRGV9) gene promoter, a V2 δ (TRDV2) gene promoter, and the like.

Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

### IL-15

As used herein, the terms “interleukin-15,” “IL-15,” “interleukin 15” and “IL-5” refer to a cytokine that regulates T cell, natural killer cell activation and innate lymphoid cell proliferation. The encoded protein induces the activation of JAK kinases, as well as the phosphorylation and activation of transcription activator STAT5B, and mTORC1 signaling. Studies of the mouse counterpart suggested that this cytokine may increase the expression of apoptosis inhibitor BCL2L1/BCL-x(L), possibly through the transcription activation activity of STAT5B, and thus prevent apoptosis. Non-limiting exemplary sequences of this protein or the underlying gene may be found under NCBI Entrez Gene: 3600 (retrieved from www.ncbi.nlm.nih.gov/gene/3600), or UniProtKB/Swiss-Prot: P40933 (retrieved from www.uniprot.org/uniprot/P40933), which are incorporated by reference herein.

Exemplary amino acid sequences of IL-15 are set forth below:

>NP_000576.1 interleukin-15 isoform 1
preproprotein [Homo sapiens]
(SEQ ID NO: 19)
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS;
or
NP_751915.1 interleukin-15 isoform 2
preproprotein [Homo sapiens]
(SEQ ID NO: 20)
MVLGTIDLCSCFSAGLPKTEANWVNVISDLKKIEDLIQSMHIDATLYTE
SDVHPSCKVTAMKCFLLELQVISLESGDASIHDTVENLIILANNSLSSN
GNVTESGCKECEELEEKNIKEFLQSFVHIVQMFINTS

In some embodiments, the engineered ILTCks express a heterologous amino acid sequence that is at least 80%, 85%, 90%, 95%, 96%, 97%, 98%, or 99% identical to SEQ ID NO: 19, SEQ ID NO: 20, or a biological equivalent thereof. In further embodiments, the biological equivalent of SEQ ID NO: 19 or SEQ ID NO: 20 comprises one or more conservative amino acid substitutions relative to SEQ ID NO: 19 or SEQ ID NO: 20, respectively. Additionally or alternatively, in some embodiments, the cytokine function of the biological equivalent is substantially similar to or is significantly more efficient compared to the protein of SEQ ID NO: 19 or SEQ ID NO: 20.

Exemplary nucleic acid sequences of human IL-15 are set forth below:

>NM_000585.5 Homo sapiens interleukin 15 (IL15), transcript variant 3, mRNA
(SEQ ID NO: 21)
CTTTTCGCCAGGGGTTGGGACTCCGGGTGGCAGGCGCCCGGGGGAATCCCAGCTGACTCGCTCACTGCCT
TCGAAGTCCGGCGCCCCCCGGGAGGGAACTGGGTGGCCGCACCCTCCCGGCTGCGGTGGCTGTCGCCCCC
CACCCTGCAGCCAGGACTCGATGGAGAATCCATTCCAATATATGGCCATGTGGCTCTTTGGAGCAATGTT
CCATCATGTTCCATGCTGCTGACGTCACATGGAGCACAGAAATCAATGTTAGCAGATAGCCAGCCCATAC
AAGATCGTATTGTATTGTAGGAGGCATTGTGGATGGATGGCTGCTGGAAACCCCTTGCCATAGCCAGCTC
TTCTTCAATACTTAAGGATTTACCGTGGCTTTGAGTAATGAGAATTTCGAAACCACATTTGAGAAGTATT
TCCATCCAGTGCTACTTGTGTTTACTTCTAAACAGTCATTTTCTAACTGAAGCTGGCATTCATGTCTTCA
TTTTGGGCTGTTTCAGTGCAGGGCTTCCTAAAACAGAAGCCAACTGGGTGAATGTAATAAGTGATTTGAA
AAAAATTGAAGATCTTATTCAATCTATGCATATTGATGCTACTTTATATACGGAAAGTGATGTTCACCCC
AGTTGCAAAGTAACAGCAATGAAGTGCTTTCTCTTGGAGTTACAAGTTATTTCACTTGAGTCCGGAGATG
CAAGTATTCATGATACAGTAGAAAATCTGATCATCCTAGCAAACAACAGTTTGTCTTCTAATGGGAATGT
AACAGAATCTGGATGCAAAGAATGTGAGGAACTGGAGGAAAAAAATATTAAAGAATTTTTGCAGAGTTTT
GTACATATTGTCCAAATGTTCATCAACACTTCTTGATTGCAATTGATTCTTTTTAAAGTGTTTCTGTTAT
TAACAAACATCACTCTGCTGCTTAGACATAACAAAACACTCGGCATTTCAAATGTGCTGTCAAAACAAGT
TTTTCTGTCAAGAAGATGATCAGACCTTGGATCAGATGAACTCTTAGAAATGAAGGCAGAAAAATGTCAT
TGAGTAATATAGTGACTATGAACTTCTCTCAGACTTACTTTACTCATTTTTTTAATTTATTATTGAAATT
GTACATATTTGTGGAATAATGTAAAATGTTGAATAAAAATATGTACAAGTGTTGTTTTTTAAGTTGCACT
GATATTTTACCTCTTATTGCAAAATAGCATTTGTTTAAGGGTGATAGTCAAATTATGTATTGGTGGGGCT
GGGTACCAATGCTGCAGGTCAACAGCTATGCTGGTAGGCTCCTGCCAGTGTGGAACCACTGACTACTGGC
TCTCATTGACTTCCTTACTAAGCATAGCAAACAGAGGAAGAATTTGTTATCAGTAAGAAAAAGAAGAACT
ATATGTGAATCCTCTTCTTTATACTGTAATTTAGTTATTGATGTATAAAGCAACTGTTATGAAATAAAGA
AATTGCAATAACTGGCATATAATGTCCATCAGTAAATCTTGGTGGTGGTGGCAATAATAAACTTCTACTG
ATAGGTAGAATGGTGTGCAAGCTTGTCCAATCACGGATTGCAGGCCACATGCGGCCCAGGACAACTTTGA
ATGTGGCCCAACACAAATTCATAAACTTTCATACATCTCGTTTTTAGCTCATCAGCTATCATTAGCGGTA
GTGTATTTAAAGTGTGGCCCAAGACAATTCTTCTTATTCCAATGTGGCCCAGGGAAATCAAAAGATTGGA
TGCCCCTGGTATAGAAAACTAATAGTGACAGTGTTCATATTTCATGCTTTCCCAAATACAGGTATTTTAT
TTTCACATTCTTTTTGCCATGTTTATATAATAATAAAGAAAAACCCTGTTGATTTGTTGGAGCCATTGTT
ATCTGACAGAAAATAATTGTTTATATTTTTTGCACTACACTGTCTAAAATTAGCAAGCTCTCTTCTAATG
GAACTGTAAGAAAGATGAAATATTTTTGTTTTATTATAAATTTATTTCACCTTAA
NM_172175.3 Homo sapiens interleukin 15 (IL15), transcript variant 2, mRNA
(SEQ ID NO: 22)
CTTTTCGCCAGGGGTTGGGACTCCGGGTGGCAGGCGCCCGGGGGAATCCCAGCTGACTCGCTCACTGCCT
TCGAAGTCCGGCGCCCCCCGGGAGGGAACTGGGTGGCCGCACCCTCCCGGCTGCGGTGGCTGTCGCCCCC
CACCCTGCAGCCAGGACTCGATGGAGAATCCATTCCAATATATGGCCATGTGGCTCTTTGGAGCAATGTT
CCATCATGTTCCATGCTGCTGACGTCACATGGAGCACAGAAATCAATGTTAGCAGATAGCCAGCCCATAC
AAGATCGTTTTCAACTAGTGGCCCCACTGTGTCCGGAATTGATGGGTTCTTGGTCTCACTGACTTCAAGA
ATGAAGCCGCGGACCCTCGCGGTGAGTGTTACAGCTCTTAAGGTGGCGCATCTGGAGTTTGTTCCTTCTG
ATGTTCGGATGTGTTCGGAGTTTCTTCCTTCTGGTGGGTTCGTGGTCTCGCTGGCTCAGGAGTGAAGCTA
CAGACCTTCGCGGAGGCATTGTGGATGGATGGCTGCTGGAAACCCCTTGCCATAGCCAGCTCTTCTTCAA
TACTTAAGGATTTACCGTGGCTTTGAGTAATGAGAATTTCGAAACCACATTTGAGAAGTATTTCCATCCA
GTGCTACTTGTGTTTACTTCTAAACAGTCATTTTCTAACTGAAGCTGGCATTCATGTCTTCATTTTGGGA
TGCAGCTAATATACCCAGTTGGCCCAAAGCACCTAACCTATAGTTATATAATCTGACTCTCAGTTCAGTT
TTACTCTACTAATGCCTTCATGGTATTGGGAACCATAGATTTGTGCAGCTGTTTCAGTGCAGGGCTTCCT
AAAACAGAAGCCAACTGGGTGAATGTAATAAGTGATTTGAAAAAAATTGAAGATCTTATTCAATCTATGC
ATATTGATGCTACTTTATATACGGAAAGTGATGTTCACCCCAGTTGCAAAGTAACAGCAATGAAGTGCTT
TCTCTTGGAGTTACAAGTTATTTCACTTGAGTCCGGAGATGCAAGTATTCATGATACAGTAGAAAATCTG
ATCATCCTAGCAAACAACAGTTTGTCTTCTAATGGGAATGTAACAGAATCTGGATGCAAAGAATGTGAGG
AACTGGAGGAAAAAAATATTAAAGAATTTTTGCAGAGTTTTGTACATATTGTCCAAATGTTCATCAACAC
TTCTTGATTGCAATTGATTCTTTTTAAAGTGTTTCTGTTATTAACAAACATCACTCTGCTGCTTAGACAT
AACAAAACACTCGGCATTTCAAATGTGCTGTCAAAACAAGTTTTTCTGTCAAGAAGATGATCAGACCTTG
GATCAGATGAACTCTTAGAAATGAAGGCAGAAAAATGTCATTGAGTAATATAGTGACTATGAACTTCTCT
CAGACTTACTTTACTCATTTTTTTAATTTATTATTGAAATTGTACATATTTGTGGAATAATGTAAAATGT
TGAATAAAAATATGTACAAGTGTTGTTTTTTAAGTTGCACTGATATTTTACCTCTTATTGCAAAATAGCA
TTTGTTTAAGGGTGATAGTCAAATTATGTATTGGTGGGGCTGGGTACCAATGCTGCAGGTCAACAGCTAT
GCTGGTAGGCTCCTGCCAGTGTGGAACCACTGACTACTGGCTCTCATTGACTTCCTTACTAAGCATAGCA
AACAGAGGAAGAATTTGTTATCAGTAAGAAAAAGAAGAACTATATGTGAATCCTCTTCTTTATACTGTAA
TTTAGTTATTGATGTATAAAGCAACTGTTATGAAATAAAGAAATTGCAATAACTGGCATATAATGTCCAT
CAGTAAATCTTGGTGGTGGTGGCAATAATAAACTTCTACTGATAGGTAGAATGGTGTGCAAGCTTGTCCA
ATCACGGATTGCAGGCCACATGCGGCCCAGGACAACTTTGAATGTGGCCCAACACAAATTCATAAACTTT
CATACATCTCGTTTTTAGCTCATCAGCTATCATTAGCGGTAGTGTATTTAAAGTGTGGCCCAAGACAATT
CTTCTTATTCCAATGTGGCCCAGGGAAATCAAAAGATTGGATGCCCCTGGTATAGAAAACTAATAGTGAC
AGTGTTCATATTTCATGCTTTCCCAAATACAGGTATTTTATTTTCACATTCTTTTTGCCATGTTTATATA
ATAATAAAGAAAAACCCTGTTGATTTGTTGGAGCCATTGTTATCTGACAGAAAATAATTGTTTATATTTT
TTGCACTACACTGTCTAAAATTAGCAAGCTCTCTTCTAATGGAACTGTAAGAAAGATGAAATATTTTTGT
TTTATTATAAATTTATTTCACCTTAA

In some embodiments, the engineered ILTCks comprise a heterologous nucleic acid sequence that is at least 80%, 85%, 90%, 95%, 96%, 97%, 98%, or 99% identical to SEQ ID NO: 21, or SEQ ID NO: 22. Additionally or alternatively, in some embodiments, the expression levels and/or activity of IL-15 in the engineered ILTCk is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native ILTCk.

In some embodiments, the engineered ILTCk further comprises a first regulatory sequence operably linked to the nucleic acid encoding the IL-15. In further embodiments, the first regulatory sequence directs the expression of the IL-15. Additionally or alternatively, in some embodiments, the first regulatory sequence comprises, or consists essentially of, or yet further consists of a promoter, for example a constitutive promoter or a conditional promoter. In further embodiments, the conditional promoter is an immune cell specific promoter.

In one aspect, the engineered ILTCks provided herein overexpress IL-15 and/or comprise a heterologous nucleic acid encoding the IL-15 gene. In certain embodiments, the engineered ILTCks of the present disclosure target and kill a cancer cell expressing a target antigen more efficiently at a tissue site. The engineered ILTCks disclosed herein can be generated by in vitro transduction of ILTCks with a nucleic acid as disclosed herein.

### STAT5B

As used herein, the terms “STAT5B,” and “signal transducer and activator of transcription 5B” refer to a member of the STAT family of transcription factors. In response to cytokines and growth factors, STAT family members are phosphorylated by the receptor associated kinases, and then form homo- or heterodimers that translocate to the cell nucleus where they act as transcription activators.

STAT5B mediates the signal transduction triggered by various cell ligands, such as IL-2, IL-15, and different growth hormones. It has been shown to be involved in diverse biological processes, such as apoptosis, adult mammary gland development, and sexual dimorphism of liver gene expression. Non-limiting exemplary sequences of STAT5B protein or the underlying gene may be found under NCBI Entrez Gene: 6777 (retrieved from www.ncbi.nlm.nih.gov/gene/6777), or UniProtKB/Swiss-Prot: P42229 (retrieved from www.uniprot.org/uniprot/P42229), which are incorporated by reference herein.

Exemplary amino acid sequences of STAT5B are set forth below:

Constitutively active STAT5B (Stat5bCA), [Homo sapiens]
(SEQ ID NO: 9)
MAMWIQAQQPQGDALHQMQALYGQHFPIEVRHYLSQWIESQAWDSID
LDNPQENIKATQLLEGLVQELQKKAEHQVGEDGFLLKIKLGHYATQLQSTYDRCP
MELVRCIRHILYNEQRLVREANNGSSPAGSLADAMSQKHLQINQTFEELRLITQDTE
NELKKLQQTQEYFIIQYQESLRIQAQFAQLGQLNPQERMSRETALQQKQVSLETWL
QREAQTLQQYRVELAEKHQKTLQLLRKQQTIILDDELIQWKRRQQLAGNGGPPEGS
LDVLQSWCEKLAEIIWQNRQQIRRAERLCQQLPIPGPVEEMLAEVNATITDIISALVT
STFIIEKQPPQVLKTQTKFAATVRLLVGGKLNVHMNPPQVKATIISEQQAKSLLKNE
NTRNDYSGEILNNCCVMEYHQATGTLSAHFRNMSLKRIKRSDRRGAESVTEEKFTI
LFDSQFSVGGNELVFQVKTLSLPVVVIVHGSQDNNATATVLWDNAFAEPGRVPFA
VPDKVLWPQLCEALNMKFKAEVQSNRGLTKENLVFLAQKLFNISSNHLEDYNSMS
VSWSQFNRENLPGRNYTFWQWFDGVMEVLKKHLKPHWNDGAILGFVNKQQAHD
LLINKPDGTFLLRFSDSEIGGITIAWKFDSQERMFWNLMPFTTRDFSIRSLADRLGDL
NYLIYVFPDRPKDEVYSKYYTPVPCEPATAKAADGYVKPQIKQVVPEFANAFTDAG
SGATYMDQAPSPVVCPQAHYNMYPPNPDSVLDTDGDFDLEDMMDVARRVEELLG
RPMDSQWIPHAQS;
or
>NP_036580.2 signal transducer and activator of transcription
5B [Homo sapiens]
(SEQ ID NO: 23)
MAVWIQAQQLQGEALHQMQALYGQHFPIEVRHYLSQWIESQAWDSVD
LDNPQENIKATQLLEGLVQELQKKAEHQVGEDGFLLKIKLGHYATQLQNTYDRCP
MELVRCIRHILYNEQRLVREANNGSSPAGSLADAMSQKHLQINQTFEELRLVTQDT
ENELKKLQQTQEYFIIQYQESLRIQAQFGPLAQLSPQERLSRETALQQKQVSLEAWL
QREAQTLQQYRVELAEKHQKTLQLLRKQQTIILDDELIQWKRRQQLAGNGGPPEGS
LDVLQSWCEKLAEIIWQNRQQIRRAEHLCQQLPIPGPVEEMLAEVNATITDIISALVT
STFIIEKQPPQVLKTQTKFAATVRLLVGGKLNVHMNPPQVKATIISEQQAKSLLKNE
NTRNDYSGEILNNCCVMEYHQATGTLSAHFRNMSLKRIKRSDRRGAESVTEEKFTI
LFESQFSVGGNELVFQVKTLSLPVVVIVHGSQDNNATATVLWDNAFAEPGRVPFAV
PDKVLWPQLCEALNMKFKAEVQSNRGLTKENLVFLAQKLFNNSSSHLEDYSGLSV
SWSQFNRENLPGRNYTFWQWFDGVMEVLKKHLKPHWNDGAILGFVNKQQAHDL
LINKPDGTFLLRFSDSEIGGITIAWKFDSQERMFWNLMPFTTRDFSIRSLADRLGDLN
YLIYVFPDRPKDEVYSKYYTPVPCESATAKAVDGYVKPQIKQVVPEFVNASADAG
GGSATYMDQAPSPAVCPQAHYNMYPQNPDSVLDTDGDFDLEDTMDVARRVEELL
GRPMDSQWIPHAQS

In some embodiments, the engineered ILTCks express a heterologous amino acid sequence that is at least 80%, 85%, 90%, 95%, 96%, 97%, 98%, or 99% identical to SEQ ID NO: 9, SEQ ID NO: 23, or a biological equivalent thereof. In further embodiments, the biological equivalent of SEQ ID NO: 9 or SEQ ID NO: 23 comprises one or more conservative amino acid substitutions relative to SEQ ID NO: 9 or SEQ ID NO: 23, respectively. Additionally or alternatively, in some embodiments, the function of the biological equivalent is substantially similar to or is significantly more efficient compared to the protein of SEQ ID NO: 9 or SEQ ID NO: 23.

Exemplary nucleic acid sequences of human STAT5B are set forth below:

Constitutively active STAT5B (Stat5bCA), mRNA
(SEQ ID NO: 18)
ATGGCTATGTGGATACAGGCTCAGCAGCCCCAGGGCGATGCCCTTCA
CCAGATGCAGGCCTTGTACGGCCAGCATTTCCCCATCGAGGTGCGACATTATTT
ATCACAGTGGATCGAAAGCCAAGCCTGGGACTCAATAGATCTTGATAATCCAC
AGGAGAACATTAAGGCCACCCAGCTCCTGGAGGGCCTGGTGCAGGAGCTGCAG
AAGAAGGCGGAGCACCAGGTGGGGGAAGATGGGTTTTTGCTGAAGATCAAGCT
GGGGCACTATGCCACACAGCTCCAGAGCACGTACGACCGCTGCCCCATGGAGC
TGGTTCGCTGTATCCGGCACATTCTGTACAACGAACAGAGGCTGGTTCGCGAAG
CCAACAACGGCAGCTCTCCAGCTGGAAGTCTTGCTGACGCCATGTCCCAGAAGC
ACCTTCAGATCAACCAAACGTTTGAGGAGCTGCGCCTGATCACACAGGACACG
GAGAACGAGCTGAAGAAGCTGCAGCAGACCCAAGAGTACTTCATCATCCAGTA
CCAGGAGAGCCTGCGGATCCAAGCTCAGTTTGCCCAGCTGGGACAGCTGAACC
CCCAGGAGCGCATGAGCAGGGAGACGGCCCTCCAGCAGAAGCAAGTGTCCCTG
GAGACCTGGCTGCAGCGAGAGGCACAGACACTGCAGCAGTACCGAGTGGAGCT
GGCTGAGAAGCACCAGAAGACCCTGCAGCTGCTGCGGAAGCAGCAGACCATCA
TCCTGGACGACGAGCTGATCCAGTGGAAGCGGAGACAGCAGCTGGCCGGGAAC
GGGGGTCCCCCCGAGGGCAGCCTGGACGTGCTGCAGTCCTGGTGTGAGAAGCT
GGCCGAGATCATCTGGCAGAATCGGCAGCAGATCCGCAGGGCTGAGCGCCTGT
GCCAGCAGCTGCCCATCCCAGGCCCCGTGGAGGAGATGCTGGCTGAGGTCAAC
GCCACCATCACGGACATCATCTCAGCCCTGGTCACCAGCACGTTCATCATCGAG
AAGCAGCCTCCTCAGGTCCTGAAGACCCAGACCAAGTTTGCGGCCACTGTGCGC
CTGCTGGTGGGGGGGAAGCTGAATGTGCACATGAACCCCCCGCAGGTGAAGGC
GACCATCATCAGCGAGCAGCAGGCCAAGTCCCTGCTCAAGAATGAGAACACCC
GCAATGATTACAGCGGCGAGATCCTGAACAACTGTTGCGTCATGGAGTACCACC
AGGCCACTGGCACGCTCAGCGCCCACTTCAGAAACATGTCCCTGAAACGAATC
AAGAGATCTGACCGCCGTGGTGCAGAGTCAGTAACGGAAGAGAAGTTCACGAT
CCTGTTTGACTCACAGTTCAGCGTCGGTGGAAACGAGCTGGTCTTTCAAGTCAA
GACCTTGTCGCTCCCGGTGGTGGTGATTGTTCACGGCAGCCAGGACAACAATGC
CACAGCCACTGTCCTCTGGGACAACGCCTTTGCAGAGCCTGGCAGGGTGCCATT
TGCCGTGCCTGACAAGGTGCTGTGGCCGCAGCTGTGTGAAGCGCTCAACATGAA
ATTCAAGGCTGAAGTACAGAGCAACCGGGGCTTGACCAAGGAGAACCTCGTGT
TCCTGGCACAGAAACTGTTCAACATCAGCAGCAACCACCTCGAGGACTACAAC
AGCATGTCCGTGTCCTGGTCCCAGTTCAACCGGGAGAATTTGCCAGGACGGAAT
TACACTTTCTGGCAGTGGTTTGATGGCGTGATGGAAGTATTGAAAAAACATCTC
AAGCCTCACTGGAATGATGGGGCTATCCTGGGTTTCGTGAACAAGCAACAGGC
CCACGACCTGCTCATCAACAAGCCGGACGGGACCTTCCTGCTGCGCTTCAGCGA
CTCGGAAATCGGGGGCATCACCATTGCTTGGAAGTTTGACTCTCAGGAGAGAAT
GTTTTGGAATCTGATGCCTTTTACCACTAGAGACTTCTCTATCCGGTCCCTCGCT
GACCGCCTGGGGGACCTGAATTACCTCATATACGTGTTTCCTGATCGGCCAAAG
GATGAAGTATATTCTAAGTACTACACACCGGTCCCCTGTGAGCCCGCAACTGCG
AAAGCAGCTGACGGATACGTGAAGCCACAGATCAAGCAGGTGGTCCCCGAGTT
TGCAAATGCATTCACAGATGCTGGGAGTGGCGCCACCTACATGGATCAGGCTCC
TTCCCCAGTCGTGTGCCCTCAGGCTCACTACAACATGTACCCACCCAACCCGGA
CTCCGTCCTTGATACCGATGGGGACTTCGATCTGGAAGACATGATGGACGTGGC
GCGGCGCGTGGAAGAGCTCTTAGGCCGGCCCATGGACAGTCAGTGGATCCCTC
ACGCACAGTCA
>NM_012448.4 Homo sapiens signal transducer and activator of
transcription 5B (STAT5B), mRNA
(SEQ ID NO: 24)
GGCGGCCGGAGCCGTCACCCCGGGCGGGGACCCAGCGCAGGCAACT
CCGCGCGGCGGCCCGGCCGAGGGAGGGAGCGAGCGGGCGGGCGGGCAAGCCA
GACAGCTGGGCCGGAGCAGCCGCGGGCGCCCGAGGGGCCGAGCGAGATTGTAA
ACCATGGCTGTGTGGATACAAGCTCAGCAGCTCCAAGGAGAAGCCCTTCATCA
GATGCAAGCGTTATATGGCCAGCATTTTCCCATTGAGGTGCGGCATTATTTATC
CCAGTGGATTGAAAGCCAAGCATGGGACTCAGTAGATCTTGATAATCCACAGG
AGAACATTAAGGCCACCCAGCTCCTGGAGGGCCTGGTGCAGGAGCTGCAGAAG
AAGGCAGAGCACCAGGTGGGGGAAGATGGGTTTTTACTGAAGATCAAGCTGGG
GCACTATGCCACACAGCTCCAGAACACGTATGACCGCTGCCCCATGGAGCTGGT
CCGCTGCATCCGCCATATATTGTACAATGAACAGAGGTTGGTCCGAGAAGCCAA
CAATGGTAGCTCTCCAGCTGGAAGCCTTGCTGATGCCATGTCCCAGAAACACCT
CCAGATCAACCAGACGTTTGAGGAGCTGCGACTGGTCACGCAGGACACAGAGA
ATGAGTTAAAAAAGCTGCAGCAGACTCAGGAGTACTTCATCATCCAGTACCAG
GAGAGCCTGAGGATCCAAGCTCAGTTTGGCCCGCTGGCCCAGCTGAGCCCCCA
GGAGCGTCTGAGCCGGGAGACGGCCCTCCAGCAGAAGCAGGTGTCTCTGGAGG
CCTGGTTGCAGCGTGAGGCACAGACACTGCAGCAGTACCGCGTGGAGCTGGCC
GAGAAGCACCAGAAGACCCTGCAGCTGCTGCGGAAGCAGCAGACCATCATCCT
GGATGACGAGCTGATCCAGTGGAAGCGGCGGCAGCAGCTGGCCGGGAACGGCG
GGCCCCCCGAGGGCAGCCTGGACGTGCTACAGTCCTGGTGTGAGAAGTTGGCC
GAGATCATCTGGCAGAACCGGCAGCAGATCCGCAGGGCTGAGCACCTCTGCCA
GCAGCTGCCCATCCCCGGCCCAGTGGAGGAGATGCTGGCCGAGGTCAACGCCA
CCATCACGGACATTATCTCAGCCCTGGTGACCAGCACGTTCATCATTGAGAAGC
AGCCTCCTCAGGTCCTGAAGACCCAGACCAAGTTTGCAGCCACTGTGCGCCTGC
TGGTGGGCGGGAAGCTGAACGTGCACATGAACCCCCCCCAGGTGAAGGCCACC
ATCATCAGTGAGCAGCAGGCCAAGTCTCTGCTCAAGAACGAGAACACCCGCAA
TGATTACAGTGGCGAGATCTTGAACAACTGCTGCGTCATGGAGTACCACCAAGC
CACAGGCACCCTTAGTGCCCACTTCAGGAATATGTCCCTGAAACGAATTAAGAG
GTCAGACCGTCGTGGGGCAGAGTCGGTGACAGAAGAAAAATTTACAATCCTGT
TTGAATCCCAGTTCAGTGTTGGTGGAAATGAGCTGGTTTTTCAAGTCAAGACCC
TGTCCCTGCCAGTGGTGGTGATCGTTCATGGCAGCCAGGACAACAATGCGACGG
CCACTGTTCTCTGGGACAATGCTTTTGCAGAGCCTGGCAGGGTGCCATTTGCCG
TGCCTGACAAAGTGCTGTGGCCACAGCTGTGTGAGGCGCTCAACATGAAATTCA
AGGCCGAAGTGCAGAGCAACCGGGGCCTGACCAAGGAGAACCTCGTGTTCCTG
GCGCAGAAACTGTTCAACAACAGCAGCAGCCACCTGGAGGACTACAGTGGCCT
GTCTGTGTCCTGGTCCCAGTTCAACAGGGAGAATTTACCAGGACGGAATTACAC
TTTCTGGCAATGGTTTGACGGTGTGATGGAAGTGTTAAAAAAACATCTCAAGCC
TCATTGGAATGATGGGGCCATTTTGGGGTTTGTAAACAAGCAACAGGCCCATGA
CCTACTCATTAACAAGCCAGATGGGACCTTCCTCCTGAGATTCAGTGACTCAGA
AATTGGCGGCATCACCATTGCTTGGAAGTTTGATTCTCAGGAAAGAATGTTTTG
GAATCTGATGCCTTTTACCACCAGAGACTTCTCCATTCGGTCCCTAGCCGACCG
CTTGGGAGACTTGAATTACCTTATCTACGTGTTTCCTGATCGGCCAAAAGATGA
AGTATACTCCAAATACTACACACCAGTTCCCTGCGAGTCTGCTACTGCTAAAGC
TGTTGATGGATACGTGAAGCCACAGATCAAGCAAGTGGTCCCTGAGTTTGTGAA
CGCATCTGCAGATGCCGGGGGCGGCAGCGCCACGTACATGGACCAGGCCCCCT
CCCCAGCTGTGTGTCCCCAGGCTCACTATAACATGTACCCACAGAACCCTGACT
CAGTCCTTGACACCGATGGGGACTTCGATCTGGAGGACACAATGGACGTAGCG
CGGCGTGTGGAGGAGCTCCTGGGCCGGCCAATGGACAGTCAGTGGATCCCGCA
CGCACAATCGTGACCCCGCGACCTCTCCATCTTCAGCTTCTTCATCTTCACCAGA
GGAATCACTCTTGTGGATGTTTTAATTCCATGAATCGCTTCTCTTTTGAAACAAT
ACTCATAATGTGAAGTGTTAATACTAGTTGTGACCTTAGTGTTTCTGTGCATGGT
GGCACCAGCGAAGGGAGTGCGAGTATGTGTTTGTGTGTGTGTGTGTGTGTGTGT
GTGTGTGCGTGTTTGCACGTTATGGTGTTTCTCCCTCTCACTGTCTGAGAGTTTA
GTTGTAGCAGAGGGGCCACAGACAGAAGCTGTGGTGGTTTTTACTTTGTGCAAA
AAGGCAGTGAGTTTCGTGAAGCCTGGAAGTTGGCCATGTGTCTTAAGAGTGGCT
GGACTTTGACATGTGGCTGTTTGAATAAGAGAAGGACAAAGGGAGGAGAAAGC
ACATGTGCTCCAGTGAGTCTTCGTCACTCTGTCTGCCAAGCAATTGATATATAA
CCGTGATTGTCTCTGCTTTTCTTCTGAAATGTAGATAACTGCTTTTTGACAAAGA
GAGCCTTCCCTCTCCCCCACCCCTGTGTTCTTGGGTAGGAATGGGAAAAGGGGC
AACCTACAAAGATTGTTGGGGCAAGGGAAGTCACAAGCTTTCGGATGGGCGGT
GGCTTTTCACAAAACATTTAGCTCATCTTATTCTCTCTTTGTCCTCTCTCCCCTCC
TGCCCGCCCGCACCCTGGAATTGCCACTCAGTTCCTCTGGGTGTGCACATATGTT
TGGAGAAATAGAGGAGAGAAAAGAGGGCCACGTAACTGAGAGCTTACAGTGC
CAATGCCGTTTGTGTTCTGGCCAGAGTGGAGTGCGCAGCCCTGACTCCCAGGCG
CTGAGATTGTTGCCTGGTTACCCAGGAAGCTGCTGTTCCGGCTGCCCAGCCTTTC
TCTGAGCCAGCGGATGCACAGTCCGTGGCCTTCTTCAGGCTTATTGATGATGCT
TTTTGCAAATGTTGAATCATGGTTCTGTTTCTAAGTTGGATCTTTTTTGTTTTCTC
CTTGCCACCCTAATTTGACATCAAAATTCTCTCTTGTGCATTGGGCCCTGGGTCA
TTCAAACCCAGGTCACCTCATTCCCCTTCTCTGTTCACACCTAATGTCTTGAAGA
GTAGGTAGCAGCAGTGTGGGCTGAACCTAGGCCAGCTTGCTTAGCGGGTCACCC
TGCTGTGAAGTCCTGGCAGGTGTTGGTAATGTGTGGAAATGCAGTCAGCAAGTT
TGCTGGGGAGTTTGATAAAAGTATAAAACAAAACAAAAAAAGCCTCGGTATAA
TTTTGTTCCACGACTTCTTCTGTAGCTTTACACCAGAAGGAAGGAATGGGCTAC
AGCAGGTAGTGGAGGAAGAGGGGGGTGAGCAGGTGTATTAAAATAGCTTACGG
GTAAGGCCTAAAAGGTCACCCCTCGGCCCCCTCTCCAAAAGAAGGGCATGGGC
ACCCCCAGGAGAGGATGGCCCCAAAAACCTTATTTTTATACATGAGAGTAAATA
AACATATTTTTTTTACAAAAATAACTTCTGAATTTATCAGTGTTTTGCCGTTAAA
AATATTCCTCTATAGTAAATTATTTATTGGAAGATGACTTTTTTAAAGCTGCCGT
TTGCCTTGGCTTGGTTTCATACACTGATTTATTTTTCTATGCCAGGCAGTAGAGT
CTCTCTGCCTCTGAGGAGCAGGCTACCCGCATCCCACTCAGCCCCTCCCTACCC
CTCAAGATTTGATGAAAATTCCAACCATGAGGATGGGTGCATCGGGGAAGGGT
GAGAAGGAGAGCCTGCCTGCTCAGGGATCCAGGCTCGTAGAGTCACTCCCTGC
CCGTCTCCCAGAGATGCTTCACCAGCACCTGCCTCTGAGACCTCGCTCTCTGTTC
CAGCAACCCTGGTTGGGGGGTCAGACTTGATACACTTTCAGGTTGGGAGTGGAC
CCACCCCAGGGCCTGCTGAGGACAGAGCAGCCAGGCCGTCCTGGCTCACTTTGC
AGTTGGCACTGGGTTGGGGAGGAAGAGAGCTGATGAGTGTGGCTTCCCTGAGC
TGGGGTTTCCCTGCTTGTCCAGTTGTGAGCTGTCCTCGGTGTTACCGAGGCTGTG
CCTAGAGAGTGGAGATTTTTGATGAAAGGTGTGCTCGCTCTCTGCGTTCTATCTT
CTCTCTCCTCCTTGTTCCTGCAAACCACAAGATAAAGGTAGTGGTGTGTCTCGA
CCCCATCAGCCTCTCACCCACTCCCAGACACACACAAGTCCTCAAAAGTTTCAG
CTCCGTGTGTGAGATGTGCAGGTTTTTTCTAGGGGGTAGGGGGAGACTAAAATC
GAATATAACTTAAAATGAAAGTATACTTTTTATAATTTTTCTTTTTAAAACTTGG
TGAAATTATTTCAGATACATATTTTAGTGTCAAGGCAGATTAGTTATTTAGCCAC
CAAAAAAAAGTATTGTGTACAATTTGGGGCCTCAAATTTGACTCTGCCTCAAAA
AAAAGAAATATATCCTATGCAGAGTTACAGTCACAAAGTTGTGTATTTTATGTT
ACAATAAAGCCTTCCTCTGAAGGGA

In some embodiments, the engineered ILTCks comprise a heterologous nucleic acid sequence that is at least 80%, 85%, 90%, 95%, 96%, 97%, 98%, or 99% identical to SEQ ID NO: 18, or SEQ ID NO: 24. Additionally or alternatively, in some embodiments, the expression levels and/or activity of STAT5B in the engineered ILTCk is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native ILTCk.

In some embodiments, the engineered ILTCk further comprises a first regulatory sequence operably linked to the nucleic acid encoding the STAT5B. In further embodiments, the first regulatory sequence directs the expression of the STAT5B. Additionally or alternatively, in some embodiments, the first regulatory sequence comprises, or consists essentially of, or yet further consists of a promoter, for example a constitutive promoter or a conditional promoter. In further embodiments, the conditional promoter is an immune cell specific promoter.

In one aspect, the engineered ILTCks provided herein overexpress STAT5B and/or comprise a heterologous nucleic acid encoding the STAT5B gene. In certain embodiments, the engineered ILTCks of the present disclosure target and kill a cancer cell expressing a target antigen more efficiently at a tissue site. The engineered ILTCks disclosed herein can be generated by in vitro transduction of ILTCks with a nucleic acid as disclosed herein.

### Chimeric Antigen Receptors (CARs)

Typical therapeutic anti-cancer monoclonal antibody (mAb), like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by MHC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PRI, among others, have been developed. The antigen binding portions of such antibodies can be incorporated into the CARs provided herein. HLA-A2 is the most common HLA haplotype in the USA and EU (about 40% of the population) (Marsh, S., Parham, P., Barber, L., The HLA FactsBook. 1 ed. The HLA FactsBook. Vol. 1. 2000: Academic Press. 416). Therefore, potent TCRm mAb and native TCRs against tumor antigens presented in the context of HLA-A2 are useful in the treatment of a large populations. Accordingly, in some embodiments, a receptor as disclosed herein binds to a target antigen. In further embodiments, the target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule.

In some embodiments, the engineered ILTCks provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as an ILTCk. In some embodiments, transfer of the coding sequence of the CAR is facilitated by nucleic acid vector, such as a retroviral vector.

There are currently three generations of CARs. In some embodiments, the engineered ILTCks provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered ILTCks provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the ILTCk. “Second generation” CARs comprise those that provide both co-stimulation (e.g., CD28 or 4-1BB) and activation (e.g., CD3ζ).

In some embodiments, the engineered ILTCks provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered ILTCks provided herein comprise an extracellular antigen-binding domain, a transmembrane domain and an intracellular domain. Further, the activity of the engineered ILTCks can be adjusted by selection of co-stimulatory molecules included in the chimeric antigen receptor.

Extracellular Antigen-Binding Domain of a CAR. In certain embodiments, the extracellular antigen-binding domain of a CAR specifically binds a target antigen. In certain embodiments, the extracellular antigen-binding domain is derived from a monoclonal antibody (mAb) that binds to a target antigen. In some embodiments, the extracellular antigen-binding domain comprises, or consists essentially of, or yet further consists of an scFv. In some embodiments, the extracellular antigen-binding domain comprises, or consists essentially of, or yet further consists of a Fab, which is optionally crosslinked. In some embodiments, the extracellular binding domain comprises, or consists essentially of, or yet further consists of a F(ab)₂. In some embodiments, any of the foregoing molecules are included in a fusion protein with a heterologous sequence to form the extracellular antigen-binding domain. In certain embodiments, the extracellular antigen-binding domain comprises, or consists essentially of, or yet further consists of a human scFv that binds specifically to a target antigen. In certain embodiments, the scFv is identified by screening scFv phage library with a target antigen-Fc fusion protein.

In certain embodiments, the extracellular antigen-binding domain of a presently disclosed CAR has a high binding specificity and high binding affinity to a target antigen. For example, in some embodiments, the extracellular antigen-binding domain of the CAR (embodied, for example, in a human scFv or an analog thereof) binds to a particular target antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed target-antigen-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the target-antigen-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed target-antigen-specific CAR is labeled with GFP.

In some embodiments, the extracellular antigen-binding domain of the expressed CAR binds to a target antigen. In some embodiments, the extracellular antigen-binding domain of the expressed CAR binds to a target antigen presented in the context of an MHC molecule. In some embodiments, the extracellular antigen-binding domain of the expressed CAR binds to a target antigen presented in the context of an HLA-A2 molecule. In some embodiments, the extracellular antigen-binding domain of the expressed CAR binds to a target antigen not in combination with an MHC protein.

In some embodiments, the extracellular antigen-binding domain of the expressed CAR binds to a target antigen that is expressed by a tumor cell. In some embodiments, the extracellular antigen-binding domain of the expressed CAR binds to a target antigen that is expressed on the surface of a tumor cell. In some embodiments, the extracellular antigen-binding domain of the expressed CAR binds to a target antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a target antigen that is expressed on the surface of a tumor cell not in combination with an MHC protein.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 37), between the heavy chain variable (V_{H}) region and the light chain variable (V_{L}) region. In certain embodiments, the extracellular antigen-binding domain is a human scFv-Fc fusion protein or full length human IgG with V_{H} and V_{L} regions.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 37. In certain embodiments, the nucleotide sequence encoding the amino acid sequence of SEQ ID NO: 37 is set forth in SEQ ID NO: 38.

Exemplary amino acid sequences of heavy chain variable (V_{H}) region and a light chain variable (V_{L}) region include, but are not limited to:

CD19 V_{H} domain
(SEQ ID NO: 2)
EVKLQQSGAELVRPGSSVKISCKASGYAFSSYWMNWVKQRPGQGLEWIG
QIYPGDGDTNYNGKFKGQATLTADKSSSTAYMQLSGLTSEDSAVYFCAR
KTISSVVDFYFDYWGQGTTVTVSS
CD19 V_{L} domain
(SEQ ID NO: 3)
DIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTS
GGGTKLEIK
HER2 V_{H} domain
(SEQ ID NO: 41)
EVOLVESGGGLVQPGGSLRLSCAASGFNIKDTYIHWVRQAPGKGLEWV
ARIYPTNGYTRYADSVKGRFTISADTSKNTAYLQMNSLRAEDTAVYYCS
RWGGDGFYAMDYWGQGTLVTVSS
HER2 V_{L} domain
(SEQ ID NO: 42)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIK
GPC3 V_{H} domain
(SEQ ID NO: 43)
QVQLQQSGAELVRPGASVKLSCKASGYTFTDYEMHWVKQTPVHGLKW
IGALDPKTGDTAYSQKFKGKATLTADKSSSTAYMELRSLTSEDSAVYYC
TRFYSYTYWGQGTLVTVSA
GPC3 V_{L} domain
(SEQ ID NO: 44)
DVVMTQTPLSLPVSLGDQASISCRSSQSLVHSNGNTYLHWYLQKPGQSP
KLLIYKVSNRFSGVPDRFSGSGSGTDFTLKISRVEAEDLGVYFCSQNTH
VPPTFGSGTKLEIK

Exemplary nucleic acid sequences of heavy chain variable (V_{H}) region and a light chain variable V_{L} region include but are not limited to:

CD19 V_{H} domain
(SEQ ID NO: 11)
GAGGTGAAGCTGCAGCAGTCTGGGGCTGAGCTGGTGAGGCCTGGGTC
CTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTG
GACAGATTTATCCTGGAGATGGTGATACTAACTACAATGGAAAGTTCAA
GGGTCAAGCCACACTGACTGCAGACAAATCCTCCAGCACAGCCTACATG
CAGCTCAGCGGCCTAACATCTGAGGACTCTGCGGTCTATTTCTGTGCAA
GAAAGACCATTAGTTCGGTAGTAGATTTCTACTTTGACTACTGGGGCCA
AGGGACCACGGTCACCGTCTCCTCA
CD19 V_{L} domain
(SEQ ID NO: 12)
GACATTGAGCTCACCCAGTCTCCAAAATTCATGTCCACATCAGTAGG
AGACAGGGTCAGCGTCACCTGCAAGGCCAGTCAGAATGTGGGTACTAAT
GTAGCCTGGTATCAACAGAAACCAGGACAATCTCCTAAACCACTGATTT
ACTCGGCAACCTACCGGAACAGTGGAGTCCCTGATCGCTTCACAGGCAG
TGGATCTGGGACAGATTTCACTCTCACCATCACTAACGTGCAGTCTAAA
GACTTGGCAGACTATTTCTGTCAACAATATAACAGGTATCCGTACACGT
CCGGAGGGGGGACCAAGCTGGAGATCAAA
HER2 V_{H} domain
(SEQ ID NO: 45)
GAGGTCCAGTTGGTTGAATCTGGTGGAGGTTTGGTCCAGCCAGGTGG
ATCTTTGAGATTGTCTTGTGCCGCTTCTGGTTTCAACATCAAGGACACC
TACATTCATTGGGTTAGACAAGCCCCTGGTAAGGGATTGGAGTGGGTTG
CCAGAATTTACCCAACAAACGGATACACAAGATACGCTGACTCTGTCAA
GGGAAGATTCACTATCTCTGCCGACACATCTAAGAACACTGCATACTTG
CAAATGAACTCTTTGAGAGCCGAAGACACAGCCGTCTACTACTGCTCTA
GATGGGGTGGTGACGGTTTTTACGCCATGGACTATTGGGGTCAAGGAAC
ATTGGTCACAGTCTCTTCT
HER2 V_{L} domain
(SEQ ID NO: 46)
GACATTCAGATGACCCAATCTCCATCTTCTTTGTCTGCCTCTGTCGGT
GATAGAGTTACCATCACCTGCAGAGCTTCTCAAGACGTCAATACCGCAG
TTGCCTGGTATCAACAGAAGCCAGGAAAGGCACCTAAGTTGTTGATCTA
CTCTGCTTCTTTTTTGTACTCTGGAGTCCCTTCTAGATTTTCTGGATCT
AGATCTGGTACCGATTTCACATTGACCATTTCTTCTTTGCAGCCTGAGG
ACTTTGCCACATATTACTGTCAGCAGCACTACACAACCCCTCCTACTTT
TGGTCAGGGAACTAAGGTCGAGATTAAG
GPC3 V_{H} domain
(SEQ ID NO: 47)
CAGGTCCAGCTGCAGCAGTCAGGAGCCGAACTGGTGCGGCCCGGCGC
AAGTGTCAAACTGTCATGCAAGGCCAGCGGGTATACCTTCACAGACTAC
GAGATGCACTGGGTGAAACAGACCCCTGTGCACGGCCTGAAGTGGATCG
GCGCTCTGGACCCAAAAACCGGGGATACAGCATATTCCCAGAAGTTTAA
AGGAAAGGCCACTCTGACCGCTGACAAGAGCTCCTCTACTGCCTACATG
GAGCTGAGGAGCCTGACATCCGAAGATAGCGCCGTGTACTATTGCACCC
GCTTCTACTCCTATACATACTGGGGCCAGGGGACTCTGGTGACCGTCTC
TGCA
GPC3 V_{L} domain
(SEQ ID NO: 48)
GACGTGGTCATGACACAGACTCCACTGTCCCTGCCCGTGAGCCTGGG
CGATCAGGCTAGCATTTCCTGTCGAAGTTCACAGAGTCTGGTGCACTCA
AACGGAAATACCTATCTGCATTGGTACCTGCAGAAGCCAGGCCAGTCTC
CCAAACTGCTGATCTATAAGGTGAGCAACCGGTTCTCCGGGGTCCCTGA
CAGATTTTCTGGAAGTGGCTCAGGGACAGATTTCACTCTGAAAATTAGC
AGAGTGGAGGCCGAAGATCTGGGCGTCTACTTTTGTAGCCAGAATACCC
ACGTCCCACCAACATTCGGAAGCGGCACTAAACTGGAAATCAAG

In certain embodiments, the extracellular antigen-binding domain (e.g., human scFv) comprises a heavy chain variable (V_{H}) region and a light chain variable (V_{L}) region comprising SEQ ID NO: 2 and SEQ ID NO: 3, SEQ ID NO: 41 and SEQ ID NO: 42, or SEQ ID NO: 43 and SEQ ID NO: 44, respectively.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 1 as provided below: MALPVTALLLPLALLLHA (SEQ ID NO: 1).

The nucleotide sequence encoding the amino acid sequence of SEQ ID NO: 1 is set forth in SEQ ID NO: 10, which is provided below:

(SEQ ID NO: 10)
ATGGCTCTCCCAGTGACTGCCCTACTGCTTCCCCTAGCGCTTCTCCTGC
ATGCA.

In certain embodiments, the signal peptide comprises a human CD8 signal polypeptide comprising amino acids having the sequence set forth in SEQ ID NO: 25 as provided below: MALPVTALLLPLALLLHAARP (SEQ ID NO: 25).

The nucleotide sequence encoding the amino acid sequence of SEQ ID NO: 25 is set forth in SEQ ID NO: 26, which is provided below:

(SEQ ID NO: 26)
ATGGCCCTGCCAGTAACGGCTCTGCTGCTGCCACTTGCTCTGCTCCTCC
ATGCAGCCAGGCCT.

In certain embodiments, the signal peptide comprises a mouse CD8 signal polypeptide comprising amino acids having the sequence set forth in SEQ ID NO: 27 as provided below: MASPLTRFLSLNLLLLGESII (SEQ ID NO: 27).

The nucleotide sequence encoding the amino acid sequence of SEQ ID NO: 27 is set forth in SEQ ID NO: 28, which is provided below:

(SEQ ID NO: 28)
ATGGCCAGCCCCCTGACCAGGTTCCTGAGCCTGAACCTGCTGCTGCT
GGGCGAGAGCATCATC.

In certain embodiments, the signal peptide comprises a mouse CD8 signal polypeptide comprising amino acids having the sequence set forth in SEQ ID NO: 29 as provided below: MASPLTRFLSLNLLLLGE (SEQ ID NO: 29).

The nucleotide sequence encoding the amino acid sequence of SEQ ID NO: 29 is set forth in SEQ ID NO: 30, which is provided below:

(SEQ ID NO: 30)
ATGGCCAGCCCCCTGACCAGGTTCCTGAGCCTGAACCTGCTGCTGCT
GGGCGAG.

In certain embodiments, the signal peptide comprises an IL2RB signal polypeptide comprising amino acids having the sequence set forth in SEQ ID NO: 49 as provided below: MAAPALSWRLPLLILLLPLATSWASA (SEQ ID NO: 49).

The nucleotide sequence encoding the amino acid sequence of SEQ ID NO: 49 is set forth in SEQ ID NO: 50, which is provided below:

(SEQ ID NO: 50)
ATGGCGGCCCCTGCTCTGTCCTGGCGTCTGCCCCTCCTCATCCTCCT
CCTGCCCCTGGCTACCTCTTGGGCATCTGCA.

In certain embodiments, the signal peptide comprises an Ig signal polypeptide comprising amino acids having the sequence set forth in SEQ ID NO: 51 as provided below: MDWIWRILFLVGAATGAHS (SEQ ID NO: 51).

The nucleotide sequence encoding the amino acid sequence of SEQ ID NO: 51 is set forth in SEQ ID NO: 52, which is provided below:

(SEQ ID NO: 52)
ATGGATTGGATTTGGCGCATTCTGTTTCTGGTGGGAGCCGCAACCGG
AGCACATAGT

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a NKG2D polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 31), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 31 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 31. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 31. In certain embodiments, the CD28 polypeptide comprised in the transmembrane domain has an amino acid sequence of amino acids 153 to 179 of SEQ ID NO: 31.

SEQ ID NO: 31 is provided below:

(SEQ ID NO: 31)
MLRLLLALNLFPSIQVTGNKILVKQSPMLVAYDNALSCKYSYNLFSREF
RASLHKGLDSAVEVCWYGNYSQQLQVYSKTGFNCDGKLGNESVTFYLQN
LYQTDIYFCKIEVMYPPPYLDNEKSNGTIIHVKGKHLCPSPLFPGPSKP
FWVLVWGGVLACYSLLVTVAFIIFWVRSKRSRLLHSDYMNMTPRRPGPT
RKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 31 or amino acids 153 to 179 of SEQ ID NO: 31) comprises at least a portion of the sequence set forth in SEQ ID NO: 32 as provided below.

(SEQ ID NO: 32)
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCCTTTTGGGTGCTGGTGGTGGTTGGTGGAGTC
CTGGCTTGCTATAGCTTGCTAGTAACAGTGGCCTTTATTATTTTCTGGG
TGAGGAGTAAGAGGAGCAGGCTCCTGCACAGTGACTACATGAACATGAC
TCCCCGCCGCCCCGGGCCCACCCGCAAGCATTACCAGCCCTATGCCCCA
CCACGCGACTTCGCAGCCTATCGCTCC

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 33 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 33 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 33.

(SEQ ID NO: 33)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPWKSGDKPSLSARYV

In certain embodiments, the transmembrane domain comprises a CD8 polypeptide comprising amino acids having the sequence set forth in SEQ ID NO: 34 as provided below:

(SEQ ID NO: 34)
PTTTPAPRPPTPAPTIASQPLSLRPEACRPAAGGAVHTRGLDFACDIYI
WAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 34) comprises nucleic acids having the sequence set forth in SEQ ID NO: 35 as provided below:

(SEQ ID NO: 35)
CCCACCACGACGCCAGCGCCGCGACCACCAACCCCGGCGCCCACGAT
CGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACA
TCTGGGCGCCCCTGGCCGGGACTTGTGGGGTCCTTCTCCTGTCACTGGT
TATCACCCTTTACTGCAAC

In certain embodiments, the transmembrane domain comprises a NKG2D polypeptide. The NKG2D polypeptide can have an amino acid sequence that is at least about 85%, about 90%, about 95%, about 96%, about 97%, about 98%, about 99% or about 100%) homologous to SEQ ID NO: 36 (homology herein may be determined using standard software such as BLAST or FASTA) as provided below, or fragments thereof, and/or may optionally comprise up to one or up to two or up to three conservative amino acid substitutions. In certain embodiments, the NKG2D polypeptide can have an amino acid sequence that is a consecutive portion of SEQ ID NO: 36 which is at least 20, or at least 30, or at least 40, or at least 50, and up to 216 amino acids in length. Additionally or alternatively, in various embodiments, the NKG2D polypeptide has an amino acid sequence of amino acids 1 to 216, or 50 to 100 of SEQ ID NO: 36.

(SEQ ID NO: 36)
MGWIRGRRSRHSWEMSEFHNYNLDLKKSDFSTRWQKQRCPVVKSKCR
ENASPFFFCCFIAVAMGIRFIIMVTIWSAVFLNSLFNQEVQIPLTESYC
GPCPKNWICYKNNCYQFFDESKNWYESQASCMSQNASLLKVYSKEDQDL
LKLVKSYHWMGLVHIPTNGSWQWEDGSILSPNLLTIIEMQKGDCALYAS
SFKGYIENCSTPNTYICMQRTV

In certain embodiments, the transmembrane domain comprises a NKG2D polypeptide comprising amino acids having the sequence set forth in SEQ ID NO: 4 as provided below:

(SEQ ID NO: 4)
PFFFCCFIAVAMGIRFIIMVT

In accordance with the presently disclosed subject matter, a “NKG2D nucleic acid molecule” refers to a polynucleotide encoding a NKG2D polypeptide. In certain embodiments, the NKG2D nucleic acid molecule encoding the NKG2D polypeptide comprised in the transmembrane domain of the presently disclosed CAR (SEQ ID NO: 4) comprises nucleic acids having the sequence set forth in SEQ ID NO: 13 as provided below.

(SEQ ID NO: 13)
CCATTTTTTTTCTGCTGCTTCATCGCTGTAGCCATGGGAATCCGTTTC
ATTATTATGGTAACA

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 31), a portion of a CD8 polypeptide (e.g., SEQ ID NO: 34), a portion of a NKG2D polypeptide (e.g., SEQ ID NO: 4), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., an ILTCk). CD3ζ comprises 3 ITAMs, and transmits an activation signal to the cell (e.g., a cell of the lymphoid lineage, e.g., an ILTCk) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 53), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 54 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 54. In certain embodiments, the CD3ζ polypeptide has an amino acid sequence of amino acids 52 to 164 of SEQ ID NO: 54.

SEQ ID NO: 54 is provided below:

(SEQ ID NO: 54)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the CD3ζ polypeptide has the amino acid sequence set forth in SEQ ID NO: 55, which is provided below:

(SEQ ID NO: 55)
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 55) comprised in the intracellular domain of the presently disclosed CAR comprises a nucleotide sequence as set forth in SEQ ID NO: 56 as provided below.

(SEQ ID NO: 56)
AGAGTGAAGTTCAGCAGGAGCGCAGAGCCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCG

In certain embodiments, the CD3ζ polypeptide has the amino acid sequence set forth in SEQ ID NO: 8, which is provided below:

(SEQ ID NO: 8)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

The CD3ζ nucleic acid molecule encoding the CD3ζ polypeptide (SEQ ID NO: 8) comprised in the intracellular domain of the presently disclosed CAR comprises a nucleotide sequence as set forth in SEQ ID NO: 17:

(SEQ ID NO: 17)
AGAGTGAAGTTCAGCAGGAGCGCAGACGCCCCCGCGTACCAGCAGG
GCCAGAACCAGCTCTATAACGAGCTCAATCTAGGACGAAGAGAGGAGTA
CGATGTTTTGGACAAGAGACGTGGCCGGGACCCTGAGATGGGGGGAAAG
CCGAGAAGGAAGAACCCTCAGGAAGGCCTGTACAATGAACTGCAGAAAG
ATAAGATGGCGGAGGCCTACAGTGAGATTGGGATGAAAGGCGAGCGCCG
GAGGGGCAAGGGGCACGATGGCCTTTACCAGGGTCTCAGTACAGCCACC
AAGGACACCTACGACGCCCTTCACATGCAGGCCCTGCCCCCTCGC

In certain non-limiting embodiments, an intracellular domain of the CAR can comprise a truncated cytoplasmic domain of IL-2RPA. In certain embodiments, the IL2RbΔ polypeptide has an amino acid sequence of (SEQ ID NO: 7):

(SEQ ID NO: 7)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV

The IL-2RPΔ nucleic acid molecule encoding the IL-2RPΔ polypeptide (SEQ ID NO: 7) comprised in the intracellular domain of the presently disclosed CAR comprises a nucleotide sequence as set forth in SEQ ID NO: 16:

(SEQ ID NO: 16)
AACTGCAGGAACACCGGGCCATGGCTGAAGAAGGTCCTGAAGTGTA
ACACCCCAGACCCCTCGAAGTTCTTTTCCCAGCTGAGCTCAGAGCATGG
AGGAGACGTCCAGAAGTGGCTCTCTTCGCCCTTCCCCTCATCGTCCTTC
AGCCCTGGCGGCCTGGCACCTGAGATCTCGCCACTAGAAGTGCTGGAGA
GGGACAAGGTGACGCAGCTGCTCCCCCTGAACACTGATGCCTACTTGTC
CCTCCAAGAACTCCAGGGTCAGGACCCAACTCACTTGGTG

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a NKG2C polypeptide, a NKG2D polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, a 2B4 polypeptide, a BTLA polypeptide, a NKG2C polypeptide, a NKG2D polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ immune cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 57) or fragments thereof, and/or may optionally comprise up to one or up to two or up to three conservative amino acid substitutions.

SEQ ID NO: 57 is provided below:

(SEQ ID NO: 57)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
GTKERDWCGPSPADLSPGASSVTPPAPAREPGHSPQIISFFLALTSTAL
LFLLFFLTLRFSWKRGRKKLLYIFKQPFMRPVQTTQEEDGCSCRFPEEE
EGGCEL

In certain embodiments, the 4-1BB co-stimulatory domain has the amino acid sequence set forth in SEQ ID NO: 58, which is provided below: KRGRKKLLYIFKQPFMRPVQTTQEEDGCSCRFPEEEEGGCEL (SEQ ID NO: 58)

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 58) comprised in the intracellular domain of the presently disclosed CAR comprises a nucleotide sequence as set forth in SEQ ID NO: 59 as provided below.

(SEQ ID NO: 59)
AAACGGGGCAGAAAGAAGCTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 60), or fragments thereof, and/or may optionally comprise up to one or up to two or up to three conservative amino acid substitutions.

SEQ ID NO: 60 is provided below:

(SEQ ID NO: 60)
MCVGARRLGRGPCAALLLLGLGLSTVTGLHCVGDTYPSNDRCCHECRPG
NGMVSRCSRSQNTVCRPCGPGFYNDWSSKPCKPCTWCNLRSGSERKQLC
TATQDTVCRCRAGTQPLDSYKPGVDCAPCPPGHFSPGDNQACKPWTNCT
LAGKHTLQPASNSSDAICEDRDPPATQPQETQGPPARPITVQPTEAWPR
TSQGPSTRPVEVPGGRAVAAILGLGLVLGLLGPLAILLALYLLRRDQRL
PPDAHKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 61) or fragments thereof, and/or may optionally comprise up to one or up to two or up to three conservative amino acid substitutions.

SEQ ID NO: 61 is provided below:

(SEQ ID NO: 61)
MKSGLWYFFLFCLRIKVLTGEINGSANYEMFIFHNGGVQILCKYPDIVQ
QFKMQLLKGGQILCDLTKTKGSGNTVSIKSLKFCHSQLSNNSVSFFLYN
LDHSHANYYFCNLSIFDPPPFKVTLTGGYLHIYESQLCCQLKFWLPIGC
AAFVWCILGCILICWLTKKKYSSSVHDPNGEYMFMRATAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM (SEQ ID NO: 62) motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref No.: P16410.3 (SEQ ID NO: 63) (homology herein may be determined using standard software such as BLAST or FASTA) or fragments thereof, and/or may optionally comprise up to one or up to two or up to three conservative amino acid substitutions.

SEQ ID NO: 63 is provided below:

(SEQ ID NO: 63)
MACLGFQRHKAQLNLATRTWPCTLLFFLLFIPVFCKAMHVAQPAWLASS
RGIASFVCEYASPGKATEVRVTVLRQADSQVTEVCAATYMMGNELTFLD
DSICTGTSSGNQLTIQGLRAMDTGLYICKVELMYPPPYYLGIGNGTQIY
VIDPEPCPDSDFLLWILAAVSSGLFFYSFLLTAVSLSKMLKKRSPLTTG
VYVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 64) or fragments thereof, and/or may optionally comprise up to one or up to two or up to three conservative amino acid substitutions.

SEQ ID NO: 64 is provided below:

(SEQ ID NO: 64)
MQIPQAPWPVVWAVLQLGWRPGWFLDSPDRPWNPPTFSPALLWTEGDNA
TFTCSFSNTSESFVLNWYRMSPSNQTDKLAAFPEDRSQPGQDCRFRVTQ
LPNGRDFHMSVVRARRNDSGTYLCGAISLAPKAQIKESLRAELRVTERR
AEVPTAHPSPSPRPAGQFQTLVVGWGGLLGSLVLLVWVLAVICSRAARG
ELDFQWREKTPEPPVPCVPEQTEYATIVFPSGMGTSSPARRGSADGPRS
ATIGARRTGQPLKEDPSAVPVFSVDYGQPLRPEDGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 65) or fragments thereof, and/or may optionally comprise up to one or up to two or up to three conservative amino acid substitutions.

SEQ ID NO: 65 is provided below:

(SEQ ID NO: 65)
MWEAQFLGLLFLQPLWVAPVKPLQPGAEVPWWAQEGAPAQLPCSPTIPL
QDLSLLRRAGVTWQHQPDSGPPAAAPGHPLAPGPHPAAPSSWGPRPRRY
TVLSVGPGGLRSGRLPLQPRVQLDERGRQRGDFSLWLRPARRADAGEYR
AAVHLRDRALSCRLRLRLGQASMTASPPGSLRASDWVILNCSFSRPDRP
ASVHWFRNRGQGRVPVRESPHHHLAESFLFLPQVSPMDSGPWGCILTYR
DGFNVSIMYNLTVLGLEPPTPLTVYAGAGSRVGLPCRLPAGVGTRSFLT
AKWTPPGGGPDLLVTGDNGDFTLRLEDVSQAQAGTYTCHIHLQEQQLNA
TVTLAIITVTPKSFGSPGSLGKLLCEVTPVSGQERFVWSSLDTPSQRSF
SGPWLEAQEAQLLSQPWQCQLYQGERLLGAAVYFTELSSPGAQRSGRAP
GALPAGHLLLFLILGVLSLLLLVTGAFGFHLWRRQWRPRRFSALEQGIH
PPQAQSKIEELEQEPEPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 66) or fragments thereof, and/or may optionally comprise up to one or up to two or up to three conservative amino acid substitutions.

SEQ ID NO: 66 is provided below:

(SEQ ID NO: 66)
MLGQWTLILLLLLKVYQGKGCQGSADHWSISGVPLQLQPNSIQTKVDSI
AWKKLLPSQNGFHHILKWENGSLPSNTSNDRFSFIVKNLSLLIKAAQQQ
DSGLYCLEVTSISGKVQTATFQVFVFESLLPDKVEKPRLQGQGKILDRG
RCQVALSCLVSRDGNVSYAWYRGSKLIQTAGNLTYLDEEVDINGTHTYT
CNVSNPVSWESHTLNLTQDCQNAHQEFRFWPFLVIIVILSALFLGTLAC
FCVWRRKRKEKQSETSPKEFLTIYEDVKDLKTRRNHEQEQTFPGGGSTI
YSMIQSQSSAPTSQEPAYTLYSLIQPSRKSGSRKRNHSPSFNSTIYEVI
GKSQPKAQNPARLSRKELENFDVYS

In some embodiments, the 2B4 polypeptide comprises the amino acid sequence of SEQ ID NO: 5:

(SEQ ID NO: 5)
WRRKRKEKQSETSPKEFLTIYEDVKDLKTRRNHEQEQTFPGGGSTIYSM
IQSQSSAPTSQEPAYTLYSLIQPSRKSGSRKRNHSPSFNSTIYEVIGKS
QPKAQNPARLSRKELENFDVYS

The 2B4 nucleic acid molecule encoding the 2B4 polypeptide (SEQ ID NO: 5) comprised in the intracellular domain of the presently disclosed CAR comprises a nucleotide sequence as set forth in SEQ ID NO: 14:

(SEQ ID NO: 14)
TGGAGGAGAAAGAGGAAGGAGAAGCAGTCAGAGACCAGTCCCAAGG
AATTTTTGACAATTTACGAAGATGTCAAGGATCTGAAAACCAGGAGAAA
TCACGAGCAGGAGCAGACTTTTCCTGGAGGGGGGAGCACCATCTACTCT
ATGATCCAGTCCCAGTCTTCTGCTCCCACGTCACAAGAACCTGCATATA
CATTATATTCATTAATTCAGCCTTCCAGGAAGTCTGGATCCAGGAAGAG
GAACCACAGCCCTTCCTTCAATAGCACTATCTATGAAGTGATTGGAAAG
AGTCAACCTAAAGCCCAGAACCCTGCTCGATTGAGCCGCAAAGAGCTGG
AGAACTTTGATGTTTATTCC

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO: 67) or fragments thereof, and/or may optionally comprise up to one or up to two or up to three conservative amino acid substitutions.

SEQ ID NO: 67 is provided below:

(SEQ ID NO: 67)
MKTLPAMLGTGKLFWVFFLIPYLDIWNIHGKESCDVQLYIKRQSEHSIL
AGDPFELECPVKYCANRPHVTWCKLNGTTCVKLEDRQTSWKEEKNISFF
ILHFEPVLPNDNGSYRCSANFQSNLIESHSTTLYVTDVKSASERPSKDE
MASRPWLLYRLLPLGGLPLLITTCFCLFCCLRRHQGKQNELSDTAGREI
NLVDAHLKSEQTEASTRQNSQVLLSETGIYDNDPDLCFRMQEGSEVYSN
PCLEENKPGIVYASLNHSVIGPNSRLARNVKEAPTEYASICVRS

In accordance with the presently disclosed subject matter, a “BTLA nucleic acid molecule” refers to a polynucleotide encoding a BTLA polypeptide.

In some embodiments, the co-stimulatory domain may comprise a DAP10 polypeptide including the amino acid sequence of LCARPRRSPAQEDGKVYINMPGRG (SEQ ID NO: 6). The nucleic acid molecule encoding the DAP10 polypeptide (SEQ ID NO: 6) comprised in the co-stimulatory domain of the presently disclosed CAR comprises a nucleotide sequence as set forth in SEQ ID NO: 15:

(SEQ ID NO: 15)
CTGTGCGCACGCCCACGCCGCAGCCCCGCCCAAGAAGATGGCAAAGTCT
ACATCAACATGCCAGGCAGGGGC

Additionally or alternatively, in some embodiments, the heterologous nucleic acid encoding the IL-15 or STAT5B gene and/or any CAR disclosed herein is operably linked to an inducible promoter. In some embodiments, the heterologous nucleic acid encoding the IL-15 or STAT5B gene and/or any CAR disclosed herein is operably linked to a constitutive promoter.

In some embodiments, the inducible promoter is a synthetic Notch promoter that is activatable in a CAR⁺ ILTCk cell, where the intracellular domain of the CAR contains a transcriptional regulator that is released from the membrane when engagement of the CAR with the target antigen/polypeptide induces intramembrane proteolysis (see, e.g., Morsut et al., Cell 164(4): 780-791 (2016). Accordingly, further transcription of the target-antigen-specific CAR is induced upon binding of the engineered ILTCk with the antigen/polypeptide.

The presently disclosed subject matter also provides isolated nucleic acid molecules encoding the IL-15 or STAT5B gene and/or any CAR construct described herein or a functional portion thereof. In certain embodiments, the CAR construct comprises (a) an antigen binding fragment (e.g., an anti-target-antigen scFv or a fragment) that specifically binds to a target antigen, (b) a transmembrane domain comprising a CD8 polypeptide, CD28 polypeptide, or a NKG2D polypeptide, and (c) an intracellular domain comprising a truncated cytoplasmic domain of IL-2RβΔ, or a CD3ζ polypeptide, and optionally one or more of a co-stimulatory signaling region disclosed herein, a P2A self-cleaving peptide, and/or a reporter or selection marker provided herein. The at least one co-stimulatory signaling region can include a CD28 polypeptide, a CD3ζ polypeptide, a 4-1BB polypeptide, an OX40 polypeptide, an ICOS polypeptide, a DAP-10 (HCST) polypeptide, a PD-1 polypeptide, a CTLA-4 polypeptide, a LAG-3 polypeptide, a 2B4 polypeptide, a BTLA polypeptide, a NKG2C polypeptide, a NKG2D polypeptide, a synthetic peptide (not based on a protein associated with the immune response), or a combination thereof. In some embodiments, the at least one co-stimulatory signaling region includes a DAP-10 polypeptide and a 2B4 polypeptide.

In certain embodiments, the isolated nucleic acid molecule encodes an IL-15 or STAT5B gene and any CAR construct disclosed herein (such as a CAR that specifically binds a target antigen) comprising an antigen binding fragment (e.g., a scFv) that specifically binds to a target antigen/polypeptide, fused to a synthetic Notch transmembrane domain and an intracellular cleavable transcription factor. In certain embodiments, the present disclosure provides an isolated nucleic acid molecule encoding an IL-15 or STAT5B gene and a receptor (such as a CAR that specifically binds a target antigen) that is inducible by release of the transcription factor of a synthetic Notch system.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the parent CAR. For example, functional portions encompass the portions, parts or fragments of a target-antigen-specific CAR that retains the ability to recognize a target cell, to treat cancer, to a similar, same, or even a higher extent as the parent CAR. In certain embodiments, an isolated nucleic acid molecule encoding a functional portion of a target-antigen-specific CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

The presently disclosed subject matter provides engineered immune cells expressing an IL-15 or STAT5B and a target-antigen-specific CAR or other ligand that comprises an extracellular antigen-binding domain, a transmembrane domain and an intracellular domain, where the extracellular antigen-binding domain specifically binds a target antigen/polypeptide. In certain embodiments, ILTCks can be transduced with a presently disclosed CAR constructs such that the cells express the CAR. The presently disclosed subject matter also provides methods of using such cells for the treatment of cancer.

The engineered ILTCks of the presently disclosed subject matter can express an IL-15 or STAT5B and/or an extracellular antigen binding domain (e.g., an anti-target-antigen scFv, an anti-target-antigen Fab that is optionally crosslinked, an anti-target-antigen F(ab)₂ or a fragment) that specifically binds to a target antigen, for the treatment of cancer.

In some embodiments, the higher the expression level of IL-15 or STAT5B and/or the CAR in an engineered ILTCk, the greater cytotoxicity and cytokine production the engineered ILTCk exhibits.

Additionally or alternatively, the cytotoxicity and cytokine production of a presently disclosed engineered ILTCk are proportional to the expression level of target antigen in a target tissue or a target cell. Additionally or alternatively, the cytotoxicity and cytokine production of a presently disclosed engineered ILTCk are proportional to the expression level of IL-15, STAT5B or CAR in the ILTCk. For example, the higher the expression level, the greater cytotoxicity and cytokine production the engineered ILTCk exhibits.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. In certain embodiments, the CCR comprises (a) an extracellular antigen-binding domain that binds to an antigen different than the first target antigen, (b) a transmembrane domain, and (c) a co-stimulatory signaling region that comprises at least one co-stimulatory molecule, including, but not limited to, CD28, 4-1BB, OX40, ICOS, PD-1, CTLA-4, LAG-3, 2B4, NKG2C, NKG2D, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second target antigen is selected so that expression of both the first target antigen and the second target antigen is restricted to the targeted cells (e.g., cancerous cells). Similar to a CAR, the extracellular antigen-binding domain can be an scFv, a Fab, a F(ab)₂; or a fusion protein with a heterologous sequence to form the extracellular antigen-binding domain.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells. The truncated CAR functions as an adhesion molecule that enhances the avidity of a presently disclosed CAR, especially, one that has a low binding affinity to a target antigen, thereby improving the efficacy of the presently disclosed CAR or engineered ILTCk comprising the same. In certain embodiments, the truncated CAR comprises an extracellular antigen-binding domain that binds to a target antigen, and a transmembrane domain comprising a CD8 polypeptide. A presently disclosed ILTCk comprises or is transduced to express a presently disclosed CAR targeting a target antigen and a truncated CAR targeting a target antigen. In certain embodiments, the targeted cells are solid tumor cells.

### Polynucleotides, Polypeptides and Analogs

Also included in the presently disclosed subject matter are polynucleotides encoding IL-15, STAT5B, or any CAR disclosed herein (e.g., CAR including a truncated cytoplasmic domain of IL-2RPA) and their corresponding polypeptides or fragments that may be modified in ways that enhance their anti-tumor activity when expressed in an engineered ILTCk. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications can occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (p) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or can result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein. Such analogs are administered according to methods of the presently disclosed subject matter. Such analogs can exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered ILTCk. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding IL-15, STAT5B, or any CAR disclosed herein (e.g., CAR including a truncated cytoplasmic domain of IL-2RPA) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

In some embodiments, a nucleic acid as disclosed herein further comprises a regulatory sequence directing the expression of IL-15, STAT5B, or any CAR disclosed herein (e.g., CAR including a truncated cytoplasmic domain of IL-2RβΔ). In further embodiments, the nucleic acid comprises a single regulatory sequence directing the expression of both the IL-15 or STAT5B gene, and any CAR disclosed herein (e.g., CAR including a truncated cytoplasmic domain of IL-2RβΔ). In other embodiments, the nucleic acid comprises a first regulatory sequence directing the expression of the IL-15 or STAT5B gene and a second regulatory sequence directing the expression of the CAR. In other embodiments, the first regulatory sequence is the same as the second regulatory sequence. In some embodiments, the first regulatory sequence is different from the second regulatory sequence.

### Vectors

Many expression vectors are available and known to those of skill in the art and can be used for nonendogenous expression of IL-15, STAT5B, or any CAR disclosed herein (e.g., CAR including a truncated cytoplasmic domain of IL-2RβΔ). The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag or a myc tag, hemagglutinin tag or a tag for purification, for example, a GST fusion, and a sequence for directing protein secretion and/or membrane association.

Heterologous expression of IL-15, STAT5B, or any CAR disclosed herein (e.g., CAR including a truncated cytoplasmic domain of IL-2RPA) can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

In some embodiments, the expression vector is a plasmid, a cosmid, a bacmid, a bacterial artificial chromosome (BAC), a yeast artificial chromosome (YAC), a viral vector, or a retroviral vector.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered ILTCks can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding IL-15, STAT5B, or any CAR disclosed herein (e.g., CAR including a truncated cytoplasmic domain of IL-2RβΔ) can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide ILTCks expressing IL-15, STAT5B, or any CAR disclosed herein (e.g., CAR including a truncated cytoplasmic domain of IL-2RβΔ), a retroviral vector can be employed for transduction. However, any other suitable viral vector or non-viral delivery system can be used for genetic modification of cells. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller et al. (1985)Mol. Cell. Biol. 5:431-437); PA317 (Miller et al. (1986) Mol. Cell. Biol. 6:2895-2902); and CRIP (Danos et al. (1988) Proc. Natl. Acad. Sci. USA 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secrete a cytokine (e.g., 4-1BBL and/or IL-12) in an engineered ILTCk. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing IL-15, STAT5B, or any CAR disclosed herein (e.g., CAR including a truncated cytoplasmic domain of IL-2RβΔ) is a retroviral vector, e.g., an oncoretroviral vector. In some instances, the retroviral vector is a SFG retroviral vector or murine stem cell virus (MSCV) retroviral vector. In certain non-limiting embodiments, the vector expressing an IL-15, STAT5B, or CAR (e.g., CAR including a truncated cytoplasmic domain of IL-2RβΔ) nucleic acid sequence is a lentiviral vector. In certain non-limiting embodiments, the vector expressing an IL-15, STAT5B, or CAR (e.g., CAR including a truncated cytoplasmic domain of IL-2RβΔ) nucleic acid sequence is a transposon vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

In some embodiments, a vector as disclosed herein further comprises a regulatory sequence directing the expression of IL-15, STAT5B, or any CAR disclosed herein. In further embodiments, the vector comprises a single regulatory sequence directing the expression of both of the IL-15 or STAT5B, and any CAR disclosed herein. In other embodiments, the vector comprises a first regulatory sequence directing the expression of the IL-15 or STAT5B and a second regulatory sequence directing the expression of any CAR disclosed herein. In other embodiments, the first regulatory sequence is the same as the second regulatory sequence. In some embodiments, the first regulatory sequence is different from the second regulatory sequence.

### Engineered ILTCks of the Present Technology

The presently disclosed subject matter provides engineered ILTCks that exhibit heterologous expression of IL-15, or STAT5B. Additionally or alternatively, in some embodiments, the engineered ILTCks may further comprise an engineered receptor (e.g., a CAR) or other ligand that comprises an extracellular antigen-binding domain, a transmembrane domain and an intracellular domain, where the extracellular antigen-binding domain specifically binds a tumor antigen, including a tumor receptor or ligand. In another aspect, the present disclosure provides engineered ILTCks comprising a CAR including a truncated cytoplasmic domain of IL-2RβΔ. In certain embodiments, ILTCks can be transduced with a vector comprising nucleic acid sequences that encode IL-15, STAT5B, or any CAR disclosed herein (e.g., CAR including a truncated cytoplasmic domain of IL-2RβΔ).

Examples of tumor antigens include, but are not limited to, 5T4, alpha 501-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pml/RARa, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAPI, B7H3, Polysialic Acid, OX40, OX40-ligand, and peptide MHC complexes (with peptides derived from TP53, KRAS, MYC, EBNA1-6, PRAME, tyronsinase, MAGEAI-A6, pmel17, LMP2, or WT1).

The presently disclosed subject matter also provides methods of using such engineered ILTCks for the treatment of a tumor. The engineered ILTCks of the presently disclosed subject matter can express non-endogenous levels of IL-15, STAT5B, or any CAR disclosed herein (e.g., CAR including a truncated cytoplasmic domain of IL-2RβΔ) for the treatment of cancer, e.g., for treatment of tumor. Such engineered ILTCks can be administered to a subject (e.g., a human subject) in need thereof for the treatment of cancer.

The presently disclosed engineered ILTCks of the present technology may further include at least one recombinant or exogenous co-stimulatory ligand. For example, the presently disclosed engineered ILTCks can be further transduced with at least one co-stimulatory ligand, such that the engineered ILTCks co-expresses or is induced to co-express IL-15, STAT5B, or any CAR disclosed herein (e.g., CAR including a truncated cytoplasmic domain of IL-2RβΔ) and the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTa), lymphotoxin-beta 0-TO), CD257/B cell-activating factor (B AFF)/Bly s/THANK/Tall-1, glucocorticoid-induced TNF Receptor ligand (GITRL), and TF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered ILTCk comprises one recombinant co-stimulatory ligand (e.g., 4-1BBL). In certain embodiments, the engineered ILTCk comprises two recombinant co-stimulatory ligands (e.g., 4-1BBL and CD80). CARs comprising at least one co-stimulatory ligand are described in U.S. Pat. No. 8,389,282, which is incorporated by reference in its entirety.

Furthermore, the presently disclosed engineered ILTCks can further comprise at least one exogenous cytokine. For example, a presently disclosed engineered ILTCk can be further transduced with at least one cytokine, such that the engineered ILTCks secrete the at least one cytokine as well as express IL-15, STAT5B, or any CAR disclosed herein (e.g., CAR including a truncated cytoplasmic domain of IL-2RβΔ). Additionally or alternatively, in certain embodiments, the at least one cytokine is selected from the group consisting of IL-2, IL-4, IL-7, IL-12, IL-15, IL-18, IL-21 and IL-23.

The engineered ILTCks can be generated from peripheral donor lymphocytes (see Examples described herein). The engineered ILTCks can be autologous, non-autologous (e.g., allogeneic), or derived in vitro from engineered progenitor or stem cells.

In certain embodiments, the presently disclosed engineered ILTCk expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a heterologous nucleic acid encoding IL-15, STAT5B, or any CAR disclosed herein (e.g., CAR including a truncated cytoplasmic domain of IL-2RβΔ).

For example, the higher the non-endogenous levels of IL-15, STAT5B, or any CAR disclosed herein (e.g., CAR including a truncated cytoplasmic domain of IL-2RβΔ) in an engineered ILTCk, the greater cytotoxicity and/or cytokine production the engineered ILTCk exhibits.

Additionally, or alternatively, the cytotoxicity and cytokine production of a presently disclosed engineered ILTCk are proportional to the expression level of IL-15, STAT5B, or any CAR disclosed herein (e.g., CAR including a truncated cytoplasmic domain of IL-2RβΔ) in the ILTCk.

The unpurified source of ILTCks can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the engineered ILTCks comprise one or more additional modifications. For example, in some embodiments, the engineered ILTCks comprise and express (is transduced to express) a chimeric co-stimulatory receptor (CCR). CCR is described in Krause et al. (1998) J. Exp. Med. 188(4):619-626, and US20020018783, the contents of which are incorporated by reference in their entireties. CCRs mimic co-stimulatory signals, but do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell.

In some embodiments, the engineered ILTCks are further modified to suppress expression of one or more genes. In some embodiments, the engineered ILTCks are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered ILTCks are modified to result in disruption or inhibition of PD-1, PDL-1, Tim-3 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Administration

The engineered ILTCks of the presently disclosed subject matter can be provided systemically or directly to a subject for treating cancer. In certain embodiments, the engineered ILTCks described herein are directly injected into an organ of interest. Additionally or alternatively, the engineered ILTCks of the present technology are provided indirectly to the organ of interest, for example, by administration into the circulatory system (e.g., the tumor vasculature) or into the tissue of interest (e.g., solid tumor). Expansion and differentiation agents can be provided prior to, during or after administration of cells and compositions to increase production of the engineered ILTCks either in vitro or in vivo.

Engineered ILTCks of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered ILTCks can comprise a purified population of cells. Those skilled in the art can readily determine the percentage of engineered ILTCks in a cell population using various well-known methods, such as fluorescence activated cell sorting (FACS). The ranges of purity in cell populations comprising engineered ILTCks can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered ILTCks can be introduced by injection, catheter, or the like. If desired, factors can also be included, including, but not limited to, interleukins, e.g., IL-2, IL-4, IL-7, IL-12, IL-15, IL-18, IL-21, IL-23, as well as the other interleukins, the colony stimulating factors, such as G-, M- and GM-CSF, interferons, e.g., γ-interferon.

In certain embodiments, compositions of the presently disclosed subject matter comprise any and all embodiments of the engineered ILTCks of the present technology with a pharmaceutically acceptable carrier. Administration can be autologous or non-autologous. For example, the engineered ILTCks of the present technology and compositions comprising the same can be obtained from one subject, and administered to the same subject or a different, compatible subject. Peripheral blood derived ILTCks of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising any and all embodiments of the engineered ILTCks disclosed herein), it can be formulated in a unit dosage injectable form (solution, suspension, emulsion).

### Formulations

The engineered ILTCks of the present technology and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising the engineered ILTCks of the present technology, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered ILTCks of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered ILTCks as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered ILTCks of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered ILTCks of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered ILTCks of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered ILTCks are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the ILTCks of the Present Technology

In one aspect, the present disclosure provides a method for selecting a subject suffering from cancer for adoptive cell therapy with killer innate-like T cells (ILTCks) comprising (a) identifying a cancer subject harboring tumors with low mutation burden; and (b) administering to the cancer subject an effective amount of killer innate-like T cells (ILTCks). In some embodiments, the tumors are refractory to immune checkpoint blockade therapy or adoptive cell therapy with CD8⁺ T cell therapy. Additionally or alternatively, in some embodiments, the tumors with low mutation burden are identified via next-generation sequencing using a tumor biopsy sample or cell-free DNA (cfDNA) sample obtained from the cancer subject.

In one aspect, the present disclosure provides a method for treating cancer or inhibiting tumor growth in a subject in need thereof comprising administering to the subject an effective amount of killer innate-like T cells (ILTCks). The ILTCks may be native ILTCks, genetically engineered ILTCks, or a combination thereof. In some embodiments, the ILTCks are isolated from a donor subject and/or expanded ex vivo or in vitro.

In any and all embodiments of the methods disclosed herein, the methods further comprise separately, simultaneously, or sequentially administering an effective amount of IL-15 to the subject. The IL-15 may be administered to the subject prior to, during, or subsequent to administration of the ILTCks.

In any and all embodiments of the methods disclosed herein, the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Additionally or alternatively, in some embodiments of the methods disclosed herein, the ILTCks are administered pleurally, intravenously, subcutaneously, intranodally, intratumorally, intrathecally, intrapleurally or intraperitoneally. In certain embodiments, the methods of the present technology further comprise sequentially, separately, or simultaneously administering to the subject an additional cancer therapy. Examples of additional cancer therapy include, but are not limited to chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

Also disclosed herein are methods for preparing killer innate-like T cells (ILTCks) for adoptive cell therapy comprising isolating a population of immune cells from a donor subject, and collecting FCER1G⁺ cells from the isolated population of immune cells. The FCER1G⁺ cells may comprise FCER1G⁺CD122⁺ cells, FCER1G⁺NK1.1⁺GzmB^{+/-} cells, FCER1G⁺NK1.1⁻GzmB⁻ cells, and/or FCER1G⁺PD-1⁺ cells.

For treatment, the amount of the engineered ILTCks provided herein administered is an amount effective in producing the desired effect, for example, treatment of a cancer or one or more symptoms of a cancer. An effective amount can be provided in one or a series of administrations of the engineered ILTCks provided herein. An effective amount can be provided in a bolus or by continuous perfusion. For adoptive immunotherapy using ILTCks, cell doses in the range of about 10⁶ to about 10¹⁰ may be infused. Lower doses of the engineered ILTCks may be administered, e.g., about 10⁴ to about 108.

The engineered ILTCks of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered ILTCks and the compositions comprising thereof are intravenously administered to the subject in need. Methods for administering cells for adoptive cell therapies, including, for example, donor lymphocyte infusion and engineered immune cell therapies, and regimens for administration are known in the art and can be employed for administration of the engineered ILTCks provided herein.

For example, the presently disclosed subject matter provides methods of reducing tumor burden in a subject. In one non-limiting example, the method of reducing tumor burden comprises administering an effective amount of the presently disclosed engineered ILTCks to the subject and administering a suitable antibody targeted to the tumor, thereby inducing tumor cell death in the subject. In some embodiments, the engineered ILTCks and the antibody are administered at different times. For example, in some embodiments, the engineered ILTCks are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or more than 48 hours after the administration of the engineered ILTCks of the present technology.

The presently disclosed subject matter provides various methods of using any and all embodiments of the engineered ILTCks provided herein. For example, the presently disclosed subject matter provides methods of reducing tumor burden in a subject. In one non-limiting example, the method of reducing tumor burden comprises administering an effective amount of the presently disclosed engineered ILTCks to the subject, thereby inducing tumor cell death in the subject.

The presently disclosed engineered ILTCks can reduce the number of tumor cells, reduce tumor size, and/or eradicate the tumor in the subject. In certain embodiments, the method of reducing tumor burden comprises administering an effective amount of engineered ILTCks of the present technology to the subject, thereby inducing tumor cell death in the subject. Non-limiting examples of suitable tumors include adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject with cancer (e.g., a tumor). In one non-limiting example, the method of increasing or lengthening survival of a subject with cancer (e.g., a tumor) comprises administering an effective amount of the presently disclosed engineered ILTCks to the subject, thereby increasing or lengthening survival of the subject. The presently disclosed subject matter further provides methods for treating or preventing cancer (e.g., a tumor) in a subject, comprising administering the presently disclosed engineered ILTCks to the subject. Also provided herein are methods for treating of inhibiting tumor growth or metastasis in a subject comprising contacting a tumor cell with an effective amount of any of the engineered ILTCks provided herein.

Cancers whose growth may be inhibited using the engineered ILTCks of the presently disclosed subject matter include cancers typically responsive to immunotherapy. Non-limiting examples of cancers for treatment include breast cancer, endometrial cancer, ovarian cancer, colon cancer, lung cancer, stomach cancer, prostate cancer, renal cancer, pancreatic cancer, brain cancer, acute lymphoblastic leukemia (ALL), chronic lymphocytic leukemia (CLL), acute myeloid leukemia (AML), and metastases thereof.

Additionally, the presently disclosed subject matter provides methods of increasing immune-activating cytokine production in response to a cancer cell in a subject in need thereof. In one non-limiting example, the method comprises administering the presently disclosed engineered ILTCks to the subject. The immune-activating cytokine (which is also referred to herein as a cytokine) can be granulocyte macrophage colony stimulating factor (GM-CSF), IFNα, IFN-β, IFN-γ, TNFα, IL-2, IL-4, IL-7, IL-12, IL-15, IL-18, IL-21, IL-23, and combinations thereof.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

Another group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the engineered ILTCks described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the engineered ILTCks to avert or minimize the risks of immunological complications, or when healthy tissues express the same target antigens as the tumor cells, leading to outcomes similar to GvHD. Modification of the engineered ILTCks can include engineering a suicide gene into the engineered ILTCks. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable ILTCk elimination by administering anti-EGFR monoclonal antibody (e.g., cetuximab). The suicide gene can be included within the vector comprising nucleic acids encoding IL-15, STAT5B, or any CAR disclosed herein (e.g., CAR including a truncated cytoplasmic domain of IL-2RβΔ). A presently disclosed engineered ILTCk incorporated with a suicide gene can be pre-emptively eliminated at a given time point post ILTCk infusion, or eradicated at the earliest signs of toxicity.

In another aspect, the present disclosure provides a method of preparing immune cells for adoptive cell therapy (ACT) comprising: (a) isolating killer innate-like T cells (ILTCks) from a donor subject, (b) transducing the ILTCks with a nucleic acid encoding IL-15 or STAT5B or an expression vector comprising said nucleic acid, and (c) administering the transduced ILTCks to a recipient subject. In certain embodiments, the nucleic acid encodes the amino acid sequence of SEQ ID NO: 19, SEQ ID NO: 20, SEQ ID NO: 9 or SEQ ID NO: 23. Additionally or alternatively, in some embodiments, the method further comprises transducing the ILTCks with a nucleic acid encoding a chimeric antigen receptor (CAR) that binds to a tumor antigen. Also disclosed herein is a method of preparing immune cells for adoptive cell therapy (ACT) comprising: (a) isolating killer innate-like T cells (ILTCks) from a donor subject, (b) transducing the ILTCks with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains, and (c) administering the transduced ILTCks to a recipient subject. In some embodiments of the ACT methods described herein, the donor subject and the recipient subject are the same or different. Additionally or alternatively, in some embodiments of the ACT methods described herein, isolating ILTCks from the donor subject comprises isolating a population of immune cells from the donor subject, and collecting FCER1G⁺ cells from the isolated population of immune cells.

### Combination Therapy

The naturally-occurring or engineered ILTCks of the present technology may be employed in conjunction with other therapeutic agents useful in the treatment of cancers. For example, any and all embodiments of the ILTCks described herein may be separately, sequentially or simultaneously administered with at least one additional cancer therapy. Examples of additional cancer therapy include chemotherapeutic agents, immune checkpoint inhibitors, monoclonal antibodies that specifically target tumor antigens, immune activating agents (e.g., interferons, interleukins, cytokines), oncolytic virus therapy and cancer vaccines. In some embodiments, the additional cancer therapy is selected from among a chemotherapy, a radiation therapy, an immunotherapy, a monoclonal antibody, an anti-cancer nucleic acid, an anti-cancer protein, an anti-cancer virus or microorganism, a cytokine, or any combination thereof.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

In some embodiments, the methods further comprise sequentially, separately, or simultaneously administering an immunotherapy to the subject. In some embodiments, the immunotherapy regulates immune checkpoints. In further embodiments, the immunotherapy comprises, or consists essentially of, or yet further consists of an immune checkpoint inhibitor, such as a Cytotoxic T-Lymphocyte Associated Protein 4 (CTLA-4) inhibitor, or a Programmed Cell Death 1 (PD-1) inhibitor, or a Programmed Death Ligand 1 (PD-L1) inhibitor. In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA-4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-O11 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

In some embodiments, the methods further comprise sequentially, separately, or simultaneously administering a cytokine to the subject. In some embodiments, the cytokine is administered prior to, during, or subsequent to administration of the one or more ILTCks. In some embodiments, the cytokine is selected from the group consisting of interferon α, interferon 3, interferon 7, complement C5a, IL-2, TNFα, CD40L, IL-12, IL-23, IL-15, IL-18, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3C_{L}1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The methods for treating cancer may further comprise sequentially, separately, or simultaneously administering to the subject at least one chemotherapeutic agent, optionally selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11.

### Kits

The presently disclosed subject matter provides kits for the treatment or prevention of a disease, such as cancer. In certain embodiments, the kit may be used in the manufacture of a therapeutic or prophylactic composition containing an effective amount of engineered ILTCks. In some embodiments, the kits include a non-endogenous expression vector comprising a heterologous mammalian IL-15 or STAT5B nucleic acid. Additionally or alternatively, in some embodiments, the kit comprises a vector comprising any and all embodiments of the CARs disclosed herein, or other cell-surface ligand that binds to a target antigen, such as a tumor antigen. In certain embodiments, the CAR comprises (i) an extracellular antigen binding domain comprising a single chain variable fragment (scFv) that binds to the tumor antigen; (ii) a transmembrane domain; and (iii) an intracellular domain comprising a truncated cytoplasmic domain of IL-2RβΔ and one or more co-stimulatory domains.

In some embodiments, the vector comprising the heterologous mammalian IL-15 or STAT5B nucleic acid and the vector comprising the CAR or cell-surface ligand that binds to a target antigen are the same. In other embodiments, the vector comprising the heterologous mammalian IL-15 or STAT5B nucleic acid and the vector comprising the CAR or cell-surface ligand that binds to a target antigen are distinct.

In some embodiments, the kit comprises a sterile container which contains the kit components; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for preparing ILTCks for adoptive cell therapy, and methods of using ILTCks to treat cancer in a subject in need thereof. The instructions will generally include information about the use of the composition for the treatment or prevention of cancer. In other embodiments, the instructions include at least one of the following: description of the therapeutic agent; dosage schedule and administration for treatment or prevention of cancer or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

The at least one engineered ILTCk of the present technology may be provided to the subject in the form of a syringe or autoinjection pen containing a sterile, liquid formulation or lyophilized preparation (e.g., Kivitz et al., Clin. Ther. 28:1619-29 (2006)).

A device capable of delivering the engineered ILTCks of the present technology through an administrative route may be included in the kit. Examples of such devices include syringes (for parenteral administration) or inhalation devices.

The kit components may be packaged together or separated into two or more containers. In some embodiments, the containers may be vials that contain sterile, lyophilized formulations that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

### EXAMPLES

### Example 1: Materials and Methods

Human samples. All research activities were preapproved by the Institutional Ethics Review Board at Memorial Sloan Kettering Cancer Center and individuals were required to provide written informed consent to participate in the study. All histological diagnoses were confirmed by expert colorectal pathologists. Tumor and adjacent normal colon samples were directly obtained from the operating room. Tissue samples were placed in separate labeled containers containing Roswell Park Memorial Institute (RPMI) medium and transported in regular ice to the laboratory within 1 hour. The human tissues were briefly cut into pieces and subjected to enzymatic digestion using Human Tumor Dissociation Kit (130-095-929, Miltenyi Biotec) in combination with gentleMACS™ Octo Dissociator with Heaters with preset program 37C_h_TDK_2 according to the manufacturer's protocol. The resulting cell suspension was filtered through a 70 μm cell strainer and washed with PBS and centrifuge at 1600 rpm for 6 minutes. Cell pellet was further resuspended in RPMI with 2% FBS. Cells were centrifuged on a Ficoll gradient and then washed with PBS before use.

Mice. C57BL/6J (B6), B6.SJL-Ptprc^{a}Pepc^{b}/BoyJ (CD45.1), FVB/N-Tg(MMTV-PyVT)^{634Mul}/J (PyMT), C57BL/6-Tg(TRAMP)^{8247Ng}/J (TRAMP), B6.129S7-Rag1^{tm1Mom}/J (Rag1⁻), B6.129P2-B2m^{tm1Unc}/J (B2m⁻), B6.129S(C)-Batf3^{tm1Kmm}/J (Baft3⁻), B6(Cg)-Il5^{tm1.2Nsl}/J (Il15⁻), B6(Cg)-Irf8^{tm1.1Hm}/J (Ir8^{fl}), B6.Cg-Tg(Itgax-cre)^{1-1Reiz}/J (Itgax-Cre), B6.FVB-Tg(Rorc-cre)^{1Litt}/J (Rorc-Cre), B6.Cg-Tg(S100A8-cre,-EGFP)^{1llw}/J (S100a8-Cre), B6(SJL)-Zbtb16tm1.1(EGFP/cre)^{Aben}/J (Zbtb16-Cre), C57BL/6N-Fgd5^{tm3(cre/ERT2)Djr}/J (Fgd5-CreER), B6.Cg-Ndor1Tg(UBC-cre/ERT2)^{1Ejb}/1J (Ubc-CreER), B6J.129(Cg)-Gt(ROSA)26Sor^{tm1.1(CAG-cas9*,-EGFP)Fezh}/J (Rosa26^{Cas9}), B6;129S6-Gt(ROSA)26Sor^{tm9(CAG-tdTomato)Hze}/J (Rosa26^{LSL-tdTomato}), and B6.129X1-Gt(ROSA)26Sortm1(EYFP)^{Cos}/J (Rosa26^{LSL-YFP}) were purchased from the Jackson Laboratory. The H2-k1^{-/-}H2-d1^{-/-}, Rosa26^{LSL-Stat5b-CA}, and Il15^{2A-eGFP} mice were previously described^{35,36,37}. Il15^{fl} mice with exon 5 flanked by two loxP sites were generated, and kindly provided to us by K. Ikuta. All mice were backcrossed to the C57BL/6 background and maintained under specific pathogen-free conditions. Animal experimentation was conducted in accordance with procedures approved by the Institutional Animal Care and Use Committee of Memorial Sloan Kettering Cancer Center.

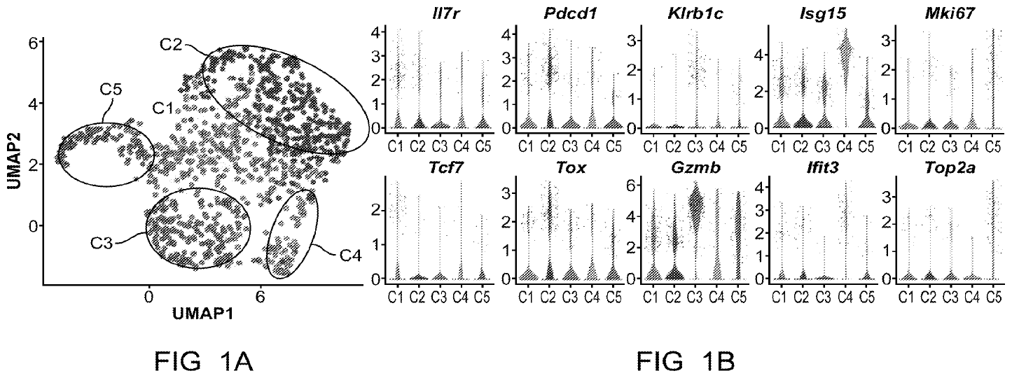

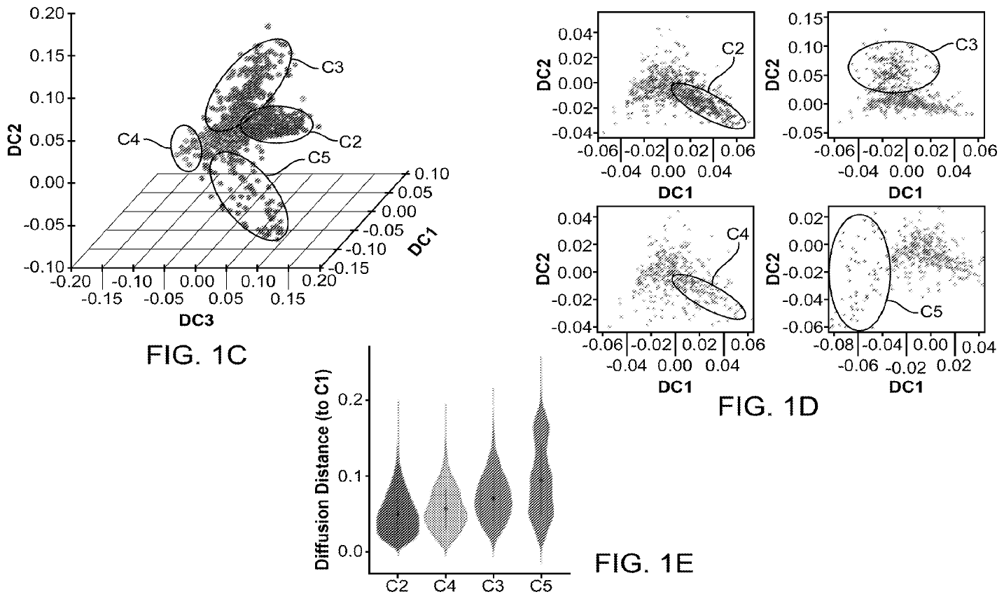

Single-cell RNA sequencing analysis. FASTQ files for single cell RNA-sequencing of tumor-infiltrating TCRβ⁺CD8⁺ T cells were demultiplexed and aligned to the mm10 genome using Cell Ranger v3.0.2 (10x genomics). The resulting count matrix of cells by genes, which contains the number of UMIs for each gene associated with each cell, was filtered as follows. First, cells with greater than 20% mitochondrial gene expression were removed. All mitochondrial and ribosomal genes were then filtered out, as well as the noncoding RNAs Neat1 and Malat1. Genes with log mean expression <2.5 were also filtered out. UMI counts were then log and library size-normalized with a scale factor of 10,000 according to the standard Seurat v2.4 pipeline^{38,39} in the R statistical environment (https://www.R-project.org/v3.6.1; “Action of the Toes”). Dimensionality reduction via PCA was then conducted on the normalized count matrix, and the top 10 principal components were used for Louvain clustering analysis using the FindClusters( ) function at resolution 0.6. A two-dimensional embedding of the data was generated using UMAP with the top 10 principal components as input, using the RunUMAP( ) function. Differential gene expression analysis was conducted using the FindMarkers( ) function, with “Wilcox” specified as the statistical test. Significantly differentially expressed genes were computed for each cluster as genes differentially expressed in each cluster versus all others at FDR P <0.05. Heatmaps for significantly differentially expressed genes between clusters were generated using the pheatmap package (Kolde, R. pheatmap: https://cran.rproject.org/web/packages/pheatmap/index.html) in R. Diffusion map analyses were conducted using the destiny package⁴⁰ in R. To visualize clusters presented in FIG. 1, we first computed a diffusion map embedding with all 5 clusters together using the destiny package⁴⁰ in R, with k=60. To quantify potential lineage transitions between the naïve cluster C1 and all other clusters, we calculated the pairwise diffusion distance⁴¹ between each cell in C1 and all other clusters. For pseudotime analysis, we first regressed out the effect of cell cycle genes, and used Monocle^{42,43,44} to estimate lineage branching using differentially expressed genes between clusters at FDR P<0.001. All other single cell datasets, including the one from Zhang et al¹² were analyzed as described above. The αβILTCk signature was constructed by performing a differential expression analysis between the αβILTCk cluster C3 and all other clusters presented in FIGS. 1A-1E, taking genes with FDR P<0.05 and log FC>0 (i.e. upregulated in αβILTCk vs all others). This signature was then applied to other datasets using the addModuleScore( ) function in Seurat.

Bulk RNA-sequencing analysis. FASTQ files for bulk RNA-sequencing of thymic αβILTCk/IEL progenitors and CD8 single positive cells (two biological replicates each) were first mapped to the mm10 genome using HiSat2 v2.0.5 (Ref⁴⁵). The genomic index along with the list of splice sites and exons were created by HiSat2 using the genome assembly GRCm38.p5 from ENSEMBL together with the comprehensive gene annotation for GRCm38.p5 (Release M13) from GENCODE⁴⁶. Gene-level counts were computed using Rsubread⁴⁷ (options isPairedEnd=TRUE, requireBothEndsMapped=TRUE, minOverlap=80, countChimericFragments=FALSE). DESeq2 (Ref⁴⁸) was used to perform differential expression analysis on the resulting count matrix. Genes were considered significantly differentially expressed at FDR P<0.05. Pathway analyses were conducted using the enrichGO( ) function in the R package clusterProfiler⁴⁹ to assess enrichment in pathways curated in Gene Ontology.

Generation of single-cell TCR-sequencing library. Amplification of TCRα and TCRβ chains from single sorted cells was performed by iRepertoire Inc. (Huntsville, AL, USA). Briefly, RT-PCR1 was performed with nested, multiplex primers covering both TCRα and TCRβ loci, and including partial Illumina adaptors. Included on the reverse primer was an in-line 6-nt barcode, which served as a plate identifier so that multiple 96-well plates could be multiplexed in the same sequencing flow cell. After RT-PCR1, the first round PCR1 products were rescued using SPRISelect Beads (Beckman Coulter). A second PCR was performed with dual-indexed primers that complete the sequencing adaptors introduced during PCR1 and provide plate positional information for the sequenced products. Sequencing was performed using the Illumina MiSeq v2 500-cycle kit with 250 paired-end reads.

Data processing of single-cell TCR-seq library. Raw data were demultiplexed by Illumina dual indices and the 6-nt internal plate barcode information for each well of the 96-well PCR plates. Data were analyzed using the previously described iRmap program^{50, 51}. Reads were trimmed according to their base qualities with a 2-base sliding window. If either quality value in this window is lower than 20, the sequence stretch from the window to the 3′ end is trimmed from the original read. Trimmed pair-end reads were joined together through overlapping alignment with a modified Needleman-Wunsch algorithm. If paired forward and reverse reads in the overlapping region were not perfectly matched, both forward and reverse reads were thrown out without further consideration. The merged reads were mapped using a Smith-Waterman algorithm to germline V, D, J and C reference sequences downloaded from the IMGT web site⁵². To define the CDR3 region, the position of CDR3 boundaries of reference sequences from the IMGT database were migrated onto reads through mapping results, and the resulting CDR3 regions were extracted and translated into amino acids. The data for each chain of the receptor pair begins from within the beginning of framework (FR) 2 and extends to the beginning of the C-region (including the isotype). Information for FR1 and CDR3 were inferred from alignments for downstream cloning and expression.

Immune cell isolation from murine tissues. Tumor-infiltrating immune cells were isolated from murine mammary tumors as previously described⁵³. Briefly, tumor tissues were minced with a razor blade then digested in 280 U/mL Collagenase Type 3 (Worthington Biochemical) and 4 g/mL DNase I (Sigma) in HBSS at 37° C. for one hour and 15 minutes with periodic vortex every 20 minutes. Digested tissues were passed through 70 m filters and pelleted. Cells were resuspended in 40% Percoll (Sigma) and layered above 60% Percoll. Sample was centrifuged at 1,900 g at 4° C. for 30 minutes without brake. Cells at interface were collected, stained and analyzed by flow cytometry or sorting. Isolation of small intestinal intraepithelial lymphocytes has been previously described⁵¹. Briefly, small intestine between distal duodenum and proximal ileum was opened longitudinally and intestinal content was cleaned by washing in ice-cold PBS, followed by incubation in PBS/10 mM EDTA/1 mM Dithiothreitol solution at 37° C. for 15 minutes with vigorous shaking. Tissues were passed through 100 μm filters and pelleted. Cells were resuspended in 40% Percoll and centrifuged at 1,200 g at room temperature for 20 minutes. Cell pellets were collected, stained and analyzed by flow cytometry.

Flow cytometry and cell sorting. For flow cytometry experiments, cells were incubated with 2.4G2 mAb to block FcγR binding, DAPI (4,6-diamidino-2-phenylindole; Sigma) or Aqua Live/Dead (Thermo Fisher Scientific) for the exclusion of dead cells and were stained with panels of antibodies for 30 minutes on ice. Granzyme B staining was carried out using the intracellular transcription factor buffer set from BD Pharmingen. All samples were acquired with an LSRII (BD) or LSR Fortessa (BD), and analyzed with FlowJo software version 9.6.2 (Tree Star). Cell sorting was performed with a FACSAria II (BD) using a 100 μm nozzle. Tumor-infiltrating NK1.1⁺ αβILTCks and PD-1⁺ T cells were sorted as CD45⁺TCRγδ-TCRβ⁺CD4⁻CD8α⁺PD-1⁻NK1.1⁺ and CD45⁺TCRγδ⁻TCRβ⁺CD4⁻ CD8α⁺PD-1⁺NK1.1⁻, respectively. Thymic αβILTCk/IEL progenitors and CD8 single positive cells were sorted as CD4^{-/dull}CD8α^{-/dull}CD1d/PBS-57⁻CD25⁻ TCRβ⁺CD122⁺CD5^{hi}PD-1⁺NK1.1⁻ and TCRβ⁺CD4⁻CD8α⁺, respectively. For sorting of LSK cells, total bone marrow cells were incubated with CD 117 MicroBeads (Miltenyi Biotec) according to the manufacturer's instruction, followed by positive selection with an LS column (Miltenyi Biotec) prior to staining with monoclonal antibodies. LSK cells were sorted as Lineage⁻ (CD3ε⁻B220⁻Gr1⁻CD11b⁻Ter119⁻) CD117⁺Sca1⁺. For single cell TCR-sequencing experiments, respective populations were single cell sorted into V-bottom 96-well plates (iRepertoire) which were flash-frozen and stored at −80° C. prior to library construction.

TCR cloning and reporter assay. Gene Blocks (Genewiz, NJ) containing the coding regions for the leader, variable and constant domains of paired TCRα and TCRβ joined by a 2A peptide-encoding sequence were inserted into MSCV-IRES-mCherry or MSCV-IRES-GFP retroviral vectors, which contain an MSCV2.2 backbone with an IRES-fluorescence protein cassette to facilitate identification of cells expressing the construct. For TCR constructs used in the ‘swapping’ experiments, a silent G to T mutation in the sequence encoding the constant region of the TCRβ chain was introduced to prevent Cas9 targeting. Production of retrovirus has been previously described⁵⁵. A mixture of CD8⁺ and CD8⁻ TCR reporter cell lines (58α⁻β⁻, gift from K. Murphy) were transduced with retroviruses expressing TCR pairs isolated from tumor-infiltrating αβILTCks and PD-1⁺ T cells. Successful pairing and expression of transduced TCRs were verified by detection of surface TCRβ in mCherry⁺ cells with flow cytometry. TCR-expressing reporter cell lines were co-cultured with sorted primary cancer cells from PyMT mice or a cortical thymic epithelial cell line, ANV-41-2 (gift from MRM. van den Brink) in the presence of 10 ng/ml of IFN-γ (Peprotech) for 24 hours, followed by analysis of GFP expression in mCherry⁺ cells.

Generation of Tap1^{-/-} and B2m^{-/-} PyMT cell lines. To generate the PyMT early passage (PyMT-EP) cell line, a piece of PyMT tumor was subjected to enzymatic digestion 280 U/mL Collagenase Type 3 and 4 g/mL DNase I in HBSS at 37° C. for 30 minutes. The cell mixture was passed through a 100 m cell strainer and were plated as a polyclonal population in a 10-cm dish in DMEM/F12 (Thermo Fisher Scientific) supplemented with 10% FBS, 1× Insulin-Transferrin-Selenium-Ethanolamine (Thermo Fisher Scientific), 100 U penicillin, 0.1 mg/ml streptomycin and 1× Normocin (Invivogen). Medium was changed regularly, and EpCAM-expressing cells were subsequently sorted.

To generate PyMT-EP cell lines lacking Tap1 or B2m, sequences encoding sgRNAs targeting Tap1 (5′-ACGGCCGTGCATGTGTCCCA (SEQ ID NO: 379)) or B2m (5′-CCGAGCCCAAGACCGTCTAG (SEQ ID NO: 380)) were cloned into a lentiCRISPR v2 plasmid (gift from F. Zhang, Addgene plasmid #52961). Packaging and production of lentivirus was described previously⁵⁶. Following lentiviral transduction, PyMT-EP cells were selected on media containing 1 μg/mL of puromycin for four days. H-2Db-deficient cells were subsequently sorted.

Generation of TCR ‘retrogenic’ bone marrow chimeras. TCR ‘retrogenic’ bone marrow chimeras were generated as previously described with slight modifications^{57, 58}. LSK cells were sorted from bone marrows of Rag1^{-/-} mice, maintained in DMEM-F12 supplemented with 15% FBS, 10 mM HEPES, 50 ng/μL SCF (Peprotech), and 50 ng/μL TPO (Peprotech) for 24 hours prior to two consecutive transductions with TCR-IRES-GFP-expressing retroviruses. A mixture of 10⁵ transduced Rag1^{-/-} LSK cells and 3×10⁶ total bone marrow cells from Rag1+^{+/+} mice were co-transferred intravenously into a lethally irradiated (9.5 Gy) 8- to 10-week old PyMT recipient mouse via retroorbital injection. Bone marrow chimeras were analyzed when palpable tumors appeared between 8 and 12 weeks post reconstitution. Donor T cells expressing a monoclonal TCR were gated as GFP⁺TCRβ⁺ whereas those expressing a polyclonal TCR repertoire were identified as GFP⁻TCRβ⁺.

For fate mapping experiments using the Zbtb16-CreRosa26^{LSL-YFP} mice, bone marrow chimeras were generated as previously described⁵⁹ to circumvent basal labeling by the Zbtb16-Cre allele. Briefly, CD45.2⁺ YFP⁻ LSKs were sorted and intravenously transferred to lethally irradiated CD45.1⁺CD45.2⁺ PyMT mice.

TCR ‘swapping’ and adoptive transfers in vivo. The TCR-targeting retroviral plasmid was constructed using pTGMP (gift from S. Lowe, Addgene plasmid #32716) as a backbone. Briefly, a sequence encoding the mCherry fluorescent protein was inserted downstream of the PGK promoter. The GFP-miR30 cassette was replaced with three consecutive hU6 promoter driven sgRNA units targeting the TCR loci. Viral supernatants were prepared by transfection of PlatE packaging cells⁵⁵ with TransIT 293 (Mirus Bio). For retroviral transduction of activated T cells, CD8⁺ T cells from the lymph nodes of CD45.1⁺CD45.2⁺ Rosa26^{Cas9/Cas9} mice were isolated using the EasySep™ Mouse CD8⁺ T Cell Isolation Kit (StemCell Technologies) and activated with 0.1 μg/mL anti-CD38 (145-2C11, Biolegend) and 1 μg/ml anti-CD28 (37.51, BioXCell) in multiwell tissue culture plates coated with goat antibody to Armenian hamster IgG (Jackson ImmunoResearch), followed by ‘spin-inoculation’ with retroviruses expressing TCR-targeting sgRNAs and TCRs of interest. Transduced T cells were ‘rested’ in the presence of 10 ng/mL IL-7 (Peprotech). T cells expressing the TCRs of interest in place of endogenously rearranged TCRs were sorted as mCherry⁺GFP⁺TCRβ⁺ and adoptively transferred into CD45.2⁺ tumor-bearing PyMT recipient mice via intravenous injection, followed by analysis seven days later. For adoptive transfer of thymic αβILTCk/IEL progenitors, approximately 200,000 or 600,000 cells sorted from pooled thymi from five to ten mice at four week of age were transferred intravenously into a Rag1^{-/-} or Rag1^{+/+} PyMT recipient, respectively. [0359]αβILTCk-based adoptive cellular transfer. For transfer into lymphocyte-deficient hosts, approximately 200,000 thymic αβILTCk/IEL progenitors sorted from Ubc-CreERRosa26^{Statsb-CA+} or Ubc-CreERRosa26^{+/+} mice were transferred intravenously into Rag1^{-/-} PyMT recipients. For transfer into lympho-replete hosts, approximately 1,000,000 thymic αβILTCk/IEL progenitors and CD8 single positive cells from CD45.1⁺CD45.2⁺ Ubc-CreERRosa26^{Statsb-C/+} mice were sorted and transferred intravenously into sublethally irradiated CD45.2⁺ PyMT recipients. All recipients subsequently receive 5 mg Tamoxifen via oral gavage one week post transfer.

Tumor measurement. Mammary tumors in female PyMT mice were measured weekly with a caliper. Tumor burden was calculated using the formula (L×W²)×(π/6), in which L is length W is width. Total tumor burden was calculated by summing up individual tumor volumes of each mouse with an end-point defined when total burden reached 3,000 mm³ or one tumor reached 2,000 mm³.

Immunofluorescence microscopy. Fresh human tumors were fixed in Periodate-Lysine-Paraformaldehyde (PLP) for 16-24 h, 30% sucrose for 24 hours, then frozen in OCT. Tissue was sectioned at 20 μm thickness, blocked and permeabilized in buffer containing 0.1 M Tris, 1% BSA, 1% FBS, 0.3% Triton-X100, 2% normal mouse/rat/goat serum for 30 minutes and stained with anti-IL-15 (MAB647, R&D), AF594-conjugated anti-CHD1 (DECMA-1, Biolegend) overnight at 4° C. Slides were washed and stained with secondary AF488-conjugated goat anti-mouse antibody (A32723, Invitrogen) and DAPI. Images were taken on confocal microscope using 3 color channels. IL-15 levels were scored accordingly as the average percentage of IL-15 staining positivity among CDH1 positive cells from 10 field of view per sample. 0: no staining. 1: 1-20%, 2: 21-40%, 3: 41-60%, 4: 61-80%, 5: 81-100% positivity.

Antibodies. The following antibodies were used in Flow cytometry: Alexa Fluor (AF) 488-conjugated anti-CD31 (MEC13.3, Biolegend), FITC-conjugated anti-PD-1 (29F.1A12, Biolegend), anti-CD80 (H35-17.2, BD Pharmingen), anti-FCER1G (FCABS400F, Mili-Mark), PE-conjugated anti-PD-1 (29F.1A12, Biolegend), anti-CD122 (TM-β1, BD Pharmingen), anti-CD 117 (2B8, Biolegend), anti-H-2D^{b} (KH95, Biolegend), PerCP-Cy5.5-conjugated anti-CD4 (GK1.5, Biolegend), PerCP-eFluor710-conjugated anti-TCRγδ (eBioGL3, Thermo Fisher Scientific), PE-Cy7-conjugated anti-CD8α (53-6.7, Biolegend), anti-PD-1 (29F.1A12, Biolegend), anti-NK1.1 (PK136, Biolegend), anti-EpCAM (G8.8, Biolegend), A647-conjugated anti-TCRβ (H57-597, Biolegend), anti-Sca1 (D7, Biolegend), anti-Granzyme B (GB11, Thermo Fisher Scientific), APC-conjugated anti-CD25 (3C7, Biolegend), anti-H-2K^{b} (AF6-88.5, Biolegend), APC-Cy7-conjugated anti-CD45.2 (104, Biolegend), anti-TCRβ (H57-597, Biolegend), APC-R700-conjugated anti-HLA-DR (G46-6, BD), Pacific Blue-conjugated anti-B220 (RA3-6B2, Biolegend), anti-CD19 (6D5, Biolegend), anti-CD38 (145-2C11, Biolegend), anti-CD11b (M1/70, Biolegend), anti-Gr1 (RB6-8C5, Biolegend), anti-Ter119 (Ter119, Biolegend), anti-CD8α (53-6.7, Biolegend), Brilliant Violet (BV) 510-conjugated anti-CD45 (30-F11, BD Pharmingen), BV 605-conjugated anti-CD5 (53-7.3, BD Pharmingen), anti-CD45 (30-F11, BD Pharmingen), BV 650-conjugated anti-CD45.1 (A20, Biolegend), anti-CD45 (30-F11, BD Pharmingen), BV 711-conjugated anti-CD49a (Ha31/8, BD Pharmingen), BV 786-conjugated anti-CD103 (2E7, BD Pharmingen), Biotinylated anti-CD38 (17A2, Biolegend), anti-Gr1 (RB6-8C5, Biolegend), anti-B220 (RA3-6B2, Biolegend), anti-Ter119 (Ter119, Biolegend), anti-CD11b (M1/70, Biolegend). Secondary reagents: Streptavidin-conjugated BV 421 (Biolegend). AF647-conjugated CD1d/PBS-57 tetramer was supplied by the NIH Tetramer Core Facility.

Statistical analysis. All statistical measurements are displayed as mean±S.D. P-values were calculated with an unpaired two-tailed Student's t-test for two-group comparisons, by one-way ANOVA for multi-group comparisons with the Turkey post hoc test, and by Kolmogorov-Smirnov test for comparison of frequency distributions using Prims 8 software. To calculate differences in diffusion distance between clusters defined in FIG. 1A, we first estimated the diffusion map for all clusters (FIG. 1C). The diffusion distance was defined as the pairwise Euclidean distance between each point in three-dimensional diffusion map space (FIG. 1C) and points in other clusters. Statistical differences between the distribution of diffusion distances for each pair of clusters were calculated using a two-sided Wilcoxon test. Adjusted P-values of <0.05 were considered significant.

### Example 2: ILTCks Display a Distinct Transcriptome

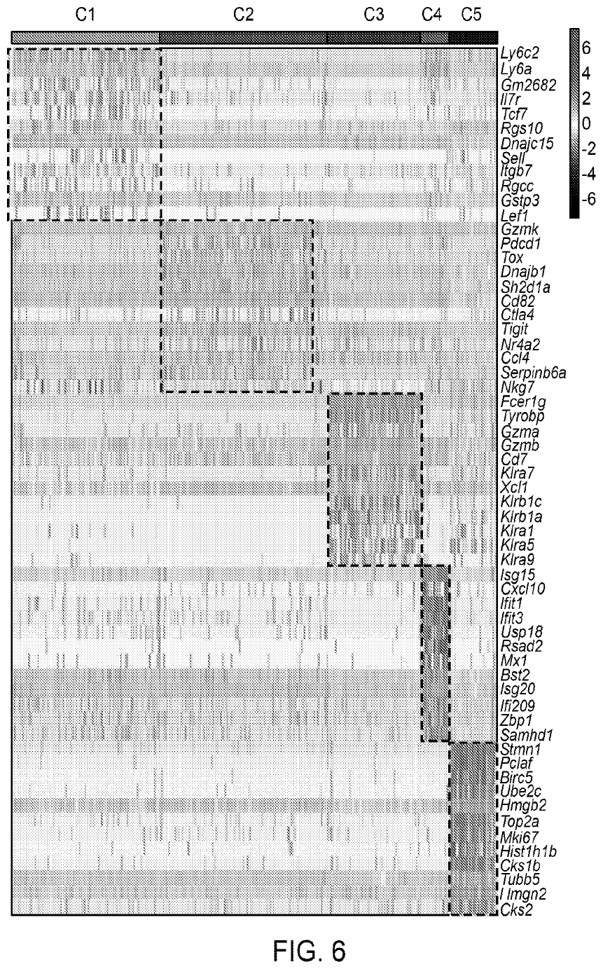

To investigate the heterogeneity among tumor-infiltrating CD8⁺ T cells, we purified CD45⁺TCRβ⁺CD8α⁺ cells from breast tumor tissues of MMTV-PyMT (PyMT) mice for single cell RNA-sequencing (scRNA-seq) analysis. After sequencing quality control, 1,015 cells were further analyzed and a total of 10,670 genes were used for dimension reduction by uniform manifold approximation and projection (UMAP) analysis (FIG. 6). Distinct aggregation pattern of CD8α⁺ T cells was observed, where cells roughly segregated into five clusters (FIG. 1A). Cluster 1 (C1) cells had relatively high expression of naïve T cell markers, such as Il7r and Tcf7 (FIG. 1B), which likely represent recently activated T cells. Markers associated with T cell dysfunction, including Pdcd1 and Tox, were highly expressed in C2 cells (FIG. 1B). This subset is likely enriched for exhausted T cells, which are abundantly found in human tumors^{9,10,11,12}. C3 cells were characterized by high expression of Gzmb and Klrb1c encoding the natural killer (NK) cell receptor NK1.1 (FIG. 1B), which represent the αβ T cell receptor (TCR) lineage killer innate-like T cells (αβ ILTCks) that we recently identified¹³. C4 cells expressed high levels of type I interferon stimulated genes (ISGs), including Isg15 and Ifit3 (Ref¹⁴) (FIG. 1B). C5 cells markedly upregulated Mki67 and Top2a, suggesting their proliferative state (FIG. 1B). Thus, tumor-infiltrating CD8α⁺ T cells in PyMT mice exhibit diverse differentiation and proliferation states.

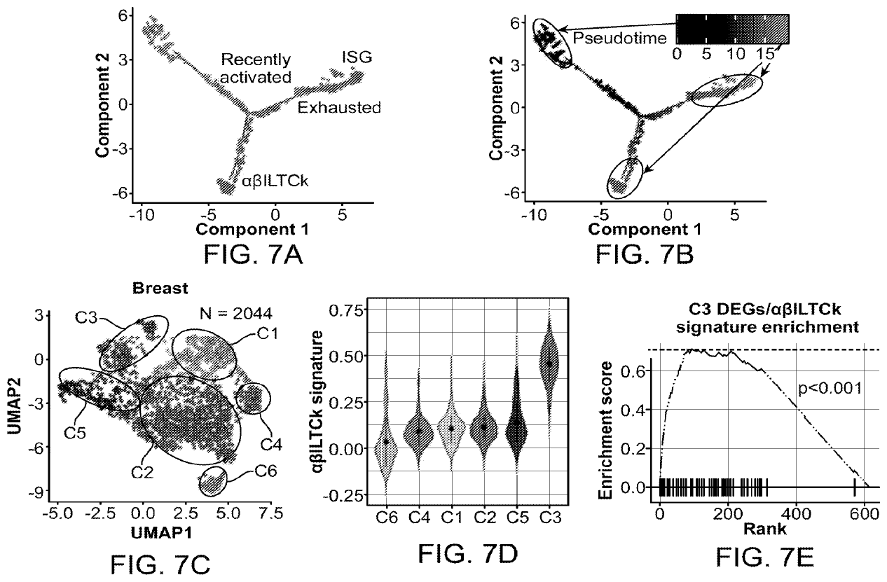

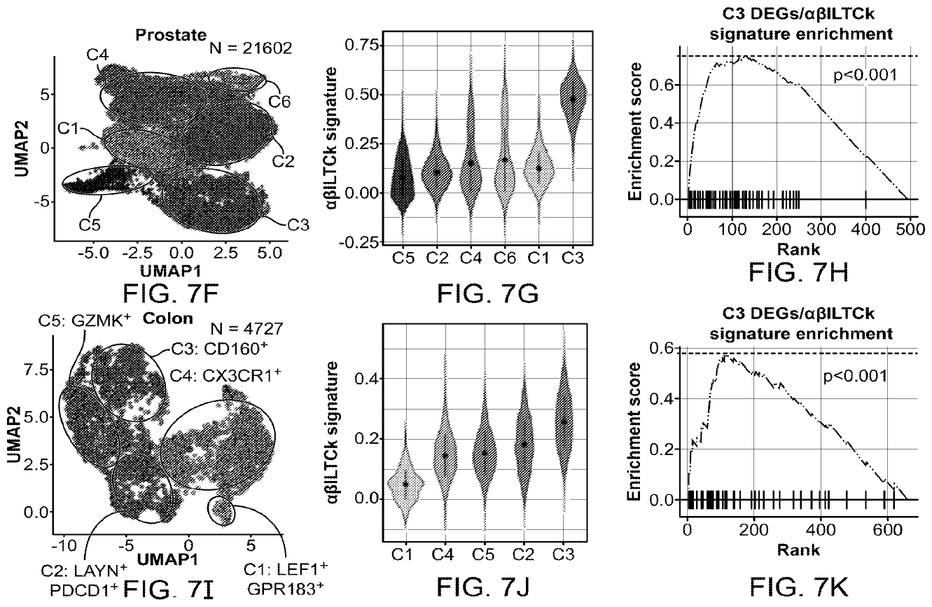

Under the classic paradigm, naïve CD8⁺ T cells upon priming by antigen presenting cells (APCs) traffic to the site of insults where further interactions with local APCs, stroma, or parenchyma continuously mold activated CD8⁺ T cells into their terminally differentiated states³. Such a multifactorial mode of cell differentiation predicts that the gene expression programs of progenitors and progenies would occupy overlapping transcriptional space. To infer the potential differentiation trajectories, we computed a three-dimensional diffusion map embedding using all five clusters (FIG. 1C). Using recently activated cells (C1) as reference, examination of cells along the diffusion components (DC) 1 and 2 revealed substantial mixing with exhausted (C2) and to a lesser extent ISG-expressing (C4) cells (FIGS. 1D-1E), suggesting direct and continuous differentiation pathways. This finding is consistent with the prevailing evidence that chronic stimulation drives phenotypic change of activated T cells⁵. In contrast, αβILTCks (C3) and proliferative (C5) cells were more distantly segregated from C1 (FIGS. 1D-1E). Such a pattern of cell state change could be due to a singular integrated cellular checkpoint, which probably occurred for C5 cells programmed to enter the cell cycle. Nevertheless, after correcting for ‘cell-cycle effects,’ the hypothetical trajectory of recently activated-to-αβILTCk transition remained disparate from the well-established recently activated-to-exhausted T cell differentiation pathway (FIGS. 7A-7B). Thus, C1 cells either give rise to C3 cells through a unique differentiation pathway, or are not their progenitors.

Despite its nebulous origin, a C3-like cluster of tumor-infiltrating CD8α T cells highly expressing the αβILTCk signature transcriptional program was reproducibly present in PyMT breast tumors (FIGS. 7C-7E) as well as in an oncogene-driven prostate cancer model (FIGS. 7F-7H). Notably, the αβILTCk gene signature was also highly enriched in the previously identified NKR⁺CD160⁺ innate-like T cells¹² in human colorectal carcinoma (FIGS. 7I-7K), together suggesting that the αβILTCk differentiation program represents an evolutionarily conserved tumor-elicited immune response.

### Example 3: ILTCks Recognize Unmutated Tumor Antigen

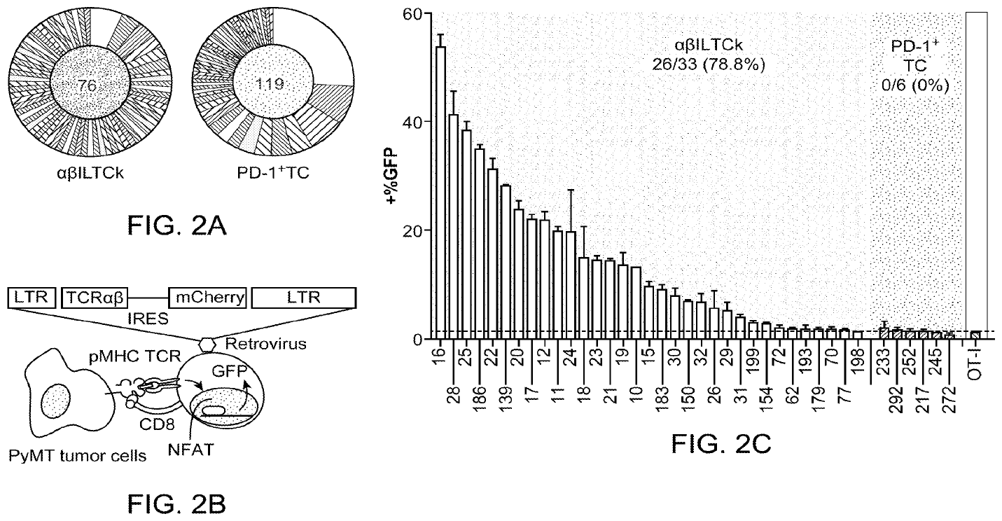

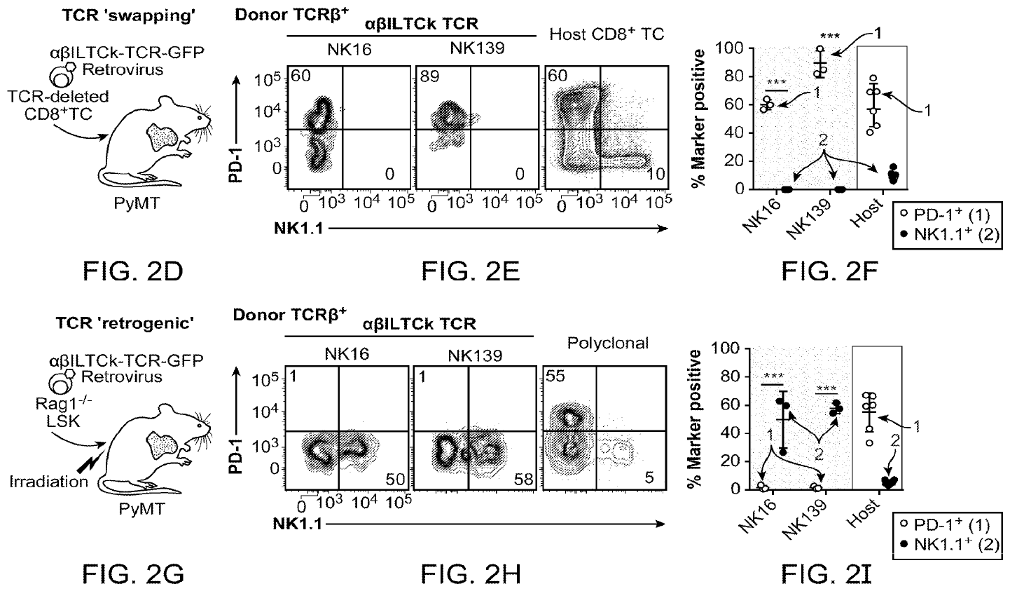

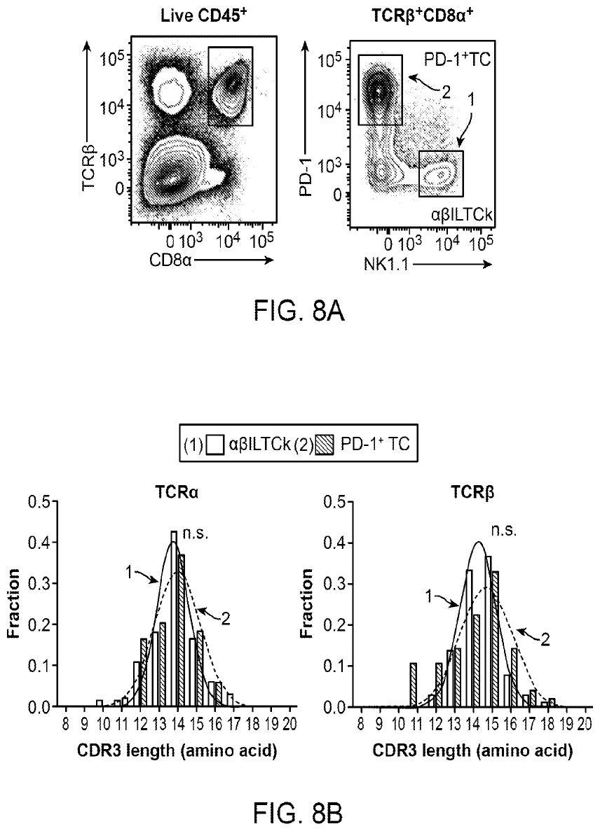

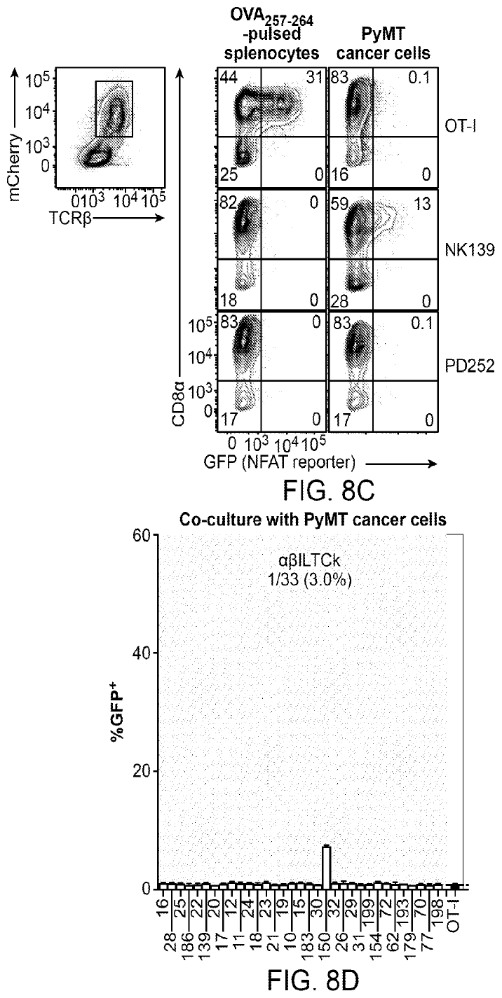

To interrogate the identity of tumor-resident NK1.1⁺ αβILTCks and how they may be distinct from conventional PD-1⁺NK1.1⁺CD8α⁺ T cells (PD-1⁺ TCs), we obtained the profiles of paired-TCR sequences utilized by each subset via single cell TCR-sequencing (FIG. 8A and Supplementary Table 1). While complementarity-determining region 3 (CDR3) lengths were comparable between NK1.1⁺ αβILTCk- and PD-1⁺ TC-derived TCRs (FIG. 8B), discrete patterns of TCR usage were noted. Over 50% of PD-1⁺ TC TCR repertoire was attributed to five unique TCR pairs (FIG. 2A), consistent with the notion that the majority of tumor-infiltrating PD-1⁺CD8α⁺ T cells arise from the expansion of a few clones^{15,16} In contrast, NK1.1⁺ αβILTCk TCRs were largely polyclonal with a moderate sign of clonal expansion (FIG. 2A). Importantly, we did not detect any TCR pairs used by both NK1.1⁺ αβILTCks and PD-1⁺ TCs, supporting the idea that they may arise from distinct precursors.

To define the specificity of NK1.1⁺ αβILTCk- and PD-1⁺ TC-derived TCRs, we modified a previously described TCR reporter assay system¹⁷ (FIG. 2B and FIG. 8C), and profiled the reactivity of NK1.1⁺ αβILTCk- and PD-1⁺ TC-derived TCRs against primary PyMT cancer cells (Supplementary Table 2). Strikingly, 26 out of 33 (78.8%) NK1.1⁺ αβILTCk-derived TCRs exhibited substantial reactivity against heterologous cancer cells (FIG. 2C). This finding suggests that NK1.1⁺ αβILTCk-derived TCRs likely recognize unmutated antigens shared by cancer cells from multiple mice. In contrast, none of the PD-1⁺ TC-derived TCRs reacted to heterologous cancer cells above the background level established by an irrelevant OT-I TCR specific to chicken ovalbumin (FIG. 2C), implying that these TCRs may recognize neoantigens unique to each tumor.

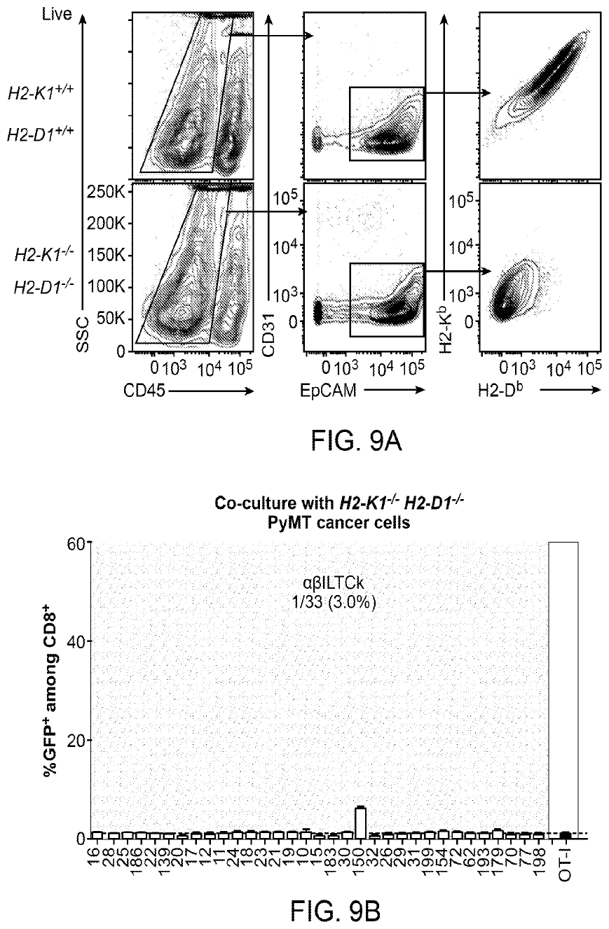

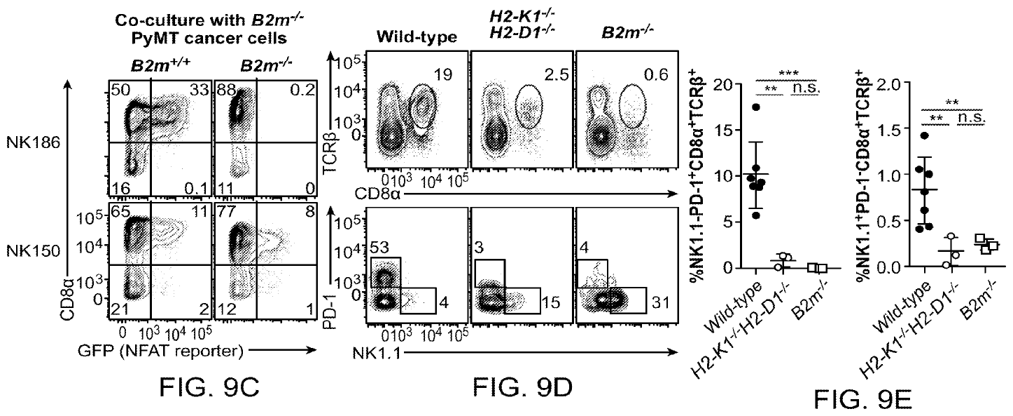

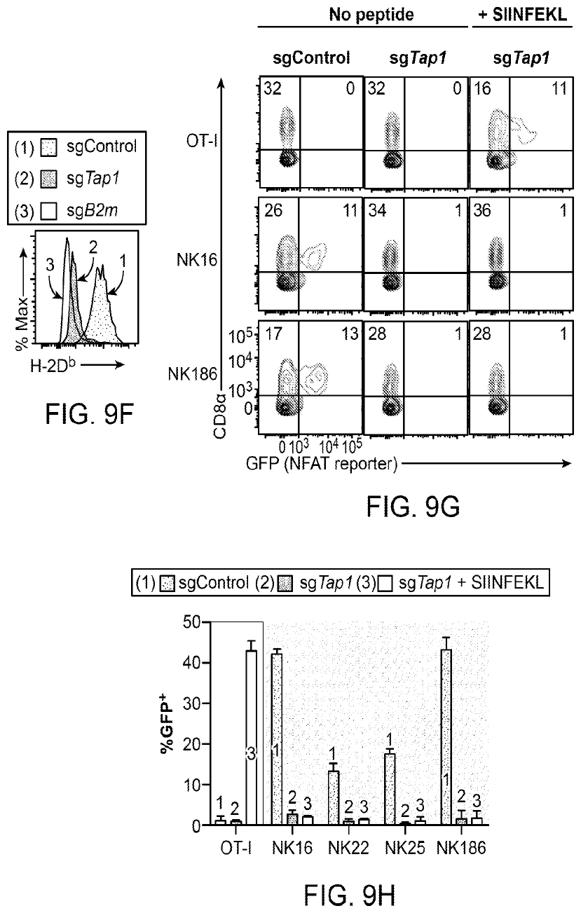

The broad cancer cell-reactivity of NK1.1⁺ αβILTCks suggests their active role in anti-tumor immune response despite lacking the hallmarks of chronic stimulation, a criterion used to define bystander CD8⁺ T cells in some human cancers^{11,18}. Notably, all except for one NK1.1⁺ αβILTCk-derived TCRs, NK150, required the co-receptor CD8 for antigen recognition (FIG. 8D), and such reactivity was lost when the cancer cells lacked classical major MHC-I-encoding genes, H2-K1 and H2-D1 (FIGS. 9A-9B). To test whether the NK150 TCR could recognize tumor-associated antigens in context of non-classical MHC-I, as reported for a subset of unconventional CD8⁺ T cells^{19,20}, we performed the TCR reporter assay using cancer cells deficient for B2m. Unexpectedly, NK150 TCR still retained its reactivity to cancer cells (FIG. 9C), indicating that NK1.1⁺ αβILTCk-TCRs may retain a certain degree of promiscuity for MHC restriction. Nevertheless, mice deficient for classical MHC-I had markedly reduced tumor-infiltrating NK1.1⁺ αβILTCks in addition to impaired conventional CD8⁺ T cell responses (FIGS. 9D-9E), suggesting that NK1.1⁺ αβILTCks, akin to their CD8⁺ TC counterparts, are predominantly classical MHC-I-restricted.

The broad tumor reactivity exhibited by NK1.1⁺ αβILTCk TCRs raises the possibility that these TCRs may in fact recognize the MHC-I molecule irrespective of the sequences of peptides presented. To this end, we repeated TCR reporter assay using a PyMT tumor-derived cancer cell line lacking the endoplasmic reticulum peptide transporter (Tap1) and therefore having a nearly undetectable level of surface MHC-I (FIG. 9F). Expectedly, the addition of SIINFEKL (SEQ ID NO: 68) peptide stabilized surface MHC-I, restoring OT-I TCR reactivity (FIGS. 9G-9H). In contrast, SIINFEKL (SEQ ID NO: 68)-stabilized MHC-I expression alone was insufficient to activate NK1.1⁺ αβILTCk TCRs (FIGS. 9G-9H), strongly supporting that NK1.1⁺ αβILTCk TCRs recognize peptide-MHC-I complex rather than the MHC-I molecule itself. Collectively, these data demonstrate that while NK1.1⁺ αβILTCks and PD-1⁺ TCs engage a similar mode of antigen recognition, they react to distinct antigen landscape in the tumor microenvironment.

### Example 4: ILTCks are Agonistically Selected

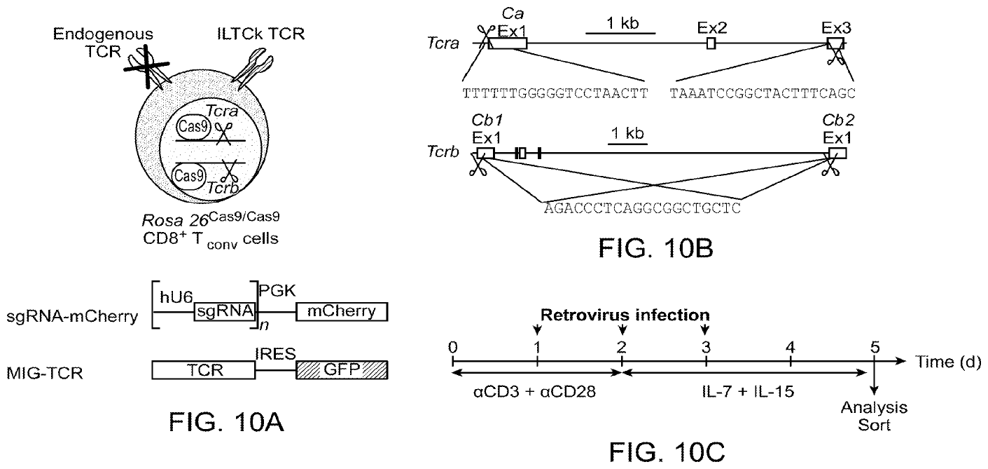

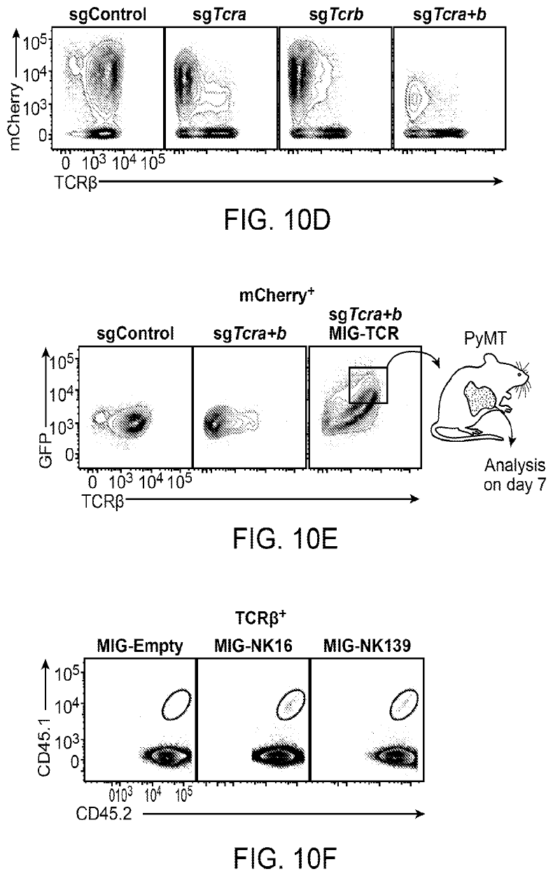

The distinct TCR specificity displayed by NK1.1⁺ αβILTCks and PD-1⁺ TCs may facilitate their localization to milieus conducive for their respective differentiation. Although our data suggests against the inter-conversion between NK1.1⁺ αβILTCks and PD-1⁺ TCs, it remains possible that cells of the conventional CD8⁺ T lineage give rise to NK1.1⁺ αβILTCks. To test this hypothesis, we ‘substituted’ the endogenously rearranged TCRs for an NK1.1⁺ αβILTCk-derived TCR in conventional CD8⁺ T cells using Cas9-mediated genome editing (FIGS. 10A-10E). Subsequently, engineered CD8⁺ T cells were adoptively transferred into congenically distinct tumor-bearing recipient mice (FIG. 2D). Intriguingly, CD8⁺ T cells expressing the NK1.1⁺ αβILTCk-derived TCRs did not upregulate NK1.1, but instead expressed PD-1 (FIG. 10F and FIGS. 2E-2F), suggesting that the exhausted T cell differentiation program may be hard-wired into its naïve precursors and under this context, NK1.1⁺ αβILTCk-derived TCRs function as any other tumor-reactive TCRs to reinforce, rather than alter this imprinted program.

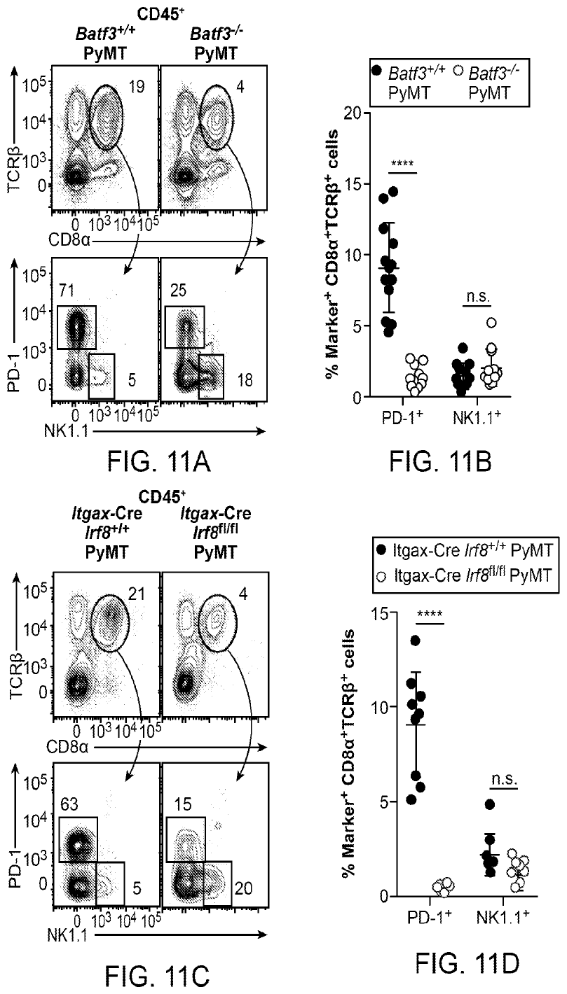

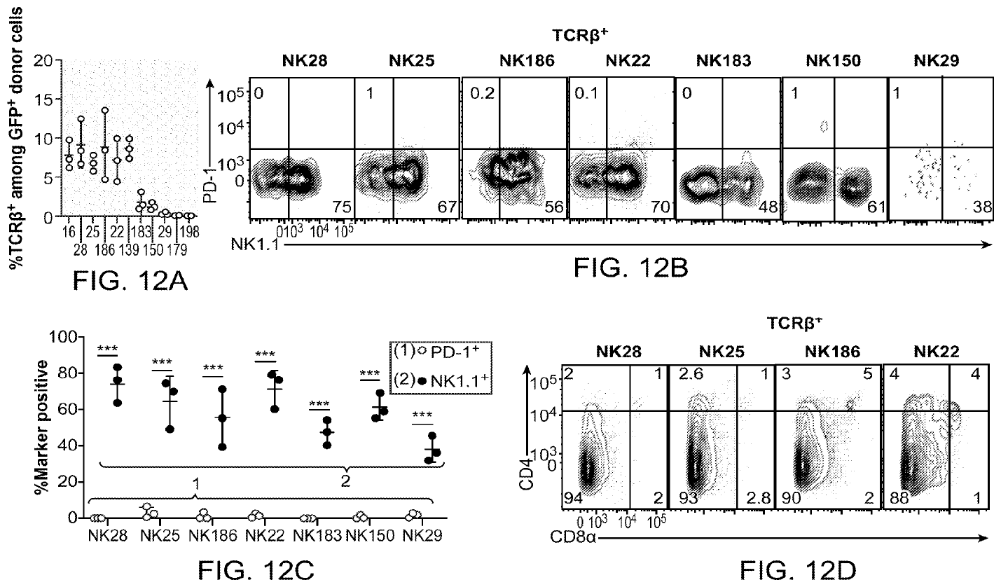

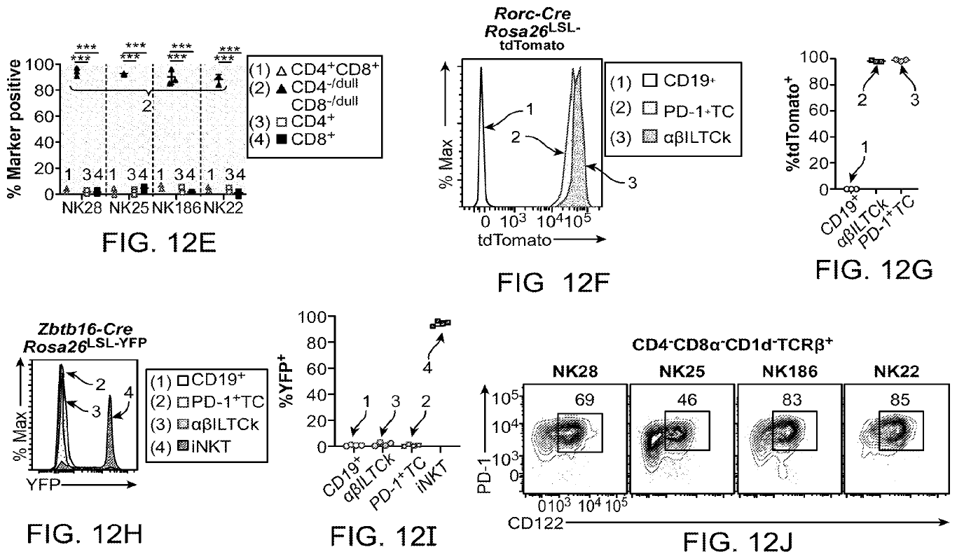

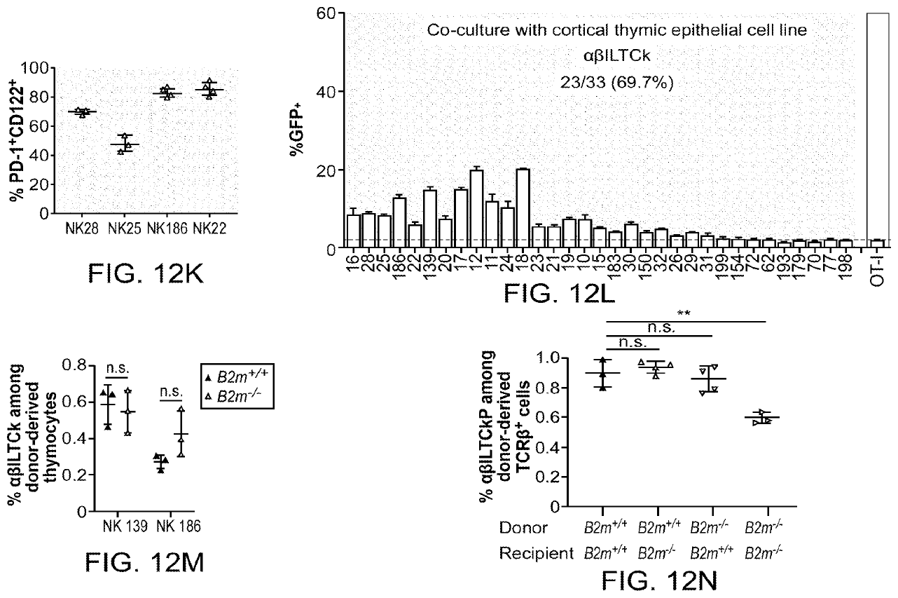

Conventional CD8⁺ T cell responses are initiated by APC priming with the Batf3- and Irf8-dependent conventional type 1 dendritic cells (cDC1) playing a critical role²¹. Consistent with previous findings, PD-1⁺ TC responses were severely impaired in cDC1-deficient PyMT mice (FIGS. 11A-11D). In contrast, NK1.1⁺ αβILTCk responses remained minimally affected (FIGS. 11A-11D), further excluding the possibility that conventional CD8⁺ T cells contribute to the NK1.1⁺ αβILTCk compartment. These observations point to an early divergence of NK1.1⁺ αβILTCk fate from their conventional counterparts. Indeed, when the NK1.1⁺ αβILTCk-derived TCRs were introduced into developing thymocytes (FIG. 2G), they consistently and specifically generated NK1.1⁺ αβILTCks, but not PD-1⁺ TCs in the tumor (FIGS. 2H-2I and FIGS. 12A-12C). Collectively, these findings suggest that NK1.1⁺ αβILTCk and PD-1⁺ TC represent two distinct and mutually exclusive fate choices, and the commitment to either lineage occurs during thymocyte development in a TCR-specificity-dependent manner.

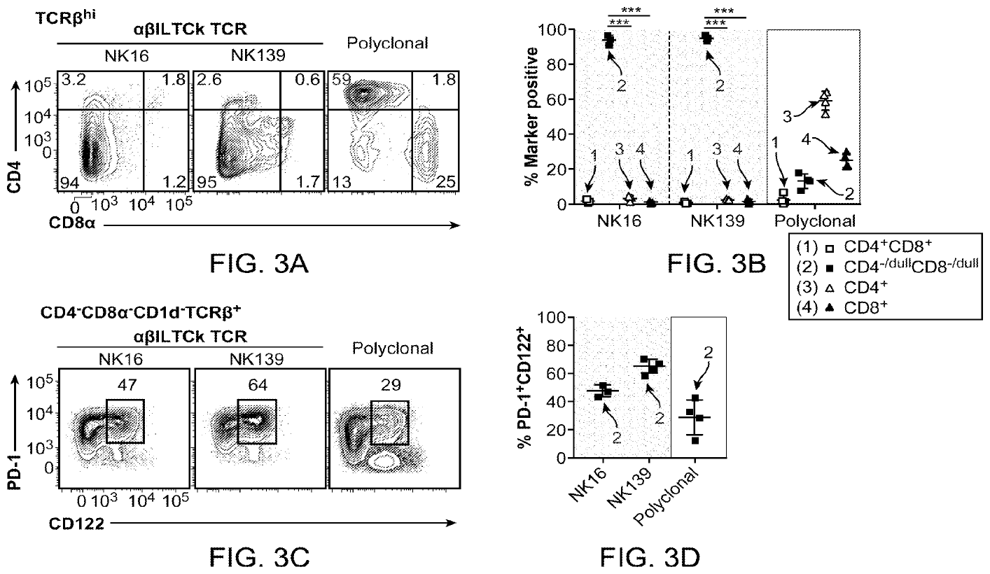

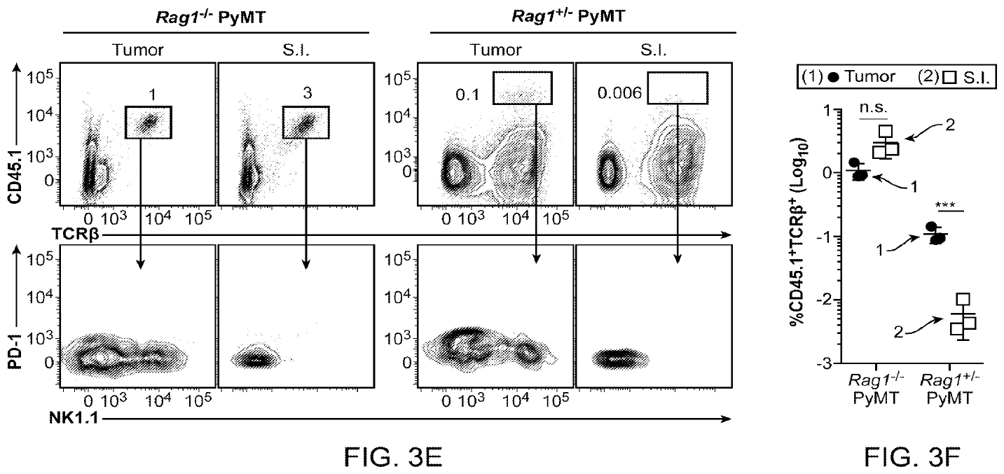

To interrogate how distinct TCR specificity displayed by NK1.1⁺ αβILTCks drives their lineage commitment, we profiled the phenotypic changes of thymocytes expressing the NK1.1⁺ αβILTCk-derived TCRs at various stages during their development. Following positive selection, thymocytes with a polyclonal TCR repertoire predominantly generated conventional CD4 and CD8 single positive (SP) T cells (FIGS. 3A-3B). In contrast, thymocytes expressing a monoclonal TCR derived from NK1.1⁺ αβILTCks yielded only CD4^{-/dull}CD8^{-/dull} cells with negligible differentiation into either CD4 or CD8 SP T cells (FIGS. 3A-3B and FIGS. 12D-12E), suggesting that αβILTCk commitment is governed by a developmental checkpoint distinct from the positive selection event.

Thus far, all known TCRαβ⁺ T cells undergo a CD4⁺CD8⁺ double positive (DP) stage in the thymus during development²², after which further functional specification into distinct lineages takes place. Using a Rorc-Cre allele which is transiently active in DP thymocytes²³, we observed that all tumor-resident NK1.1⁺ αβILTCks as well as PD-1⁺ TCs, but not CD19⁺B cells were fate-mapped in Rorc-CreRosa26^{LSL-tdTomato}PyMT mice (FIGS. 12F-12G), indicating that similar to conventional CD8⁺ T cells, NK1.1⁺ αβILTCks also undergo a DP stage. However, distinct from other innate-like T cells such as CD1d-restricted invariant natural killer T (iNKT) cells that are selected by DP thymocyte-presented antigens and marked by high expression of the transcription factor Zbtb16, NK1.1⁺ αβILTCks were not fate-mapped in Zbtb16-CreRosa26^{LSL-YFP}PyMT mice (FIGS. 12H-12I), as a likely consequence of lack of classical MHC-I expression on DP thymocytes.

Following positive selection, DP thymocytes transiently expressed low levels of PD-1. In contrast, those bearing the NK1.1⁺ αβILTCk-derived TCRs uniformly maintained high PD-1 expression (FIGS. 3C-3D and FIGS. 12J-12K). Given that PD-1 upregulation represents a sensitive gauge for the strength of antigen receptor stimulation, these findings suggest that TCRs of NK1.1⁺ αβILTCks may be strongly autoreactive. Indeed, 23 out of 33 (69.7%) of NK1.1⁺ αβILTCk-derived TCRs exhibited variable but substantial reactivity to a cortical thymic epithelial cell line, the level of which surpassed that of the OT-I TCR, which robustly drove conventional CD8⁺ T cell lineage commitment via positive selection (FIG. 12L and data not shown).

To distinguish between the role of hematopoietic and radiation-resistant stromal compartments in mediating αβILTCk selection, we generated TCR ‘retrogenic’ mice using wild-type or B2m^{-/-} animals as recipients. Intriguingly, the thymic αβILTCk progenitor compartment remained unaltered in B2m^{-/-} recipients (FIG. 12M), suggesting that antigen presentation from the hematopoietic compartment is sufficient for αβILTCk selection. Ablating B2m in the hematopoietic compartment alone mildly reduced αβILTCk progenitor generation with B2m deficiency in both compartments resulting in a substantial reduction of αβILTCk progenitors (FIG. 12N). These findings suggest that strong autoreactivity drives αβILTCk lineage commitment, akin to the ‘agonist’ selection process which specifies iNKT cell and intestinal intraepithelial lymphocyte (IEL) fates²⁵. But unlike iNKT cells, which are selected by DP thymocytes, ‘agonist’ selection signals for αβILTCks are redundantly supplied by both the hematopoietic and radiation-resistant stromal compartments.

### Example 5: ILTCks Continually Repopulate Tumor

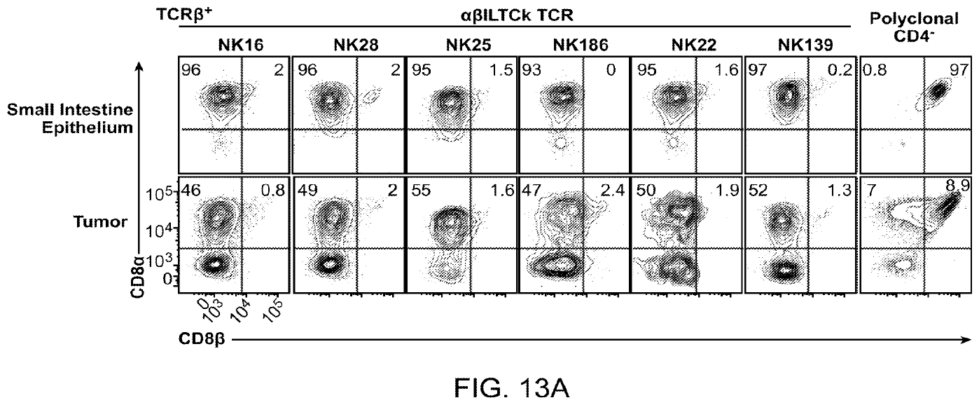

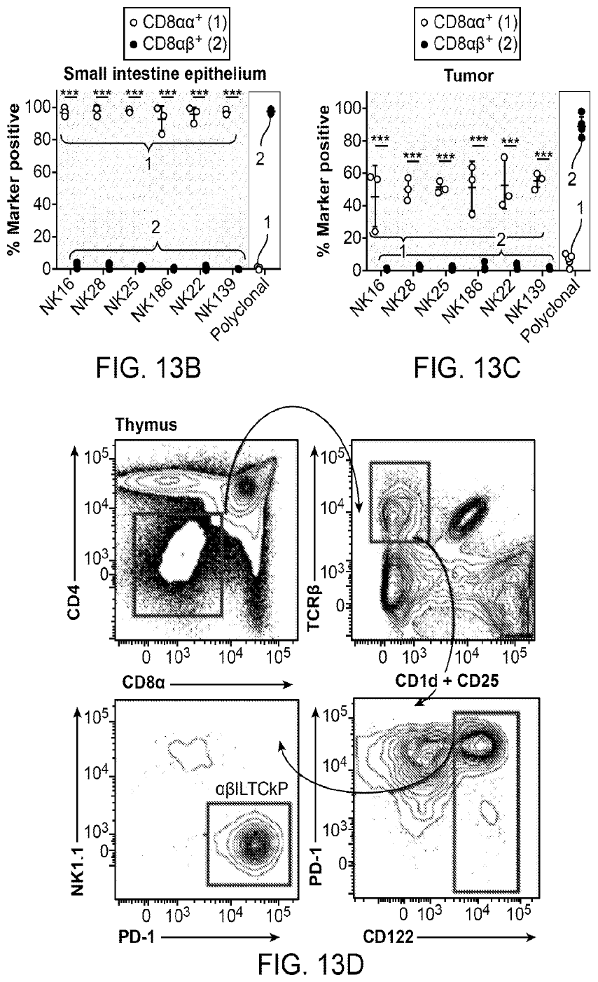

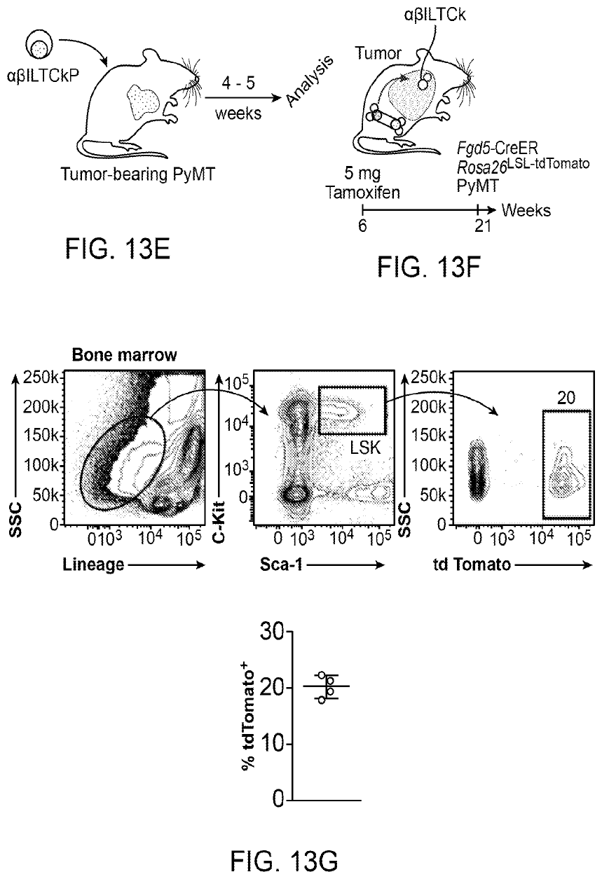

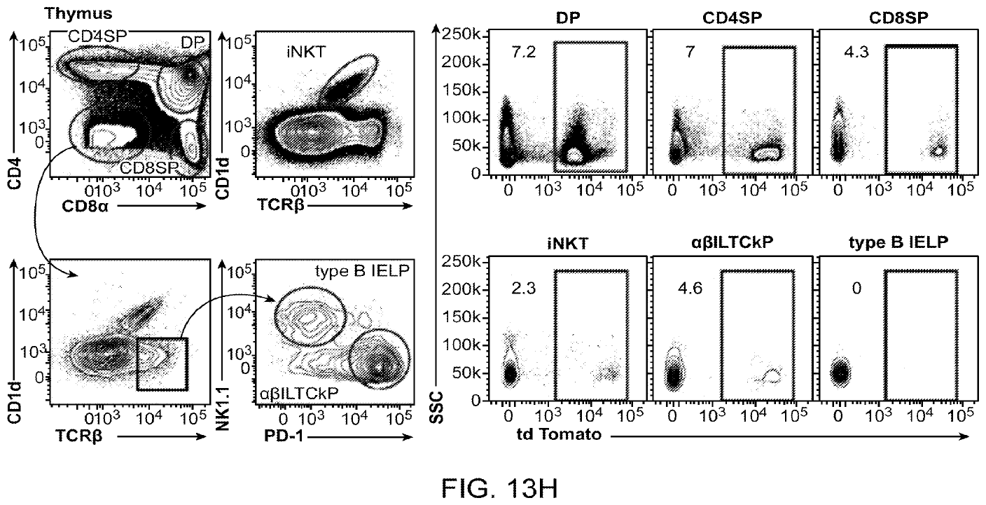

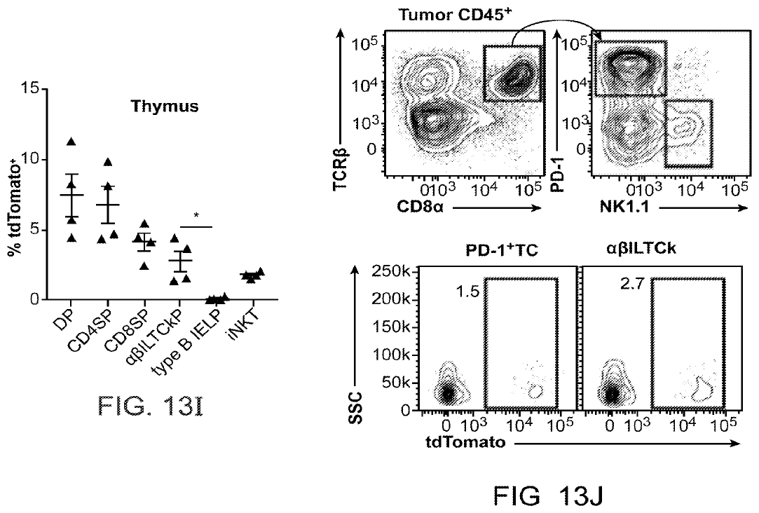

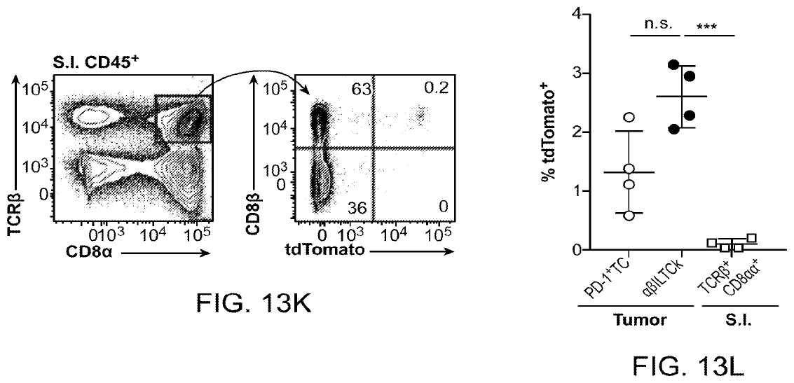

A substantial proportion of NK1.1⁺ αβILTCk-TCR-bearing thymocytes expressed CD122, the β-chain of IL-2/IL-15 receptor (FIGS. 3C-3D and FIGS. 12J-12K), which is upregulated in tumor-resident terminally differentiated αβILTCks¹³. Thus, CD122 expression may mark the most mature αβILTCk-committed thymic progenitor which is otherwise identified as CD4^{-/lo}CD8^{-/lo}PD-1⁺. Notably, these cell surface phenotypes bear striking resemblance to those identified for an IEL-committed thymic progenitor¹⁹, raising the possibility that αβILTCks and IELs may be developmentally related. Indeed, thymocytes expressing the NK1.1⁺ αβILTCk-derived TCRs differentiated into small intestinal IELs in addition to intratumoral αβILTCks with both populations expressing the CD8αα homodimer, albeit with different frequencies, in contrast to CD8αβ heterodimers on conventional CD8⁺ TCs (FIGS. 13A-13C). Consistent with this, upon adoptive transfer into lymphopenic tumor-bearing host mice, TCRβ⁺CD4^{-/lo}CD8^{-/lo}PD-1⁺CD122⁺ thymic progenitors reliably generated both intratumoral αβILTCks and intestinal IELs with NK1.1 upregulation observed only in the former (FIGS. 13D-13E and FIGS. 3E-3F). Thus, these data demonstrate that intestinal IELs and tumor-resident αβILTCks are in fact of the same lineage sharing an immediate thymic progenitor. Their disparate phenotypes in the peripheral tissues perhaps reflect the distinct differentiation signals provided by the different microenvironments the progenitors initially seed.

Although αβILTCks/IEL progenitors are present throughout life, they colonize different organs with distinct kinetics. The CD8αα⁺ intestinal IELs are most abundant at birth and the population gradually dwindles with age^{26,27}, In contrast, intratumoral αβILTCks increase in number as tumor progresses¹³. Conceivably, a growing tumor continuously creates tissue niche for αβILTCk expansion whereas the small intestine epithelium, albeit being one of the organs with the fastest cell turnover, maintains a homeostatic level of epithelial niche, which subsequently limits seeding and expansion of newly generated progenitors and existing IELs, respectively. Consistent with this notion, while αβILTCk/IEL progenitors robustly engrafted both the tumor and small intestinal epithelium in lymphopenic mice, they failed to populate the small intestinal IEL pool in lympho-replete mice (FIGS. 3E-3F). In contrast, we detected a sizable, albeit small contribution from the transferred progenitors to the tumor-resident αβILTCk compartment in the same host (FIGS. 3E-3F), suggesting that the availability of tissue niche may represent the limiting factor for progenitor seeding and expansion²⁸.

While the population expansion of αβILTCks in the tumor can be attributed, in part, to in situ proliferation of mature αβILTCks¹³, to what extent αβILTCk/IEL progenitors replenish the expanding pool of αβILTCks remains unclear. To this end, we performed ‘time-stamp’ experiments using the Fgd5-CreERRosa26^{LSL-tdTomato}PyMT mice, in which a pulse of tamoxifen administration labels a fraction of hematopoietic stem cells²⁹, allowing stable tracking of their progenies (FIG. 13F). Consistent with published results, we observed a 20% labeling efficiency in the Lineage⁻c-Kit⁺Sca1⁺ bone marrow stem cells²⁹ (FIG. 13G). Importantly, approximately 3% of thymic αβILTCk/IEL progenitors were fate-mapped, which was comparable to the labeling frequency in DP, SP, and iNKT cell compartments (FIGS. 1311-13I), indicating that αβILTCk/IEL progenitors are continuously generated by adult hematopoiesis. In contrast, the TCRβ⁺CD4^{-/lo}CD8^{-/lo}PD-1⁻ CD122⁺NK1.1⁺ thymocytes, previously termed ‘type B IEL progenitors’, were not fate-mapped (FIGS. 1311-13I), thus reflecting their embryonic/neonatal origin^{19,28}. These cells may in fact represent mature thymus-resident αβILTCks arising from in situ differentiation of αβILTCk/IEL progenitors early in life²⁸. In the periphery, comparable proportions of intratumoral PD-1⁺ TCs and αβILTCks were fate-mapped (FIGS. 13J, 13L), indicating that αβILTCks, similar to conventional CD8⁺ T cells, are continuously replenished by circulating precursors. In contrast, small intestinal CD8αα⁺ IELs showed negligible labeling (FIGS. 13K-13L), confirming early seeding and in situ proliferation as their primary means for population maintenance²⁸. Thus, like naïve T cells which are constantly generated to warrant reactivity to foreign antigens, the αβILTCk/IEL lineage is also continuously produced, potentially in anticipation of tissue distress, such as cellular transformation that may arise as animals age.

### Example 6: FCER1G Expression Marks ILTCk Lineage

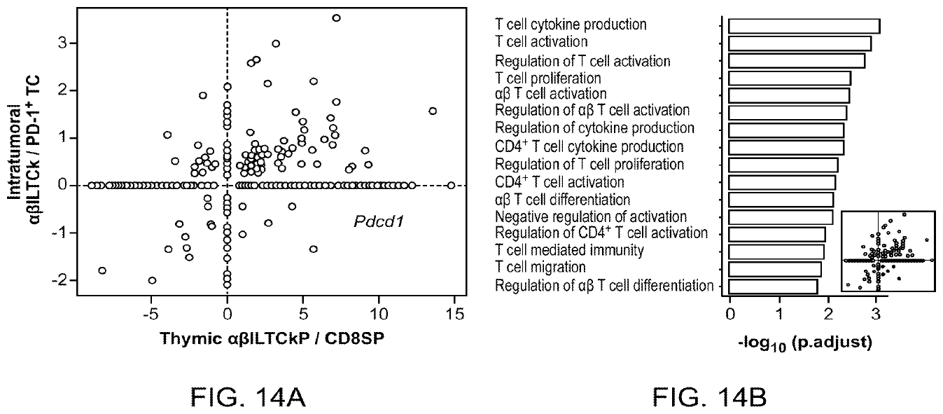

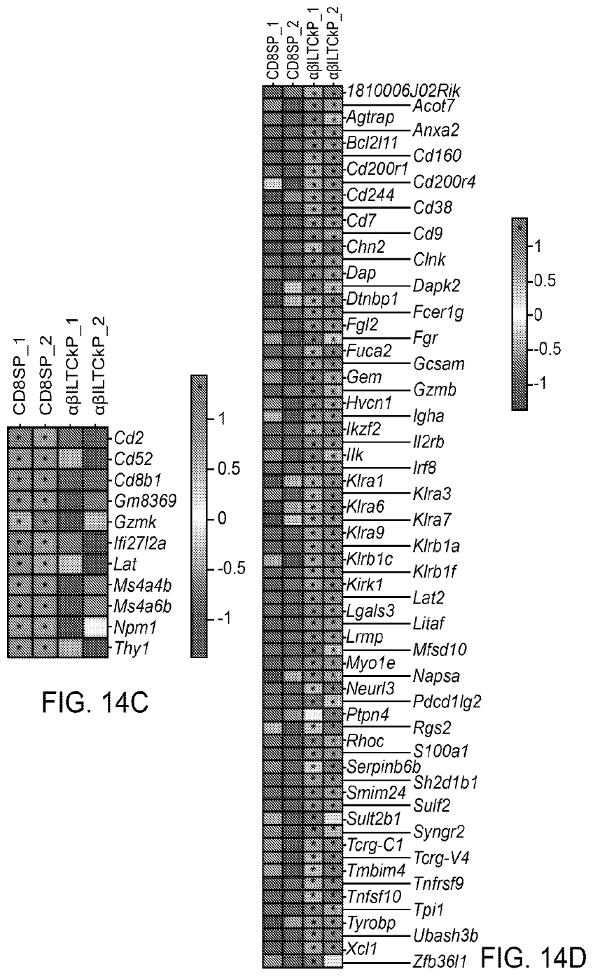

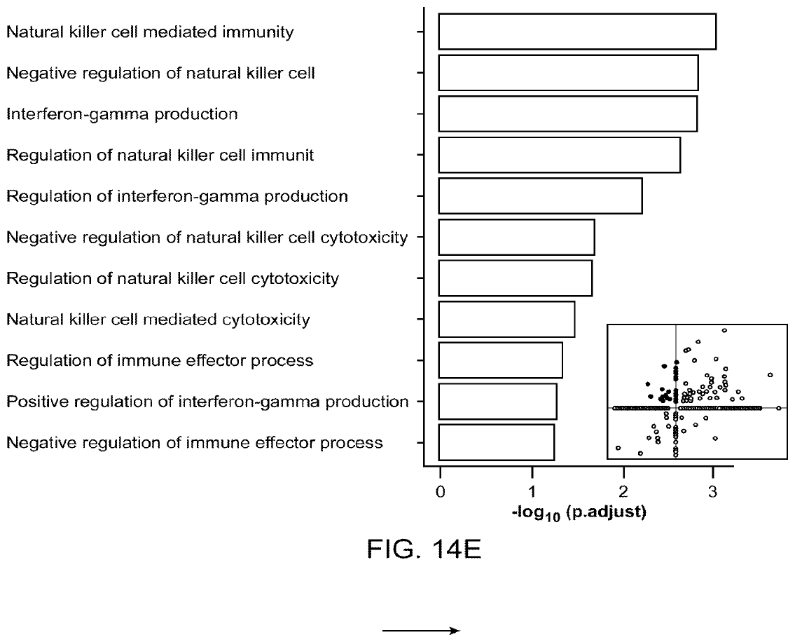

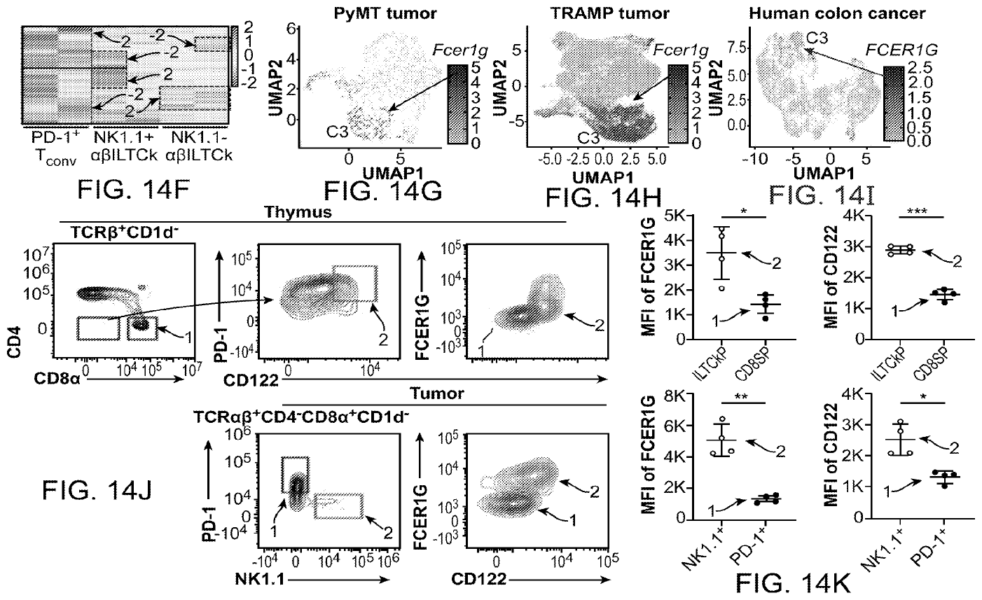

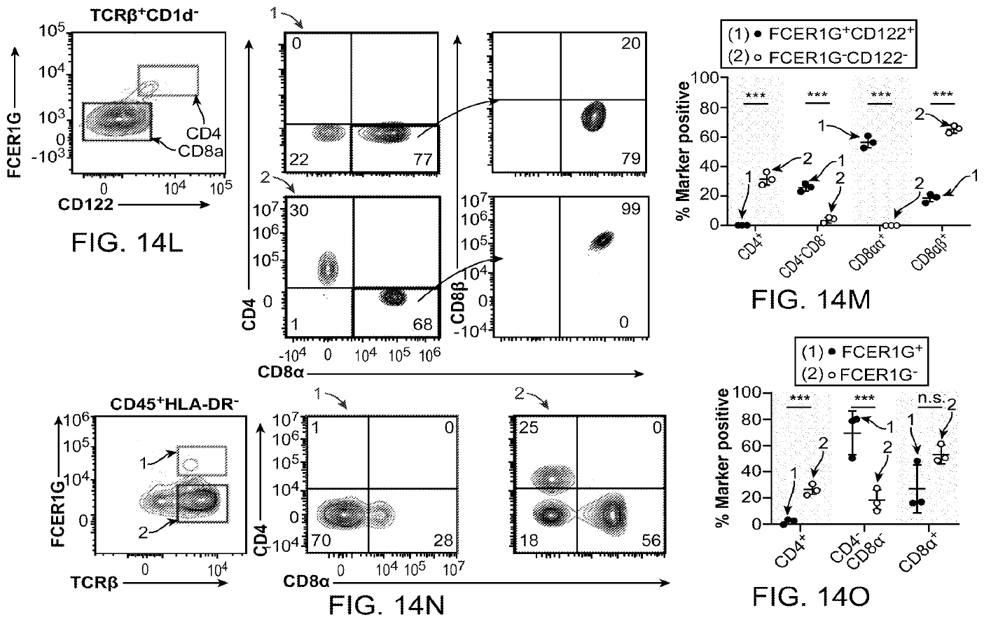

To gain further insights into the molecular mechanisms underlying the distinct tumor-elicited responses mounted by αβILTCks and conventional CD8⁺ T cells, we compared the gene expression profiles of tumor-infiltrating αβILTCks and PD-1⁺ TCs to their respective thymic progenitors (FIG. 14A). Genes upregulated in αβILTCk progenitors but not in their mature counterparts were enriched for those involved in T cell activation and differentiation including the immediate early gene Nr4a1 (FIG. 14B and Supplementary Table 3), possibly reflecting the ‘agonist’ selection event in the thymus in line with previous studies¹⁹. The transcription factor Tox, which promotes the expression of multiple inhibitory receptors^{30,31,32}, including Pdcd1 in response to TCR stimulation, was also transiently upregulated in αβILTCk/IEL progenitors but subsequently remained actively suppressed in mature NK1.1⁺ αβILTCks (Supplementary Table 3), suggesting that early encounter with cognate antigen may offset the rheostat of TCR signaling. This may be in part achieved via downregulation of Lat and Cd2, both of which function to amplify signals through the TCR (FIG. 14C and Supplementary Table 3), thereby rendering mature NK1.1⁺ αβILTCk not susceptible to exhaustion compared to conventional T cells in the tumor microenvironment. Also reflecting a history of cognate antigen encounter, genes associated with effector functions and those encoding multiple NK receptors, were already upregulated in αβILTCk/IEL progenitors (FIG. 14D and Supplementary Table 3) and remained highly expressed in mature NK1.1⁺ αβILTCks compared to PD-1⁺ TCs¹³. In contrast, pathways associated with terminal effector differentiation and tissue residency programs, including Gzmc, Itga1, and Itgae were likely acquired in response to tumor microenvironment-specific local signals (FIG. 14E and Supplementary Table 3). Thus, unlike conventional T cells which require two checkpoints, positive selection and APC priming to acquire effector programs, αβILTCk/IEL progenitors undergo selection and priming in a single step, and exit the thymus equipped with cytotoxic potential.

While adoptive transfer of committed αβILTCk/IEL progenitors consistently generated tumor-resident αβILTCks, a substantial proportion remained as NK1.1⁻ (FIG. 3E). This was unlikely a result of pre-existing heterogeneity within the αβILTCk/IEL progenitors as thymocytes expressing a monoclonal TCR also gave rise to NK1.1⁻ and NK1.1⁺ subsets (FIGS. 2G-2I and FIGS. 12B-12C). Rather, the NK1.1⁻ fraction may represent an immature state during αβILTCk differentiation. Indeed, while the NK1.1⁻ cells were transcriptionally more similar to NK1.1⁺ αβILTCks than PD-1⁺ TCs (FIG. 14F), they had higher expression of transcripts enriched in thymic αβILTCk/IEL progenitors including Pdcd1 (Supplementary Table 4). Genes associated with terminal effector differentiation and function including Tyrobp and Gzmc were subsequently co-upregulated upon acquisition of NK1.1 (Supplementary Table 4). Thus, NK1.1 marks activated αβILTCks and may not identify all αβILTCk lineage of cells in the tumor.

Single cell RNA-sequencing analyses of tumor-infiltrating CD8⁺ T cells revealed that Fcer1g expression was specifically and uniformly enriched in the transcriptionally defined αβILTCk cluster (C3) in both the PyMT breast and TRAMP prostate cancer models (FIG. 6 and FIGS. 14G-14H). Notably, in tumor tissues from patients with colorectal cancer, FCER1G marked a C3 subset transcriptionally similar to murine αβILTCks (FIGS. 7I-7K and FIG. 14I), together suggesting that Fcer1g/FCER1G may represent a conserved αβILTCk lineage-defining marker irrespective of activation state. Indeed, FCERG1 protein was already upregulated in committed PD-1^{hi}CD122^{hi} thymic αβILTCk/IEL progenitors but not in CD8 SPs, and continued to be expressed in tumor-infiltrating NK1.1⁺ αβILTCks but not PD-1⁺ T cells (FIGS. 14J-14K), indicating that FCER1G specifically and stably marks cells committed to the αβILTCk lineage.

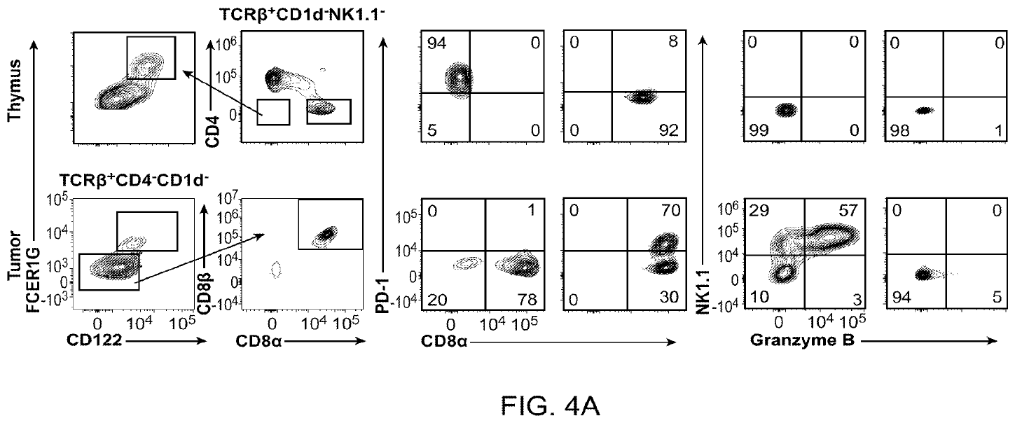

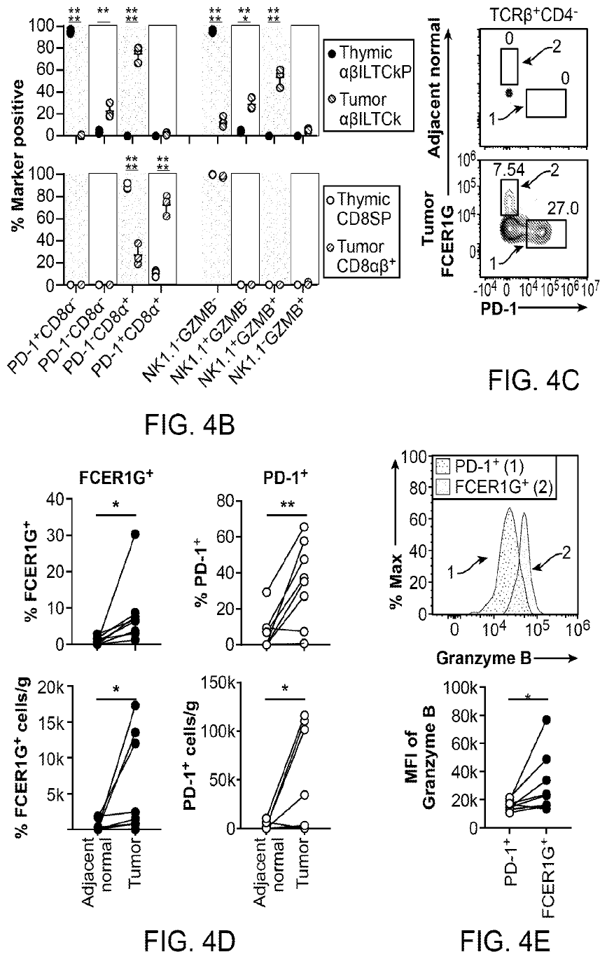

Among CD4-CD8α⁺TCRβ⁺CD1d⁻NK1.1⁻ thymocytes, the FCER1G⁺CD122⁺ population expressed high levels of PD-1, and lacked granzyme B (GzmB) expression, phenotypically identical to αβILTCk/IEL progenitors defined by CD122 and PD-1 co-expression (FIGS. 4A-4B). Among tumor-infiltrating T cells, the FCER1G⁺CD122⁺ population remained as CD4⁻ with the majority upregulating CD8αα homodimer (FIGS. 14L-14M), and uniformly lacked PD-1 expression in contrast to the FCER1G population (FIGS. 4A-4B). Notably, FCER1G⁺CD122⁺ T cells contained NK1.1⁺GzmB^{+/-} αβILTCks as well as their immature NK1.1⁻GzmB⁻ precursors (FIGS. 4A-4B). Thus, FCER1G expression sufficiently identifies tumor-infiltrating αβILTCks regardless of their activation states.

Strikingly, in patients with colorectal carcinoma, FCER1G⁺TCRβ⁺ cells were also readily detected in tumor tissues (FIG. 14N). Similar to their murine counterparts, FCER1G⁺ T cells lacked CD4 co-receptor expression with the majority remaining CD4⁻ CD8⁻ and a fraction upregulating CD8α (FIGS. 14N-14O). Not unlike PD-1⁺ T cells, FCER1G⁺ T cells were also significantly enriched in tumor tissues relative to adjacent normal colon (FIGS. 4C-4D), but expressed higher levels of granzyme B compared to their PD-1⁺ counterparts (FIG. 4E). Collectively, these findings identify FCER1G as a novel αβILTCk lineage-defining marker and demonstrate that the αβILTCk program in fact represents an evolutionarily conserved tumor-elicited immune response in both mouse and human.

### Example 7: ILTCk is Engineerable for Cancer Therapy

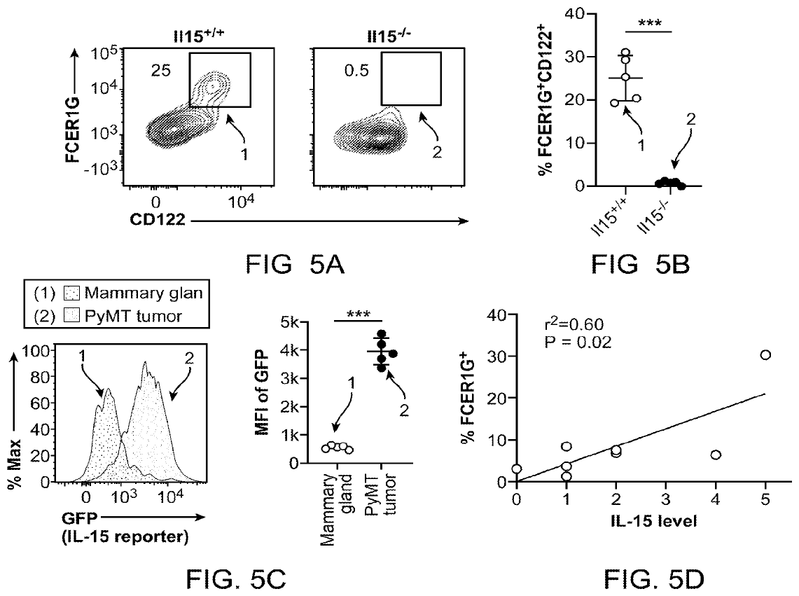

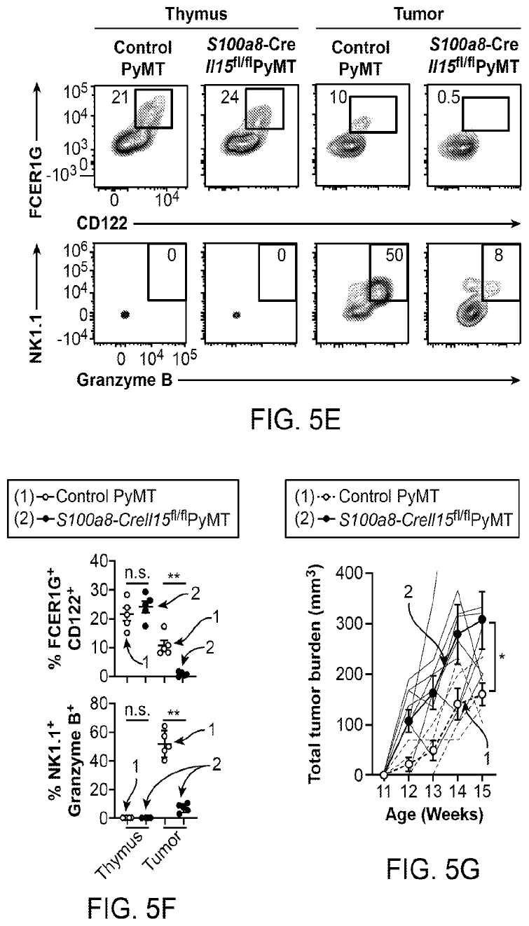

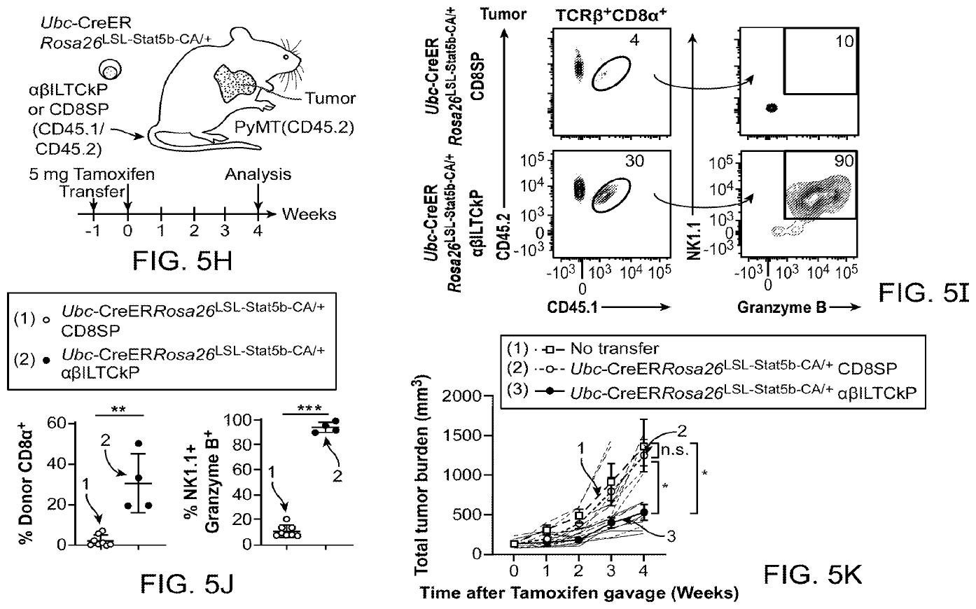

While we and others previously demonstrated that intratumoral NK1.1⁺ αβILTCks¹³ as well as small intestinal IELs³³ are critically dependent on the pro-inflammatory cytokine IL-15, these studies relied on inducible markers such as NK1.1 and CD8αα to identify mature αβILTCks and IELs. As such, whether IL-15 regulates αβILTCk/IEL development remains unclear. Using the newly identified αβILTCk lineage-defining marker, FCER1G, we observed an almost complete absence of FCER1G⁺CD122⁺ thymic αβILTCk/IEL progenitors in mice lacking Il15 compared to wild-type controls (FIGS. 5A-5B).

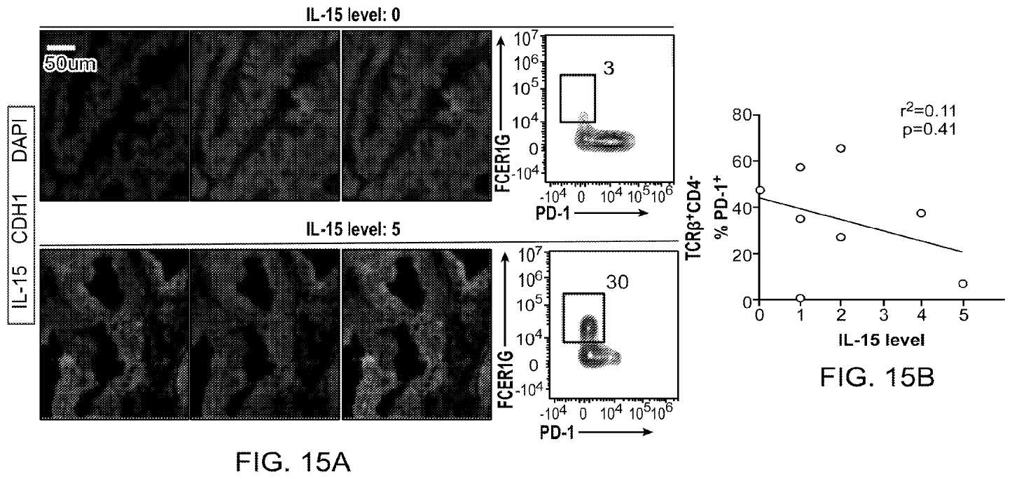

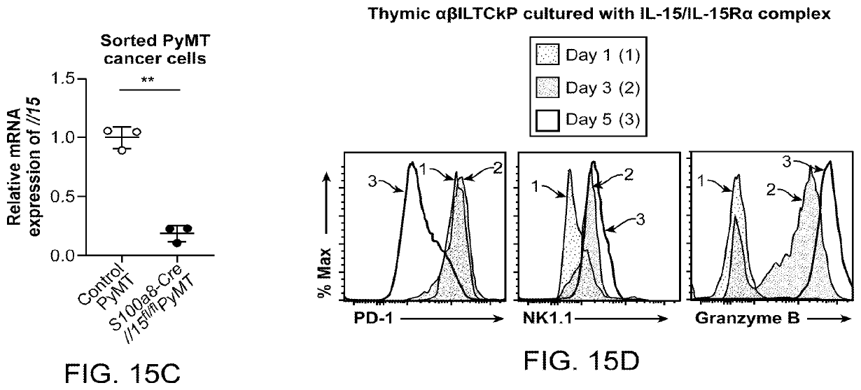

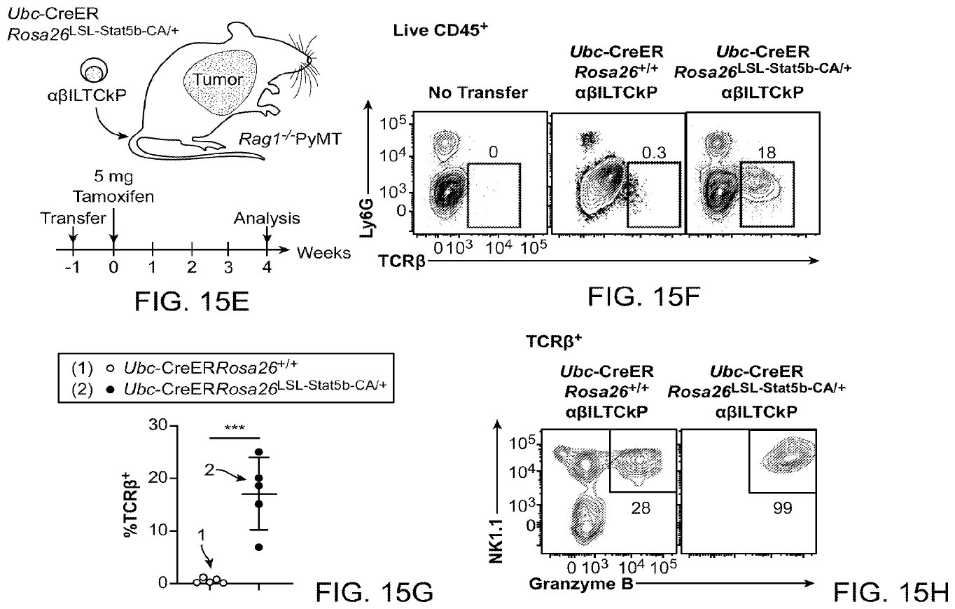

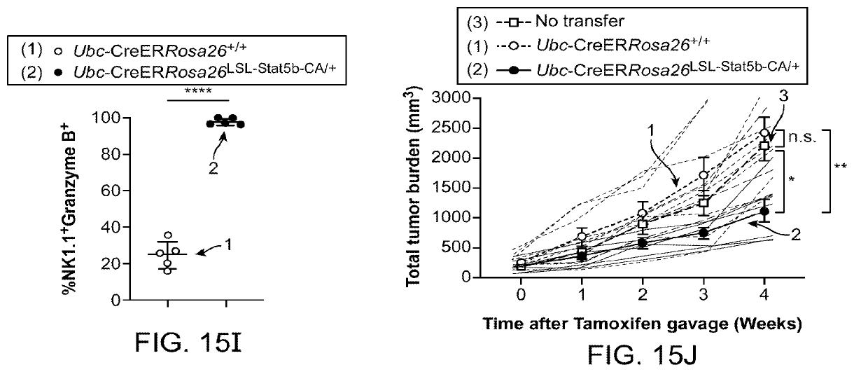

As IL-15 is expressed in both lymphoid and nonlymphoid tissues, the exact source of IL-15 that drives the expansion and activation of intratumoral αβILTCks remains elusive. Of note, ablation of Il15 in hematopoietic lineage of cells did not impair the tumor-elicited αβILTCk response in PyMT mice (data not shown). Intriguingly, transgenic expression of IL-15Ra on small intestinal epithelium restored an otherwise ablated IEL compartment in Il15ra^{-/-} mice³⁴, suggesting epithelium can be a primary source of IL-15 for the maintenance of the αβILTCk/IEL lineage. Indeed, IL-15 expression was markedly increased in transformed mammary epithelium compared to healthy mammary tissues (FIG. 5C). IL-15 was also readily detected, albeit at different levels, in tumor epithelium from patients with colorectal carcinoma (FIG. 15A). Notably, frequency of FCER1G⁺, but not PD-1⁺, T cells was positively associated with IL-15 levels (FIG. 5D and FIGS. 15A-15B), collectively suggesting that cancer cell-derived IL-15 may principally support the αβILTCk compartment.

To investigate whether cancer cell-expressed IL-15 regulated αβILTCk response, we utilized the S100a8-CreIl15^{fl/fl} PyMT mice in which Il15 was specifically deleted in transformed, but not healthy mammary epithelium (FIG. 15C and data not shown). In contrast to whole body IL-15-deficient mice (FIGS. 5A-5B), S100a8-CreIl15^{fl/fl} PyMT mice had comparable thymic FCER1G⁺CD122⁺ αβILTCk progenitors (FIGS. 5E-5F). Strikingly, tumor-infiltrating αβILTCks were markedly reduced in S100a8-CreIl15^{fl/fl} PyMT mice compared to controls, and residual αβILTCks had largely diminished expression of NK1.1 and granzyme B (FIGS. 5E-5F). Notably, S100a8-CreIl15^{fl/fl} PyMT mice exhibited accelerated tumor growth compared to wild-type controls (FIG. 5G). These findings thus indicate that cancer cell-derived IL-15 represents a primary source of IL-15 in maintaining intratumoral αβILTCks and promoting αβILTCk-mediated cancer immunosurveillance.

While conventional T cell-based adoptive cellular transfer therapy has seen tremendous success in treating liquid cancers, its therapeutic use in solid cancers was hampered in part by conventional CD8⁺ T cell's propensity to exhaustion as well as poor tissue infiltration and retention. Additionally, conventional T cell-based adoptive transfer therapies require a priori knowledge of the target antigen, rendering the approach highly personalized and time-consuming. αβILTCk's resistance to PD-1 upregulation, its intrinsic tissue homing property, and broad tumor reactivity thus make αβILTCk-based cellular transfer therapy an appealing alternative. Although adoptive transfer of committed thymic αβILTCk progenitors partially replenished the αβILTCk compartment in lymphopenic hosts, the population expansion of transferred cells was inefficient in lympho-replete hosts (FIGS. 3E-3F). Furthermore, only a portion of transferred cells differentiated into the NK1.1⁺ state (FIGS. 3E-3F). These observations suggest that the availability of tumor microenvironment-derived signals may limit the maximal expansion and terminal differentiation of adoptively transferred αβILTCk progenitors.

As cancer cell-derived IL-15 was critical for the expansion of αβILTCks and IL-15 alone was sufficient to induce NK1.1 as well as granzyme B upregulation and the concomitant PD-1 downregulation in cultured thymic αβILTCk progenitors (FIGS. 5E-5F and FIG. 15D), we hypothesized that ectopic activation of IL-15 signaling in adoptively transferred αβILTCk progenitors may overcome the paucity of growth factors and maturation signals in the tumor microenvironment, thereby promoting their maximal expansion and differentiation to achieve therapeutic effects. To this end, we purified thymic αβILTCk progenitors from Ubc-CreERRosa26^{LSL-Stat5b-CA/+} mice in which tamoxifen administration induces expression of a constitutively active form of the transcription factor Stat5b (Stat5b-CA), which principally coordinates the transcriptional program downstream of IL-15 signaling³⁵ (FIG. 15E). Following adoptive transfer into lymphocyte-deficient tumor-bearing PyMT mice, inducible activation of Stat5b resulted in a 60-fold expansion of transferred cells within four weeks (FIGS. 15F-15G). Notably, whereas only a fraction of the control donor cells co-upregulated NK1.1 and granzyme B, Stat5b-CA-armed αβILTCks uniformly expressed high levels of both (FIGS. 1511-15I), indicating that hyperactivation of IL-15 signaling was sufficient to drive robust expansion and effector differentiation of αβILTCks. Importantly, mice receiving Stat5b-CA-armed αβILTCks exhibited significantly deterred tumor growth compared to those transferred with control or no cells (FIG. 15J).

When adoptively transferred into lympho-replete PyMT hosts, Stat5b-CA-armed αβILTCk progenitors readily colonized tumor tissues and underwent robust expansion as well as effector differentiation, resulting in diminished tumor growth (FIGS. 5H-5K). In contrast, adoptively transferred Stat5b-CA-armed thymic CD8 SP T cells failed to engraft or differentiate and expectedly, tumor growth was unaltered (FIGS. 5H-5K). This was most likely due to low frequency of tumor-reactive clones in CD8 SP T cells. As conventional T cell activation requires TCR stimulation, hyperactivation of IL-15 signaling alone in the absence of TCR engagement may be insufficient to initiate conventional CD8⁺ T cell responses. While αβILTCk-TCRs were broadly reactive to unmutated tumor-associated antigens (FIG. 2C), this autoreactivity appeared to permanently dampen TCR signaling in thymocytes committed to the αβILTCk lineage (FIG. 14C and data not shown). Indeed, TAP2-deficient cancer cells are equally susceptible to killing by αβILTCk as wild-type controls¹³, implying that activating cues other than TCR-MHC/peptide interaction underlies αβILTCk cytotoxicity, with cancer cell-expressed IL-15 being possibly one of such alarmins. Thus, these findings highlight the strength of αβILTCk-based adoptive cellular transfer therapy as such an approach does not require a priori knowledge of specific target antigens.

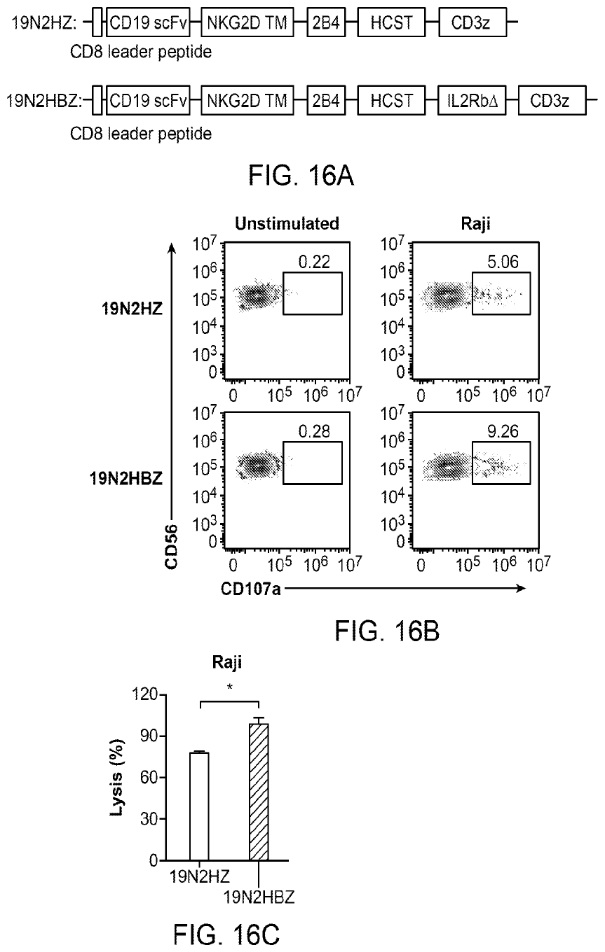

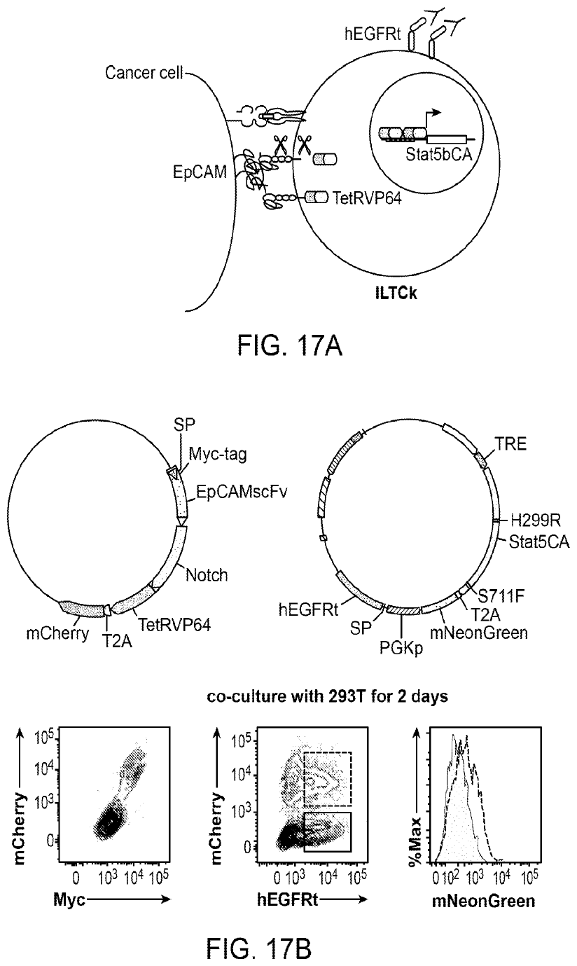

Furthermore, these proof-of-principle experiments demonstrated that IL-15 signaling axis in αβILTCk can be a powerful and exploitable substrate for the development of cancer therapies. FIGS. 16A-16C and FIGS. 17A-17B demonstrate that induction of JAK-STAT activation can be successfully extrapolated to ILTCks to achieve anti-tumor effects

Supplementary Table 1. Unique CDR3 amino acid sequences found in TCRs utilized by tumor-infiltrating αβILTCks and PD-1⁺CD8⁺ T cells (TCs).

Supplementary Table 2. Full-length nucleotide sequences of TCRs used in the TCR reporter assay.

Supplementary Table 3. Differentially expressed genes and associated biological pathways among intratumoral PD-1⁺CD8⁺ T cells, αβILTCks, and their respective thymic precursors.

Supplementary Table 4. Differentially expressed genes between intratumoarl NK1.1⁺ and NK1.1⁻ αβILTCks.

Supplementary Table 1

| | | SEQ | | SEQ | TCRa | TCRb | |
|---|---|---|---|---|---|---|---|
| | | ID | | ID | CDR3 | CDR3 | |
| Cell type | TCRa_CDR3 | NO | TCRb_CDR3 | NO | Length | Length | Count |
| PD-1+ TC | CAVTADYSNNRLTL | 69 | CASSTPGWGYQDTQYF | 184 | 14 | 16 | 18 |
| PD-1+ TC | CAPGTGGNNKLTF | 70 | CASSIDWNSQNTLYF | 185 | 13 | 15 | 13 |
| PD-1+ TC | CAVIGGTGSNRLTF | 71 | CASSFGLRPFAEQFF | 186 | 14 | 15 | 10 |
| PD-1+ TC | CAGYQGGRALIF | 72 | CASSIGLGGNYAEQDYFF | 187 | 12 | 18 | 9 |
| PD-1+ TC | CAASAIPGYQNFYF | 73 | CASSEDWGEEDTQYF | 188 | 14 | 15 | 5 |
| PD-1+ TC | CAVRAHDTNAYKVIF | 74 | CASSTPGWGYQDTQYF | 189 | 15 | 16 | 5 |
| PD-1+ TC | CAASAIPGYQNFYF | 75 | CASSEDWGEEDTQYF | 190 | 14 | 15 | 4 |
| PD-1+ TC | CAMMSNYNVLYF | 76 | CASSIGLGGNYAEF | 191 | 12 | 14 | 4 |
| PD-1+ TC | CALGDLGGSSGNKLIF | 77 | CASSPGTGVSNERF | 192 | 16 | 14 | 3 |
| PD-1+ TC | CAANSGGSNAKLTF | 78 | CASSIDWNSQNTLYF | 193 | 14 | 15 | 3 |
| PD-1+ TC | CAPGTGGNNKLTF | 79 | CASSIDWNSQNTLYF | 194 | 13 | 15 | 3 |
| PD-1+ TC | CAGYQGGRVLIF | 80 | CASSIGLGGNYAEQKFF | 195 | 12 | 17 | 2 |
| PD-1+ TC | CALSDQVNYAQGLTF | 81 | CASSLGGFYEQYF | 196 | 15 | 13 | 2 |
| PD-1+ TC | CALGDLGGSSGNKLIF | 82 | CASSFSTLKIQPTEF | 197 | 16 | 15 | 2 |
| PD-1+ TC | CAVSADYSNNRLTL | 83 | CASSLGGFYEQYF | 198 | 14 | 13 | 2 |
| PD-1+ TC | CALANSAGNKLTF | 84 | CASSPGTGVSNERLFF | 199 | 13 | 16 | 1 |
| PD-1+ TC | CAVSAPNTNKVVF | 85 | CASRVPTPSGNKVYF | 200 | 13 | 15 | 1 |
| PD-1+ TC | CALSDQGTTTRDPF | 86 | CASSLGGFYEQYF | 201 | 14 | 13 | 1 |
| PD-1+ TC | CAVSVANNAGAKLTF | 87 | CASSADRYEQYF | 202 | 15 | 12 | 1 |
| PD-1+ TC | CAPGVGGNNKLTF | 88 | CASSIDWNSQNTLYF | 203 | 13 | 15 | 1 |
| PD-1+ TC | CSVSAGNSNNRFTL | 89 | CASSLGGFYEQYF | 204 | 14 | 13 | 1 |
| PD-1+ TC | CAVIGGTGSNRLTF | 90 | CASSFGLRPFAEQF | 205 | 14 | 14 | 1 |
| PD-1+ TC | CAVSEQGGRALIF | 91 | CASSLTGTGGYTQYF | 206 | 13 | 15 | 1 |
| PD-1+ TC | CALSGYNRIFF | 92 | CASSEDWGEEDTQYF | 207 | 11 | 15 | 1 |
| PD-1+ TC | CAGYQGGRALIF | 93 | CASSPGTNSDYTF | 208 | 12 | 13 | 1 |
| PD-1+ TC | CAVSADYSNNRLTL | 94 | CASSIGLGGNYAEQKFF | 209 | 14 | 17 | 1 |
| PD-1+ TC | CALRDQGTTSQGLTF | 95 | CASSLGGFYEQYF | 210 | 15 | 13 | 1 |
| PD-1+ TC | CAPGTGGNNKLTF | 96 | CASSIDWNSQNTLYF | 211 | 13 | 15 | 1 |
| PD-1+ TC | CAGRDRGTATRDPF | 97 | CASSLGGFYEQYF | 212 | 14 | 13 | 1 |
| PD-1+ TC | CAVSANTGYQNFYF | 98 | CASSPGPREQYF | 213 | 14 | 12 | 1 |
| PD-1+ TC | CAAGYTGNYKYVF | 99 | CASSIGNTEVFF | 214 | 13 | 12 | 1 |
| PD-1+ TC | CAASGNNYAQGLTF | 100 | CAVKRGAHRAAAYF | 215 | 14 | 14 | 1 |
| PD-1+ TC | CRAYQGGRALIF | 101 | CASSIGLGGNYAEF | 216 | 12 | 14 | 1 |
| PD-1+ TC | CAPGTGGNNKLTF | 102 | CASSIDWNSQNTLYF | 217 | 13 | 15 | 1 |
| PD-1+ TC | CAANSGRSNAKLTF | 103 | CASSIDWNSQNTLYF | 218 | 14 | 15 | 1 |
| PD-1+ TC | CAGYQGGRALKF | 104 | CASIGLGGNYAEQFF | 219 | 12 | 15 | 1 |
| PD-1+ TC | CGVRATNYAQGLTF | 105 | CASSLGGRGAETLYF | 220 | 14 | 15 | 1 |
| PD-1+ TC | CACPQTNTGKLIF | 106 | CASSPRGTEVFF | 221 | 13 | 12 | 1 |
| PD-1+ TC | CVLRYMTAMLTKSSF | 107 | CASSTPGWGYQDYF | 222 | 15 | 14 | 1 |
| PD-1+ TC | CVVRYMTQIITKSSF | 108 | CASSTPGWGYQDYF | 223 | 15 | 14 | 1 |
| PD-1+ TC | CAAMNYGSSGNKLIF | 109 | CANSSLAGDYEQYF | 224 | 15 | 14 | 1 |
| PD-1+ TC | CATNNYAQGLTF | 110 | CASSSLAGDYEQYF | 225 | 12 | 14 | 1 |
| PD-1+ TC | CALGASSGSWQLIF | 111 | CASSSLAGDYEQYF | 226 | 14 | 14 | 1 |
| PD-1+ TC | CAASPSTDNSKLIWF | 112 | CASSTPGWGYQDTQYF | 227 | 15 | 16 | 1 |
| PD-1+ TC | CAVTADYSNNRLTLF | 113 | CASSTPGWGYQDTQYF | 228 | 16 | 16 | 1 |
| PD-1+ TC | CVRYATTMPTKSSF | 114 | CASSLRHSDYTF | 229 | 14 | 12 | 1 |
| PD-1+ TC | CAVRYATQAITALSF | 115 | CASSTPGWGYQDTQYF | 230 | 15 | 16 | 1 |
| PD-1+ TC | CVPTTNAYKVIF | 116 | CASSTPGWGYQDTQYF | 231 | 12 | 16 | 1 |
| PD-1+ TC | CAVRHDTNAYKVIF | 117 | CASSPRTNQDTQYF | 232 | 14 | 14 | 1 |
| αβILTCK | CALGPWGSAGNKLIF | 118 | CASSRGGHTEVFF | 233 | 15 | 13 | 5 |
| αβILTCK | CAASENNNAPRF | 119 | CASSRKNQDTQYF | 234 | 12 | 13 | 3 |
| αβILTCK | CALGDRDSGGSNYKLT | 120 | CASSLSWGDAETLYF | 235 | 17 | 15 | 3 |
| | F | | | | | | |
| αβILTCK | CAIDAASSGSWQLIF | 121 | CASRMGGANTEVFF | 236 | 15 | 14 | 2 |
| αβILTCK | CAPRVSGSWQLIF | 122 | CASSQGVISNERLFF | 237 | 13 | 15 | 2 |
| αβILTCK | CAASGGYNQGKLIF | 123 | CAAWVCTPGQYTYVLF | 238 | 14 | 16 | 2 |
| αβILTCK | CAVSANTGYQNFYF | 124 | CASSPRDWGYEQYF | 239 | 14 | 14 | 1 |
| αβILTCK | CAAPNVGDNSKLIW | 125 | CASSPLGGLREQYF | 240 | 14 | 14 | 1 |
| αβILTCK | CAVSAGGSNAKLTF | 126 | CASSPRDWGYEQYF | 241 | 14 | 14 | 1 |
| αβILTCK | CSVVLAVRAAKAMVF | 127 | CASSPDRANTEVFF | 242 | 15 | 14 | 1 |
| αβILTCK | CAVSANNNNAPRF | 128 | CASSLTGGHNERLFF | 243 | 13 | 15 | 1 |
| αβILTCK | CAADTNAYKVIF | 129 | CASRGLGANQDTQYF | 244 | 12 | 15 | 1 |
| αβILTCK | CALSDRGDSNYQLIW | 130 | CASSADWVSNERLFF | 245 | 15 | 15 | 1 |
| αβILTCK | CVLSSNYNQGKLIF | 131 | CASSILAYEQYF | 246 | 14 | 12 | 1 |
| αβILTCK | CAVSAVSGSWVLIF | 132 | CASIVETSRHNSLVF | 247 | 14 | 15 | 1 |
| αβILTCK | CAMRDYNYAQGLTF | 133 | CASSRTGGSQNTLYF | 248 | 14 | 15 | 1 |
| αβILTCK | CAMRFDTNAYKVIF | 134 | CASSWDRGAGNTLYF | 249 | 14 | 15 | 1 |
| αβILTCK | CAASPNVGYVLTF | 135 | CASSLEENTGQLYF | 250 | 13 | 14 | 1 |
| αβILTCK | CAVSRLDSNYQLIW | 136 | CASSGTGGLQNTLYF | 251 | 14 | 15 | 1 |
| αβILTCK | CVLSGSASSNTNKVVF | 137 | CASSPGTNYAEQFF | 252 | 16 | 14 | 1 |
| αβILTCK | CALRHQANYAQGLTF | 138 | CASSDWGGEQYF | 253 | 15 | 12 | 1 |
| αβILTCK | CAADASSGSWQLIF | 139 | CAVPAVGTGSSSAF | 254 | 14 | 14 | 1 |
| αβILTCK | CALSEGTGYQNFYF | 140 | CASGGGTGWEQYF | 255 | 14 | 13 | 1 |
| αβILTCK | CAVITNTGKLTF | 141 | CASPSGIGRSGASTLYF | 256 | 12 | 17 | 1 |
| αβILTCK | CAMERNTGANTGKLTF | 142 | CASTCSATGYEQYF | 257 | 16 | 14 | 1 |
| αβILTCK | CAVSAAGSALGRLHF | 143 | CASSPGTLYAEQFF | 258 | 15 | 14 | 1 |
| αβILTCK | CALSEATRDNNKLTF | 144 | SASSHRSGGYAEQFF | 259 | 15 | 15 | 1 |
| αβILTCK | CALSEGNYAQGLTF | 145 | CASSRTGGGYAEQFF | 260 | 14 | 15 | 1 |
| αβILTCK | CALSYPNQGGSAKLIF | 146 | CASSRDWGTYEQYF | 261 | 16 | 14 | 1 |
| αβILTCK | CAMRENQGGSAKLIF | 147 | CAVVTGTGGKHLVSF | 262 | 15 | 15 | 1 |
| αβILTCK | CAPSTGGNNKLTF | 148 | CASSLSRSNTEVFF | 263 | 13 | 14 | 1 |
| αβILTCK | CALGEAYNQGKLIF | 149 | CASGDEDRYSQYF | 264 | 14 | 13 | 1 |
| αβILTCK | CAMREAQGGRALIF | 150 | CGARGRTGRNTLYF | 265 | 14 | 14 | 1 |
| αβILTCK | CAGNTEGADRLTF | 151 | CASSHDPGDYAEQFF | 266 | 13 | 15 | 1 |
| αβILTCK | CALSRGSAGNKLTF | 152 | CASSLTGGYAEQFF | 267 | 14 | 14 | 1 |
| αβILTCK | CAMREGNYAQGLTF | 153 | CASSLGTANTGQLYF | 268 | 14 | 15 | 1 |
| αβILTCK | CAASGNYAQGLTF | 154 | CASGVRDWGDAEQFF | 269 | 13 | 15 | 1 |
| αβILTCK | CAVSTSNMGYKLTF | 155 | CASSPRQNNYAEQFF | 270 | 14 | 15 | 1 |
| αβILTCK | CAVSTSNMGYKLTF | 156 | CASSPRQNNYAEQFF | 271 | 14 | 15 | 1 |
| αβILTCK | CAVSGGASNNRIFF | 157 | CASSLWGGREQYF | 272 | 14 | 13 | 1 |
| αβILTCK | CAVSMPNTNKVVF | 158 | CASGPRLGTSQNTLYF | 273 | 13 | 16 | 1 |
| αβILTCK | CAVRDSNYQLIW | 159 | CASTPTGGYYAEQFF | 274 | 12 | 15 | 1 |
| αβILTCK | CALGANYNQGKLIF | 160 | CASSFDWESAETLYF | 275 | 14 | 15 | 1 |
| αβILTCK | CAVTTNSGTYQRF | 161 | CASSVGDRANERLFF | 276 | 13 | 15 | 1 |
| αβILTCK | CAASPNMGYKLTF | 162 | CASSLEENTGQLYF | 277 | 13 | 14 | 1 |
| αβILTCK | CAASPNMGYKLTF | 163 | CASSLEENTGQLYF | 278 | 13 | 14 | 1 |
| αβILTCK | CAASPNMGYKLTF | 164 | CASSLEENTGQLYF | 279 | 13 | 14 | 1 |
| αβILTCK | CALGGITGNTGKLIF | 165 | CASSLSGGSYEQYF | 280 | 15 | 14 | 1 |
| αβILTCK | CAGSNSGTYQRF | 166 | CASSFSRGDSDYTF | 281 | 12 | 14 | 1 |
| αβILTCK | CAVSRGRIFF | 167 | CTCSADPENTEVFF | 282 | 10 | 14 | 1 |
| αβILTCK | CAMVGYAQGLTF | 168 | CASSPSGGAGGAETLYF | 283 | 12 | 17 | 1 |
| αβILTCK | CAAERGTGNYKYVF | 169 | CTCSVTGGANTGQLYF | 284 | 14 | 16 | 1 |
| αβILTCK | CAMRENTGYQNFYF | 170 | CASGDQGNTEVFF | 285 | 14 | 13 | 1 |
| αβILTCK | CATLASSGSWQLIF | 171 | CASSPRDWGGARAETLYF | 286 | 14 | 18 | 1 |
| αβILTCK | CAVSAAWRLAKVVF | 172 | CTCSADTTETLYF | 287 | 14 | 13 | 1 |
| αβILTCK | CAAGPASSGSWQLIF | 173 | CTCSALPGADTEVFF | 288 | 15 | 15 | 1 |
| αβILTCK | CAASASSGSWQLIF | 174 | CAARTGEVSAETLYF | 289 | 14 | 15 | 1 |
| αβILTCK | CALGDYAQGLTF | 175 | CASSFAAEANTETLYF | 290 | 12 | 16 | 1 |
| αβILTCK | CAVSGDTAAYKVIF | 176 | CASSLWGAETLYF | 291 | 14 | 13 | 1 |
| αβILTCK | CVLGDHYNQGKLIF | 177 | CASSGTPGYSDYTF | 292 | 14 | 14 | 1 |
| αβILTCK | CATEGGRALIF | 178 | CASSFGWGAQNTLYF | 293 | 11 | 15 | 1 |
| αβILTCK | CAMERSGYGSSGNKLI | 179 | CASSPRDRNTLYF | 294 | 17 | 13 | 1 |
| | F | | | | | | |
| αβILTCK | CAMRDVNTGYQNFYF | 180 | CASSPGLGGSQNTLYF | 295 | 15 | 16 | 1 |
| αβILTCK | CALSKGHKCLQSHAAF | 181 | CASSLWPDNYAEQFF | 296 | 16 | 15 | 1 |
| αβILTCK | CVKGDHYNQGKLIF | 182 | CASSGTPGYSDYTF | 297 | 14 | 14 | 1 |
| αβILTCK | CAIADNYAQGLTF | 183 | CASSLQNTLYF | 298 | 13 | 11 | 1 |

Supplementary Table 2

| | Va Sequence (SEQ ID NOs: 299-338, in order of appearance) |
|---|---|
| NK16 | ATGAAGAGGCTGCTGTGTTCTCTGCTGGGGCTTCTGTGCACCCAGGTTTGCTGGGTGAAAGGACAGCAAGTGCAGCAGAGTCCCTCGTCCTTGGTTCTG |
| | CAGGAGGGGGAGAACGCAGAGCTGCAGTGTAACTTTTCCTCCACAGCAACCCAGCTGCAGTGGTTTTACCAAAGTCCTGGGGGAAGCCTCGTCAGCCT |
| | GTTGTCCAATCCTTCTGGGACAAAGCACACTGGAAGACTGACATCCACCACAGTCACTAAGGAACGTCGCAGCTCTTTGCACATTTCCTCCTCCCAGACA |
| | ACAGACTCAGGCACTTATCTCTGTGCTATAGAGGCAAATTCTGGGACTTACCAGAGGTTTGGAACTGGGACAAAACTCCAAGTCGTTCCAA |
| NK28 | ATGAACAGATTCCTGGGAATATCTTTGGTGACTCTATGGTTTCGAGTGGCCTGGGCAAAGAGCCAATGGGGAGAAGAGAATCTTCAGGCTCTGAGCATC |
| | CAGGAGGGTGAAGATGTCACCATGAACTGCAGTTACAAGACTTATACAACTGTTGTACACTGGTACAGGCAAGACTCAGGCAGAGGCCCTGCCCTGATA |
| | ATCTTAATACGTTCAAATGAGCGAGAGAAGCGCAGTGGAAGACTAAGAGCCACCCTTGACACCTCCAGCCAAAGTAGCTCTCTGTCCATCACTGCTGCTC |
| | AGTGTGAAGACACTGCTGTGTACTTCTGTGCTACAGATGACCAGGGAGGCAGAGCTCTGATATTTGGAACAGGAACCACGGTATCAGTCAGCCCCA |
| NK25 | ATGGACAAGATCCTGACAGCATCGTTTTTACTCCTAGGCCTTCACCTAGCTGGGGTGAGTGGCCAGCAGCAGGAGAAACGTGACCAGCAGCAGGTGAG |
| | ACAAAGTCCCCAATCTCTGACAGTCTGGGAAGGAGAGACAGCAATTCTGAACTGCAGTTATGAGGACAGCACTTTTGACTACTTCCCATGGTACTGGCA |
| | GTTCCCTAGGGAAAGCCCTGCACTCCTGATAGCCATACGTCCAGTGTCCAATAAAAAGGAAGATGGACGATTCACAATCTTCTTCAATAAAAGGGAGAA |
| | AAAGCTCTCCTTGCACATCACAGACTCTCAGCCTGGAGACTCAGCTACCTACTTCTGTGCAGCAAGTGACATGGGCTACAAACTTACCTTCGGGACAGGA |
| | ACAAGCCTGTTGGTTGATCCAA |
| NK186 | ATGAATACTTCTCCAGTTTTAGTGACTGCGATGCTGCTGTTCATGCTTAAGACCCACGGAGATTCAGTGACCCAGAAACAAGGTCAAGTGACCCTTTCAG |
| | AAGATGACTTCCTATTTATAAATTGCACTTATTCTACCACAACGTACCCAACTCTTTTCTGGTATGTCCAATATCCTGGACAAGGTCCACAGCTCCTTCTGA |
| | AAGTCACAACTGCCAACAACAAGGGAATCAGCAGAGGCTTTGAAGCTACATATGACAAAGGGACCACCTCCTTCCACTTACAGAAAGCCTCAGTGCATG |
| | AGTCAGACTCAGCCGTGTACTTCTGTGCTCTGGGAGACTATGCCCAGGGATTAACCTTCGGTCTTGGCACCAGAGTATCTGTGTTTCCCT |
| NK20 | ATGAAGAGCCTGCTGAGCTCTCTGCTGGGGCTTCTGTGCACCCAGGTTTGCTGGGTGAAAGGACAGCAAGTGCAGCAGAGCCCCGCGTCCTTGGTTCTG |
| | CAGGAGGGGGAGAACGCAGAGCTGCAGTGTAACTTTTCCACATCTTTGAACAGTATGCAGTGGTTTTACCAACGTCCTGGGGGAAGTCTCGTCAGCCTG |
| | TTCTACAATCCTTCTGGGACAAAGCATAGTGGGAGACTGACATCCACTACAGTCATCAAAGAACGTCGCAGCTCTTTGCACATTTCCTCCTCCCAGACAAC |
| | AGACTCAGGCACTTATCTCTGTGCTTTGGAAACGGGTTCAGCCTTAGGGAGGCTGCATTTTGGAGCTGGGACTCAGCTGATTGTCATACCTG |
| NK22 | ATGAACAGATTCCTGGGAATATCTTTGGTGACTCTATGGTTTCAAGTGGCCTGGGCAAAGAGCCAATGGGGAGAAGAGAATCTTCAGGCTCTGAGCATC |
| | CAGGAGGGTGAAGATGTCACCATGAACTGCAGTTACAAGACTTACACAACTGTTGTTCAGTGGTACAGACAGAAGTCAGGCAAAGGCCCTGCCCAGCT |
| | AATCTTAATACGTTCAAATGAGCGAGAGAAGCGCAGTGGAAGACTCAGAGCCACCCTTGACACTTCCAGCCAGAGCAGCTCCCTGTCCATCACTGGTAC |
| | TCTAGCTACAGACACTGCTGTGTACTTCTGTGCTACTGAGCCCTATGACTCGGGATACAACAAACTCACTTTTGGAAAGGGCACGGTGCTTCTAGTCTCTC |
| | CAG |
| NK17 | ATGCGTCCTGACACCTGCTCAGTTCTTGTGCTCCTCTTAATGCTCAGAAGGAGCAATGGAGACTCAGTGACCCAGACAGAAGGCCTGGTCACTCTCACCA |
| | AGGGGTTGCCTGTGATGCTGAACTGCACCTATCAGACTACTTACTCACCTTTCCTTTTCTGGTATGTGCAACATCTCAACGAAGCCCCTAAGCTACTCCTG |
| | AAGAGCTCCACAGACAACAAGAGGACCGAGCACCAAGGGTTCTACGCCACTCTCCATAAGAGCAGCAGCTCCTTCCATCTGCAGAAGTCCTCAGTGCAG |
| | CTGTCAGACTCTGCCCTGTACTTCTGTGCTTTGAGTGATGGGACTGGAGGCTATAAAGTGGTCTTTGGAAGTGGGACTCGATTGCTGGTAAGCCCTG |
| NK139 | ATGGACAAGATCCTGACAGCAACGTTTTTACTCCTAGGCCTTCACCTAGCTGGGGTGAATGGCCAGCAGCAGGAGAAACGTGACCAGCAGCAGGTGAG |
| | ACAAAGTCCCCAATCTCTGACAGTCTGGGAAGGAGAGACCGCAATTCTGAACTGCAGTTATGAGGACAGCACTTTTAACTACTTCCCATGGTACCAGCA |
| | GTTCCCTGGGGAAGGCCCTGCACTCCTGATATCCGTACGTTCAGTGTCCGATAAAAAGGAAGATGGACGATTCACAATCTTCTTCAATAAAAGGGAGAA |
| | AAAGCTCTCCTTGCACATCACAGACTCTCAGCCTGGAGACTCAGCTACCTACTTCTGTGCAGCAGGTCCAGCATCTTCTGGCAGCTGGCAACTCATCTTTG |
| | GATCTGGAACCCAACTGACAGTTATGCCTG |
| NK12 | ATGCTGATTCTAAGCCTGTTGGGAGCAGCCTTTGGCTCCATTTCAACCAGCATGGCCCAGAAGGTAACACAGACTCAGACTTCAATTTCTGTGATGGAGA |
| | AGACAACGGTGACAATGGACTGTGTGTATGAAACCCAGGACAGTTCTTACTTCTTATTCTGGTACAAGCAAACAGCAAGTGGGGAAATAGTTTTCCTTAT |
| | TCGTCAGGACTCTTACAAAAAGGAAAATGCAACAGTGGGTCATTATTCTCTGAACTTTCAGAAGCCAAAAAGTTCCATCGGACTCATCATCACCGCCACA |
| | CAGATTGAGGACTCAGCAGTATATTTCTGTGCTATGAGACCCAACTATGGAAATGAGAAAATAACTTTTGGGGCTGGAACCAAACTCACCATTAAACCCA |
| NK11 | ATGAAATCCTTGAGTGTTTCCCTAGTGGTCCTGTGGCTCCAGTTAAACTGGGTGAAGAGCCAGCAGAAGGTGCAGCAGAGCCCAGAATCCCTCAGTGTC |
| | CCAGAGGGAGGCATGGCCTCTCTCAACTGCACTTCAAGTGATCGTAATTTTCAGTATTTCTGGTGGTACAGACAGCATTCTGGAGAAGGCCCCAAAGCA |
| | CTGATGTCCATCTTCTCTGATGGTGACAAGAAAGAAGGCAGATTCACAGCTCACCTCAATAAGGCCAGCCTGCATGTTTCCCTGCACATCAGAGACTCCC |
| | AGCCCAGTGACTCCGCTCTCTACTTCTGTGCAGTTAGTGCTGCATCTTCTGGCAGCTGGCAACTCATCTTTGGATCTGGAACCCAACTGACAGTTATGCCT |
| | G |
| NK24 | ATGGACTCTTCTCCAGGCTTCGTGGCTGTGATACTTCTCATACTTGGAAGGACCCACGGAGATTCCGTGACTCAAACAGAAGGCCAAGTGACCGTCTCAG |
| | AAAGCAAGTCCCTGATAATAAATTGCACGTATTCAACCACAAGCATAGCTTACCCTAATCTTTTCTGGTATGTTCGATATCCTGGAGAAGGTCTACAACTC |
| | CTCCTGAAAGTCATTACGGCTGGCCAGAAGGGAAGCAGCAGAGGGTTTGAAGCCACATACAATAAAGAAACCACCTCCTTCCACTTGCAGAAAGCCTCA |
| | GTGCAAGAGTCAGACTCGGCTGTGTACTACTGTGCTCTGGCCCTCATGAATTATAACCAGGGGAAGCTTATCTTTGGACAGGGAACCAAGTTATCTATCA |
| | AGCCCA |
| NK18 | ATGAACTATTCTCCAGCTTTAGTGACTGTGATGCTGTTTGTGTTTGGGAGGACCCATGGAGACTCAGTGACCCAGATGCAAGGTCAAGTGACCCTCTCAG |
| | AAGACGACTTCCTATTTATAAACTGTACTTATTCAACCACATGGTACCCGACTCTTTTCTGGTATGTCCAATATCCCGGAGAAGGTCCACAGCTCCTTTTGA |
| | AAGTCACAACAGCCAACAACAAGGGAATCAGCAGAGGTTTTGAAGCTACATATGATAAAGGAACAACGTCCTTCCACTTGCAGAAAGCCTCAGTGCAG |
| | GAGTCAGACTCTGCTGTGTACTACTGTGTTCTGGGTGGGGCTTCTGGCAGCTGGCAACTCATCTTTGGATCTGGAACCCAACTGACAGTTATGCCTG |
| NK23 | ATGCTGATTCTAAGCCTGCTGGGAGCAGCCTTTGGCTCCATTTGTTTTGCAACCAGCATGGCCCAGAAGGTAACACAGACTCAGACTTCAATTTCTGTGA |
| | TGGAGAAGACAACGGTGACAATGGACTGTGTGTATGAAACCCGGGACAGTTCTTACTTCTTATTCTGGTACAAGCAAACAGCAAGTGGGGAAATAGTTT |
| | TCCTTATTCGTCAGGACTCTTACAAAAAGGAAAATGCAACAGAAGGTCATTATTCTCTGAACTTTCAGAAGCCAAAAAGTTCCATCGGACTCATCATCACT |
| | GCCACACAGATTGAGGACTCAGCAGTATATTTCTGTGCTATGAGGGCCAACTATGGAAATGAGAAAATAACTTTTGGGGCTGGAACCAAACTCACCATT |
| | AAACCCA |
| NK21 | ATGCTGATTCTAAGCCTGCTGGGAGCAGCCTTTGGCTCCATTTGTTTTGCAACCAGCATGGCCCAGAAGGTAACACAGACTCAGACTTCAATTTCTGTGA |
| | TGGAGAAGACAACGGTGACAATGGACTGTGTGTATGAAACCCGGGACAGTTCTTACTTCTTATTCTGGTACAAGCAAACAGCAAGTGGGGAAATAGTTT |
| | TCCTTATTCGTCAGGACTCTTACAAAAAGGAAAATGCAACAGAAGGTCATTATTCTCTGAACTTTCAGAAGCCAAAAAGTTCCATCGGACTCATCATCACT |
| | GCCACACAGATTGAGGACTCAGCAGTATATTTCTGTGCTATGAGAGAGGGGGGTGGAGGCAGCAATTACAAACTGACATTTGGGAAAGGAACTCTCTT |
| | AACTGTGACTCCAA |
| NK19 | ATGAACTATTCTCCAGCTTTAGTGACTGTGATGCTGTTTGTGTTTGGGAGGACCCATGGAGACTCAGTGACCCAGATGCAAGGTCAAGTGACCCTCTCAG |
| | AAGACGACTTCCTATTTATAAACTGTACTTATTCAACCACATGGTACCCGACTCTTTTCTGGTATGTCCAATATCCCGGAGAAGGTCCACAGCTCCTTTTGA |
| | AAGTCACAACAGCCAACAACAAGGGAATCAGCAGAGGTTTTGAAGCTACATATGATAAAAGAACAACGTCCTTCCACTTGCAGAAAGCCTCAGTGCAGG |
| | AGTCAGACTCTGCTGTGTACTACTGTGTTCTGGGTGCCTCGGGATACAACAAACTCACTTTTGGAAAGGGCACGGTGCTTCTAGTCTCTCCAG |
| NK10 | ATGCACAGCCTCCTGGGGTTGTTGATGGTGTCACTGTGGCTGCAACTGACAAGGGTGAATAGTCAACTAGCAGAAGAGAATCCGTGGGCCCTGAGCGT |
| | CCACGAGGGTGAAAGTGTCACGGTGAATTGTAGTTACAAGACATCCATAACTGCCCTACAGTGGTACAGACAGAAGTCAGGCGAAGGCCCTGCCCAGC |
| | TAATCTTAATACGTTCAAATGAGAGAGAGAAGCGCAATGGAAGACTCAGAGCCACCCTTGACACCTCCAGCCAGAGCAGCTCCTTGTCCATCACTGCTAC |
| | TCGGTGTGAAGACACCGCTGTGTACTTCTGTGCTACTGATCACATGGGCTACAAACTTACCTTCGGGACAGGAACAAGCTTGTTGGTTGATCCAG |
| NK15 | ATGCTCCTGGCGCTCCTCCCAGTGCTGGGGATACACTTTGTCCTGAGAGATGCCCAAGCTCAGTCAGTGACGCAGCCCGATGCTCGCGTCACTGTCTCTG |
| | AAGGAGCCTCTCTGCAGCTGAGATGCAAGTATTCCTCCTCTGGGACACCTTATCTGTTCTGGTATGTCCAGTACCCGCGGCAGGGGCTGCAGCTGCTCCT |
| | CAAGTACTATTCCGGAGACCCAGTGGTTCAAGGAGTGAATGGCTTTGAGGCTGAGTTCAGCAAGAGTAACTCTTCCTTCCACCTGCGGAAAGCCTCTGT |
| | GCACTGGAGCGACTCGGCTGTGTACTTCTGTGTTCTGAGCGGATATGGGAGCAGTGGCAACAAGCTCATCTTTGGAATTGGGACTCTGCTTTCTGTCAA |
| | GCCAA |
| NK183 | ATGAAGAGGCTGCTGTGCTCTCTGCTGGGGCTTCTGTGCACCCAGGTTTGCTGGGTGAAAGGACAGCAAGTGCAGCAGAGTCCCACGTCCTTGGTTCTG |
| | CAGGAGGGGGAAAACGCAGAGCTGCAGTGTAACTTTTCCACATCTTTGAACAGTATGCAGTGGTTTTACCAACGTCCTGGGGGAAGTCTCGTCAGCGTG |
| | TTCTACAATCCTTCTGGGACAAAGCAGAGTGGGAGACTGACATCCACAACAGTCATTAAAGAACGCCGCAGCTCTTTGCACATTTCCTCCTCCCAGACAA |
| | CAGACTCAGGCACTTATCTCTGTGCTTTGGCCCAAGGCACTGGGTCTAAGCTGTCATTTGGGAAGGGGGCAAAGCTCACAGTGAGTCCAG |
| NK30 | ATGAAATCCTTGAGTGTTTCACTAGTGGTCCTGTGGCTCCAGTTAAACTGCGTGAGGAGCCAGCAGAAGGTGCAGCAGAGCCCAGAATCCCTCAGTGTC |
| | CCAGAGGGAGGCATGGCCTCTCTCAACTGCACTTCAAGTGATCGTAATTTTCAGTACTTCTGGTGGTACAGACAGCATTCTGGGAAAAGCCCCAAGATG |
| | CTGATGTCTATCTTCTCCAATGGTGAAAAGGAAGAAGGCAGATTCACAGTTCACCTCAATAAAGCCAGCCTGCATACTTCCCTGCACATCAGAGACTCCC |
| | AGCCCAGTGACTCTGCTCTCTACCTCTGTGCAGCAAGCAGGGATCTACCAGGCACTGGGAGTAACAGGCTCACTTTTGGGAAAGGCACCAAATTCTCACT |
| | CATCCCGA |
| NK150 | ATGAACTCTTCTCCAGGCTTCATGACTGTGATGCTCCTCATATTCACAAGGGCCCATGGAGACTCAGTGACTCAGACGGAAGGTCAAGTGGCCCTCTCAG |
| | AAGAGGACTTTCTTACGATACACTGCAACTACTCAGCCTCAGGGTACCCAGCTCTGTTCTGGTATGTGCAGTATCCCGGAGAAGGTCCACAGCTCCTCTTT |
| | AGAGCCTCAAGGGACAAAGAGAAAGGAAGCAGCAGAGGTTTTGAAGCTACATATGATAAAGGGACCACCTCCTTCCACTTGCGGAAAGCCTCAGTGCA |
| | TGAGTCAGACTCGGCTGTGTACTACTGTGCTCTGGGTGATTTAGAATCTGGGACTTACCAGAGGTTTGGAACTGGGACAAAACTCCAAGTCGTTCCAA |
| NK32 | ATGCGTCCTGACACCTGCTCAGTTCTTGTGCTCCTCTTAATGCTCAGAAGGAACAATGGAGACTCTGTGACCCAGACAGAAGGCCTGGTCACTCTCACCG |
| | AGGGGTTGCCTGTGATGCTGAACTGCACCTATCAGAGTACTTACTCACCTTTCCTTTTCTGGTATGTGCAACATCTCAACGAAGCCCCTAAGCTACTTTTG |
| | AAGAGCTTCACAGACAACAAGAGGCCCGAGCACCAAGGGTTCCACGCCACTCTCCATAAGAGCAGCAGCTCCTTCCATCTGCAGAAGTCCTCAGCGCAG |
| | CTGTCAGACTCTGCCCTGTACTACTGTGCTTTGAGTGGAGGCAATACCAACAAAGTCGTCTTTGGAACAGGGACCAGATTACAAGTATTACCAG |
| NK26 | ATGATTGTGATGCTCCTCATATTCACAAGGGCCCATGGAGACTCAGTGACTCAGACGGAAGGTCAAGTGGCCCTCTCAGAAGAGGACTTTCTTACGATA |
| | CACTGCAACTACTCAGCCTCAGGGTACCCAGCTCTGTTCTGGTACGTGCAGTATCCCGGAGAAGGTCCACAGTTCCTCTTTAGAGCCTCAAGGGACAAAG |
| | AGAAAGGAAGCAGCAGAGGTTTTGAAGCTACATATGATAAAGGGACCACCTCCTTCCACTTGCGGAAAGCCTCAGTGCAAGAGTCAGACTCGGCTGTG |
| | TACTACTGTGCTCTGGTCCCGAACACCAATACAGGCAAATTAACCTTTGGGGATGGGACCGTGCTCACAGTGAAGCCAA |
| NK29 | ATGAAGAGGCTGCTGTGCTCTCTGCTGGGGCTCCTGTGCACCCAGGTTTGCTGGGTGAAAGGACAGCAAGTGGAGCAGAGTCCCGCGTCCTTGGTTCT |
| | GCAGGAGGGGGAGAACGCAGAGCTGCAGTGCACCTATTCCACAACTTTGAACAGTATGCAGTGGTTTTACCAACGTCCTGGGGGAAGACTCGTCAGCC |
| | TGTTGTACAATCCTTCTGGGACAAAGCAGAGTGGGAGACTGACATCCACAACAGTCATTAAAGAACGTCGCAGCTCTTTGCACATTTCCTCCTCCCAGAT |
| | CACAGACTCAGGCACTTATCTCTGTGCTATGGAACGGTATCTTTATGGGAGCAGTGGCAACAAGCTCATCTTTGGAATTGGGACTCTGCTTTCTGTCAAG |
| | CCAA |
| NK31 | ATGATTGTGATGCTCCTCATATTCACAAGGGCCCATGGAGACTCAGTGACTCAGACGGAAGGTCAAGTGGCCCTCTCAGAAGAGGACTTTCTTACGATA |
| | CACTGCAACTACTCAGCCTCAGGGTACCCAGCTCTGTTCTGGTACGTGCAGTATCCCGGAGAAGGTCCACAGTTCCTCTTTAGAGCCTCAAGGGACAAAG |
| | AGAAAGGAAGCAGCAGAGGTTTTGAAGCTACATATGATAAAGGGACCACCTCCTTCCACTTGCGGAAAGCCTCAGTGCAAGAGTCAGACTCGGCTGTG |
| | TACTACTGTGCTCTGGGGAATGACACAAATGCTTACAAAGTCATCTTTGGAAAAGGGACACATCTTCATGTTCTCCCTA |
| NK199 | ATGGACAAGATCCTGACAGCATCGTTTTTACTCCTAGGCCTTCACCTAGCTGGGGTGAGTGGCCAGCAGCAGGAGAAACGTGACCAGCAGCAGGTGAG |
| | ACAAAGTCCCCAATCTCTGACAGTCTGGGAAGGAGAGACAGCAATTCTGAACTGCAGTTATGAGGACAGCACTTTTGACTACTTCCCATGGTACTGGCA |
| | GTTCCCTAGGGAAAGCCCTGCACTCCTGATAGCCATACGTCCAGTGTCCAATAAAAAGGAAGATGGACGATTCACAATAGTCTTCAAGAAAAGGGAGAA |
| | AAAGCTCTCCTTGCACATCACAGACTCTCAGCCTGGAGACTCAGGTACCTACTTCTGTGCAGTATCGGCCTACCAGGGAGGCAGAGCTCTGATATTTGGA |
| | ACAGGAACCACGGTATCAGTCAGCCCCA |
| NK154 | ATGGAGAGGAGCCCGGGAACTGTGCTGGGGCTGCTGTTGGTGCCGCTTTGCTCAGGGGTAAGAGGAATGCCGGTGGAACAGAATCCTCCAGCCCTGA |
| | GTCTTTATGAAGGAGCTGACTCTGGTCTGAGATGCAATTTTTCTACCACCATGAAAAGTGTCCAGTGGTTTCAACAAAATCACAGAGGCAGACTTATCAC |
| | TCTGTTTTACCTGGCTCAAGGAACAAAGGAGAATGGAAGGCTAAAGTCAGCATTTGATTCTAAGGAGCGCTACAGCACCCTGCACATCAGGGATGCCCA |
| | GCTGGAGGACTCAGGCACTTACTTCTGTGCTGCGCACCAGGGAGGCAGAGCTCTGATATTTGGAACAGGAACCACGGTATCAGTCAGCCCCA |
| NK72 | ATGAAATCCTTGAGTGTTTCCCTAGTGGTCCTGTGGCTCCAGTTAAACTGGGTGAACAGCCAGCAGAAGGTGCAGCAGAGCCCAGAATCCCTCATTGTC |
| | CCAGAGGGAGCCATGACCTCTCTCAACTGCACTTTCAGCGACAGTGCTTCTCAGTATTTTGCATGGTACAGACAGCATTCTGGGAAAGCCCCCAAGGCAC |
| | TGATGTCCATCTTCTCTGATGGTGACAAGAAAGAAGGCAGATTCACAGCTCACCTCAATAAGGCCAGCCTGCATGTTTCCCTGCACATCAGAGACTCCCA |
| | GCCCAGTGACTCCGCTCTCTACTTCTGTGCAGCCCCTAATGTTGGTGACAACAGTAAGCTGATTTGGGGCTTGGGGACAAGTCTGGTAGTAAATCCAA |
| NK62 | ATGCATTCCTTACATGTTTCACTAGTGTTCCTCTGGCTTCAACTAGGGGTGAGCAGCCAGGAGAAGGTACAGCAGAGCCCAGAATCCCTCATTGTCCCAG |
| | AGGGAGCCATGTCCTCCCTCAACTGCACTTTCAGCAACAGTGCTTCTCAGTCCATCTGGTGGTACCAACAGCATCCTGGGAAAGGCCCCGAAGCACTAAT |
| | ATCCATATTCTCTAATGGCAACAAGAAAGAAGGCAGATTGACAGTTTACCTCAATAGAGCCAGCCTGCATGTTTCCCTGCACATCAGAGACTCCCAGCCC |
| | AGTGACTCTGCCGTCTACCTCTGTGCACCGCGAGTGTCTGGCAGCTGGCAACTCATCTTTGGATCTGGAACCCAACTGACAGTTATGCCTG |
| NK193 | ATGAAGAGGCTGCTGTGCTCTCTGCTGGGGCTTCTGTGCACCCAGGTTTGCTGGGTGAAAGGACAGCAAGTGCAGCAGAGTCCCACGTCCTTGGTTCTG |
| | CAGGAGGGGGAAAACGCAGAGCTGCAGTGTAACTTTTCCACATCTTTGAACAGTATGCAGTGGTTTTACCAACGTCCTGGGGGAAGTCTCGTCAGCGTG |
| | TTCTACAATCCTTCTGGGACAAAGCAGAGTGGGAGACTGACATCCACAACAGTCATTAAAGAACGCCGCAGCTCTTTGCACATTTCCTCCTTCCAGACAA |
| | CAGACTCAGGCACTTATTTCTGTGCTATAGATCTTAGGAATGCAGGTGCCAAGCTCACATTCGGAGGGGGAACAAGGTTAACGGTCAGACCCG |
| NK179 | ATGGACAAGATCCTGACAGCATCGTTTTTACTCCTAGGCCTTCACCTAGCTGGGGTGAGTGGCCAGCAGCAGGAGAAACGTGACCAGCAGCAGGTGAG |
| | ACAAAGTCCCCAATCTCTGACAGTCTGGGAAGGAGAGACAGCAATTCTGAACTGCAGTTATGAGGACAGCACTTTTGACTACTTCCCATGGTACTGGCA |
| | GTTCCCTAGGGAAAGCCCTGCACTCCTGATAGCCATACGTCCAGTGTCCAATAAAAAGGAAGATGGACGATTCACAATCTTCTTCAATAAAAGGGAGAA |
| | AAAGCTCTCCTTGCACATCATAGACTCTCAGCCTGGAGACTCAGCCACCTACTTCTGTGCAGCAAGTAGGACTGGCAGTGGTGGAAAACTCACTTTGGG |
| | GGCTGGAACAAGACTTCAGGTCAACCTTG |
| NK70 | ATGCTCCTGGCACTCCTCCCAGTGCTGGGGATACACTTTGTCCTGAGAGATGCCCAAGCTCAGTCAGTGACACAGCCCGATGCTCGCGTCACTGTCTCTG |
| | AAGGAGCCTCTCTGCAGCTGAGATGCAAGTATTCCTACTCTGGGACGCCTTATCTGTTCTGGTATGTCCAGTACCCGCGGCAGGGGCTGCAGCTGCTCCT |
| | CAAGTACTATTCCGGAGACCCAGTGGTTCAAGGAGTGAATGGCTTTGAGGCTGAGTTCATCAAGAGTAACTCTTCCTTCCACCTGCGGAAAGCCTCTGTG |
| | CACTGGAGCGACTCGGCTGTGTACTTCTGTGCTGTGAGGGGTTTTAATACAGGAAACTACAAATACGTCTTTGGAGCAGGTACCAGACTGAAGGTTATA |
| | GCAC |
| NK77 | ATGCAGAGGAACCTGGTTGCTGTGCTGGGGATTCTGTGGGTGCAGATTTGCTGGGTGAGCGGAGATAAGGTGAAGCAAAGTCCCTCAGCGCTGAGTCT |
| | CCAAGAGGGAACCAATTCTGCTCTGAGATGCAATTTTTCTATCGCCGCGACAACTGTGCAGTGGTTCCTACAGAATCCCAGGGGCAGCCTCATCAATCTT |
| | TTTTACCTGGTTCCAGGAACAAAGGAGAATGGAAGGTTAAAGTCAACATTCAATTCTAAGGAGAGCTACAGCACCCTGCACATCAGGGATGCCCAGCTG |
| | GAGGACTCAGGCACTTACTTCTGTGCTGCTGACACAAATGCTTACAAAGTCATCTTTGGAAAAGGGACACATCTTCATGTTCTCCCTA |
| NK198 | ATGCCTCCTCACAGCCTGCTCTGTGTGCTGGTGGCCTTGGCTATCTCTGGATCTAATGTGGCCCAGAAAGTGATTCAGGTCTGGTCAACAGCAAGCAGGC |
| | AGGAGGGCGAAGAACTCACCCTGGACTGTTCATATGAGACAAGTCAGGTCTTATACCATCTTTTCTGGTACAAGCACCTTCTTAGTGGAGAGATGGTTTT |
| | CCTTATTCGACAAATGTCTTCTTCTACTGCAAAAGAGAGGAGCGGCCGCTATTCTGTAGTCTTCCAGAAATCATTCAAGTCCATCAGCCTTGTCATTTCAG |
| | CCTCACAGCCAGAGGATTCAGGGACGTACTTCTGCGCTCTCTCGGAACTGAGAAATTACAACGTGCTTTACTTCGGATCTGGCACCAAACTCACTGTAGA |
| | GCCAA |
| PD233 | ATGGACTCTTCTCCAGGCTTCGTGGCTGTGATACTTCTCATACTTGGAAGGACCCACGGAGATTCCGTGACTCAAACAGAAGGCCAAGTGACCGTCTCAG |
| | AAAGCAAGTCCCTGATAATAAATTGCACGTATTCAACCACAAGCATAGCTTACCCTAATCTTTTCTGGTATGTTCGATATCCTGGAGAAGGTCTACAACTC |
| | CTCCTGAAAGTCATTACGGCTGGCCAGAAGGGAAGCAGCAGAGGGTTTGAAGCCACATACAATAAAGAAACCACCTCCTTCCACTTGCAGAAAGCCTCA |
| | GTGCATGAGTCAGACTCGGCTGTGTACTACTGTGCTCTGGGTGATCTCGGAGGGAGCAGTGGCAACAAGCTCATCTTTGGAATTGGGACTCTGCTTTCT |
| | GTCAAGCCAA |
| PD292 | ATGCACAGCCTCCTGGGGTTGTTGTTGTGGCTGCAACTGACAAGGGTGAATAGTCAACTAGCAGAAGAGAATTCGTGGGCCCTGAGCGTCCACGAGGG |
| | TGAAAGTGTCACGGTGAATTGTAGTTACAAGACATCCATAACTGCCCTACAGTGGTACAGACAGAAGTCAGGCAAAGGCCCTGCCCAGCTAATCTTAAT |
| | ACGTTCAAATGAGAGAGAGAAGCGCAGTGGAAGACTAAGAGCCACCCTTGACACCTCCAGCCAGAGCAGCTCCCTGTCCATCACTGCTGCTCAGTGTGA |
| | AGACACTGCTGTGTACTTCTGTGCCGCCAATTCTGGAAGAAGCAATGCAAAGCTAACCTTCGGGAAAGGCACTAAACTCTCTGTTAAATCAAACATCCAG |
| | AACCC |
| PD252 | ATGAAATCCTTGAGTGTTTCCCTAGTGGTCCTGTGGCTCCAGTTAAACTGGGTGAACAGCCAGCAGAAGGTGCAGCAGAGCCCAGAATCCCTCATTGTC |
| | CCAGAGGGAGCCATGACCTCTCTCAACTGCACTTTCAGCGACAGTGCTTCTCAGTATTTTGCATGGTACAGACAGCATTCTGGGAAAGCCCCCAAGGCAC |
| | TGATGTCCATCTTCTCCAATGGTGAAAAAGAAGAAGGCAGATTCACAATTCACCTCAATAAAGCCAGTCTGCATTTCTCCCTGCACATCAGAGACTCCCA |
| | GCCCAGTGACTCTGCTCTCTACCTCTGTGCAGTGATAGGAGGCACTGGGAGTAACAGGCTCACTTTTGGGAAAGGCACCAAATTCTCACTCATCCCGA |
| PD217 | ATGAACAGATTCCTGGGAATATCTTTGGTGACTCTATGGTTTCAAGTGGCCTGGGCAAAGAGCCAATGGGGAGAAGAGAATCTTCAGGCTCTGAGCATC |
| | CAGGAGGGTGAAGATGTCACCATGAACTGCAGTTACAAGACTTACACAACTGTTGTTCAGTGGTACAGACAGAAGTCAGGCAAAGGCCCTGCCCTGATA |
| | ATCTTAATACGTTCAAATGAGCGAGAGAAGCGCAGTGGAAGACTCAGAGCCACCCTTGACACTTCCAGCCAGAGCAGCTCCCTGTCCATCACTGGTACT |
| | CTAGCTACAGACACTGCTGTGTACTTCTGTGCTATGATGTCTAATTACAACGTGCTTTACTTCGGATCTGGCACCAAACTCACTGTAGAGCCAA |
| PD245 | ATGAAATCCTTTAGTATTTCCCTAGTGGTCCTGTGGCTTCAGCTAAACTGGGTGAACAGCCAACAGAAGGTGCAGCAGAGCCCAGAATCCCTCATTGTTC |
| | CAGAGGGAGGCATGGCCTCTCTCAACTGCACTTCCAGTGATCGTAATGTTGACTACTTCTGGTGGTACAGACAGCACTCTGGGAAAAGCCCCAAGATGC |
| | TGATGTCTATCTTCTCCAATGGTGAAAAGGAAGAAGGCAGATTGACAGTTTACCTCAATAGAGCCAGCCAGCATGTTTCCCTGCACATCAGAGAGTCCCA |
| | GCCCAGTGACTCTGCCGTCTACCTCTGTGCAGGGTACCAGGGAGGCAGAGCTCTGATATTTGGAACAGGAACCAAAGTATCAGTCAGCCCCA |
| PD272 | ATGCACAGCCTCCTGGGGTTGTTGTTGTGGCTGCAACTGACAAGGGTGAATAGTCAACTAGCAGAAGAGAATTCGTGGGCCCTGAGCGTCCACGAGGG |
| | TGAAAGTGTCACGGTGAATTGTAGTTACAAGACATCCATAACTGCCCTACAGTGGTACAGACAGAAGTCAGGCAAAGGCCCTGCCCAGCTAATCTTAAT |
| | ACGTTCAAATGAGAGAGAGAAGCGCAGTGGAAGACTAAGAGCCACCCTTGACACCTCCAGCCAGAGCAGCTCCCTGTCCATCACTGCTGCTCAGTGTGA |
| | AGACACTGCTGTGTACTTCTGTGCC |
| OT-I | ATGGACAAGATCCTGACAGCATCGTTTTTACTCCTAGGCCTTCACCTAGCTGGGGTGAATGGCCAGCAGCAGGAGAAACGTGACCAGCAGCAGGTGAG |
| | ACAAAGTCCCCAATCTCTGACAGTCTGGGAAGGAGAGACCGCAATTCTGAACTGCAGTTATGAGGACAGCACTTTTAACTACTTCCCATGGTACCAGCA |
| | GTTCCCTGGGGAAGGCCCTGCACTCCTGATATCCATACGTTCAGTGTCCGATAAAAAGGAAGATGGACGATTCACAATCTTCTTCAATAAAAGGGAGAA |
| | AAAGCTCTCCTTGCACATCACAGACTCTCAGCCTGGAGACTCAGCTACCTACTTCTGTGCAGCAAGTGACAACTATCAGTTGATCTGGGGCTCTGGGACC |
| | AAGCTAATTATAAAGCCAG |
| | Vb Sequence (SEQ ID NOs: 339-378, in order of appearance) |
| NK16 | ATGGGCTCCATTTTCCTCAGTTGCCTGGCCGTTTGTCTCCTGGTGGCAGGTCCAGTCGACCCGAAAATTATCCAGAAACCAAAATATCTGGTGGCAGTCA |
| | CAGGGAGCGAAAAAATCCTGATATGCGAACAGTATCTAGGCCACAATGCTATGTATTGGTATAGACAAAGTGCTAAGAAGCCTCTAGAGTTCATGTTTT |
| | CCTACAGCTATCAAAAACTTATGGACAATCAGACTGCCTCAAGTCGCTTCCAACCTCAAAGTTCAAAGAAAAACCATTTAGACCTTCAGATCACAGCTCTA |
| | AAGCCTGATGACTCGGCCACATACTTCTGTGCCAGCAGCCAAGAACGGGCCCAGGGTAGTGCAGAAACGCTGTATTTTGGCTCAGGAACCAGACTGACT |
| | GTTCTCG |
| NK28 | ATGGGCTGTAGGCTCCTAAGCTGTGTGGCCTTCTGCCTCTTGGGAATAGGCCCTTTGGAGACGGCTGTTTTCCAGACTCCAAACTATCATGTCACACAGG |
| | TGGGAAATGAAGTGTCTTTCAATTGTAAGCAAACTCTGGGCCACGATACTATGTATTGGTACAAGCAAGACTCTAAGAAATTGCTGAAGATTATGTTTAG |
| | CTACAATAATAAGCAACTCATTGTAAACGAAACAGTTCCAAGGCGCTTCTCACCTCAGTCTTCAGATAAAGCTCATTTGAATCTTCGAATCAAGTCTGTAG |
| | AGCCGGAGGACTCTGCTGTGTATCTCTGTGCCAGCAGCTTCGACTGGGGGGGCGATGAACAGTACTTCGGTCCCGGCACCAGGCTCACGGTTTTAG |
| NK25 | ATGTCTAACACTGTCCTCGCTGATTCTGCCTGGGGCATCACCCTGCTATCTTGGGTTACTGTCTTTCTCTTGGGAACAAGTTCAGCAGATTCTGGGGTTGT |
| | CCAGTCTCCAAGACACATAATCAAAGAAAAGGGAGGAAGGTCCGTTCTGACGTGTATTCCCATCTCTGGACATAGCAATGTGGTCTGGTACCAGCAGAC |
| | TCTGGGGAAGGAATTAAAGTTCCTTATTCAGCATTATGAAAAGGTGGAGAGAGACAAAGGATTCCTACCCAGCAGATTCTCAGTCCAACAGTTTGATGA |
| | CTATCACTCTGAAATGAACATGAGTGCCTTGGAACTGGAGGACTCTGCTATGTACTTCTGTGCCAGCTCTCTCGGCAATAACCAAGACACCCAGTACTTT |
| | GGGCCAGGCACTCGGCTCCTCGTGTTAG |
| NK186 | ATGGGCTCCATTTTCCTCAGTTGCCTGGCCGTTTGTCTCCTGGTGGCAGGTCCAGTCGACCCGAAAATTATCCAGAAACCAAAATATCTGGTGGCAGTCA |
| | CAGGGAGCGAAAAAATCCTGATATGCGAACAGTATCTAGGCCACAATGCTATGTATTGGTATAGACAAAGTGCTAAGAAGCCTCTAGAGTTCATGTTTT |
| | CCTACAGCTATCAAAAACTTATGGACAATCAGACTGCCTCAAGTCGCTTCCAACCTCAAAGTTCAAAGAAAAACCATTTAGACCTTCAGATCACAGCTCTA |
| | AAGCCTGATGACTCGGCCACATACTTCTGTGCCAGCAGCCGAGGACTGGAGAACTATGCTGAGCAGTTCTTCGGACCAGGGACACGACTCACCGTCCTA |
| | G |
| NK20 | ATGTCTAACACTGCCTTCCCTGACCCCGCCTGGAACACCACCCTGCTATCTTGGGTTGCTCTCTTTCTCCTGGGAACAAGTTCAGCAAATTCTGGGGTTGT |
| | CCAGTCTCCAAGATACATAATCAAAGGAAAGGGAGAAAGGTCCATTCTAAAATGTATTCCCATCTCTGGACATCTCTCTGTGGCCTGGTATCAACAGACT |
| | CAGGGGCAGGAACTAAAGTTCTTCATTCAGCATTATGATAAAATGGAGAGAGATAAAGGAAACCTGCCCAGCAGATTCTCAGTCCAACAGTTTGATGAC |
| | TATCACTCTGAGATGAACATGAGTGCCTTGGAGCTAGAGGACTCTGCCGTGTACTTCTGTGCCAGCTCTCTCGGGCAGGACAGCTCCTATGAACAGTACT |
| | TCGGTCCCGGCACCAGGCTCACGGTTTTAG |
| NK22 | ATGGGCTCCAGACTCTTCTTTGTGGTTTTGATTCTCCTGTGTGCAAAACACATGGAGGCTGCAGTCACCCAAAGTCCAAGAAGCAAGGTGGCAGTAACA |
| | GGAGGAAAGGTGACATTGAGCTGTCACCAGACTAATAACCATGACTATATGTACTGGTATCGGCAGGACACGGGGCATGGGCTGAGGCTGATCCATTA |
| | CTCATATGTCGCTGACAGCACGGAGAAAGGAGATATCCCTGATGGGTACAAGGCCTCCAGACCAAGCCAAGAGAATTTCTCTCTCATTCTGGAGTTGGC |
| | TTCCCTTTCTCAGACAGCTGTATATTTCTGTGCCAGCAGTGATCAGGGCACAGAAGTCTTCTTTGGTAAAGGAACCAGACTCACAGTTGTAG |
| NK17 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGTGATGCTACAGTTGGCAACCAGGCTCCGCTTTTTGGAGAGGGGACTCGACTCTCTGTTCTA |
| | G |
| NK139 | ATGTGGCAGTTTTGCATTCTGTGCCTCTGTGTACTCATGGCTTCTGTGGCTACAGACCCCACAGTGACTTTGCTGGAGCAAAACCCAAGGTGGCGTCTGG |
| | TACCACGTGGTCAAGCTGTGAACCTACGCTGCATCTTGAAGAATTCCCAGTATCCCTGGATGAGCTGGTATCAGCAGGATCTCCAAAAGCAACTACAGTG |
| | GCTGTTCACTCTGCGGAGTCCTGGGGACAAAGAGGTCAAATCTCTTCCCGGTGCTGATTACCTGGCCACACGGGTCACTGATACGGAGCTGAGGCTGCA |
| | AGTGGCCAACATGAGCCAGGGCAGAACCTTGTACTGCACCTGCAGTGCTCTTCCGGGGGCTGACACAGAAGTCTTCTTTGGTAAAGGAACCAGACTCAC |
| | AGTTGTAG |
| NK12 | ATGAGCTGCAGGCTTCTCCTCTATGTTTCCCTATGTCTTGTGGAAACAGCACTCATGAACACTAAAATTACTCAGTCACCAAGATATCTAATCCTGGGAAG |
| | AGCAAATAAGTCTTTGGAATGTGAGCAACATCTGGGACATAATGCTATGTACTGGTATAAACAGAGCGCTGAGAAGCCGCCAGAGCTCATGTTTCTCTA |
| | CAATCTTAAACAGTTGATTCGAAATGAGACGGTGCCCAGTCGTTTTATACCTGAATGCCCAGACAGCTCCAAGCTACTTTTACATATATCTGCCGTGGATC |
| | CAGAAGACTCAGCTGTCTATTTTTGTGCCAGCAGCCAAGATTGGCTGGGGGTTGCTGAGCAGTTCTTCGGACCAGGGACACGACTCACCGTCCTAG |
| NK11 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGTGATCTCCGGGACAAATTCCAAGACACCCAGTACTTTGGGCCAGGCACTCGGCTCCTCGTG |
| | TTAG |
| NK24 | ATGTCTAACACTGTCCTCGCTGATTCTGCCTGGGGCATCACCCTGCTATCTTGGGTTACTGTCTTTCTCTTGGGAACAAGTTCAGCAGATTCTGGGGTTGT |
| | CCAGTCTCCAAGACACATAATCAAAGAAAAGGGAGGAAGGTCCGTTCTGACGTGTATTCCCATCTCTGGACATAGCAATGTGGTCTGGTACCAGCAGAC |
| | TCTGGGGAAGGAATTAAAGTTCCTTATTCAGCATTATGAAAAGGTGGAGAGAGACAAAGGATTCCTACCCAGCAGATTCTCAGTCCAACAGTTTGATGA |
| | CTATCACTCTGAAATGAACATGAGTGCCTTGGAACTGGAGGACTCTGCTATGTACTTCTGTGCCAGCTCTCTCGATGACAGGGGGCGGGGAAATACGCT |
| | CTATTTTGGAGAAGGAAGCCGGCTCATTGTTGTAG |
| NK18 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGTGATGGGCTGGGGCACTATGAACAGTACTTCGGTCCCGGCACCAGGCTCACGGTTTTAG |
| NK23 | ATGGCCCCCAGGCTCCTTTTCTGTCTGGTTCTTTGCTTCTTGAGAGCAGAACCAACAAATGCTGGTGTCATCCAAACACCTAGGCACAAGGTGACAGGGA |
| | AGGGACAAGAAGCAACTCTGTGGTGTGAGCCAATTTCAGGACATAGTGCTGTTTTCTGGTACAGACAGACCATTGTGCAGGGCCTGGAGTTCCTGACTT |
| | ACTTTCGAAATCAAGCTCCTATAGATGATTCAGGGATGCCCAAGGAACGATTCTCAGCTCAGATGCCCAATCAGTCGCACTCAACTCTGAAGATCCAGAG |
| | CACGCAACCCCAGGACTCAGCGGTGTATCTTTGTGCAAGCCGACAGTCCTATGCTGAGCAGTTCTTCGGACCAGGGACACGACTCACCGTCCTAG |
| NK21 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGGACTGGGGGCGCTAGTGCAGAAACGCTGTATTTTGGCTCAGGAACCAGACTGACTGTTCTC |
| | G |
| NK19 | ATGGGCACCAGGCTTCTTGGCTGGGCAGTGTTCTGTCTCCTTGACACAGTACTGTCTGAAGCTGGAGTCACCCAGTCTCCCAGATATGCAGTCCTACAGG |
| | AAGGGCAAGCTGTTTCCTTTTGGTGTGACCCTATTTCTGGACATGATACCCTTTACTGGTATCAGCAGCCCAGAGACCAGGGGCCCCAGCTTCTAGTTTA |
| | CTTTCGGGATGAGGCTGTTATAGATAATTCACAGTTGCCCTCGGATCGATTTTCTGCTGTGAGGCCTAAAGGAACTAACTCCACTCTCAAGATCCAGTCTG |
| | CAAAGCAGGGCGACACAGCCACCTATCTCTGTGCCAGCAGATCCGGGACAGGGTCCGAAACGCTGTATTTTGGCTCAGGAACCAGACTGACTGTTCTCG |
| NK10 | ATGGGCTCCAGGCTCTTTCTGGTCTTGAGCCTCCTGTGTACAAAACACATGGAGGCTGCAGTCACCCAAAGCCCTAGAAACAAGGTGACAGTAACAGGA |
| | GGAAACGTGACATTGAGCTGTCGCCAGACTAATAGCCACAACTACATGTACTGGTATCGGCAGGACACTGGGCATGGGCTGAGGCTGATCCATTACTCA |
| | TATGGTGCTGGCAACCTTCAAATAGGAGATGTCCCTGATGGGTACAAGGCCACCAGAACAACGCAAGAAGACTTCTTCCTCCTGCTGGAATTGGCTTCTC |
| | CCTCTCAGACATCTTTGTACTTCTGTGCCAGCAGTGATAACATCTATGAACAGTACTTCGGTCCCGGCACCAGGCTCACGGTTTTAG |
| NK15 | ATGTCTAACACTGCCTTCCCTGACCCCGCCTGGAACACCACCCTGCTATCTTGGGTTGCTCTCTTTCTCCTGGGAACAAGTTCAGCAAATTCTGGGGTTGT |
| | CCAGTCTCCAAGATACATAATCAAAGGAAAGGGAGAAAGGTCCATTCTAAAATGTATTCCCATCTCTGGACATCTCTCTGTGGCCTGGTATCAACAGACT |
| | CAGGGGCAGGAACTAAAGTTCTTCATTCAGCATTATGATAAAATGGAGAGAGATAAAGGAAACCTGCCCAGCAGATTCTCAGTCCAACAGTTTGATGAC |
| | TATCACTCTGAGATGAACATGAGTGCCTTGGAGCTAGAGGACTCTGCCGTGTACTTCTGTGCCAGCTCTCTCGACACAAACTCCGACTACACCTTCGGCTC |
| | AGGGACCAGGCTTTTGGTAG |
| NK183 | ATGTCTAACACTGCCTTCCCTGACCCCGCCTGGAACACCACCCTGCTATCTTGGGTTGCTCTCTTTCTCCTGGGAACAAGTTCAGCAAATTCTGGGGTTGT |
| | CCAGTCTCCAAGATACATAATCAAAGGAAAGGGAGAAAGGTCCATTCTAAAATGTATTCCCATCTCTGGACATCTCTCTGTGGCCTGGTATCAACAGACT |
| | CAGGGGCAGGAACTAAAGTTCTTCATTCAGCATTATGATAAAATGGAGAGAGATAAAGGAAACCTGCCCAGCAGATTCTCAGTCCAACAGTTTGATGAC |
| | TATCACTCTGAGATGAACATGAGTGCCTTGGAGCTAGAGGACTCTGCCGTGTACTTCTGTGCCAGCTCTCTCGCCCATCCCTATGAACAGTACTTCGGTCC |
| | CGGCACCAGGCTCACGGTTTTAG |
| NK30 | ATGGGCTCCAGGCTCTTTCTGGTCTTGAGCCTCCTGTGTACAAAACACATGGAGGCTGCAGTCACCCAAAGCCCTAGAAACAAGGTGACAGTAACAGGA |
| | GGAAACGTGACATTGAGCTGTCGCCAGACTAATAGCCACAACTACATGTACTGGTATCGGCAGGACACTGGGCATGGGCTGAGGCTGATCCATTACTCA |
| | TATGGTGCTGGCAACCTTCAAATAGGAGATGTCCCTGATGGGTACAAGGCCACCAGAACAACGCAAGAAGACTTCTTCCTCCTGCTGGAATTGGCTTCTC |
| | CCTCTCAGACATCTTTGTACTTCTGTGCCAGCAGGGACTGGGGGAACTATGAACAGTACTTCGGTCCCGGCACCAGGCTCACGGTTTTAG |
| NK150 | ATGGGCTCCAGGCTCTTTCTGGTCTTGAGCCTCCTGTGTACAAAACACATGGAGGCTGCAGTCACCCAAAGCCCTAGAAACAAGGTGACAGTAACAGGA |
| | GGAAACGTGACATTGAGCTGTCGCCAGACTAATAGCCACAACTACATGTACTGGTATCGGCAGGACACTGGGCATGGGCTGAGGCTGATCCATTACTCA |
| | TATGGTGCTGGCAACCTTCAAATAGGAGATGTCCCTGATGGGTACAAGGCCACCAGAACAACGCAAGAAGACTTCTTCCTCCTGCTGGAATTGGCTTCTC |
| | CCTCTCAGACATCTTTGTACTTCTGTGCCAGCAGTGATGAAGGGGGGGCCAGTCAAAACACCTTGTACTTTGGTGCGGGCACCCGACTATCGGTGCTAG |
| NK32 | ATGTGGCAGTTTTGCATTCTGTGCCTCTGTGTACTCATGGCTTCTGTGGCTACAGACCCCACAGTGACTTTGCTGGAGCAAAACCCAAGGTGGCGTCTGG |
| | TACCACGTGGTCAAGCTGTGAACCTACGCTGCATCTTGAAGAATTCCCAGTATCCCTGGATGAGCTGGTATCAGCAGGATCTCCAAAAGCAACTACAGTG |
| | GCTGTTCACTCTGCGGAGTCCTGGGGACAAAGAGGTCAAATCTCTTCCCGGTGCTGATTACCTGGCCACACGGGTCACTGATACGGAGCTGAGGCTGCA |
| | AGTGGCCAACATGAGCCAGGGCAGAACCTTGTACTGCACCTGCAGTGCCGGAGGGAGAGAAGTCTTCTTTGGTAAAGGAACCAGACTCACAGTTGTAG |
| NK26 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGTGATGCTACAGTTGGCAACCAGGCTCCGCTTTTTGGAGAGGGGACTCGACTTTCTGTTCTA |
| | G |
| NK29 | ATGGGCTCCAGGCTCTTCTTCGTGCTCTCCAGTCTCCTGTGTTCAAAACACATGGAGGCTGCAGTCACCCAAAGCCCAAGAAACAAGGTGGCAGTAACA |
| | GGAGGAAAGGTGACATTGAGCTGTAATCAGACTAATAACCACAACAACATGTACTGGTATCGGCAGGACACGGGGCATGGGCTGAGGCTGATCCATTA |
| | TTCATATGGTGCTGGCAGCACTGAGAAAGGAGATATCCCTGATGGATACAAGGCCTCCAGACCAAGCCAAGAGAACTTCTCCCTCATTCTGGAGTTGGC |
| | TACCCCCTCTCAGACATCAGTGTACTTCTGTGCCAGCGGTGATGCCGCGGACAGGGGGCGGAAAGTCTTCTTTGGTAAAGGAACCAGACTCACAGTTGT |
| | AG |
| NK31 | ATGTCTAACACTGTCCTCGCTGATTCTGCCTGGGGCATCACCCTGCTATCTTGGGTTACTGTCTTTCTCTTGGGAACAAGTTCAGCAGATTCTGGGGTTGT |
| | CCAGTCTCCAAGACACATAATCAAAGAAAAGGGAGGAAGGTCCGTTCTGACGTGTATTCCCATCTCTGGACATAGCAATGTGGTCTGGTACCAGCAGAC |
| | TCTGGGGAAGGAATTAAAGTTCCTTATTCAGCATTATGAAAAGGTGGAGAGAGACAAAGGATTCCTACCCAGCAGATTCTCAGTCCAACAGTTTGATGA |
| | CTATCACTCTGAAATGAACATGAGTGCCTTGGAACTGGAGGACTCTGCTATGTACTTCTGTGCCAGCTCTCTCCGGGGGTACAGGGAAGTCTTCTTTGGT |
| | AAAGGAACCAGACTCACAGTTGTAG |
| NK199 | ATGGGCTCCAGACTCTTCTTTGTGGTTTTGATTCTCCTGTGTGCAAAACACATGGAGGCTGCAGTCACCCAAAGTCCAAGAAGCAAGGTGGCAGTAACA |
| | GGAGGAAAGGTGACATTGAGCTGTCACCAGACTAATAACCATGACTATATGTACTGGTATCGGCAGGACACGGGGCATGGGCTGAGGCTGATCCATTA |
| | CTCATATGTCGCTGACAGCACGGAGAAAGGAGATATCCCTGATGGGTACAAGGCCTCCAGACCAAGCCAAGAGAATTTCTCTCTCATTCTGGAGTTGGC |
| | TTCCCTTTCTCAGACAGCTGTATATTTCTGTGCCAGCAGCTTTGGGGGCCTTCAAAACACCTTGTACTTTGGTGCGGGCACCCGACTATCGGTGCTAG |
| NK154 | ATGGCCCCCAGGCTCCTTTTCTGTCTGGTTCTTTGCTTCTTGAGAGCAGAACCAACAAATGCTGGTGTCATCCAAACACCTAGGCACAAGGTGACAGGGA |
| | AGGGACAAGAAGCAACTCTGTGGTGTGAGCCAATTTCAGGACATAGTGCTGTTTTCTGGTACAGACAGACCATTGTGCAGGGCCTGGAGTTCCTGACTT |
| | ACTTTCGAAATCAAGCTCCTATAGATGATTCAGGGATGCCCAAGGAACGATTCTCAGCTCAGATGCCCAATCAGTCGCACTCAACTCTGAAGATCCAGAG |
| | CACGCAACCCCAGGACTCAGCGGTGTATCTTTGTGCAAGCAGCCCCCAGGGGGCTATAGGGTACTTCGGTCCCGGCACCAGGCTCACGGTTTTAG |
| NK72 | ATGGGCTCCAGACTCTTCTTTGTGGTTTTGATTCTCCTGTGTGCAAAACACATGGAGGCTGCAGTCACCCAAAGTCCAAGAAGCAAGGTGGCAGTAACA |
| | GGAGGAAAGGTGACATTGAGCTGTCACCAGACTAATAACCATGACTATATGTACTGGTATCGGCAGGACACGGGGCATGGGCTGAGGCTGATCCATTA |
| | CTCATATGTCGCTGACAGCACGGAGAAAGGAGATATCCCTGATGGGTACAAGGCCTCCAGACCAAGCCAAGAGAATTTCTCTCTCATTCTGGAGTTGGC |
| | TTCCCTTTCTCAGACAGCTGTATATTTCTGTGCCAGCAGTCCACTGGGGGGGCTTCGGGAACAGTACTTCGGTCCCGGCACCAGGCTCACGGTTTTAG |
| NK62 | ATGGGCTCCATTTTCCTCAGTTGCCTGGCCGTTTGTCTCCTGGTGGCAGGTCCAGTCGACCCGAAAATTATCCAGAAACCAAAATATCTGGTGGCAGTCA |
| | CAGGGAGCGAAAAAATCCTGATATGCGAACAGTATCTAGGCCACAATGCTATGTATTGGTATAGACAAAGTGCTAAGAAGCCTCTAGAGTTCATGTTTT |
| | CCTACAGCTATCAAAAACTTATGGACAATCAGACTGCCTCAAGTCGCTTCCAACCTCAAAGTTCAAAGAAAAACCATTTAGACCTTCAGATCACAGCTCTA |
| | AAGCCTGATGACTCGGCCACATACTTCTGTGCCAGCAGCCAAGGGGTCATTTCCAACGAAAGATTATTTTTCGGTCATGGAACCAAGCTGTCTGTCCTGG |
| NK193 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTATAGCAGGGGGTCAAAACACCTTGTACTTTGGTGCGGGCACCCGACTATCG |
| | GTGCTAG |
| NK179 | ATGGATATCTGGCTTCTAGGTTGGATAATTTTTAGTTTCTTGGAAGCAGGACACACAGGACCCAAAGTCTTACAGATCCCAAGTCATCAAATAATAGATA |
| | TGGGGCAGATGGTGACCCTCAATTGTGACCCAGTTTCTAATCACCTATATTTTTATTGGTATAAACAGATTTTAGGACAGCAGATGGAGTTTCTGGTTAAT |
| | TTCTACAATGGTAAAGTCATGGAGAAGTTTAAACTGTTTAAGGATCAGTTTTCAGTTGAAAGACCAGATGGTTCATATTTCACTCTGAAAATCCAACCCAC |
| | AGCACTGGAGGACTCAGCTGTGTACTTCTGTGCCAGCAGCTCGACTGGGGGGGGTTATGAACAGTACTTCGGTCCCGGCACCAGGCTCACGGTTTTAG |
| NK70 | ATGCTGTACTCTCTCCTTGCCTTTCTCCTGGGCATGTTCTTGGGTGTTAGTGCTCAGACTATCCATCAATGGCCAGTTGCCGAGATCAAGGCTGTGGGCAG |
| | CCCACTGTCTCTGGGGTGTACCATAAAGGGGAAATCAAGCCCTAACCTCTACTGGTACTGGCAGGCCACAGGAGGCACCCTCCAGCAACTCTTCTACTCT |
| | ATTACTGTTGGCCAGGTAGAGTCGGTGGTGCAACTGAACCTCTCAGCTTCCAGGCCGAAGGACGACCAATTCATCCTAAGCACGGAGAAGCTGCTTCTC |
| | AGCCACTCTGGCTTCTACCTCTGTGCCTGGAGTCTACCCCGGACTGGGGGGTGGGAACAGTACTTCGGTCCCGGCACCAGGCTCACGGTTTTAG |
| NK77 | ATGGGCTCCAGACTCTTCTTTGTGGTTTTGATTCTCCTGTGTGCAAAACACATGGAGGCTGCAGTCACCCAAAGTCCAAGAAGCAAGGTGGCAGTAACA |
| | GGAGGAAAGGTGACATTGAGCTGTCACCAGACTAATAACCATGACTATATGTACTGGTATCGGCAGGACACGGGGCATGGGCTGAGGCTGATCCATTA |
| | CTCATATGTCGCTGACAGCACGGAGAAAGGAGATATCCCTGATGGGTACAAGGCCTCCAGACCAAGCCAAGAGAATTTCTCTCTCATTCTGGAGTTGGC |
| | TTCCCTTTCTCAGACAGCTGTATATTTCTGTGCCAGCAGGGGACTGGGGGCTAACCAAGACACCCAGTACTTTGGGCCAGGCACTCGGCTCCTCGTGTTA |
| | G |
| NK198 | ATGTGGCAGTTTTGCATTCTGTGCCTCTGTGTACTCATGGCTTCTGTGGCTACAGACCCCACAGTGACTTTGCTGGAGCAAAACCCAAGGTGGCGTCTGG |
| | TACCACGTGGTCAAGCTGTGAACCTACGCTGCATCTTGAAGAATTCCCAGTATCCCTGGATGAGCTGGTATCAGCAGGATCTCCAAAAGCAACTACAGTG |
| | GCTGTTCACTCTGCGGAGTCCTGGGGACAAAGAGGTCAAATCTCTTCCCGGTGCTGATTACCTGGCCACACGGGTCACTGATACGGAGCTGAGGCTGCA |
| | AGTGGCCAACATGAGCCAGGGCAGAACCTTGTACTGCACCTGCAGTGCGACAGATTCTGGAAATACGCTCTATTTTGGAGAAGGAAGCCGGCTCATTGT |
| | TGTAG |
| PD233 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTATAGACTGGAATAGTCAAAACACCTTGTACTTTGGTGCGGGCACCCGACTAT |
| | CGGTGCTAG |
| PD292 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTACCCCGGGCTGGGGATACCAAGACACCCAGTACTTTGGGCCAGGCACTCGG |
| | CTCCTCGTGTTAG |
| PD252 | ATGGATATCTGGCTTCTAGGTTGGATAATTTTTAGTTTCTTGGAAGCAGGACACACAGGACCCAAAGTCTTACAGATCCCAAGTCATCAAATAATAGATA |
| | TGGGGCAGATGGTGACCCTCAATTGTGACCCAGTTTCTAATCACCTATATTTTTATTGGTATAAACAGATTTTAGGACAGCAGATGGAGTTTCTGGTTAAT |
| | TTCTACAATGGTAAAGTCATGGAGAAGTCTAAACTGTTTAAGGATCAGTTTTCAGTTGAAAGACCAGATGGTTCATATTTCACTCTGAAAATCCAACCCAC |
| | AGCACTGGAGGACTCAGCTGTGTACTTCTGTGCCAGCAGCTTCGGACTCAGGCCCTTTGCTGAGCAGTTCTTCGGACCAGGGACACGACTCACCGTCCTA |
| | G |
| PD217 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTATTGGACTGGGGGGTAACTATGCTGAGCAGTTCTTCGGACCAGGGACACGA |
| | CTCACCGTCCTAG |
| PD245 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTATTGGACTGGGGGGTAACTATGCTGAGCAGTTCTTCGGACCAGGGACACGA |
| | CTCACCGTCCTAG |
| PD272 | ATGAACAAGTGGGTTTTCTGCTGGGTAACCCTTTGTCTCCTTACTGTAGAGACCACACATGGTGATGGTGGCATCATTACTCAGACACCCAAATTCCTGAT |
| | TGGTCAGGAAGGGCAAAAACTGACCTTGAAATGTCAACAGAATTTCAATCATGATACAATGTACTGGTACCGACAGGATTCAGGGAAAGGATTGAGAC |
| | TGATCTACTATTCAATAACTGAAAACGATCTTCAAAAAGGCGATCTATCTGAAGGCTATGATGCGTCTCGAGAGAAGAAGTCATCTTTTTCTCTCACTGTG |
| | ACATCTGCCCAGAAGAACGAGATGGCCGTTTTTCTCTGTGCCAGCAGTATAGACTGGAATAGTCAAAACACCTTGTACTTTGGTGCGGGCACCCGACTAT |
| | CGGTGCTAG |
| OT-I | ATGTCTAACACTGTCCTCGCTGATTCTGCCTGGGGCATCACCCTGCTATCTTGGGTTACTGTCTTTCTCTTGGGAACAAGTTCAGCAGATTCTGGGGTTGT |
| | CCAGTCTCCAAGACACATAATCAAAGAAAAGGGAGGAAGGTCCGTTCTGACGTGTATTCCCATCTCTGGACATAGCAATGTGGTCTGGTACCAGCAGAC |
| | TCTGGGGAAGGAATTAAAGTTCCTTATTCAGCATTATGAAAAGGTGGAGAGAGACAAAGGATTCCTACCCTCGCGATTCTCAGTCCAACAGTTTGATGA |
| | CTATCACTCTGAAATGAACATGAGTGCCTTGGAACTGGAGGACTCTGCTATGTACTTCTGTGCCAGCTCTCGGGCCAATTATGAACAGTACTTCGGTCCC |
| | GGCACCAGGCTCACGGTTTTAG |

SUPPLEMENTARY TABLE 3
High in ILTCkP, low in ILTCk
geneID
Ccr2/Cd81/Foxp3/Gata3/Il18r1/Il18rap/Il1r1/Il4/Slc11a1/Tbx21/Tnfrsf1b
Adk/Adora2a/Ap3b1/Arg2/Bcl6/Cblb/Ccl5/Ccr2/Ccr6/Cd40lg/Cd81/Eif2ak4/Foxp3/Fut7/Gata3/Gpnmb/Icosl/Igf2/Il12rb1/Il18r1/Il4/I16st/
Itgad/Jag2/Kit/Lag3/Nr4a1/Mpzl2/P2rx7/Pik3r6/Pnp/Prdx2/Prex1/Psen2/Rorc/Sdc4/Sh3rf1/Slc11a1/Smad3/Sox13/Stat3/Tbx21/Tigit/Tnfrsf1b/
Tnfrsf21/Tnfsf14/Tnfsf8/Twsg1/Zbtb16/Zbtb7b
Adk/Adora2a/Ap3b1/Arg2/Bcl6/Cblb/Ccl5/Ccr2/Cd40lg/Cd81/Foxp3/Gata3/Gpnmb/Icosl/Igf2/Il12rb1/Il4/Il6st/Lag3/Pik3r6/Pnp/Prdx2/Rorc/
Sdc4/Sh3rf1/Sox13/Tbx21/Tigit/Tnfrsf1b/Tnfrsf21/Tnfsf14/Twsg1/Zbtb16/Zbtb7b
Adk/Arg2/Cblb/Ccl5/Ccr2/Cd40lg/Cd81/Foxp3/Gpnmb/Icosl/Igf2/Il12rb1/Il4/Il6st/Itgad/P2rx7/Pnp/Prdx2/Sdc4/Sh3rf1/Slc11a1/Tnfrsf1b/
Tnfrsf21/Twsg1/Zbtb7b
Adora2a/Ap3b1/Arg2/Bcl6/Cblb/Ccr2/Cd81/Foxp3/Fut7/Gata3/Il18r1/Il4/Pnp/Rorc/Sh3rf1/Stat3/Tbx21/Tnfsf8/Twsg1/Zbtb16/Zbtb7b
Adora2a/Ap3b1/Arg2/Bcl6/Cblb/Ccr2/Cd81/Foxp3/Gata3/Il4/Pnp/Sh3rf1/Tbx21/Twsg1/Zbtb16/Zbtb7b
Ccr2/Cd81/Foxp3/Gata3/Il18r1/Il1r1/Tbx21/Tnfrsf1b
Cd81/Gata3/Il18r1/Il18rap/Il1r1/Il4/Tbx21
Adk/Arg2/Cblb/Ccl5/Ccr2/Cd40lg/Cd81/Foxp3/Gpnmb/Icosl/Igf2/Il12rb1/Il4/Il6st/Pnp/Sdc4/Sh3rf1/Tnfrsf1b/Tnfrsf21/Twsg1/Zbtb7b
Arg2/Bcl6/Ccr2/Cd81/Foxp3/Fut7/Gata3/Il18r1/Il4/Rorc/Sh3rf1/Stat3/Tbx21/Twsg1/Zbtb7b
Ap3b1/Bcl6/Ccr2/Foxp3/Fut7/Gata3/Il18r1/Il4/Pnp/Rorc/Sh3rf1/Stat3/Tbx21/Tnfsf8/Zbtb16/Zbtb7b
Adora2a/Arg2/Bcl6/Cblb/Foxp3/Gpnmb/I14/Lag3/Pdcd1/Prdx2/Sdc4/Tbx21/Tigit/Tnfrsf21/Tox/Twsg1/Zbtb7b
Arg2/Bcl6/Ccr2/Cd81/Foxp3/Gata3/Il4/Sh3rf1/Tbx21/Twsg1/Zbtb7b
Ccr2/Cd81/Foxp3/Gata3/H2-Q2/H2-Q6/Il18r1/Il18rap/Il1r1/Il4/Myo1g/Nectin2/P2rx7/Pnp/Slc11a1/Tbx21/Tnfrsf1b
Ccl5/Ccr2/Ccr6/Cxcr3/Ecm1/Gpr15/Itgb3/Myo1g/Stk39/Tnfsf14
Ap3b1/Bcl6/Ccr2/Foxp3/Gata3/Il4/Pnp/Sh3rf1/Tbx21/Zbtb16/Zbtb7b

High in ILTCkP and ILTCk
geneID
Bcl2l11/Cd160/Klrb1c/Klrk1/Sh2d1b1/Tnfsf10/Tyrobp/Xcl1
Cd160/Cd200r1/Fcer1g/Fgl2/Gcsam/Lgals3/Pdcd1lg2/Sh2d1b1/Tyrobp/Ubash3b/Xcl1/Zfp36l1
Bcl2l11/Cd160/Gzmb/Klrb1c/Klrk1/Sh2d1b1/Tyrobp/Xcl1
Bcl2l11/Cd160/Klrb1c/Klrk1/Sh2d1b1/Tyrobp/Xcl1
Cd160/Klrb1c/Klrk1/Sh2d1b1/Tyrobp/Xcl1
Cd160/Gzmb/Klrb1c/Klrk1/Sh2d1b1/Tyrobp/Xcl1
Cd160/Fcer1g/Fgl2/Fgr/Gzmb/Igha/Klrb1c/Klrk1/Lat2/Sh2d1b1/Xcl1
Fcer1g/Pdcd1lg2/Tnfrsf9/Tyrobp/Xcl1
Cd160/Fcer1g/Klrb1c/Klrk1/Sh2d1b1/Xcl1
Cd160/Cd38/Fcer1g/Gcsam/Igha/Klrk1/Lat2/Lgals3
Cd160/Klrb1c/Klrk1/Sh2d1b1/Tyrobp/Xcl1
Cd160/Irf8/Klrk1/Pdcd1lg2/Sh2d1b1/Xcl1
Fcer1g/Pdcd1lg2/Tnfrsf9/Tyrobp/Xcl1
Cd160/Gzmb/Klrb1c/Klrk1/Sh2d1b1
Cd160/Fcer1g/Fgl2/Fgr/Lat2/Lgals3/Sh2d1b1/Tyrobp

Low in ILTCkP, High in ILTCk
geneID
Cd226/Cd96/Gzmc/Klrd1/Klre1/Lgals9/Serpinb9
Cd96/Klre1/Lgals9/Serpinb9
Cd226/Cd96/Gadd45g/Klre1/Lgals9/Pycard/Txk
Cd226/Cd96/Itga1/Klre1/Lgals9/Serpinb9
Cd226/Cd96/Klre1/Lgals9/Pycard/Txk
Klre1/Lgals9/Serpinb9
Cd226/Klre1/Lgals9/Serpinb9
Cd226/Gzmc/Klre1/Lgals9/Serpinb9
Cd226/Cd96/Cr11/Ighm/Itga1/Klre1/Lgals9/Pycard/Serpinb9/Zfp683
Cd226/Klre1/Pycard/Txk
Cd96/Cr1l/Itgae/Klre1/Lgals9/Serpinb9

SUPPLEMENTARY TABLE 4

| gene | pvalue |
|---|---|
| Igkv1-99 | 2.97E−07 |
| Ighv1-42 | 1.03E−06 |
| Igkv17-127 | 1.06E−06 |
| Ccl22 | 4.64E−08 |
| Esp18 | 0.000466379 |
| Dpep1 | 0.0002204 |
| Cntn2 | 0.000137844 |
| Kcnj16 | 0.000278746 |
| Asgr1 | 0.000232408 |
| Lum | 4.19E−06 |
| Gzmk | 1.62E−11 |
| Efemp1 | 4.94E−06 |
| Postn | 6.01E−07 |
| Gbp10 | 0.000252452 |
| Acta1 | 0.000110594 |
| Slamf1 | 4.01E−09 |
| Serpina3n | 0.000190114 |
| St8sia1 | 1.62E−06 |
| Themis | 1.54E−05 |
| Gm28439 | 9.89E−05 |
| C1s1 | 4.42E−05 |
| Fmo2 | 0.000362603 |
| Cd40lg | 3.01E−05 |
| Cd5 | 1.37E−05 |
| Glrb | 0.0001521 |
| Tnfrsf4 | 2.53E−07 |
| Icos | 1.27E−07 |
| Lama3 | 0.000220255 |
| Cd8b1 | 3.64E−07 |
| Ccr7 | 6.24E−05 |
| Apln | 3.26E−05 |
| Gm16025 | 0.000244881 |
| Gm8369 | 2.59E−05 |
| Cd28 | 9.36E−05 |
| Rapgef4 | 0.000329137 |
| Tnc | 9.54E−05 |
| Ppp2r3a | 0.000265466 |
| Sdpr | 0.00016844 |
| Slfn9 | 0.000201588 |
| Krt17 | 1.64E−05 |
| Gm18194 | 7.01E−05 |
| Plcb4 | 0.000419276 |
| Il7r | 5.01E−05 |
| Pdcd1 | 0.000447152 |
| Ms4a4b | 0.000349359 |
| Lamb1 | 0.000217747 |
| Slco3a1 | 0.000373742 |
| Apc | 0.000335726 |
| Ppp1r3b | 0.00019269 |
| Lair1 | 0.000210863 |
| Gdf3 | 0.000195513 |
| Tgfbi | 7.31E−05 |
| Ccl3 | 0.000326394 |
| Il10ra | 0.000433558 |
| Hck | 0.000101852 |
| Itga5 | 0.000232764 |
| Ctsc | 0.000451006 |
| Rasgef1b | 0.000410178 |
| Ncf1 | 0.000323123 |
| Cd300lf | 0.000260426 |
| Cd300c2 | 5.44E−05 |
| Ccr5 | 0.000415884 |
| C130050018Rik | 0.000399184 |
| Slamf9 | 9.26E−05 |
| Tlr13 | 0.000161016 |
| Ncf2 | 0.000345033 |
| Sesn1 | 0.000156396 |
| Slc43a2 | 0.000432528 |
| Ly86 | 9.79E−05 |
| Cyp4f18 | 7.01E−05 |
| Slc28a2 | 0.000323287 |
| Aif1 | 2.18E−06 |
| Pfkfb4 | 0.000166868 |
| Adap2 | 0.00031989 |
| Sirpa | 0.000162282 |
| Cd38 | 0.000144329 |
| Csf2rb2 | 0.000405912 |
| Ms4a7 | 9.10E−07 |
| Tyrobp | 8.76E−06 |
| Hexa | 0.000183614 |
| Arl11 | 1.62E−05 |
| Abcc3 | 6.51E−05 |
| Clec4a3 | 4.99E−05 |
| Spi1 | 0.00017297 |
| Tlr1 | 2.30E−06 |
| Fcer1g | 4.36E−07 |
| Cd33 | 1.77E−05 |
| Fgd2 | 0.000211735 |
| Unc93b1 | 4.42E−05 |
| Tmem106a | 0.000256971 |
| Cd300lb | 0.0004364 |
| Lst1 | 2.02E−07 |
| Gas7 | 0.000200951 |
| Cd68 | 3.22E−05 |
| Fcgr3 | 8.16E−05 |
| Hk3 | 8.58E−05 |
| Syk | 0.000408235 |
| Apoe | 1.09E−05 |
| Lgmn | 1.62E−05 |
| Pld4 | 1.29E−05 |
| Nrros | 0.000311818 |
| Slc7a8 | 1.21E−05 |
| Tmem86a | 2.68E−06 |
| Pirb | 0.000122957 |
| Dok3 | 1.12E−05 |
| Rubcnl | 4.08E−05 |
| Fcgr1 | 3.85E−06 |
| Themis2 | 1.63E−05 |
| Sh3tc1 | 6.36E−05 |
| Il4ra | 8.17E−05 |
| Dab2 | 0.00017458 |
| Slc11a1 | 3.39E−06 |
| Ptafr | 0.000373628 |
| G530011006Rik | 2.92E−05 |
| C1qb | 9.42E−07 |
| Slc37a2 | 1.64E−05 |
| Adgre1 | 7.17E−08 |
| Fcgr2b | 0.000329345 |
| Fcgr4 | 5.32E−11 |
| Trem2 | 9.29E−05 |
| Cmklr1 | 3.81E−05 |
| Gzmc | 7.10E−05 |
| Lrrc25 | 0.000305037 |
| Csf3r | 5.92E−06 |
| Kcnk13 | 0.000112772 |
| Pla2g15 | 2.35E−06 |
| B3galnt1 | 0.000161304 |
| P2ry6 | 2.79E−06 |
| Ticam2 | 6.54E−05 |
| Rgl1 | 0.000351874 |
| Hlx | 3.56E−05 |
| Spic | 0.000167158 |
| Ccl12 | 1.93E−07 |
| Al427809 | 5.86E−06 |
| Hmox1 | 9.75E−05 |
| Csf1r | 9.06E−07 |
| Rab3il1 | 3.08E−06 |
| Sult1a1 | 0.000454093 |
| C3ar1 | 2.66E−07 |
| C1qc | 4.28E−08 |
| Clec4a1 | 5.56E−05 |
| Aoah | 5.58E−08 |
| Cyp27a1 | 9.30E−06 |
| C1qa | 8.22E−08 |
| Gbgt1 | 3.08E−05 |
| Ccl7 | 0.000384817 |
| Klrb1c | 2.42E−05 |
| Ms4a4a | 3.02E−06 |
| Il10 | 0.00019564 |
| Pltp | 0.000271444 |
| Stab1 | 8.20E−07 |
| B430306N03Rik | 4.48E−05 |
| Ncr1 | 2.53E−05 |
| Slco2b1 | 6.64E−09 |
| F630028010Rik | 2.49E−06 |
| Hpse | 0.000176995 |
| Pf4 | 0.000115577 |
| Stard8 | 5.49E−05 |
| Wfdc17 | 4.63E−05 |
| C5ar1 | 6.22E−07 |
| Gm42793 | 8.84E−05 |
| Prune2 | 7.72E−07 |
| Fcgrt | 2.63E−05 |
| Pigz | 2.52E−06 |
| Nxpe5 | 1.07E−05 |
| Hrh1 | 5.29E−05 |
| BC049352 | 4.66E−05 |
| Siglec1 | 2.17E−08 |
| Cd5l | 3.37E−09 |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. DuPage, M., Mazumdar, C., Schmidt, L. M., Cheung, A. F. & Jacks, T. Expression of tumour-specific antigens underlies cancer immunoediting. Nature 482, 405-409 (2012).
2. Matsushita, H. et al. Cancer exome analysis reveals a T-cell-dependent mechanism of cancer immunoediting. Nature 482, 400-404 (2012).
3. Chen, D. S. & Mellman, I. Elements of cancer immunity and the cancer-immune set point. Nature 541, 321-330 (2017).
4. Schumacher, T. N. & Schreiber, R. D. Neoantigens in cancer immunotherapy. Science 348, 69-74 (2015).
5. McLane, L. M., Abdel-Hakeem, M. S. & Wherry, E. J. CD8 T Cell Exhaustion During Chronic Viral Infection and Cancer. Annu Rev Immunol 37, 457-495 (2019).
6. Sharma, P. & Allison, J. P. The future of immune checkpoint therapy. Science 348, 56-61 (2015).
7. Topalian, S. L., Taube, J. M., Anders, R. A. & Pardoll, D. M. Mechanism-driven biomarkers to guide immune checkpoint blockade in cancer therapy. Nature reviews. Cancer 16, 275-287 (2016).
8. Baumeister, S. H., Freeman, G. J., Dranoff, G. & Sharpe, A. H. Coinhibitory Pathways in Immunotherapy for Cancer. Annu Rev Immunol 34, 539-573 (2016).
9. Zheng, C. et al. Landscape of Infiltrating T Cells in Liver Cancer Revealed by Single-Cell Sequencing. Cell 169, 1342-1356 e1316 (2017).
10. Azizi, E. et al. Single-Cell Map of Diverse Immune Phenotypes in the Breast Tumor Microenvironment. Cell 174, 1293-1308 e1236 (2018).
11. Simoni, Y. et al. Bystander CD8(+) T cells are abundant and phenotypically distinct in human tumour infiltrates. Nature 557, 575-579 (2018).
12. Zhang, L. et al. Lineage tracking reveals dynamic relationships of T cells in colorectal cancer. Nature 564, 268-272 (2018).
13. Dadi, S. et al. Cancer Immunosurveillance by Tissue-Resident Innate Lymphoid Cells and Innate-like T Cells. Cell 164, 365-377 (2016).
14. Schneider, W. M., Chevillotte, M. D. & Rice, C. M. Interferon-stimulated genes: a complex web of host defenses. Annu Rev Immunol 32, 513-545 (2014).
15. Yost, K. E. et al. Clonal replacement of tumor-specific T cells following PD-1 blockade. Nature medicine 25, 1251-1259 (2019).
16. Savage, P. A. et al. Recognition of a ubiquitous self antigen by prostate cancer-infiltrating CD8⁺ T lymphocytes. Science 319, 215-220 (2008).
17. Ise, W. et al. CTLA-4 suppresses the pathogenicity of self antigen-specific T cells by cell-intrinsic and cell-extrinsic mechanisms. Nature immunology 11, 129-135 (2010).
18. Scheper, W. et al. Low and variable tumor reactivity of the intratumoral TCR repertoire in human cancers. Nature medicine 25, 89-94 (2019).
19. Ruscher, R., Kummer, R. L., Lee, Y. J., Jameson, S. C. & Hogquist, K. A. CD8alphaalpha intraepithelial lymphocytes arise from two main thymic precursors. Nature immunology 18, 771-779 (2017).
20. Kim, H. J., Verbinnen, B., Tang, X., Lu, L. & Cantor, H. Inhibition of follicular T-helper cells by CD8(+) regulatory T cells is essential for self tolerance. Nature 467, 328-332 (2010).
21. Hildner, K. et al. Batf3 deficiency reveals a critical role for CD8alpha+ dendritic cells in cytotoxic T cell immunity. Science 322, 1097-1100 (2008).
22. Starr, T. K., Jameson, S. C. & Hogquist, K. A. Positive and negative selection of T cells. Annu Rev Immunol 21, 139-176 (2003).
23. Eberl, G. & Littman, D. R. Thymic origin of intestinal alphabeta T cells revealed by fate mapping of RORgammat+ cells. Science 305, 248-251 (2004).
24. Georgiev, H., Peng, C., Huggins, M. A., Jameson, S. C. & Hogquist, K. A. Classical MHC expression by DP thymocytes impairs the selection of non-classical MHC restricted innate-like T cells. Nat Commun 12, 2308 (2021).
25. Stritesky, G. L., Jameson, S. C. & Hogquist, K. A. Selection of self-reactive T cells in the thymus. Annu Rev Immunol 30, 95-114 (2012).
26. Cheroutre, H., Lambolez, F. & Mucida, D. The light and dark sides of intestinal intraepithelial lymphocytes. Nat Rev Immunol 11, 445-456 (2011).
27. McDonald, B. D., Jabri, B. & Bendelac, A. Diverse developmental pathways of intestinal intraepithelial lymphocytes. Nat Rev Immunol 18, 514-525 (2018).
28. Ruscher, R. et al. Intestinal CD8alphaalpha IELs derived from two distinct thymic precursors have staggered ontogeny. The Journal of experimental medicine 217 (2020).
29. Gazit, R. et al. Fgd5 identifies hematopoietic stem cells in the murine bone marrow. The Journal of experimental medicine 211, 1315-1331 (2014).
30. Khan, O. et al. TOX transcriptionally and epigenetically programs CD8(+) T cell exhaustion. Nature 571, 211-218 (2019).
31. Alfei, F. et al. TOX reinforces the phenotype and longevity of exhausted T cells in chronic viral infection. Nature 571, 265-269 (2019).
32. Scott, A. C. et al. TOX is a critical regulator of tumour-specific T cell differentiation. Nature 571, 270-+ (2019).
33. Suzuki, H., Duncan, G. S., Takimoto, H. & Mak, T. W. Abnormal development of intestinal intraepithelial lymphocytes and peripheral natural killer cells in mice lacking the IL-2 receptor beta chain. The Journal of experimental medicine 185, 499-505 (1997).
34. Ma, L. J., Acero, L. F., Zal, T. & Schluns, K. S. Trans-presentation of IL-15 by intestinal epithelial cells drives development of CD8alphaalpha IELs. J Immunol 183, 1044-1054 (2009).
35. Chinen, T. et al. An essential role for the IL-2 receptor in Treg cell function. Nature immunology 17, 1322-1333 (2016).
36. Perarnau, B. et al. Single H2Kb, H2Db and double H2KbDb knockout mice: peripheral CD8+ T cell repertoire and anti-lymphocytic choriomeningitis virus cytolytic responses. Eur J Immunol 29, 1243-1252 (1999).
37. Sosinowski, T. et al. CD8alpha+ dendritic cell trans presentation of IL-15 to naive CD8+ T cells produces antigen-inexperienced T cells in the periphery with memory phenotype and function. J Immunol 190, 1936-1947 (2013).
38. Butler, A., Hoffman, P., Smibert, P., Papalexi, E. & Satija, R. Integrating single-cell transcriptomic data across different conditions, technologies, and species. Nat Biotechnol 36, 411-420 (2018).
39. Stuart, T. et al. Comprehensive Integration of Single-Cell Data. Cell 177, 1888-1902 e1821 (2019).
40. Angerer, P. et al. destiny: diffusion maps for large-scale single-cell data in R. Bioinformatics 32, 1241-1243 (2016).
41. Haghverdi, L., Buettner, F. & Theis, F. J. Diffusion maps for high-dimensional single-cell analysis of differentiation data. Bioinformatics 31, 2989-2998 (2015).
42. Trapnell, C. et al. The dynamics and regulators of cell fate decisions are revealed by pseudotemporal ordering of single cells. Nat Biotechnol 32, 381-386 (2014).
43. Qiu, X. et al. Single-cell mRNA quantification and differential analysis with Census. Nat Methods 14, 309-315 (2017).
44. Qiu, X. et al. Reversed graph embedding resolves complex single-cell trajectories. Nat Methods 14, 979-982 (2017).
45. Kim, D., Langmead, B. & Salzberg, S. L. HISAT: a fast spliced aligner with low memory requirements. Nat Methods 12, 357-360 (2015).
46. Mudge, J. M. & Harrow, J. Creating reference gene annotation for the mouse C57BL6/J genome assembly. Mamm Genome 26, 366-378 (2015).
47. Liao, Y., Smyth, G. K. & Shi, W. The Subread aligner: fast, accurate and scalable read mapping by seed-and-vote. Nucleic Acids Res 41, e108 (2013).
48. Love, M. I., Huber, W. & Anders, S. Moderated estimation of fold change and dispersion for RNA-seq data with DESeq2. Genome Biol 15, 550 (2014).
49. Yu, G., Wang, L. G., Han, Y. & He, Q. Y. clusterProfiler: an R package for comparing biological themes among gene clusters. OMICS 16, 284-287 (2012).
50. Yang, Y. et al. Distinct mechanisms define murine B cell lineage immunoglobulin heavy chain (IgH) repertoires. Elife 4, e09083 (2015).
51. Wang, C. et al. High-throughput, high-fidelity HLA genotyping with deep sequencing. Proceedings of the National Academy of Sciences of the United States of America 109, 8676-8681 (2012).
52. Lefranc, M. P. et al. IMGT, the international ImMunoGeneTics database. Nucleic Acids Res 27, 209-212 (1999).
53. Franklin, R. A. et al. The cellular and molecular origin of tumor-associated macrophages. Science 344, 921-925 (2014).
54. Gasteiger, G., Fan, X., Dikiy, S., Lee, S. Y. & Rudensky, A. Y. Tissue residency of innate lymphoid cells in lymphoid and nonlymphoid organs. Science 350, 981-985 (2015).
55. Morita, S., Kojima, T. & Kitamura, T. Plat-E: an efficient and stable system for transient packaging of retroviruses. Gene Ther 7, 1063-1066 (2000).
56. Sanjana, N. E., Shalem, O. & Zhang, F. Improved vectors and genome-wide libraries for CRISPR screening. Nat Methods 11, 783-784 (2014).
57. Mayans, S. et al. alpha beta T Cell Receptors Expressed by CD4(−)CD8 alpha beta Intraepithelial T Cells Drive Their Fate into a Unique Lineage with Unusual MIC Reactivities. Immunity 41, 207-218 (2014).
58. McDonald, B. D., Bunker, J. J., Ishizuka, I. E., Jabri, B. & Bendelac, A. Elevated T Cell Receptor Signaling Identifies a Thymic Precursor to the TCR alpha beta(+)CD4(−)CD8 beta(−) Intraepithelial Lymphocyte Lineage. Immunity 41, 219-229 (2014).
59. Constantinides, M. G., McDonald, B. D., Verhoef, P. A. & Bendelac, A. A committed precursor to innate lymphoid cells. Nature 508, 397-+(2014).

1. An engineered killer innate-like T cell (ILTCk) comprising a non-endogenous expression vector including a mammalian TL-15 nucleic acid sequence or a mammalian STAT5B nucleic acid sequence, wherein the IL-15 nucleic acid sequence or the STAT5B nucleic acid sequence is operably linked to an expression control sequence, optionally wherein the engineered TLC is derived from an autologous donor or an allogenic donor.

2. The engineered ILTCk of claim 1, wherein the expression control sequence comprises an inducible promoter, a constitutive promoter, a native IL-15 or STAT5B promoter, or a heterologous promoter; or
wherein the non-endogenous expression vector is a plasmid, a cosmid, a bacmid, a bacterial artificial chromosome (BAC), a yeast artificial chromosome (YAC), a viral vector, or a retroviral vector; or
wherein the IL-15 nucleic acid sequence encodes the amino acid sequence of SEQ ID NO: 19 or SEQ ID NO: 20; or
wherein the STAT5B nucleic acid sequence encodes the amino acid sequence of SEQ ID NO: 9 or SEQ ID NO: 23.

3. (canceled)

4. (canceled)

5. (canceled)

6. The engineered ILTCk of claim 1, further comprising a chimeric antigen receptor (CAR) that binds to a tumor antigen and/or a nucleic acid encoding the CAR, optionally wherein the heterologous promoter is induced by binding of the CAR to the tumor antigen, or wherein binding of the CAR to the tumor antigen results in antigen-dependent JAK-STAT5 pathway activation: or
wherein the CAR comprises (i) an extracellular antigen binding domain: (ii) a transmembrane domain; and (iii) an intracellular domain comprising one or more co-stimulatory domains, wherein the extracellular antigen binding domain binds to the tumor antigen.

7. (canceled)

8. (canceled)

9. An engineered killer innate-like T cell (ILTCk) comprising a chimeric antigen receptor (CAR) that binds to a tumor antigen, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains, optionally wherein the truncated cytoplasmic domain of IL-2RβΔ comprises the amino acid sequence of SEQ ID NO: 7.

10. (canceled)

11. The engineered ILTCk of claim 6, wherein the extracellular antigen binding fragment comprises a single-chain variable fragment (scFv), optionally wherein the scFv is human: or
wherein the tumor antigen is selected from the group consisting of 5T4, alpha 5P1-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pml/RARa, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAPI, B7H3, Polysialic Acid, OX40, OX40-ligand, and peptide MHC complexes (with peptides derived from TP53, KRAS, MYC, EBNA1-6, PRAME, tyronsinase, MAGEAI-A6, pmel17, LMP2, or WT1).

12. (canceled)

13. (canceled)

14. The engineered ILTCk of claim 6, wherein the transmembrane domain comprises a CD8 transmembrane domain, a CD28 transmembrane domain, a NKG2D transmembrane domain, a CD3ζ transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, or a BTLA transmembrane domain; or
wherein the one or more co-stimulatory domains are selected from the group consisting of a CD28 co-stimulatory domain, a 4-1BB co-stimulatory domain, an OX40 co-stimulatory domain, an ICOS co-stimulatory domain, a DAP-10 co-stimulatory domain, a PD-1 co-stimulatory domain, a CTLA-4 co-stimulatory domain, a LAG-3 co-stimulatory domain, a 2B4 co-stimulatory domain, a BTLA co-stimulatory domain, a NKG2C co-stimulatory domain, a NKG2D co-stimulatory domain, and any combination thereof; or
wherein the one or more co-stimulatory domains comprise a DAP-10 co-stimulatory domain and a 2B4 co-stimulatory domain.

15. (canceled)

16. (canceled)

17. (canceled)

18. A composition comprising an effective amount of the engineered ILTCk of claim 1 and a pharmaceutically acceptable carrier.

19. A method of preparing the engineered ILTCk of claim 1 for adoptive cell therapy comprising: (a) isolating killer innate-like T cells (ILTCks) from a donor subject, (b) transducing the ILTCks with a nucleic acid encoding IL-15 or STAT5B or an expression vector comprising said nucleic acid, and (c) administering the transduced ILTCks to a recipient subject, optionally wherein
the nucleic acid encodes the amino acid sequence of SEQ ID NO: 19, SEQ ID NO: 20, SEQ ID NO: 9 or SEQ ID NO: 23, or wherein the donor subject and the recipient subject are the same or different; or
isolating ILTCks from the donor subject comprises isolating a population of immune cells from the donor subject, and collecting FCER1G⁺ cells from the isolated population of immune cells.

20. (canceled)

21. The method of claim 19, further comprising
transducing the ILTCks with a nucleic acid encoding a chimeric antigen receptor (CAR) that binds to a tumor antigen; or
transducing the ILTCks with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen: (ii) a transmembrane domain; and (iii) an intracellular domain comprising a truncated cytoplasmic domain of IL-2RβΔ and one or more co-stimulatory domains.

22. (canceled)

23. (canceled)

24. A method for treating cancer or inhibiting tumor growth in a subject in need thereof comprising administering to the subject an effective amount of the engineered ILTCk of claim 1, optionally wherein the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof; or wherein the engineered ILTCk is administered pleurally, intravenously, subcutaneously, intranodally, intratumorally, intrathecally, intrapleurally or intraperitoneally.

25. (canceled)

26. (canceled)

27. The method of claim 24, further comprising sequentially, separately, or simultaneously administering to the subject an additional cancer therapy, optionally wherein the additional cancer therapy is selected from among chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

28. (canceled)

29. A kit for preparing the engineered ILTCk of claim 1 comprising an expression vector that includes a nucleic acid sequence encoding an amino acid sequence of SEQ ID NO: 19, SEQ ID NO: 20, SEQ ID NO: 9 or SEQ ID NO: 23 and instructions for transducing ILTCks with the expression vector, and optionally a vector encoding an engineered CAR or other cell-surface ligand that binds to a tumor antigen.

30. (canceled)

31. (canceled)

32. (canceled)

33. (canceled)

34. (canceled)

35. A method for treating cancer or inhibiting tumor growth in a subject in need thereof comprising administering to the subject an effective amount of killer innate-like T cells (ILTCks), optionally wherein
the ILTCks are native ILTCks, genetically engineered ILTCks, or a combination thereof; or
the ILTCks are isolated from a donor subject and/or expanded ex vivo or in vitro: or
the subject harbors tumors with low mutation burden.

36. (canceled)

37. (canceled)

38. The method of claim 32, further comprising separately, simultaneously, or sequentially administering an effective amount of IL-15 to the subject, optionally wherein the IL-15 is administered to the subject prior to, during, or subsequent to administration of the ILTCks.

39. (canceled)

40. The method of claim 32, wherein the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof; or wherein the ILTCks are administered pleurally, intravenously, subcutaneously, intranodally, intratumorally, intrathecally, intrapleurally or intraperitoneally.

41. (canceled)

42. The method of claim 32, further comprising sequentially, separately, or simultaneously administering to the subject an additional cancer therapy, optionally wherein the additional cancer therapy is selected from among chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

43. (canceled)

44. The method of claim 35, further comprising
isolating a population of immune cells from a donor subject, and
collecting FCER1G⁺ cells from the isolated population of immune cells to obtain ILTCks.

45. The method of claim 44, wherein the FCER1G⁺ cells comprise FCER1G⁺ CD122⁺ cells, FCER1G⁺ NK1.1⁺GzmB^{+/-} cells, FCER1G⁺NK1.1⁻GzmB⁻ cells, and/or FCER1G⁺ PD-1⁺ cells.

46. The method of claim 35, wherein the tumors with low mutation burden are identified via next-generation sequencing using a tumor biopsy sample or cell-free DNA (cfDNA) sample obtained from the cancer subject; or wherein the tumors are refractory to immune checkpoint blockade therapy or adoptive cell therapy with CD8⁺ T cell therapy.


SEQUENCE LISTING (383 sequences)

SEQ ID NO: 1 - protein, 18 aa, Homo sapiens
MALPVTALLLPLALLLHA

SEQ ID NO: 2 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 3 - protein, 106 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 4 - protein, 21 aa, synthetic construct
PFFFCCFIAVAMGIRFIIMVT

SEQ ID NO: 5 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 6 - protein, 24 aa, synthetic construct
LCARPRRSPAQEDGKVYINMPGRG

SEQ ID NO: 7 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 786 aa, Homo sapiens
MAMWIQAQQPQGDALHQMQALYGQHFPIEVRHYLSQWIESQAWDSIDLDNPQENIKATQLLEGLVQELQKKAEHQVGEDGFLLKIKLGHYATQLQSTYDRCPMELVRCIRHILYNEQRLVREANNGSSPAGSLADAMSQKHLQINQTFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAERLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFDSQFSVGGNELVFQVKTLSLPVVVIVHGSQDNNATATVLWDNAFAEPGRVPFAVPDKVLWPQLCEALNMKFKAEVQSNRGLTKENLVFLAQKLFNISSNHLEDYNSMSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCEPATAKAADGYVKPQIKQVVPEFANAFTDAGSGATYMDQAPSPVVCPQAHYNMYPPNPDSVLDTDGDFDLEDMMDVARRVEELLGRPMDSQWIPHAQS

SEQ ID NO: 10 - DNA, 54 bp, synthetic construct
  source 1..54: mol_type = other DNA
atggctctcccagtgactgccctactgcttcccctagcgcttctcctgcatgca

SEQ ID NO: 11 - DNA, 366 bp, synthetic construct
  source 1..366: mol_type = other DNA
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctca

SEQ ID NO: 12 - DNA, 321 bp, synthetic construct
  source 1..321: mol_type = other DNA
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaa

SEQ ID NO: 13 - DNA, 63 bp, synthetic construct
  source 1..63: mol_type = other DNA
ccattttttttctgctgcttcatcgctgtagccatgggaatccgtttcattattatggtaaca

SEQ ID NO: 14 - DNA, 360 bp, synthetic construct
  source 1..360: mol_type = other DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 15 - DNA, 72 bp, synthetic construct
  source 1..72: mol_type = other DNA
ctgtgcgcacgcccacgccgcagccccgcccaagaagatggcaaagtctacatcaacatgccaggcaggggc

SEQ ID NO: 16 - DNA, 282 bp, synthetic construct
  source 1..282: mol_type = other DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtg

SEQ ID NO: 17 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - RNA, 2358 nt, Homo sapiens
  source 1..2358: mol_type = other RNA
atggctatgtggatacaggctcagcagccccagggcgatgcccttcaccagatgcaggccttgtacggccagcatttccccatcgaggtgcgacattatttatcacagtggatcgaaagccaagcctgggactcaatagatcttgataatccacaggagaacattaaggccacccagctcctggagggcctggtgcaggagctgcagaagaaggcggagcaccaggtgggggaagatgggtttttgctgaagatcaagctggggcactatgccacacagctccagagcacgtacgaccgctgccccatggagctggttcgctgtatccggcacattctgtacaacgaacagaggctggttcgcgaagccaacaacggcagctctccagctggaagtcttgctgacgccatgtcccagaagcaccttcagatcaaccaaacgtttgaggagctgcgcctgatcacacaggacacggagaacgagctgaagaagctgcagcagacccaagagtacttcatcatccagtaccaggagagcctgcggatccaagctcagtttgcccagctgggacagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaatcggcagcagatccgcagggctgagcgcctgtgccagcagctgcccatcccaggccccgtggaggagatgctggctgaggtcaacgccaccatcacggacatcatctcagccctggtcaccagcacgttcatcatcgagaagcagcctcctcaggtcctgaagacccagaccaagtttgcggccactgtgcgcctgctggtgggggggaagctgaatgtgcacatgaaccccccgcaggtgaaggcgaccatcatcagcgagcagcaggccaagtccctgctcaagaatgagaacacccgcaatgattacagcggcgagatcctgaacaactgttgcgtcatggagtaccaccaggccactggcacgctcagcgcccacttcagaaacatgtccctgaaacgaatcaagagatctgaccgccgtggtgcagagtcagtaacggaagagaagttcacgatcctgtttgactcacagttcagcgtcggtggaaacgagctggtctttcaagtcaagaccttgtcgctcccggtggtggtgattgttcacggcagccaggacaacaatgccacagccactgtcctctgggacaacgcctttgcagagcctggcagggtgccatttgccgtgcctgacaaggtgctgtggccgcagctgtgtgaagcgctcaacatgaaattcaaggctgaagtacagagcaaccggggcttgaccaaggagaacctcgtgttcctggcacagaaactgttcaacatcagcagcaaccacctcgaggactacaacagcatgtccgtgtcctggtcccagttcaaccgggagaatttgccaggacggaattacactttctggcagtggtttgatggcgtgatggaagtattgaaaaaacatctcaagcctcactggaatgatggggctatcctgggtttcgtgaacaagcaacaggcccacgacctgctcatcaacaagccggacgggaccttcctgctgcgcttcagcgactcggaaatcgggggcatcaccattgcttggaagtttgactctcaggagagaatgttttggaatctgatgccttttaccactagagacttctctatccggtccctcgctgaccgcctgggggacctgaattacctcatatacgtgtttcctgatcggccaaaggatgaagtatattctaagtactacacaccggtcccctgtgagcccgcaactgcgaaagcagctgacggatacgtgaagccacagatcaagcaggtggtccccgagtttgcaaatgcattcacagatgctgggagtggcgccacctacatggatcaggctccttccccagtcgtgtgccctcaggctcactacaacatgtacccacccaacccggactccgtccttgataccgatggggacttcgatctggaagacatgatggacgtggcgcggcgcgtggaagagctcttaggccggcccatggacagtcagtggatccctcacgcacagtca

SEQ ID NO: 19 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 20 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - RNA, 2015 nt, Homo sapiens
  source 1..2015: mol_type = other RNA
cttttcgccaggggttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaa

SEQ ID NO: 22 - RNA, 2336 nt, Homo sapiens
  source 1..2336: mol_type = other RNA
cttttcgccaggggttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaa

SEQ ID NO: 23 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 24 - DNA, 5079 bp, Homo sapiens
  source 1..5079: mol_type = other DNA
ggcggccggagccgtcaccccgggcggggacccagcgcaggcaactccgcgcggcggcccggccgagggagggagcgagcgggcgggcgggcaagccagacagctgggccggagcagccgcgggcgcccgaggggccgagcgagattgtaaaccatggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtgaccccgcgacctctccatcttcagcttcttcatcttcaccagaggaatcactcttgtggatgttttaattccatgaatcgcttctcttttgaaacaatactcataatgtgaagtgttaatactagttgtgaccttagtgtttctgtgcatggtggcaccagcgaagggagtgcgagtatgtgtttgtgtgtgtgtgtgtgtgtgtgtgtgtgtgcgtgtttgcacgttatggtgtttctccctctcactgtctgagagtttagttgtagcagaggggccacagacagaagctgtggtggtttttactttgtgcaaaaaggcagtgagtttcgtgaagcctggaagttggccatgtgtcttaagagtggctggactttgacatgtggctgtttgaataagagaaggacaaagggaggagaaagcacatgtgctccagtgagtcttcgtcactctgtctgccaagcaattgatatataaccgtgattgtctctgcttttcttctgaaatgtagataactgctttttgacaaagagagccttccctctcccccacccctgtgttcttgggtaggaatgggaaaaggggcaacctacaaagattgttggggcaagggaagtcacaagctttcggatgggcggtggcttttcacaaaacatttagctcatcttattctctctttgtcctctctcccctcctgcccgcccgcaccctggaattgccactcagttcctctgggtgtgcacatatgtttggagaaatagaggagagaaaagagggccacgtaactgagagcttacagtgccaatgccgtttgtgttctggccagagtggagtgcgcagccctgactcccaggcgctgagattgttgcctggttacccaggaagctgctgttccggctgcccagcctttctctgagccagcggatgcacagtccgtggccttcttcaggcttattgatgatgctttttgcaaatgttgaatcatggttctgtttctaagttggatcttttttgttttctccttgccaccctaatttgacatcaaaattctctcttgtgcattgggccctgggtcattcaaacccaggtcacctcattccccttctctgttcacacctaatgtcttgaagagtaggtagcagcagtgtgggctgaacctaggccagcttgcttagcgggtcaccctgctgtgaagtcctggcaggtgttggtaatgtgtggaaatgcagtcagcaagtttgctggggagtttgataaaagtataaaacaaaacaaaaaaagcctcggtataattttgttccacgacttcttctgtagctttacaccagaaggaaggaatgggctacagcaggtagtggaggaagaggggggtgagcaggtgtattaaaatagcttacgggtaaggcctaaaaggtcacccctcggccccctctccaaaagaagggcatgggcacccccaggagaggatggccccaaaaaccttatttttatacatgagagtaaataaacatattttttttacaaaaataacttctgaatttatcagtgttttgccgttaaaaatattcctctatagtaaattatttattggaagatgacttttttaaagctgccgtttgccttggcttggtttcatacactgatttatttttctatgccaggcagtagagtctctctgcctctgaggagcaggctacccgcatcccactcagcccctccctacccctcaagatttgatgaaaattccaaccatgaggatgggtgcatcggggaagggtgagaaggagagcctgcctgctcagggatccaggctcgtagagtcactccctgcccgtctcccagagatgcttcaccagcacctgcctctgagacctcgctctctgttccagcaaccctggttggggggtcagacttgatacactttcaggttgggagtggacccaccccagggcctgctgaggacagagcagccaggccgtcctggctcactttgcagttggcactgggttggggaggaagagagctgatgagtgtggcttccctgagctggggtttccctgcttgtccagttgtgagctgtcctcggtgttaccgaggctgtgcctagagagtggagatttttgatgaaaggtgtgctcgctctctgcgttctatcttctctctcctccttgttcctgcaaaccacaagataaaggtagtggtgtgtctcgaccccatcagcctctcacccactcccagacacacacaagtcctcaaaagtttcagctccgtgtgtgagatgtgcaggttttttctagggggtagggggagactaaaatcgaatataacttaaaatgaaagtatactttttataatttttctttttaaaacttggtgaaattatttcagatacatattttagtgtcaaggcagattagttatttagccaccaaaaaaaagtattgtgtacaatttggggcctcaaatttgactctgcctcaaaaaaaagaaatatatcctatgcagagttacagtcacaaagttgtgtattttatgttacaataaagccttcctctgaaggga

SEQ ID NO: 25 - protein, 21 aa, Homo sapiens
MALPVTALLLPLALLLHAARP

SEQ ID NO: 26 - DNA, 63 bp, unidentified
  source 1..63: mol_type = other DNA
atggccctgccagtaacggctctgctgctgccacttgctctgctcctccatgcagccaggcct

SEQ ID NO: 27 - protein, 21 aa, unidentified
MASPLTRFLSLNLLLLGESII

SEQ ID NO: 28 - DNA, 63 bp, unidentified
  source 1..63: mol_type = other DNA
atggccagccccctgaccaggttcctgagcctgaacctgctgctgctgggcgagagcatcatc

SEQ ID NO: 29 - protein, 18 aa, unidentified
MASPLTRFLSLNLLLLGE

SEQ ID NO: 30 - DNA, 54 bp, synthetic construct
  source 1..54: mol_type = other DNA
atggccagccccctgaccaggttcctgagcctgaacctgctgctgctgggcgag

SEQ ID NO: 31 - protein, 214 aa, synthetic construct
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 32 - DNA, 321 bp, synthetic construct
  source 1..321: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 33 - protein, 234 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 34 - protein, 71 aa, synthetic construct
PTTTPAPRPPTPAPTIASQPLSLRPEACRPAAGGAVHTRGLDFACDIYIWAPLAGTCGVLLLSLVITLYCN

SEQ ID NO: 35 - DNA, 213 bp, synthetic construct
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 36 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 37 - protein, 15 aa, synthetic construct
GGGGSGGGGSGGGGS

SEQ ID NO: 38 - DNA, 45 bp, synthetic construct
  source 1..45: mol_type = other DNA
ggcggcggcggatctggaggtggtggctcaggtggcggaggctcc

SEQ ID NO: 39 - protein, 8 aa, synthetic construct
DXEXNPGP

SEQ ID NO: 40 - protein, 8 aa, synthetic construct
GSGSGSGS

SEQ ID NO: 41 - protein, 120 aa, synthetic construct [sequence omitted]

SEQ ID NO: 42 - protein, 107 aa, synthetic construct [sequence omitted]

SEQ ID NO: 43 - protein, 115 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 44 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 45 - DNA, 360 bp, synthetic construct
  source 1..360: mol_type = other DNA
gaggtccagttggttgaatctggtggaggtttggtccagccaggtggatctttgagattgtcttgtgccgcttctggtttcaacatcaaggacacctacattcattgggttagacaagcccctggtaagggattggagtgggttgccagaatttacccaacaaacggatacacaagatacgctgactctgtcaagggaagattcactatctctgccgacacatctaagaacactgcatacttgcaaatgaactctttgagagccgaagacacagccgtctactactgctctagatggggtggtgacggtttttacgccatggactattggggtcaaggaacattggtcacagtctcttct

SEQ ID NO: 46 - DNA, 321 bp, synthetic construct
  source 1..321: mol_type = other DNA
gacattcagatgacccaatctccatcttctttgtctgcctctgtcggtgatagagttaccatcacctgcagagcttctcaagacgtcaataccgcagttgcctggtatcaacagaagccaggaaaggcacctaagttgttgatctactctgcttcttttttgtactctggagtcccttctagattttctggatctagatctggtaccgatttcacattgaccatttcttctttgcagcctgaggactttgccacatattactgtcagcagcactacacaacccctcctacttttggtcagggaactaaggtcgagattaag

SEQ ID NO: 47 - DNA, 345 bp, synthetic construct
  source 1..345: mol_type = other DNA
caggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgca

SEQ ID NO: 48 - DNA, 336 bp, synthetic construct
  source 1..336: mol_type = other DNA
gacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaag

SEQ ID NO: 49 - protein, 26 aa, synthetic construct
MAAPALSWRLPLLILLLPLATSWASA

SEQ ID NO: 50 - DNA, 78 bp, synthetic construct
  source 1..78: mol_type = other DNA
atggcggcccctgctctgtcctggcgtctgcccctcctcatcctcctcctgcccctggctacctcttgggcatctgca

SEQ ID NO: 51 - protein, 19 aa, synthetic construct
MDWIWRILFLVGAATGAHS

SEQ ID NO: 52 - DNA, 57 bp, synthetic construct
  source 1..57: mol_type = other DNA
atggattggatttggcgcattctgtttctggtgggagccgcaaccggagcacatagt

SEQ ID NO: 53 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 164 aa, synthetic construct; identical to SEQ ID NO: 53

SEQ ID NO: 55 - protein, 112 aa, synthetic construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 337 bp, synthetic construct
  source 1..337: mol_type = other DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 57 - protein, 251 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 58 - protein, 42 aa, synthetic construct
KRGRKKLLYIFKQPFMRPVQTTQEEDGCSCRFPEEEEGGCEL

SEQ ID NO: 59 - DNA, 126 bp, synthetic construct
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 60 - protein, 276 aa, synthetic construct
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 61 - protein, 196 aa, synthetic construct
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 62 - protein, 4 aa, synthetic construct
YVKM

SEQ ID NO: 63 - protein, 220 aa, synthetic construct
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 64 - protein, 286 aa, synthetic construct
MQIPQAPWPVVWAVLQLGWRPGWFLDSPDRPWNPPTFSPALLWTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGWGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 65 - protein, 524 aa, synthetic construct
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 66 - protein, 368 aa, synthetic construct
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 67 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 68 - protein, 8 aa, synthetic construct
SIINFEKL

SEQ ID NO: 69 - protein, 14 aa, synthetic construct
CAVTADYSNNRLTL

SEQ ID NO: 70 - protein, 13 aa, synthetic construct
CAPGTGGNNKLTF

SEQ ID NO: 71 - protein, 14 aa, synthetic construct
CAVIGGTGSNRLTF

SEQ ID NO: 72 - protein, 12 aa, synthetic construct
CAGYQGGRALIF

SEQ ID NO: 73 - protein, 14 aa, synthetic construct
CAASAIPGYQNFYF

SEQ ID NO: 74 - protein, 15 aa, synthetic construct
CAVRAHDTNAYKVIF

SEQ ID NO: 75 - protein, 14 aa, synthetic construct
CAASAIPGYQNFYF

SEQ ID NO: 76 - protein, 12 aa, synthetic construct
CAMMSNYNVLYF

SEQ ID NO: 77 - protein, 16 aa, synthetic construct
CALGDLGGSSGNKLIF

SEQ ID NO: 78 - protein, 14 aa, synthetic construct
CAANSGGSNAKLTF

SEQ ID NO: 79 - protein, 13 aa, synthetic construct
CAPGTGGNNKLTF

SEQ ID NO: 80 - protein, 12 aa, synthetic construct
CAGYQGGRVLIF

SEQ ID NO: 81 - protein, 15 aa, synthetic construct
CALSDQVNYAQGLTF

SEQ ID NO: 82 - protein, 16 aa, synthetic construct
CALGDLGGSSGNKLIF

SEQ ID NO: 83 - protein, 14 aa, synthetic construct
CAVSADYSNNRLTL

SEQ ID NO: 84 - protein, 13 aa, synthetic construct
CALANSAGNKLTF

SEQ ID NO: 85 - protein, 13 aa, synthetic construct
CAVSAPNTNKVVF

SEQ ID NO: 86 - protein, 14 aa, synthetic construct
CALSDQGTTTRDPF

SEQ ID NO: 87 - protein, 15 aa, synthetic construct
CAVSVANNAGAKLTF

SEQ ID NO: 88 - protein, 13 aa, synthetic construct
CAPGVGGNNKLTF

SEQ ID NO: 89 - protein, 14 aa, synthetic construct
CSVSAGNSNNRFTL

SEQ ID NO: 90 - protein, 14 aa, synthetic construct
CAVIGGTGSNRLTF

SEQ ID NO: 91 - protein, 13 aa, synthetic construct
CAVSEQGGRALIF

SEQ ID NO: 92 - protein, 11 aa, synthetic construct
CALSGYNRIFF

SEQ ID NO: 93 - protein, 12 aa, synthetic construct
CAGYQGGRALIF

SEQ ID NO: 94 - protein, 14 aa, synthetic construct
CAVSADYSNNRLTL

SEQ ID NO: 95 - protein, 15 aa, synthetic construct
CALRDQGTTSQGLTF

SEQ ID NO: 96 - protein, 13 aa, synthetic construct
CAPGTGGNNKLTF

SEQ ID NO: 97 - protein, 14 aa, synthetic construct
CAGRDRGTATRDPF

SEQ ID NO: 98 - protein, 14 aa, synthetic construct
CAVSANTGYQNFYF

SEQ ID NO: 99 - protein, 13 aa, synthetic construct
CAAGYTGNYKYVF

SEQ ID NO: 100 - protein, 14 aa, synthetic construct
CAASGNNYAQGLTF

SEQ ID NO: 101 - protein, 12 aa, synthetic construct
CRAYQGGRALIF

SEQ ID NO: 102 - protein, 13 aa, synthetic construct
CAPGTGGNNKLTF

SEQ ID NO: 103 - protein, 14 aa, synthetic construct
CAANSGRSNAKLTF

SEQ ID NO: 104 - protein, 12 aa, synthetic construct
CAGYQGGRALKF

SEQ ID NO: 105 - protein, 14 aa, synthetic construct
CGVRATNYAQGLTF

SEQ ID NO: 106 - protein, 13 aa, synthetic construct
CACPQTNTGKLIF

SEQ ID NO: 107 - protein, 15 aa, synthetic construct
CVLRYMTAMLTKSSF

SEQ ID NO: 108 - protein, 15 aa, synthetic construct
CVVRYMTQIITKSSF

SEQ ID NO: 109 - protein, 15 aa, synthetic construct
CAAMNYGSSGNKLIF

SEQ ID NO: 110 - protein, 12 aa, synthetic construct
CATNNYAQGLTF

SEQ ID NO: 111 - protein, 14 aa, synthetic construct
CALGASSGSWQLIF

SEQ ID NO: 112 - protein, 15 aa, synthetic construct
CAASPSTDNSKLIWF

SEQ ID NO: 113 - protein, 15 aa, synthetic construct
CAVTADYSNNRLTLF

SEQ ID NO: 114 - protein, 14 aa, synthetic construct
CVRYATTMPTKSSF

SEQ ID NO: 115 - protein, 15 aa, synthetic construct
CAVRYATQAITALSF

SEQ ID NO: 116 - protein, 12 aa, synthetic construct
CVPTTNAYKVIF

SEQ ID NO: 117 - protein, 14 aa, synthetic construct
CAVRHDTNAYKVIF

SEQ ID NO: 118 - protein, 15 aa, synthetic construct
CALGPWGSAGNKLIF

SEQ ID NO: 119 - protein, 12 aa, synthetic construct
CAASENNNAPRF

SEQ ID NO: 120 - protein, 17 aa, synthetic construct
CALGDRDSGGSNYKLTF

SEQ ID NO: 121 - protein, 15 aa, synthetic construct
CAIDAASSGSWQLIF

SEQ ID NO: 122 - protein, 13 aa, synthetic construct
CAPRVSGSWQLIF

SEQ ID NO: 123 - protein, 14 aa, synthetic construct
CAASGGYNQGKLIF

SEQ ID NO: 124 - protein, 14 aa, synthetic construct
CAVSANTGYQNFYF

SEQ ID NO: 125 - protein, 14 aa, synthetic construct
CAAPNVGDNSKLIW

SEQ ID NO: 126 - protein, 14 aa, synthetic construct
CAVSAGGSNAKLTF

SEQ ID NO: 127 - protein, 15 aa, synthetic construct
CSVVLAVRAAKAMVF

SEQ ID NO: 128 - protein, 13 aa, synthetic construct
CAVSANNNNAPRF

SEQ ID NO: 129 - protein, 12 aa, synthetic construct
CAADTNAYKVIF

SEQ ID NO: 130 - protein, 15 aa, synthetic construct
CALSDRGDSNYQLIW

SEQ ID NO: 131 - protein, 14 aa, synthetic construct
CVLSSNYNQGKLIF

SEQ ID NO: 132 - protein, 14 aa, synthetic construct
CAVSAVSGSWVLIF

SEQ ID NO: 133 - protein, 14 aa, synthetic construct
CAMRDYNYAQGLTF

SEQ ID NO: 134 - protein, 14 aa, synthetic construct
CAMRFDTNAYKVIF

SEQ ID NO: 135 - protein, 13 aa, synthetic construct
CAASPNVGYVLTF

SEQ ID NO: 136 - protein, 14 aa, synthetic construct
CAVSRLDSNYQLIW

SEQ ID NO: 137 - protein, 16 aa, synthetic construct
CVLSGSASSNTNKVVF

SEQ ID NO: 138 - protein, 15 aa, synthetic construct
CALRHQANYAQGLTF

SEQ ID NO: 139 - protein, 14 aa, synthetic construct
CAADASSGSWQLIF

SEQ ID NO: 140 - protein, 14 aa, synthetic construct
CALSEGTGYQNFYF

SEQ ID NO: 141 - protein, 12 aa, synthetic construct
CAVITNTGKLTF

SEQ ID NO: 142 - protein, 16 aa, synthetic construct
CAMERNTGANTGKLTF

SEQ ID NO: 143 - protein, 15 aa, synthetic construct
CAVSAAGSALGRLHF

SEQ ID NO: 144 - protein, 15 aa, synthetic construct
CALSEATRDNNKLTF

SEQ ID NO: 145 - protein, 14 aa, synthetic construct
CALSEGNYAQGLTF

SEQ ID NO: 146 - protein, 16 aa, synthetic construct
CALSYPNQGGSAKLIF

SEQ ID NO: 147 - protein, 15 aa, synthetic construct
CAMRENQGGSAKLIF

SEQ ID NO: 148 - protein, 13 aa, synthetic construct
CAPSTGGNNKLTF

SEQ ID NO: 149 - protein, 14 aa, synthetic construct
CALGEAYNQGKLIF

SEQ ID NO: 150 - protein, 14 aa, synthetic construct
CAMREAQGGRALIF

SEQ ID NO: 151 - protein, 13 aa, synthetic construct
CAGNTEGADRLTF

SEQ ID NO: 152 - protein, 14 aa, synthetic construct
CALSRGSAGNKLTF

SEQ ID NO: 153 - protein, 14 aa, synthetic construct
CAMREGNYAQGLTF

SEQ ID NO: 154 - protein, 13 aa, synthetic construct
CAASGNYAQGLTF

SEQ ID NO: 155 - protein, 14 aa, synthetic construct
CAVSTSNMGYKLTF

SEQ ID NO: 156 - protein, 14 aa, synthetic construct
CAVSTSNMGYKLTF

SEQ ID NO: 157 - protein, 14 aa, synthetic construct
CAVSGGASNNRIFF

SEQ ID NO: 158 - protein, 13 aa, synthetic construct
CAVSMPNTNKVVF

SEQ ID NO: 159 - protein, 12 aa, synthetic construct
CAVRDSNYQLIW

SEQ ID NO: 160 - protein, 14 aa, synthetic construct
CALGANYNQGKLIF

SEQ ID NO: 161 - protein, 13 aa, synthetic construct
CAVTTNSGTYQRF

SEQ ID NO: 162 - protein, 13 aa, synthetic construct
CAASPNMGYKLTF

SEQ ID NO: 163 - protein, 13 aa, synthetic construct
CAASPNMGYKLTF

SEQ ID NO: 164 - protein, 13 aa, synthetic construct
CAASPNMGYKLTF

SEQ ID NO: 165 - protein, 15 aa, synthetic construct
CALGGITGNTGKLIF

SEQ ID NO: 166 - protein, 12 aa, synthetic construct
CAGSNSGTYQRF

SEQ ID NO: 167 - protein, 10 aa, synthetic construct
CAVSRGRIFF

SEQ ID NO: 168 - protein, 12 aa, synthetic construct
CAMVGYAQGLTF

SEQ ID NO: 169 - protein, 14 aa, synthetic construct
CAAERGTGNYKYVF

SEQ ID NO: 170 - protein, 14 aa, synthetic construct
CAMRENTGYQNFYF

SEQ ID NO: 171 - protein, 14 aa, synthetic construct
CATLASSGSWQLIF

SEQ ID NO: 172 - protein, 14 aa, synthetic construct
CAVSAAWRLAKVVF

SEQ ID NO: 173 - protein, 15 aa, synthetic construct
CAAGPASSGSWQLIF

SEQ ID NO: 174 - protein, 14 aa, synthetic construct
CAASASSGSWQLIF

SEQ ID NO: 175 - protein, 12 aa, synthetic construct
CALGDYAQGLTF

SEQ ID NO: 176 - protein, 14 aa, synthetic construct
CAVSGDTAAYKVIF

SEQ ID NO: 177 - protein, 14 aa, synthetic construct
CVLGDHYNQGKLIF

SEQ ID NO: 178 - protein, 11 aa, synthetic construct
CATEGGRALIF

SEQ ID NO: 179 - protein, 17 aa, synthetic construct
CAMERSGYGSSGNKLIF

SEQ ID NO: 180 - protein, 15 aa, synthetic construct
CAMRDVNTGYQNFYF

SEQ ID NO: 181 - protein, 16 aa, synthetic construct
CALSKGHKCLQSHAAF

SEQ ID NO: 182 - protein, 14 aa, synthetic construct
CVKGDHYNQGKLIF

SEQ ID NO: 183 - protein, 13 aa, synthetic construct
CAIADNYAQGLTF

SEQ ID NO: 184 - protein, 16 aa, synthetic construct
CASSTPGWGYQDTQYF

SEQ ID NO: 185 - protein, 15 aa, synthetic construct
CASSIDWNSQNTLYF

SEQ ID NO: 186 - protein, 15 aa, synthetic construct
CASSFGLRPFAEQFF

SEQ ID NO: 187 - protein, 18 aa, synthetic construct
CASSIGLGGNYAEQDYFF

SEQ ID NO: 188 - protein, 15 aa, synthetic construct
CASSEDWGEEDTQYF

SEQ ID NO: 189 - protein, 16 aa, synthetic construct
CASSTPGWGYQDTQYF

SEQ ID NO: 190 - protein, 15 aa, synthetic construct
CASSEDWGEEDTQYF

SEQ ID NO: 191 - protein, 14 aa, synthetic construct
CASSIGLGGNYAEF

SEQ ID NO: 192 - protein, 14 aa, synthetic construct
CASSPGTGVSNERF

SEQ ID NO: 193 - protein, 15 aa, synthetic construct
CASSIDWNSQNTLYF

SEQ ID NO: 194 - protein, 15 aa, synthetic construct
CASSIDWNSQNTLYF

SEQ ID NO: 195 - protein, 17 aa, synthetic construct
CASSIGLGGNYAEQKFF

SEQ ID NO: 196 - protein, 13 aa, synthetic construct
CASSLGGFYEQYF

SEQ ID NO: 197 - protein, 15 aa, synthetic construct
CASSFSTLKIQPTEF

SEQ ID NO: 198 - protein, 13 aa, synthetic construct
CASSLGGFYEQYF

SEQ ID NO: 199 - protein, 16 aa, synthetic construct
CASSPGTGVSNERLFF

SEQ ID NO: 200 - protein, 15 aa, synthetic construct
CASRVPTPSGNKVYF

SEQ ID NO: 201 - protein, 13 aa, synthetic construct
CASSLGGFYEQYF

SEQ ID NO: 202 - protein, 12 aa, synthetic construct
CASSADRYEQYF

SEQ ID NO: 203 - protein, 15 aa, synthetic construct
CASSIDWNSQNTLYF

SEQ ID NO: 204 - protein, 13 aa, synthetic construct
CASSLGGFYEQYF

SEQ ID NO: 205 - protein, 14 aa, synthetic construct
CASSFGLRPFAEQF

SEQ ID NO: 206 - protein, 15 aa, synthetic construct
CASSLTGTGGYTQYF

SEQ ID NO: 207 - protein, 15 aa, synthetic construct
CASSEDWGEEDTQYF

SEQ ID NO: 208 - protein, 13 aa, synthetic construct
CASSPGTNSDYTF

SEQ ID NO: 209 - protein, 17 aa, synthetic construct
CASSIGLGGNYAEQKFF

SEQ ID NO: 210 - protein, 13 aa, synthetic construct
CASSLGGFYEQYF

SEQ ID NO: 211 - protein, 15 aa, synthetic construct
CASSIDWNSQNTLYF

SEQ ID NO: 212 - protein, 13 aa, synthetic construct
CASSLGGFYEQYF

SEQ ID NO: 213 - protein, 12 aa, synthetic construct
CASSPGPREQYF

SEQ ID NO: 214 - protein, 12 aa, synthetic construct
CASSIGNTEVFF

SEQ ID NO: 215 - protein, 14 aa, synthetic construct
CAVKRGAHRAAAYF

SEQ ID NO: 216 - protein, 14 aa, synthetic construct
CASSIGLGGNYAEF

SEQ ID NO: 217 - protein, 15 aa, synthetic construct
CASSIDWNSQNTLYF

SEQ ID NO: 218 - protein, 15 aa, synthetic construct
CASSIDWNSQNTLYF

SEQ ID NO: 219 - protein, 15 aa, synthetic construct
CASIGLGGNYAEQFF

SEQ ID NO: 220 - protein, 15 aa, synthetic construct
CASSLGGRGAETLYF

SEQ ID NO: 221 - protein, 12 aa, synthetic construct
CASSPRGTEVFF

SEQ ID NO: 222 - protein, 14 aa, synthetic construct
CASSTPGWGYQDYF

SEQ ID NO: 223 - protein, 14 aa, synthetic construct
CASSTPGWGYQDYF

SEQ ID NO: 224 - protein, 14 aa, synthetic construct
CANSSLAGDYEQYF

SEQ ID NO: 225 - protein, 14 aa, synthetic construct
CASSSLAGDYEQYF

SEQ ID NO: 226 - protein, 14 aa, synthetic construct
CASSSLAGDYEQYF

SEQ ID NO: 227 - protein, 16 aa, synthetic construct
CASSTPGWGYQDTQYF

SEQ ID NO: 228 - protein, 16 aa, synthetic construct
CASSTPGWGYQDTQYF

SEQ ID NO: 229 - protein, 12 aa, synthetic construct
CASSLRHSDYTF

SEQ ID NO: 230 - protein, 16 aa, synthetic construct
CASSTPGWGYQDTQYF

SEQ ID NO: 231 - protein, 16 aa, synthetic construct
CASSTPGWGYQDTQYF

SEQ ID NO: 232 - protein, 14 aa, synthetic construct
CASSPRTNQDTQYF

SEQ ID NO: 233 - protein, 13 aa, synthetic construct
CASSRGGHTEVFF

SEQ ID NO: 234 - protein, 13 aa, synthetic construct
CASSRKNQDTQYF

SEQ ID NO: 235 - protein, 15 aa, synthetic construct
CASSLSWGDAETLYF

SEQ ID NO: 236 - protein, 14 aa, synthetic construct
CASRMGGANTEVFF

SEQ ID NO: 237 - protein, 15 aa, synthetic construct
CASSQGVISNERLFF

SEQ ID NO: 238 - protein, 16 aa, synthetic construct
CAAWVCTPGQYTYVLF

SEQ ID NO: 239 - protein, 14 aa, synthetic construct
CASSPRDWGYEQYF

SEQ ID NO: 240 - protein, 14 aa, synthetic construct
CASSPLGGLREQYF

SEQ ID NO: 241 - protein, 14 aa, synthetic construct
CASSPRDWGYEQYF

SEQ ID NO: 242 - protein, 14 aa, synthetic construct
CASSPDRANTEVFF

SEQ ID NO: 243 - protein, 15 aa, synthetic construct
CASSLTGGHNERLFF

SEQ ID NO: 244 - protein, 15 aa, synthetic construct
CASRGLGANQDTQYF

SEQ ID NO: 245 - protein, 15 aa, synthetic construct
CASSADWVSNERLFF

SEQ ID NO: 246 - protein, 12 aa, synthetic construct
CASSILAYEQYF

SEQ ID NO: 247 - protein, 15 aa, synthetic construct
CASIVETSRHNSLVF

SEQ ID NO: 248 - protein, 15 aa, synthetic construct
CASSRTGGSQNTLYF

SEQ ID NO: 249 - protein, 15 aa, synthetic construct
CASSWDRGAGNTLYF

SEQ ID NO: 250 - protein, 14 aa, synthetic construct
CASSLEENTGQLYF

SEQ ID NO: 251 - protein, 15 aa, synthetic construct
CASSGTGGLQNTLYF

SEQ ID NO: 252 - protein, 14 aa, synthetic construct
CASSPGTNYAEQFF

SEQ ID NO: 253 - protein, 12 aa, synthetic construct
CASSDWGGEQYF

SEQ ID NO: 254 - protein, 14 aa, synthetic construct
CAVPAVGTGSSSAF

SEQ ID NO: 255 - protein, 13 aa, synthetic construct
CASGGGTGWEQYF

SEQ ID NO: 256 - protein, 17 aa, synthetic construct
CASPSGIGRSGASTLYF

SEQ ID NO: 257 - protein, 14 aa, synthetic construct
CASTCSATGYEQYF

SEQ ID NO: 258 - protein, 14 aa, synthetic construct
CASSPGTLYAEQFF

SEQ ID NO: 259 - protein, 15 aa, synthetic construct
SASSHRSGGYAEQFF

SEQ ID NO: 260 - protein, 15 aa, synthetic construct
CASSRTGGGYAEQFF

SEQ ID NO: 261 - protein, 14 aa, synthetic construct
CASSRDWGTYEQYF

SEQ ID NO: 262 - protein, 15 aa, synthetic construct
CAVVTGTGGKHLVSF

SEQ ID NO: 263 - protein, 14 aa, synthetic construct
CASSLSRSNTEVFF

SEQ ID NO: 264 - protein, 13 aa, synthetic construct
CASGDEDRYSQYF

SEQ ID NO: 265 - protein, 14 aa, synthetic construct
CGARGRTGRNTLYF

SEQ ID NO: 266 - protein, 15 aa, synthetic construct
CASSHDPGDYAEQFF

SEQ ID NO: 267 - protein, 14 aa, synthetic construct
CASSLTGGYAEQFF

SEQ ID NO: 268 - protein, 15 aa, synthetic construct
CASSLGTANTGQLYF

SEQ ID NO: 269 - protein, 15 aa, synthetic construct
CASGVRDWGDAEQFF

SEQ ID NO: 270 - protein, 15 aa, synthetic construct
CASSPRQNNYAEQFF

SEQ ID NO: 271 - protein, 15 aa, synthetic construct
CASSPRQNNYAEQFF

SEQ ID NO: 272 - protein, 13 aa, synthetic construct
CASSLWGGREQYF

SEQ ID NO: 273 - protein, 16 aa, synthetic construct
CASGPRLGTSQNTLYF

SEQ ID NO: 274 - protein, 15 aa, synthetic construct
CASTPTGGYYAEQFF

SEQ ID NO: 275 - protein, 15 aa, synthetic construct
CASSFDWESAETLYF

SEQ ID NO: 276 - protein, 15 aa, synthetic construct
CASSVGDRANERLFF

SEQ ID NO: 277 - protein, 14 aa, synthetic construct
CASSLEENTGQLYF

SEQ ID NO: 278 - protein, 14 aa, synthetic construct
CASSLEENTGQLYF

SEQ ID NO: 279 - protein, 14 aa, synthetic construct
CASSLEENTGQLYF

SEQ ID NO: 280 - protein, 14 aa, synthetic construct
CASSLSGGSYEQYF

SEQ ID NO: 281 - protein, 14 aa, synthetic construct
CASSFSRGDSDYTF

SEQ ID NO: 282 - protein, 14 aa, synthetic construct
CTCSADPENTEVFF

SEQ ID NO: 283 - protein, 17 aa, synthetic construct
CASSPSGGAGGAETLYF

SEQ ID NO: 284 - protein, 16 aa, synthetic construct
CTCSVTGGANTGQLYF

SEQ ID NO: 285 - protein, 13 aa, synthetic construct
CASGDQGNTEVFF

SEQ ID NO: 286 - protein, 18 aa, synthetic construct
CASSPRDWGGARAETLYF

SEQ ID NO: 287 - protein, 13 aa, synthetic construct
CTCSADTTETLYF

SEQ ID NO: 288 - protein, 15 aa, synthetic construct
CTCSALPGADTEVFF

SEQ ID NO: 289 - protein, 15 aa, synthetic construct
CAARTGEVSAETLYF

SEQ ID NO: 290 - protein, 16 aa, synthetic construct
CASSFAAEANTETLYF

SEQ ID NO: 291 - protein, 13 aa, synthetic construct
CASSLWGAETLYF

SEQ ID NO: 292 - protein, 14 aa, synthetic construct
CASSGTPGYSDYTF

SEQ ID NO: 293 - protein, 15 aa, synthetic construct
CASSFGWGAQNTLYF

SEQ ID NO: 294 - protein, 13 aa, synthetic construct
CASSPRDRNTLYF

SEQ ID NO: 295 - protein, 16 aa, synthetic construct
CASSPGLGGSQNTLYF

SEQ ID NO: 296 - protein, 15 aa, synthetic construct
CASSLWPDNYAEQFF

SEQ ID NO: 297 - protein, 14 aa, synthetic construct
CASSGTPGYSDYTF

SEQ ID NO: 298 - protein, 11 aa, synthetic construct
CASSLQNTLYF

SEQ ID NO: 299 - DNA, 388 bp, synthetic construct
  source 1..388: mol_type = other DNA
atgaagaggctgctgtgttctctgctggggcttctgtgcacccaggtttgctgggtgaaaggacagcaagtgcagcagagtccctcgtccttggttctgcaggagggggagaacgcagagctgcagtgtaacttttcctccacagcaacccagctgcagtggttttaccaaagtcctgggggaagcctcgtcagcctgttgtccaatccttctgggacaaagcacactggaagactgacatccaccacagtcactaaggaacgtcgcagctctttgcacatttcctcctcccagacaacagactcaggcacttatctctgtgctatagaggcaaattctgggacttaccagaggtttggaactgggacaaaactccaagtcgttccaa

SEQ ID NO: 300 - DNA, 394 bp, synthetic construct
  source 1..394: mol_type = other DNA
atgaacagattcctgggaatatctttggtgactctatggtttcgagtggcctgggcaaagagccaatggggagaagagaatcttcaggctctgagcatccaggagggtgaagatgtcaccatgaactgcagttacaagacttatacaactgttgtacactggtacaggcaagactcaggcagaggccctgccctgataatcttaatacgttcaaatgagcgagagaagcgcagtggaagactaagagccacccttgacacctccagccaaagtagctctctgtccatcactgctgctcagtgtgaagacactgctgtgtacttctgtgctacagatgaccagggaggcagagctctgatatttggaacaggaaccacggtatcagtcagcccca

SEQ ID NO: 301 - DNA, 418 bp, synthetic construct
  source 1..418: mol_type = other DNA
atggacaagatcctgacagcatcgtttttactcctaggccttcacctagctggggtgagtggccagcagcaggagaaacgtgaccagcagcaggtgagacaaagtccccaatctctgacagtctgggaaggagagacagcaattctgaactgcagttatgaggacagcacttttgactacttcccatggtactggcagttccctagggaaagccctgcactcctgatagccatacgtccagtgtccaataaaaaggaagatggacgattcacaatcttcttcaataaaagggagaaaaagctctccttgcacatcacagactctcagcctggagactcagctacctacttctgtgcagcaagtgacatgggctacaaacttaccttcgggacaggaacaagcctgttggttgatccaa

SEQ ID NO: 302 - DNA, 391 bp, synthetic construct
  source 1..391: mol_type = other DNA
atgaatacttctccagttttagtgactgcgatgctgctgttcatgcttaagacccacggagattcagtgacccagaaacaaggtcaagtgaccctttcagaagatgacttcctatttataaattgcacttattctaccacaacgtacccaactcttttctggtatgtccaatatcctggacaaggtccacagctccttctgaaagtcacaactgccaacaacaagggaatcagcagaggctttgaagctacatatgacaaagggaccacctccttccacttacagaaagcctcagtgcatgagtcagactcagccgtgtacttctgtgctctgggagactatgcccagggattaaccttcggtcttggcaccagagtatctgtgtttccct

SEQ ID NO: 303 - DNA, 391 bp, synthetic construct
  source 1..391: mol_type = other DNA
atgaagagcctgctgagctctctgctggggcttctgtgcacccaggtttgctgggtgaaaggacagcaagtgcagcagagccccgcgtccttggttctgcaggagggggagaacgcagagctgcagtgtaacttttccacatctttgaacagtatgcagtggttttaccaacgtcctgggggaagtctcgtcagcctgttctacaatccttctgggacaaagcatagtgggagactgacatccactacagtcatcaaagaacgtcgcagctctttgcacatttcctcctcccagacaacagactcaggcacttatctctgtgctttggaaacgggttcagccttagggaggctgcattttggagctgggactcagctgattgtcatacctg

SEQ ID NO: 304 - DNA, 400 bp, synthetic construct
  source 1..400: mol_type = other DNA
atgaacagattcctgggaatatctttggtgactctatggtttcaagtggcctgggcaaagagccaatggggagaagagaatcttcaggctctgagcatccaggagggtgaagatgtcaccatgaactgcagttacaagacttacacaactgttgttcagtggtacagacagaagtcaggcaaaggccctgcccagctaatcttaatacgttcaaatgagcgagagaagcgcagtggaagactcagagccacccttgacacttccagccagagcagctccctgtccatcactggtactctagctacagacactgctgtgtacttctgtgctactgagccctatgactcgggatacaacaaactcacttttggaaagggcacggtgcttctagtctctccag

SEQ ID NO: 305 - DNA, 397 bp, synthetic construct
  source 1..397: mol_type = other DNA
atgcgtcctgacacctgctcagttcttgtgctcctcttaatgctcagaaggagcaatggagactcagtgacccagacagaaggcctggtcactctcaccaaggggttgcctgtgatgctgaactgcacctatcagactacttactcacctttccttttctggtatgtgcaacatctcaacgaagcccctaagctactcctgaagagctccacagacaacaagaggaccgagcaccaagggttctacgccactctccataagagcagcagctccttccatctgcagaagtcctcagtgcagctgtcagactctgccctgtacttctgtgctttgagtgatgggactggaggctataaagtggtctttggaagtgggactcgattgctggtaagccctg

SEQ ID NO: 306 - DNA, 427 bp, synthetic construct
  source 1..427: mol_type = other DNA
atggacaagatcctgacagcaacgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccgtacgttcagtgtccgataaaaaggaagatggacgattcacaatcttcttcaataaaagggagaaaaagctctccttgcacatcacagactctcagcctggagactcagctacctacttctgtgcagcaggtccagcatcttctggcagctggcaactcatctttggatctggaacccaactgacagttatgcctg

SEQ ID NO: 307 - DNA, 400 bp, synthetic construct
  source 1..400: mol_type = other DNA
atgctgattctaagcctgttgggagcagcctttggctccatttcaaccagcatggcccagaaggtaacacagactcagacttcaatttctgtgatggagaagacaacggtgacaatggactgtgtgtatgaaacccaggacagttcttacttcttattctggtacaagcaaacagcaagtggggaaatagttttccttattcgtcaggactcttacaaaaaggaaaatgcaacagtgggtcattattctctgaactttcagaagccaaaaagttccatcggactcatcatcaccgccacacagattgaggactcagcagtatatttctgtgctatgagacccaactatggaaatgagaaaataacttttggggctggaaccaaactcaccattaaaccca

SEQ ID NO: 308 - DNA, 400 bp, synthetic construct
  source 1..400: mol_type = other DNA
atgaaatccttgagtgtttccctagtggtcctgtggctccagttaaactgggtgaagagccagcagaaggtgcagcagagcccagaatccctcagtgtcccagagggaggcatggcctctctcaactgcacttcaagtgatcgtaattttcagtatttctggtggtacagacagcattctggagaaggccccaaagcactgatgtccatcttctctgatggtgacaagaaagaaggcagattcacagctcacctcaataaggccagcctgcatgtttccctgcacatcagagactcccagcccagtgactccgctctctacttctgtgcagttagtgctgcatcttctggcagctggcaactcatctttggatctggaacccaactgacagttatgcctg

SEQ ID NO: 309 - DNA, 406 bp, synthetic construct
  source 1..406: mol_type = other DNA
atggactcttctccaggcttcgtggctgtgatacttctcatacttggaaggacccacggagattccgtgactcaaacagaaggccaagtgaccgtctcagaaagcaagtccctgataataaattgcacgtattcaaccacaagcatagcttaccctaatcttttctggtatgttcgatatcctggagaaggtctacaactcctcctgaaagtcattacggctggccagaagggaagcagcagagggtttgaagccacatacaataaagaaaccacctccttccacttgcagaaagcctcagtgcaagagtcagactcggctgtgtactactgtgctctggccctcatgaattataaccaggggaagcttatctttggacagggaaccaagttatctatcaagccca

SEQ ID NO: 310 - DNA, 397 bp, synthetic construct
  source 1..397: mol_type = other DNA
atgaactattctccagctttagtgactgtgatgctgtttgtgtttgggaggacccatggagactcagtgacccagatgcaaggtcaagtgaccctctcagaagacgacttcctatttataaactgtacttattcaaccacatggtacccgactcttttctggtatgtccaatatcccggagaaggtccacagctccttttgaaagtcacaacagccaacaacaagggaatcagcagaggttttgaagctacatatgataaaggaacaacgtccttccacttgcagaaagcctcagtgcaggagtcagactctgctgtgtactactgtgttctgggtggggcttctggcagctggcaactcatctttggatctggaacccaactgacagttatgcctg

SEQ ID NO: 311 - DNA, 406 bp, synthetic construct
  source 1..406: mol_type = other DNA
atgctgattctaagcctgctgggagcagcctttggctccatttgttttgcaaccagcatggcccagaaggtaacacagactcagacttcaatttctgtgatggagaagacaacggtgacaatggactgtgtgtatgaaacccgggacagttcttacttcttattctggtacaagcaaacagcaagtggggaaatagttttccttattcgtcaggactcttacaaaaaggaaaatgcaacagaaggtcattattctctgaactttcagaagccaaaaagttccatcggactcatcatcactgccacacagattgaggactcagcagtatatttctgtgctatgagggccaactatggaaatgagaaaataacttttggggctggaaccaaactcaccattaaaccca

SEQ ID NO: 312 - DNA, 412 bp, synthetic construct
  source 1..412: mol_type = other DNA
atgctgattctaagcctgctgggagcagcctttggctccatttgttttgcaaccagcatggcccagaaggtaacacagactcagacttcaatttctgtgatggagaagacaacggtgacaatggactgtgtgtatgaaacccgggacagttcttacttcttattctggtacaagcaaacagcaagtggggaaatagttttccttattcgtcaggactcttacaaaaaggaaaatgcaacagaaggtcattattctctgaactttcagaagccaaaaagttccatcggactcatcatcactgccacacagattgaggactcagcagtatatttctgtgctatgagagaggggggtggaggcagcaattacaaactgacatttgggaaaggaactctcttaactgtgactccaa

SEQ ID NO: 313 - DNA, 394 bp, synthetic construct
  source 1..394: mol_type = other DNA
atgaactattctccagctttagtgactgtgatgctgtttgtgtttgggaggacccatggagactcagtgacccagatgcaaggtcaagtgaccctctcagaagacgacttcctatttataaactgtacttattcaaccacatggtacccgactcttttctggtatgtccaatatcccggagaaggtccacagctccttttgaaagtcacaacagccaacaacaagggaatcagcagaggttttgaagctacatatgataaaagaacaacgtccttccacttgcagaaagcctcagtgcaggagtcagactctgctgtgtactactgtgttctgggtgcctcgggatacaacaaactcacttttggaaagggcacggtgcttctagtctctccag

SEQ ID NO: 314 - DNA, 391 bp, synthetic construct
  source 1..391: mol_type = other DNA
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatcacatgggctacaaacttaccttcgggacaggaacaagcttgttggttgatccag

SEQ ID NO: 315 - DNA, 403 bp, synthetic construct
  source 1..403: mol_type = other DNA
atgctcctggcgctcctcccagtgctggggatacactttgtcctgagagatgcccaagctcagtcagtgacgcagcccgatgctcgcgtcactgtctctgaaggagcctctctgcagctgagatgcaagtattcctcctctgggacaccttatctgttctggtatgtccagtacccgcggcaggggctgcagctgctcctcaagtactattccggagacccagtggttcaaggagtgaatggctttgaggctgagttcagcaagagtaactcttccttccacctgcggaaagcctctgtgcactggagcgactcggctgtgtacttctgtgttctgagcggatatgggagcagtggcaacaagctcatctttggaattgggactctgctttctgtcaagccaa

SEQ ID NO: 316 - DNA, 388 bp, synthetic construct
  source 1..388: mol_type = other DNA
atgaagaggctgctgtgctctctgctggggcttctgtgcacccaggtttgctgggtgaaaggacagcaagtgcagcagagtcccacgtccttggttctgcaggagggggaaaacgcagagctgcagtgtaacttttccacatctttgaacagtatgcagtggttttaccaacgtcctgggggaagtctcgtcagcgtgttctacaatccttctgggacaaagcagagtgggagactgacatccacaacagtcattaaagaacgccgcagctctttgcacatttcctcctcccagacaacagactcaggcacttatctctgtgctttggcccaaggcactgggtctaagctgtcatttgggaagggggcaaagctcacagtgagtccag

SEQ ID NO: 317 - DNA, 406 bp, synthetic construct
  source 1..406: mol_type = other DNA
atgaaatccttgagtgtttcactagtggtcctgtggctccagttaaactgcgtgaggagccagcagaaggtgcagcagagcccagaatccctcagtgtcccagagggaggcatggcctctctcaactgcacttcaagtgatcgtaattttcagtacttctggtggtacagacagcattctgggaaaagccccaagatgctgatgtctatcttctccaatggtgaaaaggaagaaggcagattcacagttcacctcaataaagccagcctgcatacttccctgcacatcagagactcccagcccagtgactctgctctctacctctgtgcagcaagcagggatctaccaggcactgggagtaacaggctcacttttgggaaaggcaccaaattctcactcatcccga

SEQ ID NO: 318 - DNA, 397 bp, synthetic construct
  source 1..397: mol_type = other DNA
atgaactcttctccaggcttcatgactgtgatgctcctcatattcacaagggcccatggagactcagtgactcagacggaaggtcaagtggccctctcagaagaggactttcttacgatacactgcaactactcagcctcagggtacccagctctgttctggtatgtgcagtatcccggagaaggtccacagctcctctttagagcctcaagggacaaagagaaaggaagcagcagaggttttgaagctacatatgataaagggaccacctccttccacttgcggaaagcctcagtgcatgagtcagactcggctgtgtactactgtgctctgggtgatttagaatctgggacttaccagaggtttggaactgggacaaaactccaagtcgttccaa

SEQ ID NO: 319 - DNA, 394 bp, synthetic construct
  source 1..394: mol_type = other DNA
atgcgtcctgacacctgctcagttcttgtgctcctcttaatgctcagaaggaacaatggagactctgtgacccagacagaaggcctggtcactctcaccgaggggttgcctgtgatgctgaactgcacctatcagagtacttactcacctttccttttctggtatgtgcaacatctcaacgaagcccctaagctacttttgaagagcttcacagacaacaagaggcccgagcaccaagggttccacgccactctccataagagcagcagctccttccatctgcagaagtcctcagcgcagctgtcagactctgccctgtactactgtgctttgagtggaggcaataccaacaaagtcgtctttggaacagggaccagattacaagtattaccag

SEQ ID NO: 320 - DNA, 376 bp, synthetic construct
  source 1..376: mol_type = other DNA
atgattgtgatgctcctcatattcacaagggcccatggagactcagtgactcagacggaaggtcaagtggccctctcagaagaggactttcttacgatacactgcaactactcagcctcagggtacccagctctgttctggtacgtgcagtatcccggagaaggtccacagttcctctttagagcctcaagggacaaagagaaaggaagcagcagaggttttgaagctacatatgataaagggaccacctccttccacttgcggaaagcctcagtgcaagagtcagactcggctgtgtactactgtgctctggtcccgaacaccaatacaggcaaattaacctttggggatgggaccgtgctcacagtgaagccaa

SEQ ID NO: 321 - DNA, 400 bp, synthetic construct
  source 1..400: mol_type = other DNA
atgaagaggctgctgtgctctctgctggggctcctgtgcacccaggtttgctgggtgaaaggacagcaagtggagcagagtcccgcgtccttggttctgcaggagggggagaacgcagagctgcagtgcacctattccacaactttgaacagtatgcagtggttttaccaacgtcctgggggaagactcgtcagcctgttgtacaatccttctgggacaaagcagagtgggagactgacatccacaacagtcattaaagaacgtcgcagctctttgcacatttcctcctcccagatcacagactcaggcacttatctctgtgctatggaacggtatctttatgggagcagtggcaacaagctcatctttggaattgggactctgctttctgtcaagccaa

SEQ ID NO: 322 - DNA, 376 bp, synthetic construct
  source 1..376: mol_type = other DNA
atgattgtgatgctcctcatattcacaagggcccatggagactcagtgactcagacggaaggtcaagtggccctctcagaagaggactttcttacgatacactgcaactactcagcctcagggtacccagctctgttctggtacgtgcagtatcccggagaaggtccacagttcctctttagagcctcaagggacaaagagaaaggaagcagcagaggttttgaagctacatatgataaagggaccacctccttccacttgcggaaagcctcagtgcaagagtcagactcggctgtgtactactgtgctctggggaatgacacaaatgcttacaaagtcatctttggaaaagggacacatcttcatgttctcccta

SEQ ID NO: 323 - DNA, 424 bp, synthetic construct
  source 1..424: mol_type = other DNA
atggacaagatcctgacagcatcgtttttactcctaggccttcacctagctggggtgagtggccagcagcaggagaaacgtgaccagcagcaggtgagacaaagtccccaatctctgacagtctgggaaggagagacagcaattctgaactgcagttatgaggacagcacttttgactacttcccatggtactggcagttccctagggaaagccctgcactcctgatagccatacgtccagtgtccaataaaaaggaagatggacgattcacaatagtcttcaagaaaagggagaaaaagctctccttgcacatcacagactctcagcctggagactcaggtacctacttctgtgcagtatcggcctaccagggaggcagagctctgatatttggaacaggaaccacggtatcagtcagcccca

SEQ ID NO: 324 - DNA, 388 bp, synthetic construct
  source 1..388: mol_type = other DNA
atggagaggagcccgggaactgtgctggggctgctgttggtgccgctttgctcaggggtaagaggaatgccggtggaacagaatcctccagccctgagtctttatgaaggagctgactctggtctgagatgcaatttttctaccaccatgaaaagtgtccagtggtttcaacaaaatcacagaggcagacttatcactctgttttacctggctcaaggaacaaaggagaatggaaggctaaagtcagcatttgattctaaggagcgctacagcaccctgcacatcagggatgcccagctggaggactcaggcacttacttctgtgctgcgcaccagggaggcagagctctgatatttggaacaggaaccacggtatcagtcagcccca

SEQ ID NO: 325 - DNA, 397 bp, synthetic construct
  source 1..397: mol_type = other DNA
atgaaatccttgagtgtttccctagtggtcctgtggctccagttaaactgggtgaacagccagcagaaggtgcagcagagcccagaatccctcattgtcccagagggagccatgacctctctcaactgcactttcagcgacagtgcttctcagtattttgcatggtacagacagcattctgggaaagcccccaaggcactgatgtccatcttctctgatggtgacaagaaagaaggcagattcacagctcacctcaataaggccagcctgcatgtttccctgcacatcagagactcccagcccagtgactccgctctctacttctgtgcagcccctaatgttggtgacaacagtaagctgatttggggcttggggacaagtctggtagtaaatccaa

SEQ ID NO: 326 - DNA, 391 bp, synthetic construct
  source 1..391: mol_type = other DNA
atgcattccttacatgtttcactagtgttcctctggcttcaactaggggtgagcagccaggagaaggtacagcagagcccagaatccctcattgtcccagagggagccatgtcctccctcaactgcactttcagcaacagtgcttctcagtccatctggtggtaccaacagcatcctgggaaaggccccgaagcactaatatccatattctctaatggcaacaagaaagaaggcagattgacagtttacctcaatagagccagcctgcatgtttccctgcacatcagagactcccagcccagtgactctgccgtctacctctgtgcaccgcgagtgtctggcagctggcaactcatctttggatctggaacccaactgacagttatgcctg

SEQ ID NO: 327 - DNA, 391 bp, synthetic construct
  source 1..391: mol_type = other DNA
atgaagaggctgctgtgctctctgctggggcttctgtgcacccaggtttgctgggtgaaaggacagcaagtgcagcagagtcccacgtccttggttctgcaggagggggaaaacgcagagctgcagtgtaacttttccacatctttgaacagtatgcagtggttttaccaacgtcctgggggaagtctcgtcagcgtgttctacaatccttctgggacaaagcagagtgggagactgacatccacaacagtcattaaagaacgccgcagctctttgcacatttcctccttccagacaacagactcaggcacttatttctgtgctatagatcttaggaatgcaggtgccaagctcacattcggagggggaacaaggttaacggtcagacccg

SEQ ID NO: 328 - DNA, 424 bp, synthetic construct
  source 1..424: mol_type = other DNA
atggacaagatcctgacagcatcgtttttactcctaggccttcacctagctggggtgagtggccagcagcaggagaaacgtgaccagcagcaggtgagacaaagtccccaatctctgacagtctgggaaggagagacagcaattctgaactgcagttatgaggacagcacttttgactacttcccatggtactggcagttccctagggaaagccctgcactcctgatagccatacgtccagtgtccaataaaaaggaagatggacgattcacaatcttcttcaataaaagggagaaaaagctctccttgcacatcatagactctcagcctggagactcagccacctacttctgtgcagcaagtaggactggcagtggtggaaaactcactttgggggctggaacaagacttcaggtcaaccttg

SEQ ID NO: 329 - DNA, 403 bp, synthetic construct
  source 1..403: mol_type = other DNA
atgctcctggcactcctcccagtgctggggatacactttgtcctgagagatgcccaagctcagtcagtgacacagcccgatgctcgcgtcactgtctctgaaggagcctctctgcagctgagatgcaagtattcctactctgggacgccttatctgttctggtatgtccagtacccgcggcaggggctgcagctgctcctcaagtactattccggagacccagtggttcaaggagtgaatggctttgaggctgagttcatcaagagtaactcttccttccacctgcggaaagcctctgtgcactggagcgactcggctgtgtacttctgtgctgtgaggggttttaatacaggaaactacaaatacgtctttggagcaggtaccagactgaaggttatagcac

SEQ ID NO: 330 - DNA, 385 bp, synthetic construct
  source 1..385: mol_type = other DNA
atgcagaggaacctggttgctgtgctggggattctgtgggtgcagatttgctgggtgagcggagataaggtgaagcaaagtccctcagcgctgagtctccaagagggaaccaattctgctctgagatgcaatttttctatcgccgcgacaactgtgcagtggttcctacagaatcccaggggcagcctcatcaatcttttttacctggttccaggaacaaaggagaatggaaggttaaagtcaacattcaattctaaggagagctacagcaccctgcacatcagggatgcccagctggaggactcaggcacttacttctgtgctgctgacacaaatgcttacaaagtcatctttggaaaagggacacatcttcatgttctcccta

SEQ ID NO: 331 - DNA, 406 bp, synthetic construct
  source 1..406: mol_type = other DNA
atgcctcctcacagcctgctctgtgtgctggtggccttggctatctctggatctaatgtggcccagaaagtgattcaggtctggtcaacagcaagcaggcaggagggcgaagaactcaccctggactgttcatatgagacaagtcaggtcttataccatcttttctggtacaagcaccttcttagtggagagatggttttccttattcgacaaatgtcttcttctactgcaaaagagaggagcggccgctattctgtagtcttccagaaatcattcaagtccatcagccttgtcatttcagcctcacagccagaggattcagggacgtacttctgcgctctctcggaactgagaaattacaacgtgctttacttcggatctggcaccaaactcactgtagagccaa

SEQ ID NO: 332 - DNA, 409 bp, synthetic construct
  source 1..409: mol_type = other DNA
atggactcttctccaggcttcgtggctgtgatacttctcatacttggaaggacccacggagattccgtgactcaaacagaaggccaagtgaccgtctcagaaagcaagtccctgataataaattgcacgtattcaaccacaagcatagcttaccctaatcttttctggtatgttcgatatcctggagaaggtctacaactcctcctgaaagtcattacggctggccagaagggaagcagcagagggtttgaagccacatacaataaagaaaccacctccttccacttgcagaaagcctcagtgcatgagtcagactcggctgtgtactactgtgctctgggtgatctcggagggagcagtggcaacaagctcatctttggaattgggactctgctttctgtcaagccaa

SEQ ID NO: 333 - DNA, 401 bp, synthetic construct
  source 1..401: mol_type = other DNA
atgcacagcctcctggggttgttgttgtggctgcaactgacaagggtgaatagtcaactagcagaagagaattcgtgggccctgagcgtccacgagggtgaaagtgtcacggtgaattgtagttacaagacatccataactgccctacagtggtacagacagaagtcaggcaaaggccctgcccagctaatcttaatacgttcaaatgagagagagaagcgcagtggaagactaagagccacccttgacacctccagccagagcagctccctgtccatcactgctgctcagtgtgaagacactgctgtgtacttctgtgccgccaattctggaagaagcaatgcaaagctaaccttcgggaaaggcactaaactctctgttaaatcaaacatccagaaccc

SEQ ID NO: 334 - DNA, 397 bp, synthetic construct
  source 1..397: mol_type = other DNA
atgaaatccttgagtgtttccctagtggtcctgtggctccagttaaactgggtgaacagccagcagaaggtgcagcagagcccagaatccctcattgtcccagagggagccatgacctctctcaactgcactttcagcgacagtgcttctcagtattttgcatggtacagacagcattctgggaaagcccccaaggcactgatgtccatcttctccaatggtgaaaaagaagaaggcagattcacaattcacctcaataaagccagtctgcatttctccctgcacatcagagactcccagcccagtgactctgctctctacctctgtgcagtgataggaggcactgggagtaacaggctcacttttgggaaaggcaccaaattctcactcatcccga

SEQ ID NO: 335 - DNA, 391 bp, synthetic construct
  source 1..391: mol_type = other DNA
atgaacagattcctgggaatatctttggtgactctatggtttcaagtggcctgggcaaagagccaatggggagaagagaatcttcaggctctgagcatccaggagggtgaagatgtcaccatgaactgcagttacaagacttacacaactgttgttcagtggtacagacagaagtcaggcaaaggccctgccctgataatcttaatacgttcaaatgagcgagagaagcgcagtggaagactcagagccacccttgacacttccagccagagcagctccctgtccatcactggtactctagctacagacactgctgtgtacttctgtgctatgatgtctaattacaacgtgctttacttcggatctggcaccaaactcactgtagagccaa

SEQ ID NO: 336 - DNA, 391 bp, synthetic construct
  source 1..391: mol_type = other DNA
atgaaatcctttagtatttccctagtggtcctgtggcttcagctaaactgggtgaacagccaacagaaggtgcagcagagcccagaatccctcattgttccagagggaggcatggcctctctcaactgcacttccagtgatcgtaatgttgactacttctggtggtacagacagcactctgggaaaagccccaagatgctgatgtctatcttctccaatggtgaaaaggaagaaggcagattgacagtttacctcaatagagccagccagcatgtttccctgcacatcagagagtcccagcccagtgactctgccgtctacctctgtgcagggtaccagggaggcagagctctgatatttggaacaggaaccaaagtatcagtcagcccca

SEQ ID NO: 337 - DNA, 321 bp, synthetic construct
  source 1..321: mol_type = other DNA
atgcacagcctcctggggttgttgttgtggctgcaactgacaagggtgaatagtcaactagcagaagagaattcgtgggccctgagcgtccacgagggtgaaagtgtcacggtgaattgtagttacaagacatccataactgccctacagtggtacagacagaagtcaggcaaaggccctgcccagctaatcttaatacgttcaaatgagagagagaagcgcagtggaagactaagagccacccttgacacctccagccagagcagctccctgtccatcactgctgctcagtgtgaagacactgctgtgtacttctgtgcc

SEQ ID NO: 338 - DNA, 415 bp, synthetic construct
  source 1..415: mol_type = other DNA
atggacaagatcctgacagcatcgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccag

SEQ ID NO: 339 - DNA, 406 bp, synthetic construct
  source 1..406: mol_type = other DNA
atgggctccattttcctcagttgcctggccgtttgtctcctggtggcaggtccagtcgacccgaaaattatccagaaaccaaaatatctggtggcagtcacagggagcgaaaaaatcctgatatgcgaacagtatctaggccacaatgctatgtattggtatagacaaagtgctaagaagcctctagagttcatgttttcctacagctatcaaaaacttatggacaatcagactgcctcaagtcgcttccaacctcaaagttcaaagaaaaaccatttagaccttcagatcacagctctaaagcctgatgactcggccacatacttctgtgccagcagccaagaacgggcccagggtagtgcagaaacgctgtattttggctcaggaaccagactgactgttctcg

SEQ ID NO: 340 - DNA, 397 bp, synthetic construct
  source 1..397: mol_type = other DNA
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagcttcgactgggggggcgatgaacagtacttcggtcccggcaccaggctcacggttttag

SEQ ID NO: 341 - DNA, 427 bp, synthetic construct
  source 1..427: mol_type = other DNA
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctctcggcaataaccaagacacccagtactttgggccaggcactcggctcctcgtgttag

SEQ ID NO: 342 - DNA, 400 bp, synthetic construct
  source 1..400: mol_type = other DNA
atgggctccattttcctcagttgcctggccgtttgtctcctggtggcaggtccagtcgacccgaaaattatccagaaaccaaaatatctggtggcagtcacagggagcgaaaaaatcctgatatgcgaacagtatctaggccacaatgctatgtattggtatagacaaagtgctaagaagcctctagagttcatgttttcctacagctatcaaaaacttatggacaatcagactgcctcaagtcgcttccaacctcaaagttcaaagaaaaaccatttagaccttcagatcacagctctaaagcctgatgactcggccacatacttctgtgccagcagccgaggactggagaactatgctgagcagttcttcggaccagggacacgactcaccgtcctag

SEQ ID NO: 343 - DNA, 430 bp, synthetic construct
  source 1..430: mol_type = other DNA
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcgggcaggacagctcctatgaacagtacttcggtcccggcaccaggctcacggttttag

SEQ ID NO: 344 - DNA, 388 bp, synthetic construct
  source 1..388: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagtgatcagggcacagaagtcttctttggtaaaggaaccagactcacagttgtag

SEQ ID NO: 345 - DNA, 397 bp, synthetic construct
  source 1..397: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatgctacagttggcaaccaggctccgctttttggagaggggactcgactctctgttctag

SEQ ID NO: 346 - DNA, 406 bp, synthetic construct
  source 1..406: mol_type = other DNA
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtgctcttccgggggctgacacagaagtcttctttggtaaaggaaccagactcacagttgtag

SEQ ID NO: 347 - DNA, 397 bp, synthetic construct
  source 1..397: mol_type = other DNA
atgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaagattggctgggggttgctgagcagttcttcggaccagggacacgactcaccgtcctag

SEQ ID NO: 348 - DNA, 400 bp, synthetic construct
  source 1..400: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatctccgggacaaattccaagacacccagtactttgggccaggcactcggctcctcgtgttag

SEQ ID NO: 349 - DNA, 433 bp, synthetic construct
  source 1..433: mol_type = other DNA
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctctcgatgacagggggcggggaaatacgctctattttggagaaggaagccggctcattgttgtag

SEQ ID NO: 350 - DNA, 394 bp, synthetic construct
  source 1..394: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatgggctggggcactatgaacagtacttcggtcccggcaccaggctcacggttttag

SEQ ID NO: 351 - DNA, 394 bp, synthetic construct
  source 1..394: mol_type = other DNA
atggcccccaggctccttttctgtctggttctttgcttcttgagagcagaaccaacaaatgctggtgtcatccaaacacctaggcacaaggtgacagggaagggacaagaagcaactctgtggtgtgagccaatttcaggacatagtgctgttttctggtacagacagaccattgtgcagggcctggagttcctgacttactttcgaaatcaagctcctatagatgattcagggatgcccaaggaacgattctcagctcagatgcccaatcagtcgcactcaactctgaagatccagagcacgcaaccccaggactcagcggtgtatctttgtgcaagccgacagtcctatgctgagcagttcttcggaccagggacacgactcaccgtcctag

SEQ ID NO: 352 - DNA, 397 bp, synthetic construct
  source 1..397: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcgggactgggggcgctagtgcagaaacgctgtattttggctcaggaaccagactgactgttctcg

SEQ ID NO: 353 - DNA, 400 bp, synthetic construct
  source 1..400: mol_type = other DNA
atgggcaccaggcttcttggctgggcagtgttctgtctccttgacacagtactgtctgaagctggagtcacccagtctcccagatatgcagtcctacaggaagggcaagctgtttccttttggtgtgaccctatttctggacatgataccctttactggtatcagcagcccagagaccaggggccccagcttctagtttactttcgggatgaggctgttatagataattcacagttgccctcggatcgattttctgctgtgaggcctaaaggaactaactccactctcaagatccagtctgcaaagcagggcgacacagccacctatctctgtgccagcagatccgggacagggtccgaaacgctgtattttggctcaggaaccagactgactgttctcg

SEQ ID NO: 354 - DNA, 385 bp, synthetic construct
  source 1..385: mol_type = other DNA
atgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcagtgataacatctatgaacagtacttcggtcccggcaccaggctcacggttttag

SEQ ID NO: 355 - DNA, 421 bp, synthetic construct
  source 1..421: mol_type = other DNA
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcgacacaaactccgactacaccttcggctcagggaccaggcttttggtag

SEQ ID NO: 356 - DNA, 424 bp, synthetic construct
  source 1..424: mol_type = other DNA
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcgcccatccctatgaacagtacttcggtcccggcaccaggctcacggttttag

SEQ ID NO: 357 - DNA, 388 bp, synthetic construct
  source 1..388: mol_type = other DNA
atgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcagggactgggggaactatgaacagtacttcggtcccggcaccaggctcacggttttag

SEQ ID NO: 358 - DNA, 397 bp, synthetic construct
  source 1..397: mol_type = other DNA
atgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcagtgatgaagggggggccagtcaaaacaccttgtactttggtgcgggcacccgactatcggtgctag

SEQ ID NO: 359 - DNA, 397 bp, synthetic construct
  source 1..397: mol_type = other DNA
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtgccggagggagagaagtcttctttggtaaaggaaccagactcacagttgtag

SEQ ID NO: 360 - DNA, 397 bp, synthetic construct
  source 1..397: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatgctacagttggcaaccaggctccgctttttggagaggggactcgactttctgttctag

SEQ ID NO: 361 - DNA, 397 bp, synthetic construct
  source 1..397: mol_type = other DNA
atgggctccaggctcttcttcgtgctctccagtctcctgtgttcaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatgccgcggacagggggcggaaagtcttctttggtaaaggaaccagactcacagttgtag

SEQ ID NO: 362 - DNA, 424 bp, synthetic construct
  source 1..424: mol_type = other DNA
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctctccgggggtacagggaagtcttctttggtaaaggaaccagactcacagttgtag

SEQ ID NO: 363 - DNA, 394 bp, synthetic construct
  source 1..394: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagctttgggggccttcaaaacaccttgtactttggtgcgggcacccgactatcggtgctag

SEQ ID NO: 364 - DNA, 394 bp, synthetic construct
  source 1..394: mol_type = other DNA
atggcccccaggctccttttctgtctggttctttgcttcttgagagcagaaccaacaaatgctggtgtcatccaaacacctaggcacaaggtgacagggaagggacaagaagcaactctgtggtgtgagccaatttcaggacatagtgctgttttctggtacagacagaccattgtgcagggcctggagttcctgacttactttcgaaatcaagctcctatagatgattcagggatgcccaaggaacgattctcagctcagatgcccaatcagtcgcactcaactctgaagatccagagcacgcaaccccaggactcagcggtgtatctttgtgcaagcagcccccagggggctatagggtacttcggtcccggcaccaggctcacggttttag

SEQ ID NO: 365 - DNA, 394 bp, synthetic construct
  source 1..394: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagtccactgggggggcttcgggaacagtacttcggtcccggcaccaggctcacggttttag

SEQ ID NO: 366 - DNA, 400 bp, synthetic construct
  source 1..400: mol_type = other DNA
atgggctccattttcctcagttgcctggccgtttgtctcctggtggcaggtccagtcgacccgaaaattatccagaaaccaaaatatctggtggcagtcacagggagcgaaaaaatcctgatatgcgaacagtatctaggccacaatgctatgtattggtatagacaaagtgctaagaagcctctagagttcatgttttcctacagctatcaaaaacttatggacaatcagactgcctcaagtcgcttccaacctcaaagttcaaagaaaaaccatttagaccttcagatcacagctctaaagcctgatgactcggccacatacttctgtgccagcagccaaggggtcatttccaacgaaagattatttttcggtcatggaaccaagctgtctgtcctgg

SEQ ID NO: 367 - DNA, 406 bp, synthetic construct
  source 1..406: mol_type = other DNA
atgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcagtatagcagggggtcaaaacaccttgtactttggtgcgggcacccgactatcggtgctag

SEQ ID NO: 368 - DNA, 400 bp, synthetic construct
  source 1..400: mol_type = other DNA
atggatatctggcttctaggttggataatttttagtttcttggaagcaggacacacaggacccaaagtcttacagatcccaagtcatcaaataatagatatggggcagatggtgaccctcaattgtgacccagtttctaatcacctatatttttattggtataaacagattttaggacagcagatggagtttctggttaatttctacaatggtaaagtcatggagaagtttaaactgtttaaggatcagttttcagttgaaagaccagatggttcatatttcactctgaaaatccaacccacagcactggaggactcagctgtgtacttctgtgccagcagctcgactggggggggttatgaacagtacttcggtcccggcaccaggctcacggttttag

SEQ ID NO: 369 - DNA, 394 bp, synthetic construct
  source 1..394: mol_type = other DNA
atgctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtctaccccggactggggggtgggaacagtacttcggtcccggcaccaggctcacggttttag

SEQ ID NO: 370 - DNA, 397 bp, synthetic construct
  source 1..397: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcaggggactgggggctaaccaagacacccagtactttgggccaggcactcggctcctcgtgttag

SEQ ID NO: 371 - DNA, 403 bp, synthetic construct
  source 1..403: mol_type = other DNA
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtgcgacagattctggaaatacgctctattttggagaaggaagccggctcattgttgtag

SEQ ID NO: 372 - DNA, 409 bp, synthetic construct
  source 1..409: mol_type = other DNA
atgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcagtatagactggaatagtcaaaacaccttgtactttggtgcgggcacccgactatcggtgctag

SEQ ID NO: 373 - DNA, 412 bp, synthetic construct
  source 1..412: mol_type = other DNA
atgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcagtaccccgggctggggataccaagacacccagtactttgggccaggcactcggctcctcgtgttag

SEQ ID NO: 374 - DNA, 403 bp, synthetic construct
  source 1..403: mol_type = other DNA
atggatatctggcttctaggttggataatttttagtttcttggaagcaggacacacaggacccaaagtcttacagatcccaagtcatcaaataatagatatggggcagatggtgaccctcaattgtgacccagtttctaatcacctatatttttattggtataaacagattttaggacagcagatggagtttctggttaatttctacaatggtaaagtcatggagaagtctaaactgtttaaggatcagttttcagttgaaagaccagatggttcatatttcactctgaaaatccaacccacagcactggaggactcagctgtgtacttctgtgccagcagcttcggactcaggccctttgctgagcagttcttcggaccagggacacgactcaccgtcctag

SEQ ID NO: 375 - DNA, 412 bp, synthetic construct
  source 1..412: mol_type = other DNA
atgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcagtattggactggggggtaactatgctgagcagttcttcggaccagggacacgactcaccgtcctag

SEQ ID NO: 376 - DNA, 412 bp, synthetic construct; identical to SEQ ID NO: 375
  source 1..412: mol_type = other DNA

SEQ ID NO: 377 - DNA, 409 bp, synthetic construct; identical to SEQ ID NO: 372
  source 1..409: mol_type = other DNA

SEQ ID NO: 378 - DNA, 421 bp, synthetic construct
  source 1..421: mol_type = other DNA
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctaccctcgcgattctcagtccaacagtttgatgactatcactctgaaatgaacatgagtgccttggaactggaggactctgctatgtacttctgtgccagctctcgggccaattatgaacagtacttcggtcccggcaccaggctcacggttttag

SEQ ID NO: 379 - DNA, 20 bp, synthetic construct
  source 1..20: mol_type = other DNA
acggccgtgcatgtgtccca

SEQ ID NO: 380 - DNA, 20 bp, synthetic construct
  source 1..20: mol_type = other DNA
ccgagcccaagaccgtctag

SEQ ID NO: 381 - DNA, 20 bp, unidentified
  source 1..20: mol_type = other DNA
ttttttgggggtcctaactt

SEQ ID NO: 382 - DNA, 20 bp, unidentified
  source 1..20: mol_type = other DNA
taaatccggctactttcagc

SEQ ID NO: 383 - DNA, 20 bp, unidentified
  source 1..20: mol_type = other DNA
agaccctcaggcggctgctc